(19) United States
(12) Patent Application Publication
(10) Pub. No.: US 2001/0020100 A1
(43) Pub. Date: Sep. 6, 2001

(54) N-substituted-1, 2, 4-triazolone compounds for treatment of cardiovascular disorders

(75) Inventors: Robert E. Manning, St. Louis, MO (US); David B. Reitz, Chesterfield, MO (US); Horn-Chih Huang, Chesterfield, MO (US)

Correspondence Address: J. Timothy Keane, Pharmacia Corporation, Corporate Patent Department, P.O. Box 5110, Chicago, IL, 60680, US

(73) Assignee: G.D. Searle & Co., Chicago, IL (US)

(21) Appl. No.: 09/742,818

(22) Filed: Dec. 20, 2000

Related U.S. Application Data
(63) Continuation of application No. 09/526,497, filed on Mar. 15, 2000, now abandoned.
Application No. 09/526,497 is a continuation of application No. 09/237,220, filed on Jan. 25, 1999, now abandoned.
Application No. 09/237,220 is a continuation of application No. 08/847,330, filed on Apr. 23, 1997, now abandoned.
Application No. 08/847,330 is a continuation of application No. 08/618,447, filed on Mar. 19, 1996, now abandoned.
Application No. 08/618,447 is a continuation of application No. 08/259,420, filed on Jun. 14, 1994, now abandoned.

(51) Int. Cl. C07D 249/12

(52) U.S. Cl. 548/263.2; 546/210; 540/603

(57) ABSTRACT

A class of N-substituted-1,2,4-triazolone compounds is described for use in treatment of cardiovascular disorders. Compounds of particular interest are angiotensin II antagonists of the formula

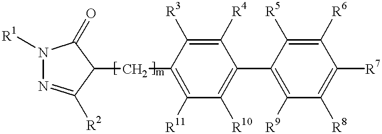

wherein R¹ is selected from methyl, ethyl, n-propyl, isopropyl, n-butyl, sec-butyl, isobutyl, tert-butyl, 4-methylbutyl, n-pentyl, neopentyl, phenyl, benzyl, phenethyl, cyclohexyl, cyclohexylmethyl, 1-oxoethyl, 1-oxopropyl, 1-oxobutyl, 1-oxopentyl and hydroxyalkyl; wherein R² is selected from ethyl, n-propyl, isopropyl, n-butyl, sec-butyl, isobutyl, 4-methylbutyl, tert-butyl, n-pentyl, neo-pentyl, propylthio and butylthio; wherein each of R³ through R¹¹ is hydrido with the proviso that at least one of R⁵ and R⁹ must be selected from COOH, SH, PO₃H₂, SO₃H, CONHNH₂, CONHNHSO₂CF₃, OH,

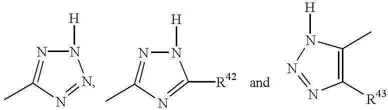

wherein each of R⁴² and R⁴³ is independently selected from chloro, cyano, nitro, trifluoromethyl, methoxycarbonyl and trifluoromethylsulfonyl. These compounds are particularly useful in treatment or control of hypertension and congestive heart failure.

### FIELD OF THE INVENTION

Non-peptidic N-substituted-1,2,4-triazolone compounds are described for use in treatment of cardiovascular disorders such as hypertension and congestive heart failure. Of particular interest are angiotensin II antagonist compounds provided by 1,2,4-triazolones having a biphenylmethyl moiety attached to the nitrogen atom at the four-position of the 1,2,4-triazolone.

### BACKGROUND OF THE INVENTION

The renin-angiotensin system is one of the hormonal mechanisms involved in regulation of pressure/volume homeostasis and in expression of hypertension. Activation of the renin-angiotensin cascade begins with renin secretion from the juxtaglomerular apparatus of the kidney and culminates in the formation of angiotensin II, the primary active species of this system. This octapeptide, angiotensin II, is a potent vasoconstrictor agent and also produces other physiological effects such as promoting aldosterone secretion, promoting sodium and fluid retention, inhibiting renin secretion, increasing sympathetic nervous system activity, increasing vasopressin secretion, causing positive cardiac inotropic effect and modulating other hormonal systems. angiotensin II at its receptors is a viable approach to inhibit the renin-angiotensin system, given the pivotal role of this octapeptide which mediates the actions of the renin-angiotensin system through interaction with various tissue receptors. There are several known angiotensin II antagonists, most of which are peptidic in nature. Such peptidic compounds are of limited use due to their lack of oral bioavailability or their short duration of action. Also, commercially-available peptidic angiotensin II antagonists (e.g., Saralasin) have a significant residual agonist activity which further limit their therapeutic application.

Non-peptidic compounds with angiotensin II antagonist properties are known. For example, the sodium salt of 2-n-butyl-4-chloro-1-(2-chlorobenzyl)imidazole-5-acetic acid has specific competitive angiotensin II antagonist activity as shown in a series of binding experiments, functional assays and in vivo tests [P. C. Wong et al, J. Pharmacol. Exp. Ther., 247(1), 1-7 (1988)]. Also, the sodium salt of 2-butyl-4-choloro-1-(2-nitrobenzyl) imidazole-5-acetic acid has specific competitive angiotensin II antagonist activity as shown in a series of binding experiments, functional assays and in vivo tests [A. T. Chiu et al, European J. Pharmacol., 157, 3121 (1988)]. A family of 1-benzylimidazole-5-acetate derivatives has been shown to have competitive angiotensin II antagonist properties [A. T. Chiu et al, J. Pharmacol. Exp. Ther., 250(3), 867-874 (1989)]. U.S. Pat. No. 4,816,463 to Blankey et al describes a family of 4,5,6,7-tetrahydro-1H-imidazo (4,5-c)-tetrahydro-pyridine derivatives useful as antihypertensives, some of which are reported to antagonize the binding of labelled angiotensin II to rat adrenal receptor preparation and thus cause a significant decrease in mean arterial blood pressure in conscious hypertensive rats. EP No. 253,310, published Jan. 20, 1988, describes a series of aralkyl imidazole compounds, including in particular a family of biphenylmethyl substituted imidazoles, as antagonists to the angiotensin II receptor. EP No. 323,841 published Jul. 12, 1989 describes four classes of angiotensin II antagonists, namely, biphenylmethylpyrroles, biphenylmethylpyrazoles, biphenylmethyl-1,2,3-triazoles and biphenylmethyl 4-substituted-4H-1,2,4-triazoles, including the compound 3,5-dibutyl-4-[(2′-carboxybiphenyl-4-yl) methyl]-4H-1,2,4-triazole. U.S. Pat. No. 4,880,804 to Carini et al describes a family of biphenylmethylbenzimidazole compounds as angiotensin II receptor blockers for use in treatment of hypertension and congestive heart failure.

There are several families of known compounds having one or two oxo substituents on the triazole ring. For example, East German Patent No. 160,447 published Aug. 3, 1983 describes a family of 1,2,4-triazolin-5-one compounds, specifically 2,4-dihydro-4,5-bis(phenylmethyl)-3H-1,2,4-triazol-3-one, for use as herbicides. Belgian Patent No. 806,146 published Oct. 16, 1972 describes a family of triazolinone compounds, including the compound (3-(4-m-chlorophenyl-1-piperazinyl)-propyl)-3,4-diethyl-1,2,4-triazolin-5-one, having tranquilizer, hypotensive and analgesic activities. Belgian Patent No. 631,842 published Feb. 28, 1963 describes a family of 1,2,4-triazolones having hypnotic, tranquilizer, narcotic, sedative and analgetic activities, which includes a class of 4-N-aralkyl-1,2,4-triazol-5-one compounds. E.P. #7,180 published Jun. 15, 1978 describes a family of 1,2-disubstituted-4-alkyl-1,2,4-triazolidine-3,5-dione compounds having a wide variety of activities, such as antiulcer, bronchodilator, antifertility and cardiovascular-related activities which include antihypertensive, antiarrhythmic, platelet aggregation inhibition and smooth muscle activities. E.P. #283,310 published Mar. 18, 1987 describes a family of N¹-diarylmethyl-N²-aminoalkyl-diaza-heterocyclic derivatives for treating cerebral vascular and ischmic diseases and for protecting against anoxia.

### DESCRIPTION OF THE INVENTION

A class of biphenylalkyl N-substituted-1,2,4-triazole compounds useful in treating circulatory and cardiovascular disorders is defined by Formula I:

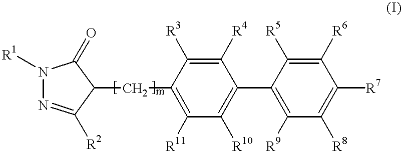

wherein m is a number selected from one to four, inclusive;

wherein R¹ is selected from alkyl, hydroxyalkyl, haloalkyl, cycloalkyl, cycloalkylalkyl, formyl, aralkyl, aryl, aroyl, alkoxyalkyl, alkylcarbonyl, alkylcarbonylalkyl, alkoxycarbonyl, alkenyl, cycloalkenyl, aralkoxycarbonyl, alkynyl, cyano, carboxyl, mercaptocarbonyl, mercaptothiocarbonyl, alkylthiocarbonyl, alkylthiothiocarbonyl, arylthiocarbonyl, arylthiothiocarbonyl, aralkylthiocarbonyl, alkylthiocarbonyl, alkylsulfinyl, alkylsulfonyl, aralkylsulfinyl, aralkylsulfonyl, arylsulfinyl, arylsulfonyl, heteroaryl having one or more ring atoms selected from oxygen, sulfur and nitrogen atoms, and amido radicals of the formula

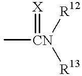

wherein X is oxygen atom or sulfur atom; wherein each of R¹² and R¹³ is independently selected from hydrido, alkyl, cycloalkyl, cyano, hydroxyalkyl, cycloalkylalkyl, alkoxyalkyl, aralkyl and aryl, and wherein R¹² and R¹³ taken together may form a heterocyclic group having five to seven ring members including the nitrogen atom of said amino or amido radical and which heterocyclic group may further contain one or more hetero atoms as ring members selected from oxygen, nitrogen and sulfur atoms and which heterocyclic group may be saturated or partially unsaturated; wherein each of R¹² and R¹³ taken together may form an aromatic heterocyclic group having five ring members including the nitrogen atom of said amino or amido radical and which aromatic heterocyclic group may further contain one or more hetero atoms as ring atoms selected from oxygen, nitrogen and sulfur atoms;

wherein each of R² through R¹¹ is independently selected from hydrido, alkyl, hydroxyalkyl, halo, haloalkyl, cycloalkyl, cycloalkylalkyl, formyl, alkoxy, aralkyl, aryl, aroyl, aryloxy, aralkoxy, aralkoxycarbonyl, alkoxyalkyl, alkylcarbonyl, alkylcarbonylalkyl, alkoxycarbonyl, alkenyl, cycloalkenyl, alkynyl, cyano, nitro, carboxyl, alkylcarbonyloxy, mercaptocarbonyl, mercaptothiocarbonyl, alkoxycarbonyloxy, alkylthio, alkylthiocarbonyl, alkylcarbonylthio, alkylthiocarbonyloxy, alkylthiocarbonylthio, alkylthiothiocarbonyl, alkylthiothiocarbonylthio, arylthio, arylthiocarbonyl, arylcarbonylthio, arylthiocarbonyloxy, arylthiocarbonylthio, arylthiothiocarbonyl, arylthiothiocarbonylthio, aralkylthio, aralkylthiocarbonyl, aralkylcarbonylthio, aralkylthiocarbonyloxy, aralkylthiocarbonylthio, alkylthiocarbonyl, aralkylthiocarbonylthio, mercapto, alkylsulfinyl, alkylsulfonyl, aralkylsulfinyl, aralkylsulfonyl, arylsulfinyl, arylsulfonyl, heteroaryl having one or more ring atoms selected from oxygen, sulfur and nitrogen atoms, and amino and amido radicals of the formula

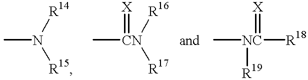

wherein X is oxygen atom or sulfur atom; wherein each of R¹⁴, R¹⁵, R¹⁶, R¹⁷, R¹⁸ and R¹⁹ is independently selected from hydrido, alkyl, cycloalkyl, cyano, hydroxyalkyl, cycloalkylalkyl, alkoxyalkyl, aralkyl and aryl, and wherein R¹⁴ and R¹⁵ taken together, R¹⁶ and R¹⁷ taken together and R¹⁸ and R¹⁹ taken together may form a heterocyclic group having five to seven ring members including the nitrogen atom of said amino or amido radical and which heterocyclic group may further contain one or more hetero atoms as ring members selected from oxygen, nitrogen and sulfur atoms and which heterocyclic group may be saturated or partially unsaturated; wherein each of R¹⁴ and R¹⁵ taken together and each of R¹⁶ and R¹⁷ taken together may form an aromatic heterocyclic group having five ring members including the nitrogen atom of said amino or amido radical and which aromatic heterocyclic group may further contain one or more hetero atoms as ring atoms selected from oxygen, nitrogen and sulfur atoms;

and wherein each of R³ through R¹¹ may be further independently selected from hydroxy and acidic moieties of the formula

−YₙA

wherein n is a number selected from zero through three, inclusive, and wherein A is an acidic group selected to contain at least one acidic hydrogen atom, and the amide, ester and salt derivatives of said acidic moieties; wherein Y is a spacer group independently selected from one or more of alkyl, cycloalkyl, cycloalkylalkyl, alkenyl, alkynyl, aryl, aralkyl and heteroaryl having one or more ring atoms selected from oxygen, sulfur and nitrogen atoms; and wherein any of the foregoing R¹ through R¹⁹, Y and A groups having a substitutable position may be substituted by one or more groups selected from hydroxy, alkyl, alkenyl, alkynyl, aralkyl, hydroxyalkyl, haloalkyl, halo, oxo, alkoxy, aryloxy, aralkoxy, aralkylthio, alkoxyalkyl, cycloalkyl, cycloalkylalkyl, aryl, aroyl, cycloalkenyl, cyano, cyanoamino, nitro, alkylcarbonyloxy, alkoxycarbonyloxy, alkylcarbonyl, alkoxycarbonyl, aralkoxycarbonyl, carboxyl, mercapto, mercaptocarbonyl, alkylthio, arylthio, alkylthiocarbonyl, alkylsulfinyl, alkylsulfonyl, haloalkylsulfonyl, aralkylsulfinyl, aralkylsulfonyl, arylsulfinyl, arylsulfonyl, heteroaryl having one or more ring atoms selected from oxygen, sulfur and nitrogen atoms, and amino and amido radicals of the formula

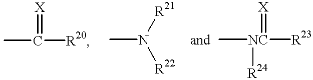

wherein X is oxygen atom or sulfur atom; wherein R²⁰ is selected from hydrido, alkyl, cycloalkyl, cycloalkylalkyl, aralkyl, aryl, DR²⁵ and

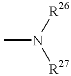

wherein D is selected from oxygen atom and sulfur atom and R²⁵ is selected from hydrido, alkyl, cycloalkyl, cycloalkylalkyl, aralkyl and aryl; wherein each of R²¹, R²², R²³, R²⁴, R²⁶ and R²⁷ is independently selected from hydrido, alkyl, cycloalkyl, cyano, hydroxyalkyl, haloalkyl, cycloalkylalkyl, alkoxyalkyl, alkylcarbonyl, alkoxycarbonyl, carboxyl, alkylsulfinyl, alkylsulfonyl, arylsulfinyl, arylsulfonyl, haloalkylsulfinyl, haloalkylsulfonyl, aralkyl and aryl, and wherein each of R²¹, R²², R²³, R²⁴, R²⁶ and R²⁷ is further independently selected from amino and amido radicals of the formula

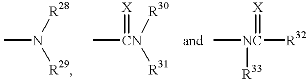

wherein X is oxygen atom or sulfur atom; wherein each of R²⁸, R²⁹, R³⁰, R³¹ R³² and R³³ is independently selected from hydrido, alkyl, cycloalkyl, cyano, hydroxyalkyl, cycloalkylalkyl, alkoxyalkyl, haloalkylsulfinyl, haloalkylsulfonyl, aralkyl and aryl, and wherein each of R²¹ and R²² taken together and each of R²³ and R²⁴ taken together may form a heterocyclic group having five to seven ring members including the nitrogen atom of said amino or amido radical, which heterocyclic group may further contain one or more hetero atoms as ring members selected from oxygen, nitrogen and sulfur atoms and which heterocyclic group may be saturated or partially unsaturated; wherein each of R²¹ and R²² taken together and each of R²⁶ and R²⁷ taken together may form an aromatic heterocyclic group having five ring members including the nitrogen atom of said amino or amido radical and which aromatic heterocyclic group may further contain one or more hetero atoms as ring atoms selected from oxygen, nitrogen and sulfur atoms; or a tautomer thereof or a pharmaceutically-acceptable salt thereof.

Compounds of Formula I would be useful in treating a variety of circulatory disorders including cardiovascular disorders, such as hypertension, congestive heart failure and arteriosclerosis, and to treat other disorders such as glaucoma. These compounds would also be useful as adjunctive therapies. For example, compounds of Formula I may be used in combination with other drugs, such as a diuretic, to treat hypertension. Also, compounds of Formula I could be used in conjunction with certain surgical procedures. For example, these compounds could be used to prevent post-angioplasty re-stenosis. Compounds of Formula I are therapeutically effective in treatment of cardiovascular disorders by acting as antagonists to, or blockers of, the angiotensin II (AII) receptor. Compounds of Formula I would be therapeutically effective in treatment of the above-mentioned circulatory and cardiovascular disorders or would be precursors to, or prodrugs of, therapeutically-effective compounds.

The phrase “acidic group selected to contain at least one acidic hydrogen atom”, as used to define the −YₙA moiety, is intended to embrace chemical groups which, when attached to any of the R³ through R¹¹ positions of Formula I, confers acidic character to the compound of Formula I. “Acidic character” means proton-donor capability, that is, the capacity of the compound of Formula I to be a proton donor in the presence of a proton-receiving substance such as water. Typically, the acidic group should be selected to have proton-donor capability such that the product compound of Formula I has a pKₐ in a range from about one to about twelve. More typically, the Formula I compound would have a pKₐ in a range from about two to about seven. An example of an acidic group containing at least one acidic hydrogen atom is carboxyl group (—COOH). Where n is zero and A is —COOH, in the −YₙA moiety, such carboxyl group would be attached directly to one of the R³ through R¹¹ positions. The Formula I compound may have one −YₙA moiety attached at one of the R³ through R¹¹ positions, or may have a plurality of such −YₙA moieties attached at more than one of the R³ through R¹¹ positions, up to a maximum of nine such −YₙA moieties. There are many examples of acidic groups other than carboxyl group, selectable to contain at least one acidic hydrogen atom. Such other acidic groups may be collectively referred to as “bioisosteres of carboxylic acid” or referred to as “acidic bioisosteres”. Specific examples of such acidic bioisosteres are described hereinafter. Compounds of Formula I having the −YₙA moiety attached at one of positions R⁵, R⁶, R⁸ and R⁹ would be expected to have preferred properties, while attachment at R⁵ or R⁹ would be more preferred.

A preferred class of compounds consists of those compounds of Formula I wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkoxycarbonyl, alkenyl, cycloalkenyl, alkynyl, cycloalkynyl, carboxyl, mercaptocarbonyl, mercaptothiocarbonyl, alkylthiocarbonyl, arylthiocarbonyl, arylthiothiocarbonyl, aralkylthiocarbonyl, alkylsulfonyl, aralkylsulfonyl, arylsulfonyl, heteroaryl having one or more ring atoms selected from oxygen, sulfur and nitrogen atoms, and amido radicals of the formula

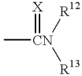

wherein x is oxygen atom or sulfur atom; wherein each of R¹² and R¹³ is independently selected from hydrido, alkyl, cycloalkyl, cyano, hydroxyalkyl, cycloalkylalkyl, alkoxyalkyl, aralkyl and aryl;

wherein R² is selected from hydrido, alkyl, hydroxyalkyl, halo, haloalkyl, cycloalkyl, cycloalkylalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cycloalkynyl, cyano, carboxyl, alkylcarbonyloxy, alkylthio, alkylthiocarbonyl, alkylcarbonylthio, alkylthiocarbonyloxy, alkylthiocarbonylthio, alkylthiothiocarbonyl, alkylthiothiocarbonylthio, arylthio, arylthiocarbonyl, arylcarbonylthio, arylthiocarbonyloxy, arylthiocarbonylthio, arylthiothiocarbonyl, arylthiothiocarbonylthio, aralkylthio, aralkylthiocarbonyl, aralkylcarbonylthio, aralkylthiocarbonyloxy, aralkylthiocarbonylthio, aralkylthiocarbonyl, aralkylthiocarbonylthio, mercapto, alkylsulfonyl, aralkylsulfonyl, arylsulfonyl and heteroaryl having one or more ring atoms selected from oxygen, sulfur and nitrogen atoms;

wherein each of R³ through R¹¹ is independently selected from hydrido, hydroxy, alkyl, hydroxyalkyl, halo, haloalkyl, cycloalkyl, cycloalkylalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cycloalkynyl, cyano, nitro, carboxyl, alkylcarbonyloxy, mercaptocarbonyl, mercaptothiocarbonyl, alkoxycarbonyloxy, alkylthio, alkylthiocarbonyl, alkylcarbonylthio, alkylthiocarbonyloxy, alkylthiocarbonylthio, alkylthiothiocarbonyl, arylthio, arylthiocarbonyl, arylcarbonylthio, arylthiocarbonyloxy, arylthiothiocarbonyl, aralkylthio, aralkylthiocarbonyl, aralkylcarbonylthio, aralkylthiocarbonyloxy, aralkylthiocarbonylthio, aralkylthiocarbonyl, aralkylthiocarbonylthio, mercapto, alkylsulfonyl, aralkylsulfonyl and arylsulfonyl, and amino and amido radicals of the formula

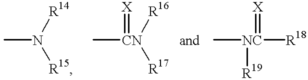

wherein X is oxygen atom or sulfur atom; wherein each of R¹⁴, R¹⁵, R¹⁶, R¹⁷, R¹⁸ and R¹⁹ is independently selected from hydrido, alkyl, cycloalkyl, cyano, hydroxyalkyl, cycloalkylalkyl, alkoxyalkyl, aralkyl and aryl;

and wherein each of R³ through R¹¹ may be further independently selected from acidic moieties of the formula

−YₙA

wherein n is a number selected from zero through three, inclusive; wherein A is an acidic group selected from acids containing one or more atoms selected from oxygen, sulfur, phosphorus and nitrogen atoms, and wherein said acidic group is selected to contain at least one acidic hydrogen atom, and the amide, ester and salt derivatives of said acidic moieties; wherein Y is a spacer group independently selected from one or more of alkyl, cycloalkyl, cycloalkylalkyl, alkenyl, alkynyl, aryl, aralkyl and heteroaryl having one or more ring atoms selected from oxygen sulfur and nitrogen atoms;

and wherein any of the foregoing R¹ through R¹⁹, Y and A groups having a substitutable position may be substituted by one or more groups selected from hydroxy, alkyl, alkenyl, aralkyl, hydroxyalkyl, halo, haloalkyl, oxo, alkoxy, aryloxy, aralkoxy, alkoxyalkyl, alkylcarbonyl, alkoxycarbonyl, carboxyl, cyano, nitro, alkylsulfonyl, haloalkylsulfonyl, aryl, aralkyl, mercaptocarbonyl, alkylthio and alkylthiocarbonyl, and amino and amido radicals of the formula

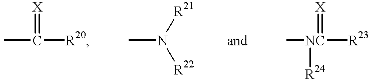

wherein X is oxygen atom or sulfur atom; wherein R¹⁹ is selected from hydrido, alkyl, cycloalkyl, cycloalkylalkyl, aralkyl, aryl, and DR²³ and

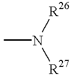

wherein D is selected from oxygen atom and sulfur atom, and R²⁵ is selected from hydrido, alkyl, cycloalkyl, cycloalkylalkyl, aralkyl and aryl; wherein each of R²¹, R²², R²³, R²⁴, R²⁶ and R²⁷ is independently selected from hydrido, alkyl, cycloalkyl, cyano, hydroxyalkyl, haloalkyl, cycloalkylalkyl, alkoxyalkyl, alkanoyl, alkoxycarbonyl, carboxyl, haloalkylsulfinyl, haloalkylsulfonyl, aralkyl and aryl;

or a tautomer thereof or a pharmaceutically-acceptable salt thereof.

A more preferred class of compounds consists of those compounds of Formula I wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkoxycarbonyl, alkenyl, cycloalkenyl, alkynyl, cycloalkynyl, carboxyl, alkylsulfonyl, aralkylsulfonyl and arylsulfonyl, and amido radicals of the formula

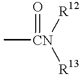

wherein each of R¹² and R¹³ is independently selected from hydrido, alkyl, cycloalkyl, cyano, hydroxyalkyl, cycloalkylalkyl, alkoxyalkyl, aralkyl and aryl;

wherein R² is selected from hydrido, alkyl, hydroxyalkyl, halo, haloalkyl, cycloalkyl, cycloalkylalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cycloalkynyl, carboxyl, alkylcarbonyloxy, alkylthio, arylthio, aralkylthio, aralkylthiocarbonylthio, alkylsulfonyl, aralkylsulfonyl and arylsulfonyl;

wherein each of R³ through R¹¹ is independently selected from hydrido, hydroxy, alkyl, hydroxyalkyl, halo, haloalkyl, cycloalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cyano, nitro, carboxyl, alkylcarbonyloxy, mercaptocarbonyl, alkoxycarbonyloxy, alkylthio, arylthio, aralkylthio, mercapto, alkylsulfonyl, aralkylsulfonyl and arylsulfonyl, and amino and amido radicals of the formula

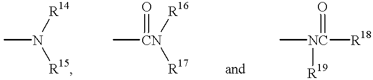

wherein each of R¹⁴, R¹⁵, R¹⁶, R¹⁷, R¹⁸ and R¹⁹ is independently selected from hydrido, alkyl, cycloalkyl, cyano, amino, monoalkylamino, dialkylamino, hydroxyalkyl, cycloalkylalkyl, alkoxyalkyl, aralkyl and aryl;

and wherein each of R³ through R¹¹ may be an acidic moiety further independently selected from acidic moieties of the formula

−YₙA

wherein n is a number selected from zero through three, inclusive;

wherein A is selected from carboxylic acid and bioisosteres of carboxylic acid selected from

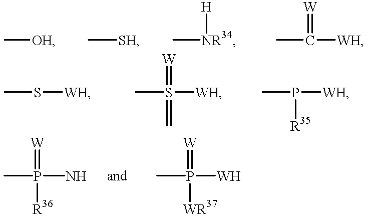

wherein each W is independently selected from oxygen atom, sulfur atom and NR³⁸; wherein each of R³⁴, R³⁵, R³⁶, R³⁷ and R³⁸ is independently selected from hydrido, alkyl, haloalkyl, haloalkylsulfonyl, haloalkylcarbonyl, cycloalkyl, cycloalkylalkyl, aryl and aralkyl; wherein each of R³⁴, R³⁵, R³⁶ and R³⁷ may be further independently selected from amino radical of the formula

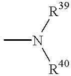

wherein each of R³⁹ and R⁴⁰ is independently selected from hydrido, alkyl, cycloalkyl, hydroxyalkyl, haloalkyl, cycloalkylalkyl, alkoxyalkyl, aralkyl and aryl, and wherein R³⁹ and R⁴⁰ taken together may form a heterocyclic group having five to seven ring members including the nitrogen atom of said amino radical, which heterocyclic group may further contain one or more hetero atoms as ring members selected from oxygen, nitrogen and sulfur atoms and which heterocyclic group may be saturated or partially unsaturated; wherein R³⁹ and R⁴⁰ taken together may form an aromatic heterocyclic group having five ring members including the nitrogen atom of said amino radical and which aromatic heterocyclic group may further contain one or more hetero atoms as ring atoms selected from oxygen, nitrogen and sulfur atoms; wherein each of R³⁵ and R³⁶ may be further independently selected from hydroxy, alkoxy, alkylthio, aryloxy, arylthio, aralkylthio and aralkoxy; and the amide, ester and salt derivatives of said acidic groups;

wherein said bioisostere of carboxylic acid may be further selected from heterocyclic acidic groups consisting of heterocyclic rings of four to about nine ring members, which heterocyclic ring contains at least one hetero atom selected from oxygen, sulfur and nitrogen atoms, which heterocyclic ring may be saturated, fully unsaturated or partially unsaturated, and which heterocyclic ring may be attached at a single position selected from R³ through R¹¹ or may be attached at any two adjacent positions selected from R³ through R¹¹ so as to form a fused-ring system with one of the phenyl rings of Formula I; and the amide, ester and salt derivatives of said heterocyclic acidic groups;

wherein Y is a spacer group independently selected from one or more of alkyl, cycloalkyl, cycloalkylalkyl, alkenyl, aryl and aralkyl;

and wherein any of the foregoing R¹ through R¹⁹ and R³⁴ through R⁴⁰, Y and A groups having a substitutable position may be substituted by one or more groups selected from hydroxy, alkyl, alkenyl, aralkyl, hydroxyalkyl, halo, oxo, haloalkyl, alkoxy, aryloxy, aralkoxy, alkoxyalkyl, alkylcarbonyl, alkoxycarbonyl, carboxyl, cyano, nitro, alkylsulfonyl, haloalkylsulfonyl, aryl, aralkyl, mercaptocarbonyl, alkylthio and alkylthiocarbonyl, and amino and amido radicals of the formula

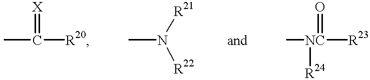

wherein X is selected from oxygen atom and sulfur atom; wherein R²⁰ is selected from hydrido, alkyl, cycloalkyl, cycloalkylalkyl, aralkyl, aryl and DR²⁵ and

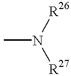

wherein D is selected from oxygen atom and sulfur atom, wherein R²⁵ is selected from hydrido, alkyl, cycloalkyl, cycloalkylalkyl, aralkyl and aryl;

wherein each of R²¹, R²², R²³, R²⁴, R²⁶ and R²⁷ is independently selected from hydrido, alkyl, cycloalkyl, cyano, hydroxyalkyl, haloalkyl, cycloalkylalkyl, alkoxyalkyl, alkanoyl, alkoxycarbonyl, carboxyl, haloalkylsulfinyl, haloalkylsulfonyl, aralkyl and aryl;

or a tautomer thereof or a pharmaceutically-acceptable salt thereof.

An even more preferred class of compounds consists of those compounds of Formula I wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkenyl, cycloalkenyl, alkynyl, mercaptocarbonyl, alkylsulfonyl, aralkylsulfonyl, arylsulfonyl and amido radicals of the formula

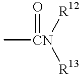

wherein each of R¹² and R¹³ is independently selected from hydrido, alkyl, cycloalkyl, cyano, amino, monoalkylamino, dialkylamino, hydroxyalkyl, cycloalkylalkyl, alkoxyalkyl, aralkyl and aryl;

wherein each of R² is selected from hydrido, alkyl, hydroxyalkyl, halo, halooalkyl, cycloalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, carboxyl, alkylcarbonyloxy, alkylthio, arylthio, aralkylthio, alkylsulfonyl, aralkylsulfonyl and arylsulfonyl;

wherein each of R³ through R¹¹ is independently selected from hydrido, hydroxy, alkyl, hydroxyalkyl, halo, haloalkyl, cycloalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cyano, nitro, carboxyl, alkylthio, aralkylthio and mercapto;

and wherein each of R³ through R¹¹ may be an acidic moiety further independently selected from acidic moieties of the formula

−YₙA

wherein n is a number selected from zero through three, inclusive; wherein A is selected from carboxylic acid and bioisosteres of carboxylic acid selected from

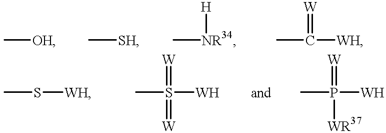

wherein each W is independently selected from oxygen atom, sulfur atom and NR³⁸; wherein each of R³⁴, R³⁷ and R³⁸ is independently selected from hydrido, alkyl, haloalkyl, haloalkylsulfonyl, haloalkylcarbonyl, cycloalkyl, cycloalkylalkyl, aryl and aralkyl; wherein each of R³⁴ and R³⁷ may be further independently selected from amino radical of the formula

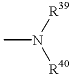

wherein each of R³⁹ and R⁴⁰ is independently selected from hydrido, alkyl, cycloalkyl, hydroxyalkyl, haloalkyl, cycloalkylalkyl, alkoxyalkyl, aralkyl and aryl, and wherein R³⁹ and R⁴⁰ taken together may form a heterocyclic group having five to seven ring members including the nitrogen atom of said amino radical, which heterocyclic group may further contain one or more hetero atoms as ring members selected from oxygen, nitrogen and sulfur atoms, and which heterocyclic group may be saturated or partially unsaturated; wherein R³⁹ and R⁴⁰ taken together may form an aromatic heterocyclic group having five ring members including the nitrogen atom of said amino radical and which aromatic heterocyclic group may further contain one or more hetero atoms as ring atoms selected from oxygen, nitrogen and sulfur atoms; and the amide, ester and salt derivatives of said acidic groups; wherein said bioisostere of carboxylic acid may be further selected from heterocyclic acidic groups consisting of heterocyclic rings of four to about nine ring members, which ring contains at least one hetero atom, selected from oxygen, sulfur and nitrogen atoms, which heterocyclic ring may be saturated, fully unsaturated or partially unsaturated, and which heterocyclic ring may be attached at a single position selected from R³ through R¹¹ or may be attached at any two adjacent positions selected from R³ through R¹¹ so as to form a fused-ring system with one of the phenyl rings of Formula I; and the amide, ester and salt derivatives of said heterocyclic acidic groups;

wherein Y is a spacer group independently selected from one or more of alkyl, cycloalkyl, cycloalkylalkyl, alkenyl, aryl and aralkyl;

wherein each of R¹ through R¹⁹, R³⁴ and R³⁷ through R⁴⁰, Y and A independently may be substituted at any substitutable position with one or more groups selected from alkyl, hydroxy, halo, oxo, haloalkyl, alkoxycarbonyl, cyano, nitro, alkylsulfonyl, haloalkylsulfonyl, aryl, aralkyl, alkoxy, aryloxy and aralkoxy;

or a tautomer thereof or a pharmaceutically-acceptable salt thereof.

A highly preferred class of compounds within Formula I consists of those compounds wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkenyl, alkynyl, alkylsulfonyl, aralkylsulfonyl, arylsulfonyl and amido radicals of the formula

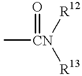

wherein each of R¹² and R¹³ is independently selected from hydrido, alkyl, cycloalkyl, cyano, amino, hydroxyalkyl, alkoxyalkyl, phenalkyl and phenyl;

wherein R² is selected from hydrido, alkyl, hydroxyalkyl, halo, haloalkyl, cycloalkyl, alkoxy, aralkyl, aryl, aryloxy, aralkoxy, alkoxyalkyl, alkylcarbonyl, alkoxycarbonyl, alkenyl, cycloalkenyl, alkynyl, alkylthio, arylthio, aralkylthio and arylsulfonyl,

wherein each of R³ through R¹¹ is independently selected from hydrido, hydroxy, alkyl, hydroxyalkyl, halo, haloalkyl, cycloalkyl, alkoxy, phenalkyl, phenyl, benzoyl, phenoxy, phenalkyloxy, alkoxyalkyl, alkylcarbonyl, alkoxycarbonyl, alkenyl, cyano, nitro, carboxyl, alkylthio and mercapto;

and wherein each of R³ through R¹¹ may be an acidic moiety further independently selected from acidic moieties of the formula

−YₙA

wherein n is a number selected from zero through two, inclusive; wherein A is selected from carboxylic acid and bioisosteres of carboxylic acid selected from

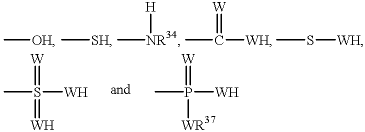

wherein each W is independently selected from oxygen atom, sulfur atom and NR³⁸; wherein each of R³⁴, R³⁷ and R³⁸ is independently selected from hydrido, alkyl, haloalkyl, haloalkylsulfonyl, haloalkylcarbonyl, cycloalkyl, phenyl and benzyl; wherein each of R³⁴ and R³⁷ may be further independently selected from amino radical of the formula

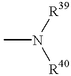

wherein each of R³⁹ and R⁴⁰ is independently selected from hydrido, alkyl, cycloalkyl, hydroxyalkyl, haloalkyl, alkoxyalkyl, benzyl and phenyl; and the amide, ester and salt derivatives of said acidic groups;

wherein said bioisostere of carboxylic acid may be further selected from heterocyclic acidic groups consisting of heterocyclic rings of four to about nine ring members, which ring contains at least one hetero atom, selected from oxygen, sulfur and nitrogen atoms, which heterocyclic ring may be saturated, fully unsaturated or partially unsaturated, and which heterocyclic ring may be attached at a single position selected from R³ through R¹¹ or may be attached at any two adjacent positions selected from R³ through R¹¹ so as to form a fused-ring system with one of the phenyl rings of Formula I; and the amide, ester and salt derivatives of said heterocyclic acidic groups;

wherein Y is a spacer group independently selected from one or more of alkyl, cycloalkyl, cycloalkylalkyl, alkenyl, phenyl, phenalkyl and aralkyl;

wherein each of R¹ through R^{19,} R³⁴ and R³⁷ through R⁴⁰, Y and A and independently may be substituted at any substitutable position with one or more groups selected from alkyl, cycloalkyl, cycloalkylalkyl, hydroxy, halo, oxo, haloalkyl, alkoxycarbonyl, cyano, nitro, alkylsulfonyl, haloalkylsulfonyl, aryl, aralkyl, alkoxy, aryloxy and aralkoxy;

or a tautomer thereof or a pharmaceutically-acceptable salt thereof.

An even more highly preferred class of compounds consists of those compounds of Formula I wherein m is one; wherein R¹ is selected from alkyl, hydroxyalkyl, haloalkyl, cycloalkyl, cycloalkylalkyl, aralkyl, aryl, benzoyl, alkoxyalkyl, alkylcarbonyl, alkoxycarbonyl, alkenyl and alkynyl;

where R² is selected from alkyl, hydroxyalkyl, cycloalkyl, haloalkyl, alkoxy, phenalkyl, phenyl, phenoxy, phenalkoxy, alkoxyalkyl, alkylcarbonyl, alkoxycarbonyl, alkenyl, cycloalkenyl, alkynyl, alkylthio, phenthio and phenalkylthio;

wherein each of R³ through R¹¹ is independently selected from hydrido, hydroxy, alkyl, hydroxyalkyl, halo, haloalkyl, alkoxy, phenyl, benzoyl, phenoxy, alkoxyalkyl, acetyl, alkoxycarbonyl, alkenyl, cyano, nitro, carboxyl, alkylthio and mercapto;

and wherein each of R³ through R¹¹ may be an acidic moiety further independently selected from acidic moieties consisting of CO₂H, CO₂CH₃, SH, CH₂SH, C₂H₄SH, PO₃H₂, NHSO₂CF₃, NHSO₂C₆F₅, SO₃H, CONHNH₂, CONHNHSO₂CF₃, CONHOCH₃, CONHOC₂H₅, CONHCF₃, OH, CH₂OH, C₂H₄OH, OPO₃H₂, OSO₃H ,

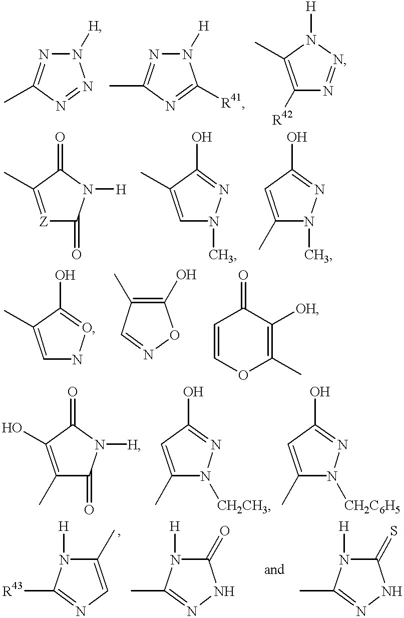

wherein each of R⁴¹, R⁴² and R⁴³ is independently selected from H, Cl, CN, NO₂, CF₃, C₂F₅, C₃F₇, CHF₂, CH₂F, CO₂CH₃, CO₂C₂H₅, SO₂CH₃, SO₂CF₃ and SO₂C₆F₅; wherein Z is selected from O, S, NR⁴⁴ and CH₂; wherein R⁴⁴ is selected from hydrido, CH₃ and CH₂C₆H₅; and wherein said acidic moiety may be a heterocyclic acidic group attached at any two adjacent positions of R³ through R¹¹ so as to form a fused ring system with one of the phenyl rings of the biphenyl moiety of Formula I, said biphenyl fused ring system selected from

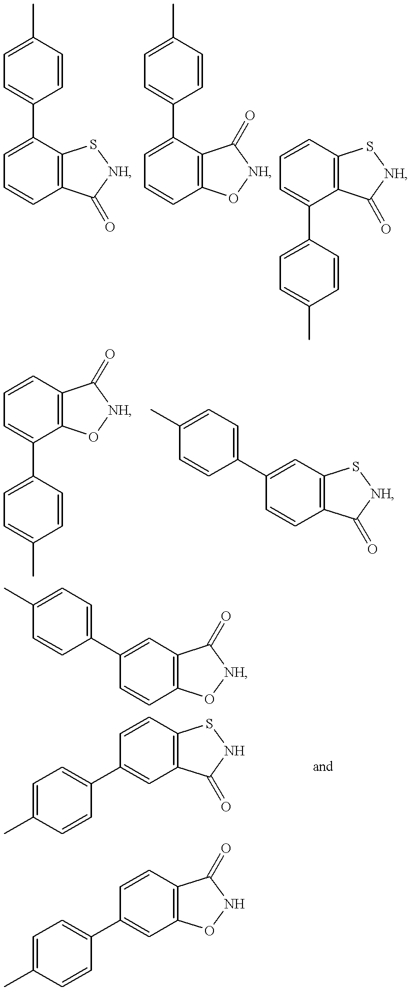

and the esters, amides and salts of said acidic moieties; or a tautomer thereof or a pharmaceutically-acceptable salt thereof.

A class of compounds of particular interest consists of those compounds of Formula I wherein m is one; wherein R¹ is selected from methyl, ethyl, n-propyl, isopropyl, n-butyl, sec-butyl, isobutyl, tert-butyl, n-pentyl, isopentyl, neopentyl, phenyl, benzyl, phenethyl, cyclohexyl, cyclohexylmethyl, 1-oxoethyl, 1-oxopropyl, 1-oxobutyl, 1-oxopentyl and hydroxyalkyl; wherein R² is selected from hydroxy, methyl, ethyl, n-propyl, isopropyl, n-butyl, sec-butyl, isobutyl, tert-butyl, n-pentyl, isopentyl, neopentyl, phenyl, benzyl, phenethyl, cyclohexyl, cyclohexylmethyl, propylthio, butylthio, 1-oxoethyl, 1-oxopropyl, 1-oxobutyl, 1-oxopentyl, 1,1-dimethoxypropyl, 1,1-dimethoxybutyl, 1,1-dimethoxypentyl, hydroxyalkyl, difluoromethyl, 1,1-difluoroethyl, 1,1-difluoropropyl, 1,1-difluorobutyl and 1,1-difluoropentyl; wherein at least one of R⁵, R⁶, R⁸ and R⁹ is an acidic group selected from CO₂H, SH, PO₃H₂, SO₃H, CONHNH₂, CONHNHSO₂CF₃, OH,

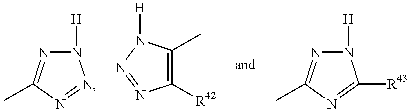

wherein each of R⁴² and R⁴³ is independently selected from Cl, CN, NO₂, CF₃, CO₂CH₃ and SO₂CF₃;

or a tautomer thereof or a pharmaceutically-acceptable salt thereof.

A class of compounds of more particular interest consists of those compounds of Formula I wherein m is one; wherein R¹ is selected from methyl, ethyl, n-propyl, isopropyl, n-butyl, sec-butyl, isobutyl, tert-butyl, 4-methylbutyl, n-pentyl, neopentyl, phenyl, benzyl, phenethyl, cyclohexyl, cyclohexylmethyl, 1-oxoethyl, 1-oxopropyl, 1-oxobutyl, 1-oxopentyl and hydroxyalkyl; wherein R² is selected from ethyl, n-propyl, isopropyl, n-butyl, sec-butyl, isobutyl, 4-methylbutyl, tert-butyl, n-pentyl, neopentyl, propylthio and butylthio; wherein at least one of R⁵, R⁶, R⁸ and R⁹ is an acidic group selected from CO₂H, SH, PO₃H₂, SO₃H, CONHNH₂, CONHNHSO₂CF₃, OH,

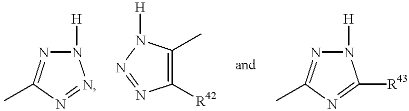

wherein each of R⁴² and R⁴³ is independently selected from Cl, CN, NO₂, CF_{3,} CO₂CH₃ and SO₂CF₃;

or a tautomer thereof or a pharmaceutically-acceptable salt thereof.

A class of compounds of even more particular interest consists of those compounds of Formula I wherein m is one; wherein R¹ is selected from methyl, ethyl, n-propyl, isopropyl, n-butyl, sec-butyl, isobutyl, tert-butyl, 4-methylbutyl, n-pentyl, neopentyl, phenyl, benzyl, phenethyl, cyclohexyl, cyclohexylmethyl and hydroxyalkyl; wherein R² is selected from ethyl, n-propyl, isopropyl, n-butyl, sec-butyl, isobutyl, 4-methylbutyl, n-pentyl, propylthio and butylthio; wherein each of R³, R⁴, R⁶, R⁷, R⁸, R¹⁰ and R¹¹ is hydrido; wherein one of R⁵ and R⁹ is hydrido and the other of R⁵ and R⁹ is an acidic group selected from CO₂H and

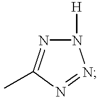

or a tautomer thereof or a pharmaceutically-acceptable salt thereof.

A family of specific compounds of particular interest within Formula I consists of the following compounds:

4′-[(1,3-diethyl-4,5-dihydro-5-oxo-1H-1,2,4-triazol-4-yl) methyl] [1,1′-biphenyl]-2-carboxylic acid;

4′-[(3-ethyl-1-propyl-4,5-dihydro-5-oxo-1H-1,2,4-triazol-4-yl) methyl] [1,1′-biphenyl]-2-carboxylic acid;

4′-[(3-ethyl-1-isopropyl-4,5-dihydro-5-oxo-1H-1,2,4-triazol-4-yl) methyl] [1,1′-biphenyl]-2-carboxylic acid;

4′-[(3-ethyl-1-butyl-4,5-dihydro-5-oxo-1H-1,2,4-triazol-4-yl) methyl] [1,1′-biphenyl]-2-carboxylic acid;

4′-[(3-ethyl-1-secbutyl-4,5-dihydro-5-oxo-1H-1,2,4-triazol-4-yl) methyl] [1,1′-biphenyl]-2-carboxylic acid;

4′-[(3-ethyl-1-isobutyl-4,5-dihydro-5-oxo-1H-1,2,4-triazol-4-yl) methyl] [1,1′-biphenyl]-2-carboxylic acid;

4′-[(3-ethyl-1-tertbutyl-4,5-dihydro-5-oxo-1H-1,2,4-triazol-4-yl) methyl] [1,1′-biphenyl]-2-carboxylic acid;

4′-[(3-ethyl-1-pentyl-4,5-dihydro-5-oxo-1H-1,2,4-triazol-4-yl) methyl] [1,1′-biphenyl]-2-carboxylic acid;

4′-[(3-ethyl-1-isopentyl-4,5-dihydro-5-oxo-1H-1,2,4-triazol-4-yl) methyl] [1,1′-biphenyl]-2-carboxylic acid;

4′-[(3-ethyl-1-hexyl-4,5-dihydro-5-oxo-1H-1,2,4-triazol-4-yl) methyl] [1,1′-biphenyl]-2-carboxylic acid;

4′-[(3-ethyl-1-heptyl-4,5-dihydro-5-oxo-1H-1,2,4-triazol-4-yl) methyl] [1,1′-biphenyl]-2-carboxylic acid;

4′-[(3-ethyl-1-octyl-4,5-dihydro-5-oxo-1H-1,2,4-triazol-4-yl) methyl] [1,1′-biphenyl]-2-carboxylic acid;

4′-[(3-ethyl-1-cyclohexyl-4,5-dihydro-5-oxo-1H-1,2,4-triazol-4-yl) methyl] [1,1′-biphenyl]-2-carboxylic acid;

4′-[(3-ethyl-1-cyclohexylmethyl-4,5-dihydro-5-oxo-1H-1,2,4-triazol-4-yl) methyl] [1,1′-biphenyl]-2-carboxylic acid;

4′-[[3-ethyl-1-(2-cyclohexylethyl)-4,5-dihydro-5-oxo-1H-1,2,4-triazol-4-yl] methyl] [1,1′-biphenyl]-2-carboxylic acid;

4′-[(3-ethyl-1-cyclohexanoyl-4,5-dihydro-5-oxo-1H-1,2,4-triazol-4-yl) methyl] [1,1′-biphenyl]-2-carboxylic acid;

4′-[[3-ethyl-1-(1-oxo-2-cyclohexylethyl)-4,5-dihydro-5-oxo-1H-1,2,4-triazol-4-yl] methyl] [1,1′-biphenyl]-2-carboxylic acid;

4′-[(3-ethyl-1-phenyl-4,5-dihydro-5-oxo-1H-1,2,4-triazol-4-yl) methyl] [1,1′-biphenyl]-2-carboxylic acid;

4′-[(3-ethyl-1-phenylmethyl-4,5-dihydro-5-oxo-1H-1,2,4-triazol-4-yl) methyl] [1,1′-biphenyl]-2-carboxylic acid;

4′-[[3-ethyl-1-(2-phenylethyl)-4,5-dihydro-5-oxo-1H-1,2,4-triazol-4-yl] methyl] [1,1′-biphenyl]-2-carboxylic acid;

4′-[(3-ethyl-1-benzoyl-4,5-dihydro-5-oxo-1H-1,2,4-triazol-4-yl) methyl] [1,1′-biphenyl]-2-carboxylic acid;

4′-[[3-ethyl-1-(1-oxo-2-phenylethyl)-4,5-dihydro-5-oxo-1H-1,2,4-triazol-4-yl] methyl] [1,1′-biphenyl]-2-carboxylic acid;

4′-[[3-ethyl-1-(1-oxopropyl)-4,5-dihydro-5-oxo-1H-1,2,4-triazol-4-yl] methyl] [1,1′-biphenyl]-2-carboxylic acid;

4′-[[3-ethyl-1-(1-oxobutyl)-4,5-dihydro-5-oxo-1H-1,2,4-triazol-4-yl] methyl] [1,1′-biphenyl]-2-carboxylic acid;

4′-[[3-ethyl-1-(1-oxopentyl)-4,5-dihydro-5-oxo-1H-1,2,4-triazol-4-yl] methyl] [1,1′-biphenyl]-2-carboxylic acid;

4′-[[3-ethyl-1-(2-butenyl)-4,5-dihydro-5-oxo-1H-1,2,4-triazole-4-yl] methyl] [1,1′-biphenyl]-2-carboxylic acid;

4′-[[3-ethyl-1-(3-butenyl)-4,5-dihydro-5-oxo-1H-1,2,4-triazole-4-yl] methyl] [1,1′-biphenyl]-2-carboxylic acid;

4′-[[3-ethyl-1-(2-butynyl)-4,5-dihydro-5-oxo-1H-1,2,4-triazole-4-yl] methyl] [1,1′-biphenyl]-2-carboxylic acid;

4′-[[3-ethyl-1-(3-butynyl)-4,5-dihydro-5-oxo-1H-1,2,4-triazole-4-yl] methyl] [1,1′-biphenyl]-2-carboxylic acid;

4′-[(3-propyl-1-ethyl-4,5-dihydro-5-oxo-1H-1,2,4-triazol-4-yl) methyl] [1,1′-biphenyl]-2-carboxylic acid;

4′-[(1,3-dipropyl-4,5-dihydro-5-oxo-1H-1,2,4-triazol-4-yl) methyl] [1,1′-biphenyl]-2-carboxylic acid;

4′-[(3-propyl-1-isopropyl-4,5-dihydro-5-oxo-1H-1,2,4-triazol-4-yl) methyl] [1,1′-biphenyl]-2-carboxylic acid;

4′-[(3-propyl-1-butyl-4,5-dihydro-5-oxo-1H-1,2,4-triazol-4-yl) methyl] [1,1′-biphenyl]-2-carboxylic acid;

4′-[(3-propyl-1-secbutyl-4,5-dihydro-5-oxo-1H-1,2,4-triazol-4-yl) methyl] [1,1′-biphenyl]-2-carboxylic acid;

4′-[(3-propyl-1-isobutyl-4,5-dihydro-5-oxo-1H-1,2,4-triazol-4-yl) methyl] [1,1′-biphenyl]-2-carboxylic acid;

4′-[(3-propyl-1-tertbutyl-4,5-dihydro-5-oxo-1H-1,2,4-triazol-4-yl) methyl] [1,1′-biphenyl]-2-carboxylic acid;

4′-[(3-propyl-1-pentyl-4,5-dihydro-5-oxo-1H-1,2,4-triazol-4-yl) methyl] [1,1′-biphenyl]-2-carboxylic acid;

4′-[(3-propyl-1-isopentyl-4,5-dihydro-5-oxo-1H-1,2,4-triazol-4-yl) methyl] [1,1′-biphenyl]-2-carboxylic acid;

4′-[(3-propyl-1-hexyl-4,5-dihydro-5-oxo-1H-1,2,4-triazol-4-yl) methyl] [1,1′-biphenyl]-2-carboxylic acid;

4′-[(3-propyl-1-heptyl-4,5-dihydro-5-oxo-1H-1,2,4-triazol-4-yl) methyl] [1,1′-biphenyl]-2-carboxylic acid;

4′-[(3-propyl-1-octyl--4,5-dihydro-5-oxo-1H-1,2,4-triazol-4-yl) methyl] [1,1′-biphenyl]-2-carboxylic acid;

4′-[(3-propyl-1-cyclohexyl-4,5-dihydro-5-oxo-1H-1,2,4-triazol-4-yl) methyl] [1,1′-biphenyl]-2-carboxylic acid;

4′-[(3-propyl-1-cyclohexylmethyl-4,5-dihydro-5-oxo-1H-1,2,4-triazol-4-yl) methyl] [1,1′-biphenyl]-2-carboxylic acid;

4′-[[3-propyl-1-(2-cyclohexylethyl)-4,5-dihydro-5-oxo-1H-1,2,4-triazol-4-yl] methyl] [1,1′-biphenyl]-2-carboxylic acid;

4′-[(3-propyl-1-cyclohexanoyl-4,5-dihydro-5-oxo-1H-1,2,4-triazol-4-yl) methyl] [1,1′-biphenyl]-2-carboxylic acid;

4′-[[3-propyl-1-(1-oxo-2-cyclohexylethyl)-4,5-dihydro-5-oxo-1H-1,2,4-triazol-4-yl] methyl] [1,1′-biphenyl]-2-carboxylic acid;

4′-[(3-propyl-1-phenyl-4,5-dihydro-5-oxo-1H-1,2,4-triazol-4-yl) methyl] [1,1′-biphenyl)-2-carboxylic acid;

4′-[(3-propyl-1-phenylmethyl-4,5-dihydro-5-oxo-1H-1,2,4-triazol-4-yl) methyl] [1,1′-biphenyl]-2-carboxylic acid;

4′-[(3-propyl-1-(2-phenylethyl)-4,5-dihydro-5-oxo-1H-1,2,4-triazol-4-yl) methyl] [1,1′-biphenyl]-2-carboxylic acid;

4′-[(3-propyl-1-benzoyl-4,5-dihydro-5-oxo-1H-1,2,4-triazol-4-yl) methyl] [1,1′-biphenyl]-2-carboxylic acid;

4′-[[3-propyl-1-(1-oxo-2-phenylethyl)-4,5-dihydro-5-oxo-1H-1,2,4-triazol-4-yl] methyl]1,1′-biphenyl]-2-carboxylic acid;

4′-[[3-propyl-1-(1-oxopropyl)-4,5-dihydro-5-oxo-1H-1,2,4-triazol-4-yl] methyl] [1,1′-biphenyl]-2-carboxylic acid;

4′-[[3-propyl-1-(1-oxobutyl)-4,5-dihydro-5-oxo-1H-1,2,4-triazol-4-yl] methyl] [1,1′-biphenyl]-2-carboxylic acid;

4′-[[3-propyl-1-(1-oxopentyl)-4,5-dihydro-5-oxo-1H-1,2,4-triazol-4-yl] methyl] [1,1′-biphenyl]-2-carboxylic acid;

4′-[[3-propyl-1-(2-butenyl)-4,5-dihydro-5-oxo-1H-1,2,4-triazole-4-yl] methyl] [1,1′-biphenyl]-2-carboxylic acid;

4′-[[3-propyl-1-(3-butenyl)-4,5-dihydro-5-oxo-1H-1,2,4-triazole-4-yl] methyl] [1,1′-biphenyl]-2-carboxylic acid;

4′-[[3-propyl-1-(2-butynyl)-4,5-dihydro-5-oxo-1H-1,2,4-triazole-4-yl] methyl] [1,1′-biphenyl]-2-carboxylic acid;

4′-[[3-propyl-1-(3-butynyl)-4,5-dihydro-5-oxo-1H-1,2,4-triazole-4-yl] methyl] [1,1′-biphenyl]-2-carboxylic acid;

4′-[(3-isopropyl-1-ethyl-4,5-dihydro-5-oxo-1H-1,2,4-triazol-4-yl) methyl] [1,1′-biphenyl]-2-carboxylic acid;

4′-[(3-isopropyl-1-propyl-4,5-dihydro-5-oxo-1H-1,2,4-triazol-4-yl) methyl] [1,1′-biphenyl]-2-carboxylic acid;

4′-[(1,3-isopropyl-4,5-dihydro-5-oxo-1H-1,2,4-triazol-4-yl) methyl] [1,1′-biphenyl]-2-carboxylic acid;

4′-[(3-isopropyl-1-butyl-4,5-dihydro-5-oxo-1H-1,2,4-triazol-4-yl) methyl] [1,1′-biphenyl]-2-carboxylic acid;

4′-[(3-isopropyl-1-secbutyl-4,5-dihydro-5-oxo-1H-1,2,4-triazol-4-yl) methyl] [1,1′-biphenyl]-2-carboxylic acid;

4′-[(3-isopropyl-1-isobutyl-4,5-dihydro-5-oxo-1H-1,2,4-triazol-4-yl) methyl] [1,1′-biphenyl]-2-carboxylic acid;

4′-[(3-isopropyl-1-tertbutyl-4,5-dihydro-5-oxo-1H-1,2,4-triazol-4-yl) methyl] [1,1′-biphenyl]-2-carboxylic acid;

4′-[(3-isopropyl-1-pentyl-4,5-dihydro-5-oxo-1H-1,2,4-triazol-4-yl) methyl] [1,1′-biphenyl]-2-carboxylic acid;

4′-[(3-isopropyl-1-isopentyl-4,5-dihydro-5-oxo-1H-1,2,4-triazol-4-yl) methyl] [1,1′-biphenyl]-2-carboxylic acid;

4′-[(3-isopropyl-1-hexyl-4,5-dihydro-5-oxo-1H-1,2,4-triazol-4-yl) methyl] [1,1′-biphenyl]-2-carboxylic acid;

4′-[(3-isopropyl-1-heptyl-4,5-dihydro-5-oxo-1H-1,2,4-triazol-4-yl) methyl] [1,1′-biphenyl]-2-carboxylic acid;

4′-[(3-isopropyl-1-octyl-4,5-dihydro-5-oxo-1H-1,2,4-triazol-4-yl) methyl] [1,1′-biphenyl]-2-carboxylic acid;

4′-[(3-isopropyl-1-cyclohexyl-4,5-dihydro-5-oxo-1H-1,2,4-triazol-4-yl) methyl] [1,1′-biphenyl]-2-carboxylic acid;

4′-[(3-isopropyl-1-cyclohexylmethyl-4,5-dihydro-5-oxo-1H-1,2,4-triazol-4-yl) methyl] [1,1′-biphenyl]-2-carboxylic acid;

4′-[[3-isopropyl-1-(2-cyclohexylethyl)-4,5-dihydro-5-oxo-1H-1,2,4-triazol-4-yl] methyl] [1,1′-biphenyl]-2-carboxylic acid;

4′-[(3-isopropyl-1-cyclohexanoyl-4,5-dihydro-5-oxo-1H-1,2,4-triazol-4-yl) methyl] [1,1′-biphenyl]-2-carboxylic acid;

4′-[[3-isopropyl-1-(1-oxo-2-cyclohexylethyl)-4,5-dihydro-5-oxo-1H-1,2,4-triazol-4-yl] methyl] [1,1′-biphenyl]-2-carboxylic acid;

4′-[(3-isopropyl-1-phenyl-4,5-dihydro-5-oxo-1H-1,2,4-triazol-4-yl) methyl] [1,1′-biphenyl]-2-carboxylic acid;

4′-[(3-isopropyl-1-phenylmethyl-4,5-dihydro-5-oxo-1H-1,2,4-triazol-4-yl) methyl] [1,1′-biphenyl]-2-carboxylic acid;

4′-[[3-isopropyl-1-(2-phenylethyl)-4,5-dihydro-5-oxo-1H-1,2,4-triazol-4-yl] methyl] [1,1′-biphenyl]-2-carboxylic acid;

4′-[(3-isopropyl-1-benzoyl-4,5-dihydro-5-oxo-1H-1,2,4-triazol-4-yl) methyl] [1,1′-biphenyl]-2-carboxylic acid;

4′-[[3-isopropyl-1-(1-oxo-2-phenylethyl)-4,5-dihydro-5-oxo-1H-1,2,4-triazol-4-yl] methyl] [1,1′-biphenyl]-2-carboxylic acid;

4′-[[3-isopropyl-1-(1-oxopropyl)-4,5-dihydro-5-oxo-1H-1,2,4-triazol-4-yl] methyl] [1,1′-biphenyl]-2-carboxylic acid;

4′-[[3-isopropyl-1-(1-oxobutyl)-4,5-dihydro-5-oxo-1H-1,2,4-triazol-4-yl] methyl] [1,1′-biphenyl]-2-carboxylic acid;

4′-[[3-isopropyl-1-(1-oxopentyl)-4,5-dihydro-5-oxo-1H-1,2,4-triazol-4-yl] methyl] [1,1′-biphenyl]-2-carboxylic acid;

4′-[[3-isopropyl-1-(2-butenyl)-4,5-dihydro-5-oxo-1H-1,2,4-triazole-4-yl] methyl] [1,1′-biphenyl]-2-carboxylic acid;

4′-[[3-isopropyl-1-(3-butenyl)-4,5-dihydro-5-oxo-1H-1,2,4-triazole-4-yl] methyl] [1,1′-biphenyl]-2-carboxylic acid;

4′-[[3-isopropyl-1-(2-butynyl)-4,5-dihydro-5-oxo-1H-1,2,4-triazole-4-yl] methyl] [1,1′-biphenyl]-2-carboxylic acid;

4′-[[3-isopropyl-1-(3-butynyl)-4, 5-dihydro-5-oxo-1H-1,2,4-triazole-4-yl] methyl] [1,1′-biphenyl]-2-carboxylic acid;

4′-[(3-butyl-1-ethyl-4,5-dihydro-5-oxo-1H-1,2,4-triazol-4-yl) methyl] [1,1′-biphenyl]-2-carboxylic acid;

4′-[(3-butyl-1-propyl-4,5-dihydro-5-oxo-1H-1,2,4-triazol-4-yl) methyl] [1,1′-biphenyl]-2-carboxylic acid;

4′-[(3-butyl-1-isopropyl-4,5-dihydro-5-oxo-1H-1,2,4-triazol-4-yl) methyl] [1,1′-biphenyl]-2-carboxylic acid;

4′-[(1,3-dibutyl-4,5-dihydro-5-oxo-1H-1,2,4-triazol-4-yl) methyl] [1,1′-biphenyl]-2-carboxylic acid;

4′-[(3-butyl-1-secbutyl-4,5-dihydro-5-oxo-1H-1,2,4-triazol-4-yl) methyl] [1,1′-biphenyl]-2-carboxylic acid;

4′-[(3-butyl-1-isobutyl-4,5-dihydro-5-oxo-1H-1,2,4-triazol-4-yl) methyl] [1,1′-biphenyl]-2-carboxylic acid;

4′-[(3-butyl-1-tertbutyl-4,5-dihydro-5-oxo-1H-1,2,4-triazol-4-yl) methyl] [1,1′-biphenyl]-2-carboxylic acid;

4′-[(3-butyl-1-pentyl-4,5-dihydro-5-oxo-1H-1,2,4-triazol-4-yl) methyl] [1,1′-biphenyl]-2-carboxylic acid;

4′-[(3-butyl-1-isopentyl-4,5-dihydro-5-oxo-1H-1,2,4-triazol-4-yl) methyl] [1,1′-biphenyl]-2-carboxylic acid;

4′-[(3-butyl-1-hexyl-4,5-dihydro-5-oxo-1H-1,2,4-triazol-4-yl) methyl] [1,1′-biphenyl]-2-carboxylic acid;

4′-[(3-butyl-1-heptyl-4,5-dihydro-5-oxo-1H-1,2,4-triazol-4-yl) methyl] [1,1′-biphenyl]-2-carboxylic acid;

4′-[(3-butyl-1-octyl-4,5-dihydro-5-oxo-1H-1,2,4-triazol-4-yl) methyl] [1,1′-biphenyl]-2-carboxylic acid;

4′-[(3-butyl-1-cyclohexyl-4,5-dihydro-5-oxo-1H-1,2,4-triazol-4-yl) methyl] [1,1′-biphenyl]-2-carboxylic acid;

4′-[(3-butyl-1-cyclohexylmethyl-4,5-dihydro-5-oxo-1H-1,2,4-triazol-4-yl) methyl] [1,1′-biphenyl]-2-carboxylic acid;

4′-[[3-butyl-1-(2-cyclohexylethyl)-4,5-dihydro-5-oxo-1H-1,2,4-triazol-4-yl] methyl] [1,1′-biphenyl]-2-carboxylic acid;

4′-[(3-butyl-1-cyclohexanoyl-4,5-dihydro-5-oxo-1H-1,2,4-triazol-4-yl) methyl] [1,1′-biphenyl]-2-carboxylic acid;

4′-[[3-butyl-1-(1-oxo-2-cyclohexylethyl)-4,5-dihydro-5-oxo-1H-1,2,4-triazol-4-yl] methyl] [1,1′-biphenyl]-2-carboxylic acid;

4′-[(3-butyl-1-phenyl-4,5-dihydro-5-oxo-1H-1,2,4-triazol-4-yl) methyl] [1,1′-biphenyl]-2-carboxylic acid;

4′-[(3-butyl-1-phenylmethyl-4,5-dihydro-5-oxo-1H-1,2,4-triazol-4-yl) methyl] [1,1′-biphenyl]-2-carboxylic acid;

4′-[[3-butyl-1-(2-phenylethyl)-4,5-dihydro-5-oxo-1H-1,2,4-triazol-4-yl] methyl] [1,1′-biphenyl]-2-carboxylic acid;

4′-[(3-butyl-1-benzoyl-4,5-dihydro-5-oxo-1H-1,2,4-triazol-4-yl) methyl] [1,1′-biphenyl]-2-carboxylic acid;

4′-[[3-butyl-1-(l-oxo-2-phenylethyl)-4,5-dihydro-5-oxo-1H-1,2,4-triazol-4-yl] methyl] [1,1′-biphenyl]-2-carboxylic acid;

4′-[[3-butyl-1-(1-oxopropyl)-4,5-dihydro-5-oxo-1H-1,2,4-triazol-4-yl] methyl] [1,1′-biphenyl]-2-carboxylic acid;

4′-[[3-butyl-1-(1-oxobutyl)-4,5-dihydro-5-oxo-1H-1,2,4-triazol-4-yl] methyl] [1,1? -biphenyl]-2-carboxylic acid;

4′-[[3-butyl-1-(l-oxopentyl)-4,5-dihydro-5-oxo-1H-1,2,4-triazol-4-yl] methyl] [1,1′-biphenyl]-2-carboxylic acid;

4′-[[3-butyl-1-(2-butenyl)-4,5-dihydro-5-oxo-1H-1,2,4-triazole-4-yl] methyl] [1,1′-biphenyl]-2-carboxylic acid;

4′-[[3-butyl-1-(3-butenyl)-4,5-dihydro-5-oxo-1H-1,2,4-triazole-4-yl] methyl] [1,1′-biphenyl]-2-carboxylic acid;

4′-[[3-butyl-1-(2-butynyl)-4,5-dihydro-5-oxo-1H-1,2,4-triazole-4-yl] methyl] [1,1′-biphenyl]-2-carboxylic acid;

4′-[[3-butyl-1-(3-butynyl)-4,5-dihydro-5-oxo-1H-1,2,4-triazole-4-yl] methyl] [1,1′-biphenyl]-2-carboxylic acid;

4′-[(3-secbutyl-1-ethyl-4,5-dihydro-5-oxo-1H-1,2,4-triazol-4-yl) methyl] [1,1′-biphenyl]-2-carboxylic acid;

4′-[(3-secbutyl-1-propyl-4,5-dihydro-5-oxo-1H-1,2,4-triazol-4-yl) methyl] [1,1′-biphenyl]-2-carboxylic acid;

4′-[(3-secbutyl-1-isopropyl-4,5-dihydro-5-oxo-1H-1,2,4-triazol-4-yl) methyl] [1,1′-biphenyl]-2-carboxylic acid;

4′-[(3-secbutyl-1-butyl-4,5-dihydro-5-oxo-1H-1,2,4-triazol-4-yl) methyl] [1,1′-biphenyl]-2-carboxylic acid;

4′-[(1,3-disecbutyl-4,5-dihydro-5-oxo-1H-1,2,4-triazol-4-yl) methyl] [1,1′-biphenyl]-2-carboxylic acid;

4′-[(3-secbutyl-1-isobutyl-4,5-dihydro-5-oxo-1H-1,2,4-triazol-4-yl) methyl] [1,1′-biphenyl]-2-carboxylic acid;

4′-[(3-secbutyl-1-tertbutyl-4,5-dihydro-5-oxo-1H-1,2,4-triazol-4-yl) methyl] [1,1′-biphenyl]-2-carboxylic acid;

4′-[(3-secbutyl-1-pentyl-4,5-dihydro-5-oxo-1H-1,2,4-triazol-4-yl) methyl] [1,1′-biphenyl]-2-carboxylic acid;

4′-[(3-secbutyl-1-isopentyl-4,5-dihydro-5-oxo-1H-1,2,4-triazol-4-yl) methyl] [1,1′-biphenyl]-2-carboxylic acid;

4′-[(3-secbutyl-1-hexyl-4,5-dihydro-5-oxo-1H-1,2,4-triazol-4-yl) methyl] [1,1′-biphenyl]-2-carboxylic acid;

4′-[(3-secbutyl-1-heptyl-4,5-dihydro-5-oxo-1H-1,2,4-triazol-4-yl) methyl] [1,1′-biphenyl]-2-carboxylic acid;

4′-[(3-secbutyl-1-octyl-4,5-dihydro-5-oxo-1H-1,2,4-triazol-4-yl) methyl] [1,1′-biphenyl]-2-carboxylic acid;

4′-[(3-secbutyl-1-cyclohexyl-4,5-dihydro-5-oxo-1H-1,2,4-triazol-4-yl) methyl] [1,1′-biphenyl]-2-carboxylic acid;

4′-[(3-secbutyl-1-cyclohexylmethyl-4,5-dihydro-5-oxo-1H-1,2,4-triazol-4-yl) methyl] [1,1′-biphenyl]-2-carboxylic acid;

4′-[[3-secbutyl-1-(2-cyclohexylethyl)-4,5-dihydro-5-oxo-1H-1,2,4-triazol-4-yl] methyl] [1,1′-biphenyl-2-carboxylic acid;

4′-[(3-secbutyl-1-cyclohexanoyl-4,5-dihydro-5-oxo-1H-1,2,4-triazol-4-yl) methyl] [1,1′-biphenyl]-2-carboxylic acid;

4′-[[3-secbutyl-1-(1-oxo-2-cyclohexylethyl)-4,5-dihydro-5-oxo-1H-1,2,4-triazol-4-yl] methyl] [1,1′-biphenyl]-2-carboxylic acid;

4′-[(3-secbutyl-1-phenyl-4,5-dihydro-5-oxo-1H-1,2,4-triazol-4-yl) methyl] [1,1′-biphenyl]-2-carboxylic acid;

4′-[(3-secbutyl-1-phenylmethyl-4,5-dihydro-5-oxo-1H-1,2,4-triazol-4-yl) methyl] [1,1′-biphenyl]-2-carboxylic acid;

4′-[[3-secbutyl-1-(2-phenylethyl)-4,5-dihydro-5-oxo-1H-1,2,4-triazol-4-yl] methyl] [1,1′-biphenyl]-2-carboxylic acid;

4′-[(3-secbutyl-1-benzoyl-4,5-dihydro-5-oxo-1H-1,2,4-triazol-4-yl) methyl] [1,1′-biphenyl]-2-carboxylic acid;

4′-[[3-secbutyl-1-(1-oxo-2-phenylethyl)-4,5-dihydro-5-oxo-1H-1,2,4-triazol-4-yl] methyl] [1,1′-biphenyl]-2-carboxylic acid;

4′-[[3-secbutyl-1-(1-oxopropyl)-4,5-dihydro-5-oxo-1H-1,2,4-triazol-4-yl] methyl] [1,1′-biphenyl]-2-carboxylic acid;

4′-[[3-secbutyl-1-(l-oxobutyl)-4,5-dihydro-5-oxo-1H-1,2,4-triazol-4-yl] methyl] [1,1′-biphenyl]-2-carboxylic acid;

4′-[[3-secbutyl-1-(1-oxopentyl)-4,5-dihydro-5-oxo-1H-1,2,4-triazol-4-yl] methyl] [1,1′-biphenyl]-2-carboxylic acid;

4′-[[3-secbutyl-1-(2-butenyl)-4,5-dihydro-5-oxo-1H-1,2,4-triazole-4-yl] methyl] [1,1′-biphenyl]-2-carboxylic acid;

4′-[[3-secbutyl-1-(3-butenyl)-4,5-dihydro-5-oxo-1H-1,2,4-triazole-4-yl] methyl] [1,1′ -biphenyl]-2-carboxylic acid;

4′-[[3-secbutyl-1-(2-butynyl)-4,5-dihydro-5-oxo-1H-1,2,4-triazole-4-yl] methyl] [1,1′-biphenyl]-2-carboxylic acid;

4′-[[3-secbutyl-1-(3-butynyl)-4,5-dihydro-5-oxo-1H-1,2,4-triazole-4-yl] methyl] [1,1′-biphenyl]-2-carboxylic acid;

4′-[(3-isobutyl-1-ethyl-4,5-dihydro-5-oxo-1H-1,2,4-triazol-4-yl) methyl] [1,1′-biphenyl3-2-carboxylic acid;

4′-[(3-isobutyl-1-propyl-4,5-dihydro-5-oxo-1H-1,2,4-triazol-4-yl) methyl] [1,1′-biphenyl]-2-carboxylic acid;

4′-[(3-isobutyl-1-isopropyl-4,5-dihydro-5-oxo-1H-1,2,4-triazol-4-yl) methyl] [1,1′-biphenyl]-2-carboxylic acid;

4′-[(3-isobutyl-1-butyl-4,5-dihydro-5-oxo-1H-1,2,4-triazol-4-yl) methyl] [1,1′-biphenyl]-2-carboxylic acid;

4′-[(3-isobutyl-1-secbutyl-4,5-dihydro-5-oxo-1H-1,2,4-triazol-4-yl) methyl] [1,1′-biphenyl]-2-carboxylic acid;

4′-[(1,3-diisobutyl-4,5-dihydro-5-oxo-1H-1,2,4-triazol-4-yl) methyl] [1,1′-biphenyl]-2-carboxylic acid;

4′-[(3-isobutyl-1-tertbutyl-4,5-dihydro-5-oxo-1H-1,2,4-triazol-4-yl) methyl] [1,1′-biphenyl]-2-carboxylic acid;

4′-[(3-isobutyl-1-pentyl-4,5-dihydro-5-oxo-1H-1,2,4-triazol-4-yl) methyl] [1,1′-biphenyl]-2-carboxylic acid;

4′-[(3-isobutyl-1-isopentyl-4,5-dihydro-5-oxo-1H-1,2,4-triazol-4-yl) methyl] [1,1′-biphenyl]-2-carboxylic acid;

4′-[(3-isobutyl-1-hexyl-4,5-dihydro-5-oxo-1H-1,2,4-triazol-4-yl) methyl] [1,1′-biphenyl]-2-carboxylic acid;

4′-[(3-isobutyl-1-heptyl-4,5-dihydro-5-oxo-1H-1,2,4-triazol-4-yl) methyl] [1,1′-biphenyl]-2-carboxylic acid;

4′-[(3-isobutyl-1-octyl-4,5-dihydro-5-oxo-1H-1,2,4-triazol-4-yl) methyl] [1,1′-biphenyl]-2-carboxylic acid;

4′-[(3-isobutyl-1-cyclohexyl-4,5-dihydro-5-oxo-1H-1,2,4-triazol-4-yl) methyl] [1,1′-biphenyl]-2-carboxylic acid;

4′-[(3-isobutyl-1-cyclohexylmethyl-4,5-dihydro-5-oxo-1H-1,2,4-triazol-4-yl) methyl] [1,1′-biphenyl]-2-carboxylic acid;

4′-[[3-isobutyl-1-(2-cyclohexylethyl)-4,5-dihydro-5-oxo-1H-1,2,4-triazol-4-yl] methyl] [1,1′-biphenyl]-2-carboxylic acid;

4′-[(3-isobutyl-1-cyclohexanoyl-4,5-dihydro-5-oxo-1H-1,2,4-triazol-4-yl) methyl] [1,1′-biphenyl]-2-carboxylic acid;

4′-[[3-isobutyl-1-(1-oxo-2-cyclohexylethyl)-4,5-dihydro-5-oxo-1H-1,2,4-triazol-4-yl] methyl] [1,1′-biphenyl]-2-carboxylic acid;

4′-[(3-isobutyl-1-phenyl-4,5-dihydro-5-oxo-1H-1,2,4-triazol-4-yl) methyl] [1,1′-biphenyl]-2-carboxylic acid;

4′-[(3-isobutyl-1-phenylmethyl-4,5-dihydro-5-oxo-1H-1,2,4-triazol-4-yl) methyl] [1,1′-biphenyl]-2-carboxylic acid;

4′-[[3-isobutyl-1-(2-phenylethyl)-4,5-dihydro-5-oxo-1H-1,2,4-triazol-4-yl] methyl] [1,1′-biphenyl]-2-carboxylic acid;

4′-[(3-isobutyl-1-benzoyl-4,5-dihydro-5-oxo-1H-1,2,4-triazol-4-yl) methyl] [1,1′-biphenyl]-2-carboxylic acid;

4′-[[3-isobutyl-1-(1-oxo-2-phenylethyl)-4,5-dihydro-5-oxo-1H-1,2,4-triazol-4-yl] methyl] [1,1′-biphenyl]-2-carboxylic acid;

4′-[[3-isobutyl-1-(1-oxopropyl)-4,5-dihydro-5-oxo-1H-1,2,4-triazol-4-yl] methyl] [1,1′-biphenyl]-2-carboxylic acid;

4′-[[3-isobutyl-1-(1-oxobutyl)-4,5-dihydro-5-oxo-1H-1,2,4-triazol-4-yl] methyl] [1,1′-biphenyl]-2-carboxylic acid;

4′-[[3-isobutyl-1-(1-oxopentyl)-4,5-dihydro-5-oxo-1H-1,2,4-triazol-4-yl] methyl] [1,1′-biphenyl]-2-carboxylic acid;

4′-[[3-isobutyl-1-(2-butenyl)-4,5-dihydro-5-oxo-1H-1,2,4-triazole-4-yl] methyl] [1,1′-biphenyl]-2-carboxylic acid;

4′-[[3-isobutyl-1-(3-butenyl)-4,5-dihydro-5-oxo-1H-1,2,4-triazole-4-yl] methyl] [1,1′-biphenyl-2-carboxylic acid;

4′-[[3-isobutyl-1-(2-butynyl)-4,5-dihydro-5-oxo-1H-1,2,4-triazole-4-yl] methyl] [1,1′-biphenyl]-2-carboxylic acid;

4′-[[3-isobutyl-1-(3-butynyl)-4,5-dihydro-5-oxo-1H-1,2,4-triazole-4-yl] methyl] [1,1′-biphenyl]-2-carboxylic acid;

4′-[(3-tertbutyl-1-ethyl-4,5-dihydro-5-oxo-1H-1,2,4-triazol-4-yl) methyl] [1,1′-biphenyl]-2-carboxylic acid;

4′-[(3-tertbutyl-1-propyl-4,5-dihydro-5-oxo-1H,2,4-triazol-4-yl) methyl] [1,1′-biphenyl]-2-carboxylic acid;

4′-[(3-tertbutyl-1-isopropyl-4,5-dihydro-5-oxo-1H-1,2,4-triazol-4-yl) methyl] [1,1′-biphenyl]-2-carboxylic acid;

4′-[(3-tertbutyl-1-butyl-4,5-dihydro-5-oxo-1H-1,2,4-triazol-4-yl) methyl] [1,1′-biphenyl]-2-carboxylic acid;

4′-[(3-tertbutyl-1-secbutyl-4,5-dihydro-5-oxo-1H-1,2,4-triazol-4-yl) methyl] [1,1′-biphenyl]-2-carboxylic acid;

4′-[(3-tertbutyl-1-isobutyl-4,5-dihydro-5-oxo-1H-1,2,4-triazol-4-yl) methyl] [1,1′-biphenyl]-2-carboxylic acid;

4′-[(1,3-ditertbutyl-4,5-dihydro-5-oxo-1H-1,2,4-triazol-4-yl) methyl] [1,1′-biphenyl]-2-carboxylic acid;

4′-[(3-tertbutyl-1-pentyl-4,5-dihydro-5-oxo-1H-1,2,4-triazol-4-yl) methyl] [1,1′-biphenyl]-2-carboxylic acid;

4′-[(3-tertbutyl-1-isopentyl-4,5-dihydro-5-oxo-1H-1,2,4-triazol-4-yl) methyl] [1,1′-biphenyl]-2-carboxylic acid;

4′-[(3-tertbutyl-1-hexyl-4,5-dihydro-5-oxo-1H-1,2,4-triazol-4-yl) methyl] [1,1′-biphenyl]-2-carboxylic acid;

4′-[(3-tertbutyl-1-heptyl-4,5-dihydro-5-oxo-1H-1,2,4-triazol-4-yl) methyl] [1,1′-biphenyl]-2-carboxylic acid;

4′-[(3-tertbutyl-1-octyl-4,5-dihydro-5-oxo-1H-1,2,4-triazol-4-yl) methyl] [1,1′-biphenyl]-2-carboxylic acid;

4′-[(3-tertbutyl-1-cyclohexyl-4,5-dihydro-5-oxo-1H-1,2,4-triazol-4-yl) methyl] [1,1′-biphenyl]-2-carboxylic acid;

4′-[(3-tertbutyl-1-cyclohexylmethyl-4,5-dihydro-5-oxo-1H-1,2,4-triazol-4-yl) methyl] [1,1′-biphenyl]-2-carboxylic acid;

4′-[[3-tertbutyl-1-(2-cyclohexylethyl)-4,5-dihydro-5-oxo-1H-1,2,4-triazol-4-yl] methyl] [1,1′-biphenyl]-2-carboxylic acid;

4′-[(3-tertbutyl-1-cyclohexanoyl-4,5-dihydro-5-oxo-1H-1,2,4-triazol-4-yl) methyl] [1,1′-biphenyl]-2-carboxylic acid;

4′-[[3-tertbutyl-1-(1-oxo-2-cyclohexylethyl)-4,5-dihydro-5-oxo-1H-1,2,4-triazol-4-yl] methyl] [1,1′-biphenyl]-2-carboxylic acid;

4′-[(3-tertbutyl-1-phenyl-4,5-dihydro-5-oxo-1H-1,2,4-triazol-4-yl) methyl] [1,1′-biphenyl]-2-carboxylic acid;

4′-[(3-tertbutyl-1-phenylmethyl-4,5-dihydro-5-oxo-1H-1,2,4-triazol-4-yl) methyl] [1,1′-biphenyl]-2-carboxylic acid;

4′-[[3-tertbutyl-1-(2-phenylethyl)-4,5-dihydro-5-oxo-1H-1,2,4-triazol-4-yl] methyl] [1,1′-biphenyl]-2-carboxylic acid;

4′-[(3-tertbutyl-1-benzoyl-4,5-dihydro-5-oxo-1H-1,2,4-triazol-4-yl) methyl] [1,1′-biphenyl]-2-carboxylic acid;

4′-[[3-tertbutyl-1- (1-oxo-2-phenylethyl)-4,5-dihydro-5-oxo-1H-1,2,4-triazol-4-yl] methyl] [1,1′-biphenyl]-2-carboxylic acid;

4′-[[3-tertbutyl-1-(1-oxopropyl)-4,5-dihydro-5-oxo-1H-1,2,4-triazol-4-yl) methyl] [1,1′-biphenyl]-2-carboxylic acid;

4′-[[3-tertbutyl-1-(1-oxobutyl)-4,5-dihydro-5-oxo-1H-1,2,4-triazol-4-yl] methyl] [1,1′-biphenyl]-2-carboxylic acid;

4′-[[3-tertbutyl-1-(1-oxopentyl)-4,5-dihydro-5-oxo-1H-1,2,4-triazol-4-yl] methyl] [1,1′-biphenyl]-2-carboxylic acid;

4′-[[3-tertbutyl-1-(2-butenyl)-4,5-dihydro-5-oxo-1H-1,2,4-triazole-4-yl] methyl] [1,1′-biphenyl]-2-carboxylic acid;

4′-[[3-tertbutyl-1-(3-butenyl)-4,5-dihydro-5-oxo-1H-l,2,4-triazole-4-yl] methyl] [1,1′-biphenyl]-2-carboxylic acid;

4′-[[3-tertbutyl-1-(2-butynyl)-4,5-dihydro-5-oxo-1H-1,2,4-triazole-4-yl] methyl] [1,1′-biphenyl]-2-carboxylic acid;

4′-[[3-tertbutyl-1-(3-butynyl)-4,5-dihydro-5-oxo-1H-1,2,4-triazole-4-yl] methyl] [1,1′-biphenyl]-2-carboxylic acid;

4′-[(3-pentyl-1-ethyl-4,5-dihydro-5-oxo-1H-1,2,4-triazol-4-yl) methyl] [1,1′-biphenyl]-2-carboxylic acid;

4′-[(3-pentyl-1-propyl-4,5-dihydro-5-oxo-1H-1,2,4-triazol-4-yl) methyl] [1,1′-biphenyl]-2-carboxylic acid;

4′-[(3-pentyl-1-isopropyl-4,5-dihydro-5-oxo-1H-1,2,4-triazol-4-yl) methyl] [1,1′-biphenyl]-2-carboxylic acid;

4′-[(3-pentyl-1-butyl-4,5-dihydro-5-oxo-1H-1,2,4-triazol-4-yl) methyl] [1,1′-biphenyl]-2-carboxylic acid;

4′-[(3-pentyl-1-secbutyl-4,5-dihydro-5-oxo-1H-1,2,4-triazol-4-yl) methyl] [1,1′-biphenyl]-2-carboxylic acid;

4′-[(3-pentyl-1-isobutyl-4,5-dihydro-5-oxo-1H-1,2,4-triazol-4-yl) methyl] [1,1′-biphenyl]-2-carboxylic acid;

4′-[(3-pentyl-1-tertbutyl-4,5-dihydro-5-oxo-1H-1,2,4-triazol-4-yl) methyl] [1,1′-biphenyl]-2-carboxylic acid;

4′-[(1,3-dipentyl-4,5-dihydro-5-oxo-1H-1,2,4-triazol-4-yl) methyl] [1,1′-biphenyl]-2-carboxylic acid;

4′-[(3-pentyl-1-isopentyl-4,5-dihydro-5-oxo-1H-1,2,4-triazol-4-yl) methyl] [1,1′-biphenyl]-2-carboxylic acid;

4′-[(3-pentyl-1-hexyl-4,5-dihydro-5-oxo-1H-1,2,4-triazol-4-yl) methyl] [1,1′-biphenyl]-2-carboxylic acid;

4′-[(3-pentyl-1-heptyl-4,5-dihydro-5-oxo-1H-1,2,4-triazol-4-yl) methyl] [1,1′-biphenyl]-2-carboxylic acid;

4′-[(3-pentyl-1-octyl-4,5-dihydro-5-oxo-1H-1,2,4-triazol-4-yl) methyl] [1,1′-biphenyl]-2-carboxylic acid;

4′-[(3-pentyl-1-cyclohexyl-4,5-dihydro-5-oxo-1H-1,2,4-triazol-4-yl) methyl] [1,1′-biphenyl]-2-carboxylic acid;

4′-[(3-pentyl-1-cyclohexylmethyl-4,5-dihydro-5-oxo-1H-1,2,4-triazol-4-yl) methyl] [1,1′-biphenyll-2-carboxylic acid;

4′-[[3-pentyl-1-(2-cyclohexylethyl)-4,5-dihydro-5-oxo-1H-1,2,4-triazol-4-yl] methyl] [1,1′-biphenyl]-2-carboxylic acid;

4′-[(3-pentyl-1-cyclohexanoyl-4,5-dihydro-5-oxo-1H-l,2,4-triazol-4-yl) methyl] [1,1′-biphenyl]-2-carboxylic acid;

4′-([3-pentyl-1-(1-oxo-2-cyclohexylethyl)-4,5-dihydro-5-oxo-1H-1,2,4-triazol-4-yl] methyl] [1,1′-biphenyl]-2-carboxylic acid;

4′-[(3-pentyl-1-phenyl-4,5-dihydro-5-oxo-1H-1,2,4-triazol-4-yl) methyl] [1,1′-biphenyl]-2-carboxylic acid;

4′-((3-pentyl-1-phenylmethyl-4,5-dihydro-5-oxo-1H-1,2,4-triazol-4-yl) methyl] [1,1′-biphenyl]-2-carboxylic acid;

4′-[[3-pentyl-1-(2-phenylethyl)-4,5-dihydro-5-oxo-1H-1,2,4-triazol-4-yl] methyl] [1,1′-biphenyl]-2-carboxylic acid;

4′-[(3-pentyl-1-benzoyl-4,5-dihydro-5-oxo-1H-1,2,4-triazol-4-yl) methyl] [1,1′-biphenyl]-2-carboxylic acid;

4′-[[3-pentyl-1-(1-oxo-2-phenylethyl)-4,5-dihydro-5-oxo-1H-1,2,4-triazol-4-yl] methyl] [1,1′-biphenyl]-2-carboxylic acid;

4′-[[3-pentyl-1-(1-oxopropyl)-4,5-dihydro-5-oxo-1H-1,2,4-triazol-4-yl] methyl] [1,1′-biphenyl]-2-carboxylic acid;

4′-[[3-pentyl-1-(1-oxobutyl)-4,5-dihydro-5-oxo-1H-1,2,4-triazol-4-yl] methyl] [1,1′-biphenyl]-2-carboxylic acid;

4′-[[3-pentyl-1-(l-oxopentyl)-4,5-dihydro-5-oxo-1H-1,2,4-triazol-4-yl] methyl] [1,1′-biphenyl]-2-carboxylic acid;

4′-[[3-pentyl-1-(2-butenyl)-4,5-dihydro-5-oxo-1H-1,2,4-triazole-4-yl] methyl] [1,1′-biphenyl]-2-carboxylic acid;

4′-[[3-pentyl-1-(3-butenyl)-4,5-dihydro-5-oxo-1H-1,2,4-triazole-4-yl] methyl] [1,1′-biphenyl]-2-carboxylic acid;

4′-[[3-pentyl-1-(2-butynyl)-4,5-dihydro-5-oxo-1H-1,2,4-triazole-4-yl] methyl] [1,1′-biphenyl]-2-carboxylic acid;

4′-[[3-pentyl-1-(3-butynyl)-4,5-dihydro-5-oxo-1H-1,2,4-triazole-4-yl] methyl] [1,1′-biphenyl]-2-carboxylic acid;

4′-[(3-isopentyl-1-ethyl-4,5-dihydro-5-oxo-1H-1,2,4-triazol-4-yl) methyl] [1,1′-biphenyl]-2-carboxylic acid;

4′-[(3-isopentyl-1-propyl-4,5-dihydro-5-oxo-1H-1,2,4-triazol-4-yl) methyl] [1,1′-biphenyl]-2-carboxylic acid;

4′-[(3-isopentyl-1-isopropyl-4,5-dihydro-5-oxo-1H-1,2,4-triazol-4-yl) methyl] [1,1′-biphenyl]-2-carboxylic acid;

4′-[(3-isopentyl-1-butyl-4,5-dihydro-5-oxo-1H-1,2,4-triazol-4-yl) methyl] [1,1′-biphenyl]-2-carboxylic acid;

4′-[(3-isopentyl-1-secbutyl-4,5-dihydro-5-oxo-1H-1,2,4-triazol-4-yl) methyl] [1,1′-biphenyl]-2-carboxylic acid;

4′-[(3-isopentyl-1-isobutyl-4,5-dihydro-5-oxo-1H-1,2,4-triazol-4-yl) methyl] [1,1′-biphenyl]-2-carboxylic acid;

4′-[(3-isopentyl-1-tertbutyl-4,5-dihydro-5-oxo-1H-1,2,4-triazol-4-yl) methyl] [1,1′-biphenyl]-2-carboxylic acid;

4′-[(3-isopentyl-1-pentyl-4,5-dihydro-5-oxo-1H-1,2,4-triazol-4-yl) methyl] [1,1′-biphenyl]-2-carboxylic acid;

4′-[(1,3-diisopentyl-4,5-dihydro-5-oxo-1H-1,2,4-triazol-4-yl) methyl] [1,1′-biphenyl]-2-carboxylic acid;

4′-[(3-isopentyl-1-hexyl-4,5-dihydro-5-oxo-1H-1,2,4-triazol-4-yl) methyl] [1,1′-biphenyl]-2-carboxylic acid;

4′-[(3-isopentyl-1-heptyl-4,5-dihydro-5-oxo-1H-1,2,4-triazol-4-yl) methyl] [1,1′-biphenyl]-2-carboxylic acid;

4′-[(3-isopentyl-1-octyl-4,5-dihydro-5-oxo-1H-1,2,4-triazol-4-yl) methyl] [1,1′-biphenyl]-2-carboxylic acid;

4′-[(3-isopentyl-1-cyclohexyl-4,5-dihydro-5-oxo-1H-1,2,4-triazol-4-yl) methyl] [1,1′-biphenyl]-2-carboxylic acid;

4′-[(3-isopentyl-1-cyclohexylmethyl-4,5-dihydro-5-oxo-1H-1,2,4-triazol-4-yl) methyl] [1,1′-biphenyl]-2-carboxylic acid;

4′-[[3-isopentyl-1-(2-cyclohexylethyl)-4,5-dihydro-5-oxo-1H-1,2,4-triazol-4-yl] methyl] [1,1′-biphenyl]-2-carboxylic acid;

4′-[(3-isopentyl-1-cyclohexanoyl-4,5-dihydro-5-oxo-1H-1,2,4-triazol-4-yl) methyl] [1,1′-biphenyl]-2-carboxylic acid;

4′-[[3-isopentyl-1-(1-oxo-2-cyclohexylethyl)-4,5-dihydro-5-oxo-1H-1,2,4-triazol-4-yl] methyl] [1,1′-biphenyl]-2-carboxylic acid;

4′-[(3-isopentyl-1-phenyl-4,5-dihydro-5-oxo-1H-1,2,4-triazol-4-yl) methyl] [1,1′-biphenyl]-2-carboxylic acid;

4′-[(3-isopentyl-1-phenylmethyl-4,5-dihydro-5-oxo-1H-1,2,4-triazol-4-yl) methyl] [1,1′-biphenyl]-2-carboxylic acid;

4′-[[3-isopentyl-1-(2-phenylethyl)-4,5-dihydro-5-oxo-1H-1,2,4-triazol-4-yl] methyl] [1,1′-biphenyl]-2-carboxylic acid;

4′-[(3-isopentyl-1-benzoyl-4,5-dihydro-5-oxo-1H-1,2,4-triazol-4-yl) methyl] [1,1′-biphenyl]-2-carboxylic acid;

4′-[[3-isopentyl-1-(1-oxo-2-phenylethyl)-4,5-dihydro-5-oxo-1H-1,2,4-triazol-4-yl] methyl] [1,1′-biphenyl]-2-carboxylic acid;

4′-[[3-isopentyl-1-(1-oxopropyl)-4,5-dihydro-5-oxo-1H-1,2,4-triazol-4-yl] methyl] [1,1′-biphenyl]-2-carboxylic acid;

4′-[[3-isopentyl-1-(1-oxobutyl)-4,5-dihydro-5-oxo-1H-1,2,4-triazol-4-yl] methyl] [1,1′-biphenyl]-2-carboxylic acid;

4′-[[3-isopentyl-1-(1-oxopentyl)-4,5-dihydro-5-oxo-1H-1,2,4-triazol-4-yl] methyl] [1,1′-biphenyl]-2-carboxylic acid;

4′-[[3-isopentyl-1-(2-butenyl)-4,5-dihydro-5-oxo-1H-1,2,4-triazole-4-yl] methyl] [1,1′-biphenyl]-2-carboxylic acid;

4′-[[3-isopentyl-1-(3-butenyl)-4,5-dihydro-5-oxo-1H-1,2,4-triazole-4-yl] methyl] [1,1′-biphenyl]-2-carboxylic acid;

4′-[[3-isopentyl-1-(2-butynyl)-4,5-dihydro-5-oxo-1H-1,2,4-triazole-4-yl] methyl] [1,1′-biphenyl]-2-carboxylic acid;

4′-[[3-isopentyl-1-(3-butynyl)-4,5-dihydro-5-oxo-1H-1,2,4-triazole-4-yl] methyl] [1,1′-biphenyl]-2-carboxylic acid;

4′-[(3-hexyl-1-ethyl-4,5-dihydro-5-oxo-1H-1,2,4-triazol-4-yl) methyl] [1,1′-biphenyl]-2-carboxylic acid;

4-[(3-hexyl-1-propyl-4,5-dihydro-5-oxo-1H-1,2,4-triazol-4-yl) methyl] [1,1′-biphenyl]-2-carboxylic acid;

4′-[(3-hexyl-1-isopropyl-4,5-dihydro-5-oxo-1H-1,2,4-triazol-4-yl) methyl] [1,1′-biphenyl]-2-carboxylic acid;

4′-[(1,3-hexyl-4,5-dihydro-5-oxo-1H-1,2,4-triazol-4-yl) methyl] [1,1′-biphenyl]-2-carboxylic acid;

4′-[(3-hexyl-1-secbutyl-4,5-dihydro-5-oxo-1H-1,2,4-triazol-4-yl) methyl] [1,1′-biphenyl]-2-carboxylic acid;

4′-[(3-hexyl-1-isobutyl-4,5-dihydro-5-oxo-1H-1,2,4-triazol-4-yl) methyl] [1,1′-biphenyl]-2-carboxylic acid;

4′-[(3-hexyl-1-tertbutyl-4,5-dihydro-5-oxo-1H-1,2,4-triazol-4-yl) methyl] [1,1′-biphenyl]-2-carboxylic acid;

4′-[(3-hexyl-1-pentyl-4,5-dihydro-5-oxo-1H-1,2,4-triazol-4-yl) methyl] [1,1′-biphenyl]-2-carboxylic acid;

4′-[(3-hexyl-1-isopentyl-4,5-dihydro-5-oxo-1H-1,2,4-triazol-4-yl) methyl] [1,1′-biphenyl]-2-carboxylic acid;

4′-[(3-hexyl-1-butyl-4,5-dihydro-5-oxo-1H-1,2,4-triazol-4-yl) methyl] [1,1′-biphenyl]-2-carboxylic acid;

4′-[(3-hexyl-1-heptyl-4,5-dihydro-5-oxo-1H-1,2,4-triazol-4-yl) methyl] [1,1′-biphenyl]-2-carboxylic acid;

4′-[(3-hexyl-1-octyl-4,5-dihydro-5-oxo-1H-1,2,4-triazol-4-yl) methyl] [1,1′-biphenyl]-2-carboxylic acid;

4′-[(3-hexyl-1-cyclohexyl-4,5-dihydro-5-oxo-1H-1,2,4-triazol-4-yl) methyl] [1,1′-biphenyl]-2-carboxylic acid;

4′-[(3-hexyl-1-cyclohexylmethyl-4,5-dihydro-5-oxo-1H-1,2,4-triazol-4-yl) methyl] [1,1′-biphenyl]-2-carboxylic acid;

4′-[[3-hexyl-1-(2-cyclohexylethyl)-4,5-dihydro-5-oxo-1H-1,2,4-triazol-4-yl] methyl] [1,1′-biphenyl]-2-carboxylic acid;

4′-[(3-hexyl-1-cyclohexanoyl-4,5-dihydro-5-oxo-1H-1,2,4-triazol-4-yl) methyl] [1,1′-biphenyl]-2-carboxylic acid;

4′-[[3-hexyl-1-(1-oxo-2-cyclohexylethyl)-4,5-dihydro-5-oxo-1H-1,2,4-triazol-4-yl] methyl] [1,1′-biphenyl]-2-carboxylic acid;

4′-[(3-hexyl-1-phenyl-4,5-dihydro-5-oxo-1H-1,2,4-triazol-4-yl) methyl] [1,1′-biphenyl]-2-carboxylic acid;

4′-[(3-hexyl-1-phenylmethyl-4,5-dihydro-5-oxo-1H-1,2,4-triazol-4-yl) methyl] [1,1′-biphenyl]-2-carboxylic acid;

4′-[[3-hexyl-1-(2-phenylethyl)-4,5-dihydro-5-oxo-1H-1,2,4-triazol-4-yl] methyl] [1,1′-biphenyl]-2-carboxylic acid;

4′-[(3-hexyl-1-benzoyl-4,5-dihydro-5-oxo-1H-1,2,4-triazol-4-yl) methyl] [1,1′-biphenyl]-2-carboxylic acid;

4′-[[3-hexyl-1-(1-oxo-2-phenylethyl)-4,5-dihydro-5-oxo-1H-1,2,4-triazol-4-yl] methyl] [1,1′-biphenyl]-2-carboxylic acid;

4′-[[3-hexyl-1-(1-oxopropyl)-4,5-dihydro-5-oxo-1H-1,2,4-triazol-4-yl] methyl] [1,1′-biphenyl]-2-carboxylic acid;

4′-[[3-hexyl-1-(1-oxobutyl)-4,5-dihydro-5-oxo-1H-1,2,4-triazol-4-yl] methyl] [1,1′-biphenyl]-2-carboxylic acid;

4′-[[3-hexyl-1-(1-oxopentyl)-4,5-dihydro-5-oxo-1H-1,2,4-triazol-4-yl] methyl] [1,1′-biphenyl]-2-carboxylic acid;

4′-[[3-hexyl-1-(2-butenyl)-4,5-dihydro-5-oxo-1H-1,2,4-triazole-4-yl] methyl] [1,1′-biphenyl]-2-carboxylic acid;

4′-[[3-hexyl-1-(3-butenyl)-4,5-dihydro-5-oxo-1H-1,2,4-triazole-4-′yl] methyl] [1,1′-biphenyl]-2-carboxylic acid;

4′-[[3-hexyl-1-(2-butynyl)-4,5-dihydro-5-oxo-1H-1,2,4-triazole-4-yl] methyl] [1,1′-biphenyl]-2-carboxylic acid;

4′-[[3-hexyl-1-(3-butynyl)-4,5-dihydro-5-oxo-1H-1,2,4-triazole-4-yl] methyl] [1,1′-biphenyl]-2-carboxylic acid;

4′-[(3-heptyl-1-ethyl-4,5-dihydro-5-oxo-1H-1,2,4-triazol-4-yl) methyl] [1,1′-biphenyl]-2-carboxylic acid;

4′-[(3-heptyl-1-propyl-4,5-dihydro-5-oxo-1H-1,2,4-triazol-4-yl) methyl] [1,1′-biphenyl]-2-carboxylic acid;

4′-[(3-heptyl-1-isopropyl-4,5-dihydro-5-oxo-1H-1,2,4-triazol-4-yl) methyl] [1,1′-biphenyl]-2-carboxylic acid;

4′-[(3-heptyl-1-butyl-4,5-dihydro-5-oxo-1H-1,2,4-triazol-4-yl) methyl] [1,1′-biphenyl]-2-carboxylic acid;

4′-[(3-heptyl-1-secbutyl-4,5-dihydro-5-oxo-1H-1,2,4-triazol-4-yl) methyl] [1,1′-biphenyl]-2-carboxylic acid;

4′-[(3-heptyl-1-isobutyl-4,5-dihydro-5-oxo-1H-1,2,4-triazol-4-yl) methyl] [1,1′-biphenyl]-2-carboxylic acid;

4′-[(3-heptyl-1-tertbutyl-4,5-dihydro-5-oxo-1H-1,2,4-triazol-4-yl) methyl] [1,1′-biphenyl]-2-carboxylic acid;

4′-[(3-heptyl-1-pentyl-4,5-dihydro-5-oxo-1H-1,2,4-triazol-4-yl) methyl] [1,1′-biphenyl]-2-carboxylic acid;

4′-[(3-heptyl-1-isopentyl-4,5-dihydro-5-oxo-1H-l,2,4-triazol-4-yl) methyl] [1,1′-biphenyl]-2-carboxylic acid;

4′-[(3-heptyl-1-hexyl-4,S-dihydro-5-oxo-1H-1,2,4-triazol-4-yl) methyl] [1,1′-biphenyl]-2-carboxylic acid;

4′-[(1,3-diheptyl-4,5-dihydro-5-oxo-1H-1,2,4-triazol-4-yl) methyl] [1,1′-biphenyl]-2-carboxylic acid;

4′-[(3-heptyl-1-octyl-4,5-dihydro-5-oxo-1H-1,2,4-triazol-4-yl) methyl] [1,1′-biphenyl]-2-carboxylic acid;

4′-[(3-heptyl-1-cyclohexyl-4,5-dihydro-5-oxo-1H-1,2,4-triazol-4-yl) methyl] [1,1′-biphenyl]-2-carboxylic acid;

4′-[(3-heptyl-1-cyclohexylmethyl-4,5-dihydro-5-oxo-1H-1,2,4-triazol-4-yl) methyl] [1,1′-biphenyl]-2-carboxylic acid;

4′-[[3-heptyl-1-(2-cyclohexylethyl)-4,5-dihydro-5-oxo-1H-1,2,4-triazol-4-yl] methyl] [1,1′-biphenyl]-2-carboxylic acid;

4′-[(3-heptyl-1-cyclohexanoyl-4,5-dihydro-5-oxo-1H-1,2,4-triazol-4-yl) methyl] [1,1′-biphenyl]-2-carboxylic acid;

4′-[[3-heptyl-1-(1-oxo-2-cyclohexylethyl)-4,5-dihydro-5-oxo-1H-1,2,4-triazol-4-yl] methyl] [1,1′-biphenyl]-2-carboxylic acid;

4′-[(3-heptyl-1-phenyl-4,5-dihydro-5-oxo-1H-1,2,4-triazol-4-yl) methyl] [1,1′-biphenyl]-2-carboxylic acid;

4′-[(3-heptyl-1-phenylmethyl-4,5-dihydro-5-oxo-1H-1,2,4-triazol-4-yl) methyl] [1,1′-biphenyl]-2-carboxylic acid;

4′-[[3-heptyl-1-(2-phenylethyl)-4, 5-dihydro-5-oxo-1H-1,2,4-triazol-4-yl] methyl] [1,1′-biphenyl]-2-carboxylic acid;

4′-[(3-heptyl-1-benzoyl-4,5-dihydro-5-oxo-1H-1,2,4-triazol-4-yl) methyl] [1,1′-biphenyl]-2-carboxylic acid;

4′-[[3-heptyl-1-(1-oxo-2-phenylethyl)-4,5-dihydro-5-oxo-1H-1,2,4-triazol-4-yl] methyl] [1,1′-biphenyl]-2-carboxylic acid;

4′-[[3-heptyl-1-(l-oxopropyl)-4,5-dihydro-5-oxo-1H-1,2,4-triazol-4-yl] methyl] [1,1′-biphenyl]-2-carboxylic acid;

4′-[[3-heptyl-1-(1-oxobutyl)-4,5-dihydro-5-oxo-1H-1,2,4-triazol-4-yl] methyl] [1,1′-biphenyl]-2-carboxylic acid;

4′-[[3-heptyl-1-(1-oxopentyl)-4,5-dihydro-5-oxo-1H-1,2,4-triazol-4-yl] methyl] [1,1′-biphenyl]-2-carboxylic acid;

4′-[[3-heptyl-1-(2-butenyl)-4,5-dihydro-5-oxo-1H-1,2,4-triazole-4-yl] methyl] [1,1′-biphenyl]-2-carboxylic acid;

4′-[[3-heptyl-1-(3-butenyl)-4,5-dihydro-5-oxo-1H-1,2,4-triazole-4-yl] methyl] [1,1′-biphenyl]-2-carboxylic acid;

4′-[[3-heptyl-1-(2-butynyl)-4,5-dihydro-5-oxo-1H-1,2,4-triazole-4-yl] methyl] [1,1′-biphenyl]-2-carboxylic acid;

4′-[[3-heptyl-1-(3-butynyl)-4,5-dihydro-5-oxo-1H-1,2,4-triazole-4-yl] methyl] [1,1′-biphenyl]-2-carboxylic acid;

4′-[(3-octyl-1-ethyl-4,5-dihydro-5-oxo-1H-1,2,4-triazol-4-yl) methyl] [1,1′-biphenyl]-2-carboxylic acid;

4′-[(3-octyl-1-propyl-4,5-dihydro-5-oxo-1H-1,2,4-triazol-4-yl) methyl] [1,1′-biphenyl]-2-carboxylic acid;

4′-[(3-octyl-1-isopropyl-4,5-dihydro-5-oxo-1H-1,2,4-triazol-4-yl) methyl] [1,1′-biphenyl]-2-carboxylic acid;

4′-[(3-octyl-1-butyl-4,5-dihydro-5-oxo-1H-1,2,4-triazol-4-yl) methyl] [1,1′-biphenyl]-2-carboxylic acid;

4′-[(3-octyl-1-secbutyl-4,5-dihydro-5-oxo-1H-1,2,4-triazol-4-yl) methyl] [1,1′-biphenyl]-2-carboxylic acid;

4′-[(3-octyl-1-isobutyl-4,5-dihydro-5-oxo-1H-1,2,4-triazol-4-yl) methyl] [1,1′-biphenyl]-2-carboxylic acid;

4′-[(3-octyl-1-tertbutyl-4,5-dihydro-5-oxo-1H-1,2,4-triazol-4-yl) methyl] [1,1′-biphenyl]-2-carboxylic acid;

4′-[(3-octyl-1-pentyl-4,5-dihydro-5-oxo-1H-1,2,4-triazol-4-yl) methyl] [1,1′-biphenyl]-2-carboxylic acid;

4′-[(3-octyl-1-isopentyl-4,5-dihydro-5-oxo-1H-1,2,4-triazol-4-yl) methyl] [1,1′-biphenyl]-2-carboxylic acid;

4′-[(3-octyl-1-hexyl-4,5-dihydro-5-oxo-1H-1,2,4-triazol-4-yl) methyl] [1,1′-biphenyl]-2-carboxylic acid;

4′-[(3-octyl-1-heptyl-4,5-dihydro-5-oxo-1H-1,2,4-triazol-4-yl) methyl] [1,1′-biphenyl]-2-carboxylic acid;

4′-[(1,3-dioctyl-4,5-dihydro-5-oxo-1H-1,2,4-triazol-4-yl) methyl] [1,1′-biphenyl]-2-carboxylic acid;

4′-[(3-octyl-1-cyclohexyl-4,5-dihydro-5-oxo-1H-1,2,4-triazol-4-yl) methyl] [1,1′-biphenyl]-2-carboxylic acid;

4′-[(3-octyl-1-cyclohexylmethyl-4,5-dihydro-5-oxo-1H-1,2,4-triazol-4-yl) methyl] [1,1′-biphenyl]-2-carboxylic acid;

4′-[[3-octyl-1-(2-cyclohexylethyl)-4,5-dihydro-5-oxo-1H-1,2,4-triazol-4-yl] methyl] [1,1′-biphenyl]-2-carboxylic acid;

4′-[(3-octyl-1-cyclohexanoyl-4,5-dihydro-5-oxo-1H-1,2,4-triazol-4-yl) methyl] [1,1′-biphenyl]-2-carboxylic acid;

4′-[[3-octyl-1-(1-oxo-2-cyclohexylethyl)-4,5-dihydro-5-oxo-1H-1,2,4-triazol-4-yl] methyl] [1,1′-biphenyl]-2-carboxylic acid;

4′-[(3-octyl-1-phenyl-4,5-dihydro-5-oxo-1H-1,2,4-triazol-4-yl) methyl] [1,1′-biphenyl]-2-carboxylic acid;

4′-[(3-octyl-1-phenylmethyl-4, 5-dihydro-5-oxo-1H-1,2,4-triazol-4-yl) methyl] [1,1′-biphenyl]-2-carboxylic acid;

4′-[[3-octyl-1-(2-phenylethyl)-4,5-dihydro-5-oxo-1H-1,2,4-triazol-4-yl] methyl] [1,1′-biphenyl]-2-carboxylic acid;

4′-[(3-octyl-1-benzoyl-4,5-dihydro-5-oxo-1H-1,2,4-triazol-4-yl) methyl] [1,1′-biphenyl]-2-carboxylic acid;

4′-[[3-octyl-1-(1-oxo-2-phenylethyl)-4,5-dihydro-5-oxo-1H-1,2,4-triazol-4-yl] methyl] [1,1′-biphenyl]-2-carboxylic acid;

4′-[[3-octyl-1-(1-oxopropyl)-4,5-dihydro-5-oxo-1H-1,2,4-triazol-4-yl] methyl] [1,1′-biphenyl]-2-carboxylic acid;

4′-[[3-octyl-1-(1-oxobutyl)-4,5-dihydro-5-oxo-1H-1,2,4-triazol-4-yl] methyl] [1,1′-biphenyl]-2-carboxylic acid;

4′-[[3-octyl-1-(1-oxopentyl)-4,5-dihydro-5-oxo-1H-1,2,4-triazol-4-yl] methyl] [1,1′-biphenyl]-2-carboxylic acid;

4′-[[3-octyl-1-(2-butenyl)-4,5-dihydro-5-oxo-1H-1,2,4-triazole-4-yl] methyl] [1,1′-biphenyl]-2-carboxylic acid;

4′-[[3-octyl-1-(3-butenyl)-4,5-dihydro-5-oxo-1H-1,2,4-triazole-4-yl] methyl] [1,1′-biphenyl]-2-carboxylic acid;

4′-[[3-octyl-1-(2-butynyl)-4,5-dihydro-5-oxo-1H-1,2,4-triazole-4-yl] methyl] [1,1′-biphenyl]-2-carboxylic acid;

4′-[[3-octyl-1-(3-butynyl)-4,5-dihydro-5-oxo-1H-1,2,4-triazole-4-yl]methyl] [1,1′ -biphenyl]-2-carboxylic acid;

4′-[[3-(1-butenyl)-1-ethyl-4,5-dihydro-5-oxo-1H-1,2,4-triazol-4-yl] methyl] [1,1′-biphenyl]-2-carboxylic acid;

4′-[[3-(1-butenyl)-1-propyl-4,5-dihydro-5-oxo-1H-1,2,4-triazol-4-yl] methyl] [1,1′-biphenyl]-2-carboxylic acid;

4′-[[3-(1-butenyl) -1-isopropyl-4, 5-dihydro-5-oxo-1H-1,2,4-triazol-4-yl] methyl] [1,1′-biphenyl]-2-carboxylic acid;

4′-[[3-(1-butenyl)-1-butyl-4,5-dihydro-5-oxo-1H-1,2,4-triazol-4-yl] methyl] [1,1′-biphenyl]-2-carboxylic acid;

4′-[[3-(1-butenyl)-1-secbutyl-4,5-dihydro-5-oxo-1H-1,2,4-triazol-4-yl] methyl] [1,1′-biphenyl]-2-carboxylic acid;

4′-[[3-(1-butenyl)-1-isobutyl-4,5-dihydro-5-oxo-1H-1,2,4-triazol-4-yl] methyl] [1,1′-biphenyl]-2-carboxylic acid;

4′-[[3-(1-butenyl)-1-tertbutyl-4,5-dihydro-5-oxo-1H-1,2,4-triazol-4-yl] methyl] [1,1′-biphenyl]-2-carboxylic acid;

4′-[[3-(1-butenyl)-1-pentyl-4,5-dihydro-5-oxo-1H-1,2,4-triazol-4-yl] methyl] [1,1′-biphenyl]-2-carboxylic acid;

4′-[[3-(1-butenyl)-1-isopentyl-4,5-dihydro-5-oxo-1H-1,2,4-triazol-4-yl] methyl] [1,1′-biphenyl]-2-carboxylic acid;

4′-[(3-(1-butenyl)-1-hexyl-4,5-dihydro-5-oxo-1H-1,2,4-triazol-4-yl) methyl] [1,1′-biphenyl]-2-carboxylic acid;

4′-[(3-(1-butenyl)-1-heptyl-4,5-dihydro-5-oxo-1H-1,2,4-triazol-4-yl) methyl] [1,1′-biphenyl]-2-carboxylic acid;

4′-[(3-(1-butenyl)-1-octyl-4,5-dihydro-5-oxo-1H-1,2,4-triazol-4-yl) methyl] [1,1′-biphenyl]-2-carboxylic acid;

4′-[[3-(l-butenyl)-1-cyclohexyl-4,5-dihydro-5-oxo-1H-1,2,4-triazol-4-yl] methyl] [1,1′-biphenyl]-2-carboxylic acid;

4′-[[3-(1-butenyl)-1-cyclohexylmethyl-4,5-dihydro-5-oxo-1H-1,2,4-triazol-4-yl] methyl] [1,1′-biphenyl]-2-carboxylic acid;

4′-[[3-(1-butenyl)-1-(2-cyclohexylethyl)-4,5-dihydro-5-oxo-1H-1,2,4-triazol-4-yl] methyl] [1,1′-biphenyl]-2-carboxylic acid;

4′-[[3-(1-butenyl)-1-cyclohexanoyl-4,5-dihydro-5-oxo-1H-1,2,4-triazol-4-yl] methyl] [1,1′-biphenyl]-2-carboxylic acid;

4′-[[3-(1-butenyl)-1-(1-oxo-2-cyclohexylethyl)-4,5-dihydro-5-oxo-1H-1,2,4-triazol-4-yl] methyl] [1,1′-biphenyl]-2-carboxylic acid;

4′-[[3-(1-butenyl)-1-phenyl-4,5-dihydro-5-oxo-1H-1,2,4-30 triazol-4-yl] methyl] [1,1′-biphenyl]-2-carboxylic acid;

4′-[[3-(1-butenyl)-1-phenylmethyl-4,5-dihydro-5-oxo-1H-1,2,4-triazol-4-yl] methyl] [1,1′-biphenyl]-2-carboxylic acid;

4′-[[3-(1-butenyl)-1-(2-phenylethyl)-4,5-dihydro-5-oxo-1H-1,2,4-triazol-4-yl] methyl] [1,1′-biphenyl]-2-carboxylic acid;

4′-[[3-(1-butenyl)-1-benzoyl-4,5-dihydro-5-oxo-1H-1,2,4-triazol-4-yl] methyl] [1,1′-biphenyl]-2-carboxylic acid;

4′-[[3-(1-butenyl)-1-(1-oxo-2-phenylethyl)-4,5-dihydro-5-oxo-1H-1,2,4-triazol-4-yl] methyl] [1,1′-biphenyl]-2-carboxylic acid;

4′-[[3-(1-butenyl)-1-(1-oxopropyl)-4,5-dihydro-5-oxo-1H-5 1,2,4-triazol-4-yl] methyl] [1,1′-biphenyl]-2-carboxylic acid;

4′-[[3-(1-butenyl)-1-(1-oxobutyl)-4,5-dihydro-5-oxo-1H-1,2,4-triazol-4-yl] methyl] [1,1′-biphenyl]-2-carboxylic acid;

4′-[[3-(1-butenyl)-1-(1-oxopentyl)-4,5-dihydro-5-oxo-1H-1,2,4-triazol-4-yl] methyl] [1,1′-biphenyl]-2-carboxylic acid;

4′-[[3-(1-butenyl)-1-(2-butenyl)-4,5-dihydro-5-oxo-1H-1,2,4-triazole-4-yl] methyl] [1,1′-biphenyl]-2-carboxylic acid;

4′-[[3-(1-butenyl)-1-(3-butenyl)-4,5-dihydro-5-oxo-1H-1,2,4-triazole-4-yl] methyl] [1,1′-biphenyl]-2-carboxylic acid;

4′-[[3-(1-butenyl)-1-(2-butynyl)-4,5-dihydro-5-oxo-1H-20 1,2,4-triazole-4-yl] methyl] [1,1′-biphenyl]-2-carboxylic acid;

4′-[[3-(1-butenyl)-1-(3-butynyl)-4,5-dihydro-5-oxo-1H-1,2,4-triazole-4-yl] methyl] [1,1′-biphenyl]-2-carboxylic acid;

4′-[[3-(2-butenyl)-1-ethyl-4,5-dihydro-5-oxo-1H-1,2,4-triazol-4-yl] methyl] [1,1′-biphenyl]-2-carboxylic acid;

4′-[[3-(2-butenyl)-1-propyl-4,5-dihydro-5-oxo-1H-1,2,4-triazol-4-yl] methyl] [1,1′-biphenyl]-2-carboxylic acid;

4′-[[3-(2-butenyl)-1-isopropyl-4,5-dihydro-5-oxo-1H-1,2,4-3 triazol-4-yl] methyl] [1,1′-biphenyl]-2-carboxylic acid;

4′-[[3-(2-butenyl)-1-butyl-4,5-dihydro-5-oxo-1H-1,2,4-triazol-4-yl] methyl][1,1′-biphenyl]-2-carboxylic acid;

4′-[[3-(2-butenyl)-1-secbutyl-4,5-dihydro-5-oxo-1H-1,2,4-triazol-4-yl] methyl] [1,1′-biphenyl]-2-carboxylic acid;

4′-[[3-(2-buteoyl)-1-isobutyl-4,5-dihydro-5-oxo-1H-1,2,4-triazol-4-yl] methyl] [1,1′-biphenyl]-2-carboxylic acid;

4′-[[3-(2-butenyl)-1-tertbutyl-4,5-dihydro-5-oxo-1H-1,2,4-triazol-4-yl] methyl] [1,1′-biphenyl]-2-carboxylic acid;

4′-[[3-(2-butenyl)-1-pentyl-4,5-dihydro-5-oxo-1H-1,2,4-triazol-4-yl] methyl] [1,1′-biphenyl]-2-carboxylic acid;

4′-[[3-(2-butenyl)-1-isopentyl-4,5-dihydro-5-oxo-1H-1,2,4-triazol-4-yl] methyl] [1,1′-biphenyl]-2-carboxylic acid;

4′-[(3-(2-butenyl)-1-hexyl-4,5-dihydro-5-oxo-1H-1,2,4-triazol-4-yl) methyl] [1,1′-biphenyl]-2-carboxylic acid;

4′-[(3-(2-butenyl)-1-heptyl-4,5-dihydro-5-oxo-1H-1,2,4-triazol-4-yl) methyl] [1,1′-biphenyl]-2-carboxylic acid;

4′-[(3-(2-butenyl)-1-octyl-4,5-dihydro-5-oxo-1H-1,2,4-triazol-4-yl) methyl] [1,1′-biphenyl]-2-carboxylic acid;

41-[[3-(2-butenyl)-1-cyclohexyl-4,5-dihydro-5-oxo-1H-1,2,4-triazol-4-yl] methyl] [1,1′-biphenyl]-2-carboxylic acid;

4′-[[3-(2-butenyl)-1-cyclohexylmethyl-4,5-dihydro-5-oxo-1H-1,2,4-triazol-4-yl] methyl] [1,1′-biphenyl]-2-carboxylic acid;

4′-[[3-(2-butenyl)-1-(2-cyclohexylethyl)-4,5-dihydro-5-oxo-1H-1,2,4-triazol-4-yl] methyl] [1,1′-biphenyl]-2-carboxylic acid;

4′-[[3-(2-butenyl)-1-cyclohexanoyl-4,5-dihydro-5-oxo-1H-1,2,4-triazol-4-yl] methyl] [1,1′-biphenyl]-2-carboxylic acid;

4′-[[3-(2-butenyl)-1-(1-oxo-2-cyclohexylethyl)-4,5-dihydro-5-oxo-1H-1,2,4-triazol-4-yl] methyl] [1,1′-biphenyl]-2-carboxylic acid,

4′-[[3-(2-butenyl)-1-phenyl-4,5-dihydro-5-oxo-1H-1,2,4-triazol-4-yl] methyl] [1,1′-biphenyl]-2-carboxylic acid;

4′-[[3-(2-butenyl)-1-phenylmethyl-4,5-dihydro-5-oxo-1H-1,2,4-triazol-4-yl] methyl] [1,1′-biphenyl]-2-carboxylic acid;

4′-[[3-(2-butenyl)-1-(2-phenylethyl)-4,5-dihydro-5-oxo-1H-1,2,4-triazol-4-yl] methyl] [1,1′-biphenyl]-2-carboxylic acid;

4′-[[3-(2-butenyl)-1-(1-oxopropyl)-4,5-dihydro-5-oxo-1H-1,2,4-triazol-4-yl] methyl] [1,1′-biphenyl]-2-carboxylic acid;

4′-[[3-(2-butenyl)-1-(1-oxobutyl)-4,5-dihydro-5-oxo-1H-1,2,4-triazol-4-yl] methyl] [1,1′-biphenyl]-2-carboxylic acid;

4′-[[3-(2-butenyl)-1-(1-oxopentyl)-4,5-dihydro-5-oxo-1H-1,2,4-triazol-4-yl] methyl] [1,1′-biphenyl]-2-carboxylic acid;

4′-[[1,3-di(2-butenyl)-4,5-dihydro-5-oxo-1H-1,2,4-triazole-4-yl] methyl] [1,1′-biphenyl]-2-carboxylic acid;

4′-[[3-(2-butenyl)-1-(3-butenyl)-4,5-dihydro-5-oxo-1H-1,2,4-triazole-4-yl] methyl] [1,1′-biphenyll-2-carboxylic acid;

4′-[[3-(2-butenyl)-1-(2-butynyl)-4,5-dihydro-5-oxo-1H-1,2,4-triazole-4-yl] methyl] [1,1′-biphenyl]-2-carboxylic acid;

4′-[[3-(2-butenyl)-1-(3-butynyl)-4,5-dihydro-5-oxo-1H-1,2,4-triazole-4-yl] methyl] [1,1′-biphenyl]-2-carboxylic acid;

4′-[[3-(3-butenyl)-1-ethyl-4,5-dihydro-5-oxo-1H-1,2,4-triazol-4-yl] methyl] [1,1′-biphenyl]-2-carboxylic acid;

4′-[[3-(3-butenyl)-1-propyl-4,5-dihydro-5-oxo-1H-1,2,4-triazol-4-yl] methyl] [1,1′-biphenyl]-2-carboxylic acid;

4′-[[3-(3-butenyl)-1-isopropyl-4,5-dihydro-5-oxo-1H-1,2,4-triazol-4-yl] methyl] [1,1′-biphenyl]-2-carboxylic acid;

4′-[[3-(3-butenyl)-1-butyl-4,5-dihydro-5-oxo-1H-1,2,4-triazol-4-yl] methyl] [1,1′-biphenyl]-2-carboxylic acid;

4′-[[3-(3-butenyl)-1-secbutyl-4,5-dihydro-5-oxo-1H-1,2,4-triazol-4-yl] methyl] [1,1′-biphenyl]-2-carboxylic acid;

4′-[[3-(3-butenyl)-1-isobutyl-4,5-dihydro-5-oxo-1H-1,2,4-triazol-4-yl] methyl] [1,1′-biphenyl]-2-carboxylic acid;

4′-[[3-(3-butenyl)-1-tertbutyl-4,5-dihydro-5-oxo-1H-1,2,4-triazol-4-yl] methyl] [1,1′-biphenyl]-2-carboxylic acid;

4′-[[3-(3-butenyl)-1-pentyl-4,5-dihydro-5-oxo-1H-1,2,4-triazol-4-yl] methyl] [1,1′-biphenyl]-2-carboxylic acid;

4′-[[3-(3-butenyl)-1-isopentyl-4,5-dihydro-5-oxo-1H-1,2,4-triazol-4-yl] methyl] [1,1′-biphenyl]-2-carboxylic acid;

4′-[(3-(3-butenyl)-1-hexyl-4,5-dihydro-5-oxo-1H-1,2,4-triazol-4-yl) methyl] [1,1′-biphenyl]-2-carboxylic acid;

4′-[(3-(3-butenyl)-1-heptyl-4,5-dihydro-5-oxo-1H-1,2,4-triazol-4-yl) methyl] [1,1′-biphenyl]-2-carboxylic acid;

4′-[(3-(3-butenyl)-1-octyl-4,5-dihydro-5-oxo-1H-1,2,4-triazol-4-yl) methyl] [1,1′-biphenyl]-2-carboxylic acid;

4′-[[3-(3-butenyl)-1-cyclohexyl-4,5-dihydro-5-oxo-1H-1,2,4-triazol-4-yl] methyl] [1,1′-biphenyl)-2-carboxylic acid;

4′-[[3-(3-butenyl)-1-cyclohexylmethyl-4,5-dihydro-5-oxo-1H-1,2,4-triazol-4-yl] methyl] [1,1′-biphenyl]-2-carboxylic acid;

4′-[[3-(3-butenyl)-1-(2-cyclohexylethyl)-4,5-dihydro-5-oxo-1H-1,2,4-triazol-4-yl] methyl] [1,1′-biphenyl]-2-carboxylic acid;

4′-[[3-(3-butenyl)-1-cyclohexanoyl-4,5-dihydro-5-oxo-1H-1,2,4-triazol-4-yl] methyl] [1,1′-biphenyl]-2-carboxylic acid;

4′-[[3-(3-butenyl)-1-(1-oxo-2-cyclohexylethyl)-4,5-dihydro-5-oxo-1H-1,2,4-triazol-4-yl] methyl] [1,1′-biphenyl]-2-carboxylic acid;

4′-[[3-(3-butenyl)-1-phenyl-4,5-dihydro-5-oxo-1H-1,2,4-triazol-4-yl] methyl] [1,1′-biphenyl]-2-carboxylic acid;

4′-[[3-(3-butenyl)-1-phenylmethyl-4,5-dihydro-5-oxo-1H-1,2,4-triazol-4-yl] methyl] [1,1′-biphenyl]-2-carboxylic acid;

4′-[[3-(3-butenyl)-1-(2-phenylethyl)-4,5-dihydro-5-oxo-1H-1,2,4-triazol-4-yl] methyl] [1,1′-biphenyl]-2-carboxylic acid;

4′-[[3-(3-butenyl)-1-benzoyl-4,5-dihydro-5-oxo-1H-1,2,4-triazol-4-yl] methyl] [1,1′-biphenyl]-2-carboxylic acid;

4′-[[3-(3-butenyl)-1-(1-oxo-2-phenylethyl)-4,5-dihydro-5-oxo-1H-1,2,4-triazol-4-yl] methyl] [1,1′-biphenyl]-2-carboxylic acid;

4′-[[3-(3-butenyl)-1-(1-oxopropyl)-4,5-dihydro-5-oxo-1H-1,2,4-triazol-4-yl] methyl] [1,1′-biphenyl]-2-carboxylic acid;

4′-[[3-(3-butenyl)-1-(1-oxobutyl)-4,5-dihydro-5-oxo-1H-1,2,4-triazol-4-yl] methyl] [1,1′-biphenyl]-2-carboxylic acid;

4′-[[3-(3-butenyl)-1-(1-oxopentyl)-4,5-dihydro-5-oxo-1H-1,2,4-triazol-4-yl] methyl] [1,1′-biphenyl]-2-carboxylic acid;

4′-[[3-(3-butenyl)-1-(2-butenyl)-4,5-dihydro-5-oxo-1H-1,2,4-triazole-4-yl] methyl] [1,1′-biphenyl]-2-carboxylic acid;

4′-[[1,3-di(3-butenyl)-4,5-dihydro-5-oxo-1H-1,2,4-triazole-4-yl] methyl] [1,1′-biphenyl]-2-carboxylic acid;

4′-[[3-(3-butenyl)-1-(2-butynyl)-4,5-dihydro-5-oxo-1H-1,2,4-triazole-4-yl] methyl] [1,1′-biphenyl]-2-carboxylic acid;

4′-[[3-(3-butenyl)-1-(3-butynyl)-4,5-dihydro-5-oxo-1H-1,2,4-triazole-4-yl] methyl] [1,1′-biphenyl]-2-carboxylic acid;

4′-[[3-(1-butynyl)-1-ethyl-4,5-dihydro-5-oxo-1H-1,2,4-triazol-4-yl] methyl] [1,1′-biphenyl]-2-carboxylic acid;

4′-[[3-(1-butynyl)-1-propyl-4,5-dihydro-5-oxo-1H-1, 2,4-triazol-4-yl] methyl] [1,1′-biphenyl]-2-carboxylic acid;

4′-[[3-(1-butynyl)-1-butyl-4,5-dihydro-5-oxo-1H-1,2,4-triazol-4-yl] methyl] [1,1′-biphenyl]-2-carboxylic acid;

4′- [[3-(1-butynyl)-1-secbutyl-4,5-dihydro-5-oxo-1H-1,2,4 -triazol-4-yl] methyl] [1,1 ′-biphenyl]-2-carboxylic acid;

4′-[[3-(1-butynyl)-1-isobutyl-4,5-dihydro-5-oxo-1H-1,2,4-triazol-4-yl] methyl] [1,1′-biphenyl]-2-carboxylic acid;

4 -[[3-(1-butynyl)-1-tertbutyl-4,5-dihydro-5-oxo-1H-1,2,4-triazol-4-yl] methyl] [1,1′-biphenyl]-2-carboxylic acid;

4′-[[3-(1-butynyl)-1-pentyl-4, 5-dihydro-5-oxo-1H-1,2,4-triazol-4-yl] methyl] [1,1′-biphenyl]-2-carboxylic acid;

4′-[[3-(1-butynyl)-1-isopentyl-4,5-dihydro-5-oxo-1H-1,2,4-triazol-4-yl] methyl] [1,1′-biphenyl]-2-carboxylic acid;

4′-[(3-(1-butynyl)-1-hexyl-4,5-dihydro-5-oxo-1H-1,2,4-triazol-4-yl) methyl] [1,1′-biphenyl]-2-carboxylic acid;

4′-[(3-(1-butynyl)-1-heptyl-4,5-dihydro-5-oxo-1H-1,2,4-triazol-4-yl) methyl] [1,1′-biphenyl]-2-carboxylic acid;

4′-[(3-(1-butynyl)-1-octyl-4,5-dihydro-5-oxo-1H-1,2,4-triazol-4-yl) methyl] [1,1′-biphenyl]-2-carboxylic acid;

4′-[[3-(1-butynyl)-1-cyclohexyl-4,5-dihydro-5-oxo-1H-1,2,4-triazol-4-yl] methyl] [1,1′-biphenyl]-2-carboxylic acid;

4′-[[3-(1-butynyl)-1-cyclohexylmethyl -4,5-dihydro-5 -oxo-1H-1,2,4-triazol-4-yl] methyl] [1,1′-biphenyl]-2-carboxylic acid;

4′-[[3-(1-butynyl)-1-(2-cyclohexylethyl)-4,5-dihydro-5-oxo-1H-1,2,4-triazol-4-yl] methyl] [1,1′-biphenyl]-2-carboxylic acid;

4′-[[3-(1-butynyl)-1-cyclohexanoyl-4,5-dihydro-5-oxo-1H-1,2,4-triazol-4-yl] methyl] [1,1′-biphenyl]-2-carboxylic acid;

4′-[[3-(1-butynyl)-1-(1-oxo-2-cyclohexylethyl)-4,5-dihydro-5-oxo-1H-1,2,4-triazol-4-yl] methyl] [1,1′-biphenyl]-2-carboxylic acid;

4′-[[3-(1-butynyl)-1-phenyl-4,5-dihydro-5-oxo-1H-1,2,4-triazol-4-yl] methyl] [1,1′-biphenyl]-2-carboxylic acid;

4 ′-[3-(1-butynyl)-1-phenylmethyl-4,5-dihydro-5-oxo-1H-1,2,4-triazol-4-yl] methyl] [1,1′-biphenyl]-2-carboxylic acid;

4′- [3-(1-butynyl)-1-(2-phenylethyl)-4,5-dihydro-5-oxo-1H-1,2,4-triazol-4-yl] methyl] [1,1′-biphenyl]-2-carboxylic acid;

4′-[[3-(1-butynyl)-1-benzoyl-4,5-dihydro-5-oxo-1H-1,2,4-triazol-4-yl] methyl] [1,1′-biphenyl]-2-carboxylic acid;

4′-[[3-(1-butynyl) -1-(1-oxo-2-phenylethyl)-4,5-dihydro-5-oxo-1H-1,2,4-triazol-4-yl] methyl] [1,1′-biphenyl]-2-carboxylic acid;

4′-[[3-(1-butynyl)-1-(1-oxopropyl)-4,5-dihydro-5-oxo-1H-1,2,4-triazol-4-yl]methyl] [1,1′-biphenyl]-2-carboxylic acid;

4′- [[3-(1-butynyl)-1-(1-oxobutyl)-4,5-dihydro-5-oxo-1H-1,2,4-triazol-4-yl] methyl] [1,1′-biphenyl]-2-carboxylic acid;

4′-[[3-(1-butynyl)-1-(1-oxopentyl)-4,5-dihydro-5-oxo-1H-1,2,4-triazol-4-yl] methyl] [1,1′-biphenyl]-2-carboxylic acid;

4′-[[3-(1-butynyl)-1-(2-butenyl)-4,5-dihydro-5-oxo-1H-1,2,4-triazole-4-yl] methyl] [1,1′-biphenyl]-2-carboxylic acid;

4′-[[3-(1-butynyl)-1-(3-butenyl)-4,5-dihydro-5-oxo-1H-1,2,4-triazole-4-yl] methyl] [1,1′-biphenyl]-2-carboxylic acid;

4′-[[3-(1-butynyl)-1-(2-butynyl)-4,5-dihydro-5-oxo-1H-1,2,4-triazole-4-yl] methyl] [1,1′-biphenyl]-2-carboxylic acid;

4′-[[3-(1-butynyl)-1-(3-butynyl)-4,5-dihydro-5-oxo-1H-1,2,4-triazole-4-yl] methyl] [1,1′-biphenyl]-2-carboxylic acid;

4′-[[3-(2-butynyl)-1-ethyl-4,5-dihydro-5-oxo-1H-1,2,4-triazol-4-yl] methyl] [1,1′-biphenyl]-2-carboxylic acid;

4′-[[3-(2-butynyl)-1-propyl-4,5-dihydro-5-oxo-1H-1,2,4-triazol-4-yl] methyl] [1,1′-biphenyl]-2-carboxylic acid;

4′-[[3-(2-butynyl)-1-butyl-4,5-dihydro-5-oxo-1H-1,2,4-triazol-4-yl] methyl] [1,1′-biphenyl]-2-carboxylic acid;

4′-[[3-(2-butynyl)-1-secbutyl-4,5-dihydro-5-oxo-1H-1,2,4-triazol-4-yl] methyl] [1,1′-biphenyl]-2-carboxylic acid;

4′-[[3-(2-butynyl)-1-isobutyl-4,5-dihydro-5-oxo-1H-1,2,4-triazol-4-yl] methyl] [1,1′-biphenyl]-2-carboxylic acid;

4′-[[3-(2-butynyl)-1-tertbutyl-4,5-dihydro-5-oxo-1H-1,2,4-triazol-4-yl] methyl] [1,1′-biphenyl]-2-carboxylic acid;

4′-[[3-(2-butynyl)-1-pentyl-4,5-dihydro-5-oxo-1H-1,2,4-triazol-4-yl] methyl] [1,1′-biphenyl]-2-carboxylic acid;

4′-[[3-(2-butynyl)-1-isopentyl-4,5-dihydro-5-oxo-1H-1,2,4-triazol-4-yl] methyl] [1,1′ -biphenyl]-2-carboxylic acid;

4′-[(3-(2-butynyl)-1-hexyl-4,5-dihydro-5-oxo-1H-1,2,4-triazol-4-yl) methyl] [1,1′-biphenyl]-2-carboxylic acid;

4′-[(3-(2-butynyl)-1-heptyl-4,5-dihydro-5-oxo-1H-1,2,4-triazol-4-yl) methyl] [1,1′-biphenyl]-2-carboxylic acid;

4′-[(3-(2-butynyl)-1-octyl-4,5-dihydro-5-oxo-1H-1,2,4-triazol-4-yl) methyl] [1,1′-biphenyl]-2-carboxylic acid;

4′-[[3-(2-butynyl)-1-cyclohexyl-4,5-dihydro-5-oxo-1H-1,2,4-triazol-4-yl] methyl] [1,1′-biphenyl]-2-carboxylic acid;

4′-[[3-(2-butynyl)-1-cyclohexylmethyl-4,5-dihydro-5-oxo-1H-1,2,4-triazol-4-yl] methyl] [1,1′-biphenyl]-2-carboxylic acid;

4′-[[3-(2-butynyl)-1-(2-cyclohexylethyl)-4,5-dihydro-5-oxo-1H-1,2,4-triazol-4-yl] methyl] [1,1′-biphenyl]-2-carboxylic acid;

4′-[[3-(2-butynyl)-1-cyclohexanoyl-4,5-dihydro-5-oxo-1H-1,2,4-triazol-4-yl] methyl] [1,1′-biphenyl]-2-carboxylic acid;

4′-[[3-(2-butynyl)-1-(1-oxo-2-cyclohexylethyl)-4,5-dihydro-5-oxo-1H-1,2,4-triazol-4-yl] methyl] [1,1′-biphenyl]-2-carboxylic acid;

4′-[[3-(2-butynyl)-1-phenyl-4,5-dihydro-5-oxo-1H-1,2,4-triazol-4-yl] methyl] [1,1′-biphenyl]-2-carboxylic acid;

4′-[[3-(2-butynyl)-1-phenylmethyl-4,5-dihydro-5-oxo-1H-1,2,4-triazol-4-yl] methyl] [1,1′-biphenyl]-2-carboxylic acid;

4′-[[3-(2-butynyl)-1-(2-phenylethyl)-4,5-dihydro-5-oxo-1H-1,2,4-triazol-4-yl] methyl] [1,1′-biphenyl]-2-carboxylic acid;

4′-[[3-(2-butynyl)-1-benzoyl-4,5-dihydro-5-oxo-1H-1,2,4-triazol-4-yl] methyl] [1,1′-biphenyl]-2-carboxylic acid;

4′-[[3-(2-butynyl)-1-(1-oxo-2-phenylethyl)-4,5-dihydro-5-oxo-1H-1,2,4-triazol-4-yl] methyl] [1,1′-biphenyl]-2-carboxylic acid;

4′-[[3-(2-butynyl)-i-(i-oxopropyl)-4,5-dihydro-5-oxo-1H-1,2,4-triazol-4-yl] methyl] [1,1′-biphenyl]-2-carboxylic acid;

4′-[[3-(2-butynyl)-1-(1-oxobutyl)-4,5-dihydro-5-oxo-1H-1,2,4-triazol-4-yl] methyl] [1,1′-biphenyl]-2-carboxylic acid;

4′-[[3-(2-butynyl)-1-(1-oxopentyl)-4,5-dihydro-5-oxo-1H-1,2,4-triazol-4-yl] methyl] [1,1′-biphenyl]-2-carboxylic acid;

4′-[[3-(2-butynyl)-1-(2-butenyl)-4,5-dihydro-5-oxo-1H-1,2,4-triazole-4-yl] methyl] [1,1′-biphenyl]-2-carboxylic acid;

4′-[[3-(2-butynyl)-1-(3-butenyl)-4,5-dihydro-5-oxo-1H-1,2,4-triazole-4-yl] methyl] [1,1′-biphenyl]-2-carboxylic acid;

4′-[[1,3-di(2-butynyl)-4,5-dihydro-5-oxo-1H-1,2,4-triazole-4-yl] methyl] [1,1′-biphenyl]-2-carboxylic acid;

4′-[[3-(2-butynyl)-1-(3-butynyl)-4,5-dihydro-5-oxo-1H-1,2,4-triazole-4-yl] methyl] [1,1′-biphenyl)-2-carboxylic acid;

4′-[[3-(3-butynyl)-1-ethyl-4,5-dihydro-5-oxo-1H-1,2,4-triazol-4-yl] methyl] [1,1′-biphenyl]-2-carboxylic acid;

4′-[[3-(3-butynyl)-1-propyl-4,5-dihydro-5-oxo-1H-1,2,4-triazol-4-yl] methyl] [1,1′-biphenyl]-2-carboxylic acid;

4′-[[3-(3-butynyl)-1-isopropyl-4,5-dihydro-5-oxo-1H-1,2,4-triazol-4-yl] methyl] [1,1′-biphenyl]-2-carboxylic acid;

4′-[[3-(3-butynyl)-1-butyl-4,5-dihydro-5-oxo-1H-1,2,4-triazol-4-yl] methyl] [1,1′-biphenyl]-2-carboxylic acid;

4′-[[3-(3-butynyl)-1-secbutyl-4,5-dihydro-5-oxo-1H-1,2,4-triazol-4-yl] methyl] [1,1′-biphenyl]-2-carboxylic acid;

4′-[[3-(3-butynyl)-1-isobutyl-4,5-dihydro-5-oxo-1H-1,2,4-triazol-4-yl] methyl] [1,1′-biphenyl]-2-carboxylic acid;

4′-[[3-(3-butynyl)-1-tertbutyl-4,5-dihydro-5-oxo-1H-1,2,4-triazol-4-yl] methyl] [1,1′-biphenyl]-2-carboxylic acid;

4′-[[3-(3-butynyl)-1-isopentyl-4,5-dihydro-5-oxo-1H-1,2,4-triazol-4-yl] methyl] [l,l′-biphenyl]-2-carboxylic acid;

4′-[[3-(3-butynyl)-1-pentyl-4,5-dihydro-5-oxo-1H-1,2,4-triazol-4-yl] methyl] [1,1′-biphenyl]-2-carboxylic acid;

4′-[(3-(3-butynyl)-1-hexyl-4,5-dihydro-5-oxo-1H-1,2,4-triazol-4-yl) methyl] [1,1′-biphenyl]-2-carboxylic acid;

4′-[(3-(3-butynyl)-1-heptyl-4,5-dihydro-5-oxo-1H-1,2,4-triazol-4-yl) methyl] [1,1′-biphenyl]-2-carboxylic acid;

4′-[(3-(3-butynyl)-1-octyl-4,5-dihydro-5-oxo-1H-1,2,4-triazol-4-yl) methyl] [1,1′-biphenyl]-2-carboxylic acid;

4′-[[3-(3-butynyl)-1-cyclohexyl-4,5-dihydro-5-oxo-1H-1,2,4-triazol-4-yl] methyl] [1,1′-biphenyl]-2-carboxylic acid;

4′-[[3-(3-butynyl)-1-cyclohexylmethyl-4,5-dihydro-5-oxo-1H-1,2,4-triazol-4-yl] methyl] [1,1′-biphenyl]-2-carboxylic acid;

4′-[[3-(3-butynyl)-1-(2-cyclohexylethyl)-4,5-dihydro-5-oxo-1H-1,2,4-triazol-4-yl]methyl] [1,1′-biphenyl]-2-carboxylic acid;

4′-[[3-(3-butynyl)-1-cyclohexanoyl-4,5-dihydro-5-oxo-1H-1,2,4-triazol-4-yl] methyl] [1,1′-biphenyl]-2-carboxylic acid;

4′-[[3-(3-butynyl)-1-(1-oxo-2-cyclohexylethyl)-4,5-dihydro-5-oxo-1H-1,2,4-triazol-4-yl] methyl] [1,1′-biphenyl]-2-carboxylic acid;

4′-[[3-(3-butynyl)-1-phenyl-4,5-dihydro-5-oxo-1H-1,2,4-triazol-4-yl] methyl] [1,1′-biphenyl]-2-carboxylic acid;

4′-[[3-(3-butynyl)-1-phenylmethyl-4,5-dihydro-5-oxo-1H-1,2,4-triazol-4-yl] methyl] [1,1′-biphenyl]-2-carboxylic acid;

4′-[[3-(3-butynyl)-1-(2-phenylethyl)-4,5-dihydro-5-oxo-1H-1,2,4-triazol-4-yl] methyl] [1,1′-biphenyl]-2-carboxylic acid;

4′-[[3-(3-butynyl)-1-benzoyl-4,5-dihydro-5-oxo-1H-1,2,4-triazol-4-yl] methyl] [1,1′-biphenyl]-2-carboxylic acid;

4′-[[3-(3-butynyl)-1-(1-oxo-2-phenylethyl)-4,5-dihydro-5-oxo-1H-1,2,4-triazol-4-yl] methyl] [1,1′-biphenyl]-2-carboxylic acid;

4′-[[3-(3-butynyl)-1-(1-oxopropyl)-4,5-dihydro-5-oxo-1H-1,2,4-triazol-4-yl] methyl] [1,1′-biphenyl]-2-carboxylic acid;

4′-[[3-(3-butynyl)-1-(1-oxobutyl)-4,5-dihydro-5-oxo-1H-1,2,4-triazol-4-yl] methyl] [1,1′-biphenyl]-2-carboxylic acid;

4′-[[3-(3-butynyl)-1-(1-oxopentyl)-4,5-dihydro-5-oxo-1H-1,2,4-triazol-4-yl] methyl] [1,1′-biphenyl]-2-carboxylic acid;

4′-[[3-(3-butynyl)-1-(2-butenyl)-4,5-dihydro-5-oxo-1H-1,2,4-triazole-4-yl] methyl] [1,1′-biphenyl]-2-carboxylic acid;

4′-[[3-(3-butynyl)-1-(3-butenyl) -4, 5-dihydro-5-oxo-1H-1,2,4-triazole-4-yl] methyl] [1,1′-biphenyl]-2-carboxylic acid;

4′-[[3-(3-butynyl)-1-(2-butynyl)-4,5-dihydro-5-oxo-1H-1,2,4-triazole-4-yl] methyl] [1,1′-biphenyl]-2-carboxylic acid;

4′-[[1,3-di(3-butynyl)-4,5-dihydro-5-oxo-1H-1,2,4-triazole-4-yl] methyl] [1,1′-biphenyl]-2-carboxylic acid;

2,5-diethyl-2,4-dihydro-4-[[2-(1H-tetrazol-5-yl) [1,1′-biphenyl]-4′-yl]methyl]-3H-1,2,4-triazol-3-one;

5-ethyl-2-propyl-2,4-dihydro-4-[[2-(1H-tetrazol-5-yl) [1,1′-biphenyl]-4′-yl]methyl]-3H-1,2,4-triazol-3-one;

5-ethyl-2-isopropyl-2,4-dihydro-4-[[2-(1H-tetrazol-5-yl) [1,1′-biphenyl]-4′-yl]methyl]-3H-1,2,4-triazol-3-one;

5-ethyl-2-butyl-2,4-dihydro-4-[[2-(1H-tetrazol-5-yl) [1,1′-biphenyl]-4′-yl]methyl]-3H-1,2,4-triazol-3-one;

5-ethyl-2-secbutyl-2,4-dihydro-4-[[2-(1H-tetrazol-5-yl) [1,1′-biphenyl]-4′-yl]methyl]-3H-1,2,4-triazol-3-one;

5-ethyl-2-isobutyl-2,4-dihydro-4-[[2-(1H-tetrazol-5-yl) [1,1′-biphenyl3-4′-yl]methyl]-3H-1,2,4-triazol-3-one;

5-ethyl-2-tertbutyl-2,4-dihydro-4-[[2-(1H-tetrazol-5-yl) [1,1′-biphenyl]-4′-yl]methyl]-3H-1,2,4-triazol-3-one;

5-ethyl-2-pentyl-2,4-dihydro-4-[[2-(1H-tetrazol-5-yl) [1,1′-biphenyl]-4′-yl]methyl]-3H-1,2,4-triazol-3-one;

5-ethyl-2-isopentyl-2,4-dihydro-4-[[2-(1H-tetrazol-5-yl) [1,1′-biphenyl]-4′-yl]methyl]-3H-1,2,4-triazol-3-one;

5-ethyl-2-hexyl-2,4-dihydro-4-[[2-(1H-tetrazol-5-yl) [1,1′-biphenyl]-4′-yl]methyl]-3H-1,2,4-triazol-3-one;

5-ethyl-2-heptyl-2,4-dihydro-4-[[2-(1H-tetrazol-5-yl) [1,1′-biphenyl]-4′-yl]methyl]-3H-1,2,4-triazol-3-one;

5-ethyl-2-octyl-2,4-dihydro-4-[[2-(1H-tetrazol-5-yl) [1,1′-biphenyl]-4′-yl]methyl]-3H-1,2,4-triazol-3-one;

5-ethyl-2-cyclohexyl-2,4-dihydro-4-[[2-(1H-tetrazol-5-yl) [1,1′-biphenyl]-4′-yl]methyl]-3H-1,2,4-triazol-3-one;

5-ethyl-2-cyclohexylmethyl-2,4-dihydro-4-[[2-(1H-tetrazol-5-yl) [1,1′-biphenyl]-4′-yl]methyl]-3H-1,2,4-triazol-3-one;

5-ethyl-2-(2-cyclohexylethyl)-2,4-dihydro-4-[[2-(1H-tetrazol-5-yl) [1,1′-biphenyl]-1′-yl]methyl]-3H-1,2,4-triazol-3-one;

5-ethyl-2-cyclohexanoyl-2,4-dihydro-4-[[2-(1H-tetrazol-5-yl) [1,1′-biphenyl]-4′-yl]methyl]-3H-1,2,4-triazol-3-one;

5-ethyl-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;

5-ethyl-2-phenyl-2,4-dihydro-4-[[2-(1H-tetrazol-5-yl) [1,1′-biphenyl]-4′-yl]methyl]-3H-1,2,4-triazol-3-one;

5-ethyl-2-phenylmethyl-2,4-dihydro-4-[[2-(1H-tetrazol-5-yl) [1,1′-biphenyl]-4′-yl]methyl]-3H-1,2,4-triazol-3-one;

5-ethyl-2-(2-phenylethyl)-2,4-dihydro-4-[[2-(1H-tetrazol-5-yl) [1,1′-biphenyl]-4′-yl]methyl]-3H-1,2,4-triazol-3-one;

5-ethyl-2-benzoyl-2,4-dihydro-4-[[2-(1H-tetrazol-5-yl) [1,1′-biphenyl]-4′-yl]methyl]-3H-1,2,4-triazol-3-one;

5-ethyl-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;

5-ethyl-2-(1-oxopropyl)-2,4-dihydro-4-[[2-(1H-tetrazol-5-yl) [1,1′-biphenyl]-4′-yl]methyl]-3H-1,2,4-triazol-3-one;

5-ethyl-2-(1-oxobutyl)-2,4-dihydro-4-[[2-(1H-tetrazol-5-yl) [1,1′-biphenyl]-4′-yl]methyl]-3H-1,2,4-triazol-3-one;

5-ethyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-ethyl-2-(2-butenyl)-2,4-dihydro-4-[[2-(1H-tetrazol-5-yl) [1,1′-biphenyl]-4′-yl]methyl]-3H-1,2,4-triazol-3-one;

5-ethyl-2-(3-butenyl)-2,4-dihydro-4-[[2-(1H-tetrazol-5-yl) [1,1′-biphenyl]-4′-yl]methyl]-3H-1,2,4-triazol-3-one;

5-ethyl-2-(2-butynyl)-2,4-dihydro-4-([2-(1H-tetrazol-5-yl) [1,1′-biphenyl]-4′-yl]methyl]-3H-1,2,4-triazol-3-one;

5-ethyl-2-(3-butynyl)-2,4-dihydro-4-[[2-(1H-tetrazol-5-yl) [1,1′-biphenyl]-4′-yl]methyl]-3H-1,2,4-triazol-3-one;

5-propyl-2-ethyl-2,4-dihydro-4-[[2-(1H-tetrazol-5-yl) [1,1′-biphenyl]-4′-yl]methyl]-3H-1,2,4-triazol-3-one;

2,5-dipropyl-2,4-dihydro-4-[[2-(1H-tetrazol-5-yl) [1,1′-biphenyl]-4′-yl]methyl]-3H-1,2,4-triazol-3-one;

5-propyl-2-isopropyl-2,4-dihydro-4-[[2-(1H-tetrazol-5-yl) [1,1′-biphenyl]-4′-yl]methyl]-3H-1,2,4-triazol-3-one;

5-propyl-2-butyl-2,4-dihydro-4-[[2-(1H-tetrazol-5-yl) [1,1′-biphenyl]-4′-yl]methyl]-3H-1,2,4-triazol-3-one;

5-propyl-2-secbutyl-2,4-dihydro-4-[[2-(1H-tetrazol-5-yl) [1,1′-biphenyl]-4′-yl]methyl]-3H-1,2,4-triazol-3-one;

5-propyl-2-isobutyl-2,4-dihydro-4-[[2-(1H-tetrazol-5-yl) [1,1′-biphenyl]-4′-yl]methyl]-3H-1,2,4-triazol-3-one;

5-propyl-2-tertbutyl-2,4-dihydro-4-[[2-(1H-tetrazol-5-yl) [1,1′-biphenyl]-4′-yl]methyl]-3H-1,2,4-triazol-3-one;

5-propyl-2-pentyl-2,4-dihydro-4-[[2-(1H-tetrazol-5-yl) [1,1′-biphenyl]-4′-yl]methyl]-3H-1,2,4-triazol-3-one;

5-propyl-2-isopentyl-2,4-dihydro-4-[(2-(1H-tetrazol-5-yl) [1,1′-biphenyl′-4′-yl]methyl]-3H-1,2,4-triazol-3-one;

5-propyl-2-hexyl-2,4-dihydro-4-[[2-(1H-tetrazol-5-yl) [1,1′-biphenyl]-4′-yl]methyl]-3H-1,2,4-triazol-3-one;

5-propyl-2-heptyl-2,4-dihydro-4-[[2-(1H-tetrazol-5-yl) [1,1′-biphenyl]-4′-yl]methyl]-3H-1,2,4-triazol-3-one;

5-propyl-2-octyl-2,4-dihydro-4-[[2-(1H-tetrazol-5-yl) [1,1′-biphenyl]-4′-yl]methyl]-3H-1,2,4-triazol-3-one;

5-propyl-2-cyclohexyl-2,4-dihydro-4- [[2-(1H-tetrazol-5-yl) [1,1′-biphenyl]-4′-yl]methyl]-3H-1,2,4-triazol-3-one;

5-propyl-2-cyclohexylmethyl-2,4-dihydro-4-[[2-(1H-tetrazol-5-yl) [1,1′-biphenyl]-4′-yl]methyl]-3H-1,2,4-triazol-3-one;

5-propyl-2-(2-cyclohexylethyl)-2,4-dihydro-4-[[2-(1H-tetrazol-5-yl) [1,1′-biphenyl]-4′-yl]methyl]-3H-1,2,4-triazol-3-one;

5-propyl-2-cyclohexanoyl-2,4-dihydro-4-[[2-(1H-tetrazol-5-yl) [1,1′-biphenyl]-4′-yl]methyl]-3H-1,2,4-triazol-3-one;

5-propyl-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;

5-propyl-2-phenyl-2,4-dihydro-4-[[2-(1H-tetrazol-5-yl) [1,1′-biphenyl]-4′-yl]methyl]-3H-1,2,4-triazol-3-one;

5-propyl-2-(2-phenylethyl)-2,4-dihydro-4-[[2-(1H-tetrazol-5-yl) [1,1′-biphenyl]-4′-yl]methyl]-3H-1,2,4-triazol-3-one;

5-propyl-2-benzoyl-2, 4-dihydro-4-[[2-( 1H-tetrazol-5-yl) [1,1′-biphenyl]-4′-yl]methyl]-3H-1,2,4-triazol-3-one;

5-propyl-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;

5-propyl-2-(1-oxopropyl)-2,4-dihydro-4-[[2-(1H-tetrazol-5-yl) [1,1′-biphenyl]-4′-yl methyl]-3H-1,2,4-triazol-3-one;

5-propyl-2-(1-oxobutyl)-2,4-dihydro-4-[[2-(1H-tetrazol-5-yl) [1,1′-biphenyl]-4′-yl]methyl]-3H-1,2,4-triazol-3-one;

5-propyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-propyl-2-(2-butenyl)-2,4-dihydro-4-[[2-(1H-tetrazol-5-yl) [1,1′-biphenyl′-4′-yl]methyl]-3H-1,2,4-triazol-3-one;

5-propyl-2-(3-butenyl)-2,4-dihydro-4-[[2-(1H-tetrazol-5-yl) [1,1′-biphenyl]-4′-yl]methyl)-3H-1,2,4-triazol-3-one;

5-propyl-2-(2-butynyl)-2,4-dihydro-4-[[2-(1H-tetrazol-5-yl) [1,1′-biphenyl]-4′-yl]methyl]-3H-1,2,4-triazol-3-one;

5-propyl-2-(3-butynyl)-2,4-dihydro-4-[[2-(1H-tetrazol-5-yl) [1,1′-biphenyl]-4′-yl]methyl]-3H-1,2,4-triazol-3-one;

5-isopropyl-2-ethyl-2,4-dihydro-4-[[2-(1H-tetrazol-5-yl) [1,1′-biphenyl]-4′-yl]methyl]-3H-1,2,4-triazol-3-one;

b 5-isopropyl-2-propyl-2,4-dihydro-4-[[2-(1H-tetrazol-5-yl) [1,1′-biphenyl]-4′-yl]methyl]-3H-1,2,4-triazol-3-one;

2,5-diisopropyl-2,4-dihydro-4-[[2-(1H-tetrazol-5-yl) [1,1′-biphenyl]-4′-yl]methyl]-3H-1,2,4-triazol-3-one;

5-isopropyl-2-butyl-2,4-dihydro-4-[[2-(1H-tetrazol-5-yl) [1,1′-biphenyl]-4′-yl]methyl)-3H-1,2,4-triazol-3-one;

5-isopropyl-2-secbutyl-2,4-dihydro-4-[[2-(1H-tetrazol-5-yl) [1,1′-biphenyl]-4′-yl]methyl]-3H-1,2,4-triazol-3-one;

5-isopropyl-2-isobutyl-2,4-dihydro-4-[[2-(1H-tetrazol-5-yl) [1,1′-biphenyl]-4′-yl]methyl]-3H-1,2,4-triazol-3-one;

5-isopropyl-2-tertbutyl-2,4-dihydro-4-[[2-(1H-tetrazol-5-yl) [1,1′-biphenyl]-4′-yl]methyl]-3H-1,2,4-triazol-3-one;

5-isopropyl-2-pentyl-2,4-dihydro-4-[[2-(1H-tetrazol-5-yl) [1,1′-biphenyl]-4′-yl]methyl]-3H-1,2,4-triazol-3-one;

5-isopropyl-2-isopentyl-2,4-dihydro-4-[[2-(1H-tetrazol-5-yl) [1,1′-biphenyl)-4′-yl]methyl]-3H-1,2,4-triazol-3-one;

5-isopropyl-2-hexyl-2,4-dihydro-4-[[2-(1H-tetrazol-5-yl) [1,1′-biphenyl]-4′-yl]methyl]-3H-1,2,4-triazol-3-one;

5-isopropyl-2-heptyl-2,4-dihydro-4-[[2-(1H-tetrazol-5-yl) [1,1′-biphenyl]-4′-yl]methyl]-3H-1,2,4-triazol-3-one;

5-isopropyl-2-octyl-2,4-dihydro-4-[[2-(1H-tetrazol-5-yl) [1,1′-biphenyl]-4′-yl]methyl]-3H-1,2,4-triazol-3-one;

5-isopropyl-2-cyclohexyl-2,4-dihydro-4-[[2-(1H-tetrazol-5-yl) [1,1′-biphenyl]-4t-yl]methyl]-3H-1,2,4-triazol-3-one;

5-isopropyl-2-cyclohexylmethyl-2,4-dihydro-4-[[2-(1H-tetrazol-5-yl) [1,1′-biphenyl]-4′-yl]methyl]-3H-1,2,4-triazol-3-one;

5-isopropyl-2-(2-cyclohexylethyl)-2,4-dihydro-4-[[2-(1H-tetrazol-5-yl) [1,1′-biphenyl]-4′-yl]methyl]-3H-1,2,4-triazol-3-one;

5-isopropyl-2-cyclohexanoyl-2,4-dihydro-4-[[2-(1H-tetrazol-5-yl) [1,1′-biphenyl]-4′-yl]methyl]-3H-1,2,4-triazol-3-one;

5-isopropyl-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;

5-isopropyl-2-phenyl-2,4-dihydro-4-[[2-(1H-tetrazol-5-yl) [1,1′-biphenyl]-4′-yl]methyl]-3H-1,2,4-triazol-3-one;

5-isopropyl-2-phenylmethyl-2,4-dihydro-4-[[2-(1H-tetrazol-5-yl) [1,1′-biphenyl]-4′-yl]methyl]-3H-1,2,4-triazol-3-one;

5-isopropyl-2-(2-phenylethyl)-2,4-dihydro-4-([2-(1H-tetrazol-5-yl) [1,1′-biphenyl]-4′-yl]methyl]-3H-1,2,4-triazol-3-one;

5-isopropyl-2-benzoyl-2,4-dihydro-4-[[2-(1H-tetrazol-5-yl) [1,1′-biphenyl]-4′-yl]methyl]-3H-1,2,4-triazol-3-one;

5-isopropyl-2-(1-oxo-2-phenylethyl)-2,4-dihydro-4-[[2- (1H-tetrazol-5-yl) [1,1′-biphenyl]-4′-yl]methyl]-3H-1,2,4-triazol-3-one;

5-isopropyl-2-(1-oxopropyl)-2,4-dihydro-4-[[2-(1H-tetrazol-5-yl) [1,1′-biphenyl]-4′-yl]methyl]-3H-1,2,4-triazol-3-one;

5-isopropyl-2-(1-oxobutyl)-2,4-dihydro-4-[[2-(1H-tetrazol-5-yl) [1,1′-biphenyl]-4′-yl]methyl]-3H-1,2,4-triazol-3-one;

5-isopropyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-isopropyl-2-(2-butenyl)-2,4-dihydro-4-[[2-(1H-tetrazol-5-yl) [1,1′-biphenyl]-4′-yl]methyl]-3H-1,2,4-triazol-3-one;

5-isopropyl-2-(3-butenyl)-2,4-dihydro-4-[[2-(1H-tetrazol-5-yl) [1,1′-biphenyl]-4′-yl]methyl]-3H-1,2,4-triazol-3-one;

5-isopropyl-2-(2-butynyl)-2,4-dihydro-4-[[2-(1H-tetrazol-5-yl) [1,1′-biphenyl]-4′-yl]methyl]-3H-1,2,4-triazol-3-one;

5-isopropyl-2-(3-butynyl)-2,4-dihydro-4-[[2-(1H-tetrazol-5-yl) [1,1′-biphenyl]-4′-yl]methyl]-3H-1,2,4-triazol-3-one;

5-butyl-2-ethyl-2,4-dihydro-4-[[2-(1H-tetrazol-5-yl) [1,1′-biphenyl]-4′-yl]methyl]-3H-1,2,4-triazol-3-one;

5-butyl-2-propyl-2,4-dihydro-4-[[2-(1H-tetrazol-5-yl) [1,1′-biphenyl]-4′-yl]methyl]-3H-1,2,4-triazol-3-one;

5-butyl-2-isopropyl-2,4-dihydro-4-[[2-(1H-tetrazol-5-yl) [1,1′-biphenyl]-4′-yl]methyl]-3H-1,2,4-triazol-3-one; 2,5-dibutyl-2,4-dihydro-4-[[2-(1H-tetrazol-5-yl) [1,1′-biphenyl]-4′-yl]methyl]-3H-1,2,4-triazol-3-one;

5-butyl-2-secbutyl-2,4-dihydro-4-[[2-(1H-tetrazol-5-yl) [1,1′-biphenyl]-4′-yl]methyl]-3H-1,2,4-triazol-3-one;

5-butyl-2-isobutyl-2,4-dihydro-4-([2-(1H-tetrazol-5-yl) [1,1′-biphenyl]-4′-yl]methyl]-3H-1,2,4-triazol-3-one;

5-butyl-2-tertbutyl-2,4-dihydro-4-[[2-(1H-tetrazol-5-yl) [1,1′-biphenyl]-4′-yl]methyl]-3H-1,2,4-triazol-3-one;

5-butyl-2-pentyl-2,4-dihydro-4-[[2-(1H-tetrazol-5-yl) [1,1′-biphenyl]-4′-yl]methyl]-3H-1,2,4-triazol-3-one;

5-butyl-2-isopentyl-2,4-dihydro-4-[[2-(1H-tetrazol-5-yl) [1,1′-biphenyl]-4′-yl]methyl]-3H-1,2,4-triazol-3-one;

5-butyl-2-hexyl-2,4-dihydro-4-[[2-(1H-tetrazol-5-yl) [1,1′-biphenyl]-4′-yl]methyl]-3H-1,2,4-triazol-3-one;

5-butyl-2-heptyl-2,4-dihydro-4-[[2-(1H-tetrazol-5-yl) [1,1′-biphenyl]-4′-yl]methyl]-3H-1,2,4-triazol-3-one;

5-butyl-2-octyl-2,4-dihydro-4-[[2-(1H-tetrazol-5-yl) [1,1′-biphenyl]-4′-yl]methyl]-3H-1,2,4-triazol-3-one;

5-butyl-2-cyclohexyl-2,4-dihydro-4-[[2-(1H-tetrazol-5-yl) [1,1′-biphenyl]-4′-yl]methyl]-3H-1,2,4-triazol-3-one;

5-butyl-2-cyclohexylmethyl-2,4-dihydro-4-[[2-(1H-tetrazol-5-yl) [1,1′-biphenyl]-4′-yl]methyl]-3H-1,2,4-triazol-3-one;

5-butyl-2-(2-cyclohexylethyl)-2,4-dihydro-4-[[2-(1H-tetrazol-5-yl) [1,1′-biphenyl]-4′-yl]methyl]-3H-1,2,4-triazol-3-one;

5-butyl-2-cyclohexanoyl-2,4-dihydro-4-[[2-(1H-tetrazol-5-yl) [1,1′-biphenyl]-4′-yl]methyl]-3H-1,2,4-triazol-3-one;

5-butyl-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;

5-butyl-2-phenyl-2,4-dihydro-4-[[2-(1H-tetrazol-5-yl) [1,1′-biphenyl]-4′-yl]methyl]-3H-1,2,4-triazol-3-one;

5-butyl-2-phenylmethyl-2,4-dihydro-4-[[2-(1H-tetrazol-5-yl) [1,1′-biphenyl]-4′-yl]methyl]-3H-1,2,4-triazol-3-one;

5-butyl-2-(2-phenylethyl)-2,4-dihydro-4-[[2-(1H-tetrazol-5-yl) [1,1′-biphenyl]-4′-yl]methyl]-3H-1,2,4-triazol-3-one;

5-butyl-2-benzoyl-2,4-dihydro-4-[[2-(1H-tetrazol-5-yl) [1,1′-biphenyl]-4′-yl]methyl]-3H-1,2,4-triazol-3-one;

5-butyl-2-(1-oxo-2-phenylethyl)-2,4-dihydro-4-[[2-(1H-tetrazol-5-yl) [1,1′-biphenyl]-4′-yl]methyl]-3H-1,2,4-5 triazol-3-one;

5-butyl-2-(1-oxopropyl)-2,4-dihydro-4-[[2-(1H-tetrazol-5-yl) [1,1′-biphenyl]-4′-yl]methyl]-3H-1,2,4-triazol-3-one;

5-butyl-2-(1-oxobutyl)-2,4-dihydro-4-[[2-(1H-tetrazol-5-yl) [1,1′-biphenyl]-4′-yl]methyl]-3H-1,2,4-triazol-3-one;

5-butyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-butyl-2-(2-butenyl)-2,4-dihydro-4-[[2-(1H-tetrazol-5-yl) [1,1′-biphenyl]-4′-yl]methyl]-3H-1,2,4-triazol-3-one;

5-butyl-2-(3-butenyl)-2,4-dihydro-4-[[2-(1H-tetrazol-5-yl) [1,1′-biphenyl]-4′-yl]methyl]-3H-1,2,4-triazol-3-one;

5-butyl-2-(2-butynyl)-2,4-dihydro-4-[[2-(1H-tetrazol-5-yl) [1,1′-biphenyl]-4′-yl]methyl]-3H-1,2,4-triazol-3-one;

5-butyl-2-(3-butynyl)-2,4-dihydro-4-[[2-(1H-tetrazol-5-yl) [1,1′-biphenyl]-4′-yl]methyl]-3H-1,2,4-triazol-3-one;

5-secbutyl-2-ethyl-2,4-dihydro-4-[[2-(1H-tetrazol-5-yl) [1,1′-biphenyl]-4′-yl]methyl]-3H-1,2,4-triazol-3-one;

5-secbutyl-2-propyl-2,4-dihydro-4-[[2-(1H-tetrazol-5-yl) [1,1′-biphenyl]-4′-yl]methyl]-3H-1,2,4-triazol-3-one;

5-secbutyl-2-isopropyl-2,4-dihydro-4-[[2-(1H-tetrazol-5-yl) [1,1′-biphenyl]-4′-yl]methyl]-3H-1,2,4-triazol-3-one;

5-secbutyl-2-butyl-2,4-dihydro-4-[[2-(1H-tetrazol-5-yl) [1,1′-biphenyl]-4′-yl]methyl]-3H-1,2,4-triazol-3-one; 2,5-disecbutyl-2,4-dihydro-4-[[2-(1H-tetrazol-5-yl) [1,1′-biphenyl]-4′-yl]methyl]-3H-1,2,4-triazol-3-one;

5-secbutyl-2-isobutyl-2,4-dihydro-4-[[2-(1H-tetrazol-5-yl) [1,1′-biphenyl]-4′-yl]methyl]-3H-1,2,4-triazol-3-one;

5-secbutyl-2-tertbutyl-2,4-dihydro-4-[[2-(1H-tetrazol-5-yl) [1,1′-biphenyl]-4′-yl]methyl]-3H-1,2,4-triazol-3-one;

5-secbutyl-2-pentyl-2,4-dihydro-4-([2-(1H-tetrazol-5-yl) [1,1′-biphenyl]-4′-yl]methyl]-3H-1,2,4-triazol-3-one;

5-secbutyl-2-isopentyl-2,4-dihydro-4-[[2-(1H-tetrazol-5-yl) [1,1′-biphenyl]-4′-yl]methyl]-3H-1,2,4-triazol-3-one;

5-secbutyl-2-hexyl-2,4-dihydro-4-[[2-(1H-tetrazol-5-yl) [1,1′-biphenyl]-4′-yl]methyl]-3H-1,2,4-triazol-3-one;

5-secbutyl-2-heptyl-2,4-dihydro-4-[[2-(1H-tetrazol-5-yl) [1,1-biphenyl]-4′-yl]methyl]-3H-1,2,4-triazol-3-one;

5-secbutyl-2-octyl-2,4-dihydro-4-[(2-(1H-tetrazol-5-yl) [1,1′-biphenyl]-4′-yl]methyl]-3H-1,2,4-triazol-3-one;

5-secbutyl-2-cyclohexyl-2,4-dihydro-4-[[2-(1H-tetrazol-5-yl) [1,1′-biphenyl]-4′-yl]methyl]-3H-1,2,4-triazol-3-one;

5-secbutyl-2-cyclohexylmethyl-2,4-dihydro-4-[[2-(1H-tetrazol-5-yl) [1,1′-biphenyl]-4′-yl]methyl]-3H-1,2,4-triazol-3-one;

5-secbutyl-2-(2-cyclohexylethyl)-2,4-dihydro-4-[[2-(1H-tetrazol-5yl) [1,1′-biphenyl]-4′-yl]methyl3-3H-1,2,4-triazol-3-one;

5-secbutyl-2-cyclohexanoyl-2,4-dihydro-4-[[2-(1H-tetrazol-5-yl) [1,1′-biphenyl]-4′-yl]methyl]-3H-1,2,4-triazol-3-one;

5-secbutyl-2-(1-oxo-2-cyclohexylethyl)-2,4-dihydro-4- [2-(1H-tetrazol-5-yl) [1,1′-biphenyl]-4′-yl]methyl]-3H-1,2,4-triazol-3-one;

5-secbutyl-2-phenyl-2,4-dihydro-4-[(2-(1H-tetrazol-5-yl) [1,1′-biphenyl]-4′-yl]methyl]-3H-1,2,4-triazol-3-one;

5-secbutyl-2-phenylmethyl-2,4-dihydro-4-[[2-(1H-tetrazol-5-yl) [1,1′-biphenyl]-4′-yl]methyl]-3H-1,2,4-triazol-3-one;

5-secbutyl-2-(2-phenylethyl)-2,4-dihydro-4-[(2-(1H-tetrazol-5-yl) [1,1′-biphenyl]-4′-yl]methyl]-3H-1,2,4-triazol-3-one;

5-secbutyl-2-benzoyl-2,4-dihydro-4-[[2-(1H-tetrazol-5-yl) [1,1′-biphenyl]-4′-yl]methyl]-3H-1,2,4-triazol-3-one;

5-secbutyl-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;

5-secbutyl-2-(1-oxopropyl)-2,4-dihydro-4-[[2-(1H-tetrazol-5-yl) [1,1′-biphenyl]-4′-yl]methyl]-3H-1,2,4-triazol-3-one;

5-secbutyl-2-(1-oxobutyl)-2,4-dihydro-4-[[2-(1H-tetrazol-5-yl) [1,1′-biphenyl]-4′-yl]methyl]-3H-1,2,4-triazol-3-one;

5-secbutyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-secbutyl-2-(2-butenyl)-2,4-dihydro-4-[[2-(1H-tetrazol-5-yl) [1,1′-biphenyl]-4′-yl]methyl]-3H-1,2,4-triazol-3-one;

5-secbutyl-2-(3-butenyl)-2,4-dihydro-4-[[2-(1H-tetrazol-5-yl) [1,1′-biphenyl]-4′-yl]methyl]-3H-1,2,4-triazol-3-one;

5-secbutyl-2-(2-butynyl)-2,4-dihydro-4-[[2-(1H-tetrazol-5-yl) [1,1′-biphenyl]-4′-yl]methyl]-3H-1,2,4-triazol-3-one;

5-secbutyl-2-(3-butynyl)-2,4-dihydro-4-[[2-(1H-tetrazol-5-yl) [1,1′-biphenyl]-4′-yl]methyl]-3H-1,2,4-triazol-3-one;

5-isobutyl-2-ethyl-2,4-dihydro-4-[[2-(1H-tetrazol-5-yl) [1,1′-biphenyl]-4′-yl]methyl]-3H-1,2,4-triazol-3-one;

5-isobutyl-2-propyl-2,4-dihydro-4-[[2-(1H-tetrazol-5-yl) [1,1′-biphenyl]-4′-yl]methyl]-3H-1,2,4-triazol-3-one;

5-isobutyl-2-isopropyl-2,4-dihydro-4-([2-(1H-tetrazol-5-yl) [1,1′-biphenyl]-4′-yl]methyl]-3H-1,2,4-triazol-3-one;

5-isobutyl-2- butyl-2,4-dihydro-4-[[2-(1H-tetrazol-5-yl) [1,1′-biphenyl]-4′-yl]methyl]-3H-1,2,4-triazol-3-one;

5-isobutyl-2-secbutyl-2,4-dihydro-4-[[2-(1H-tetrazol-5-yl) [1,1′-biphenyl]-4′-yl]methyl]-3H-1,2,4-triazol-3-one; 2,5-diisobutyl-2,4-dihydro-4-[[2-(1H-tetrazol-5-yl) [1,1′-biphenyl]-4′-yl]methyl]-3H-1,2,4-triazol-3-one;

5-isobutyl-2-tertbutyl-2,4-dihydro-4-[[2-(1H-tetrazol-5-yl) [1,1′-biphenyl]-4′-yl]methyl]-3H-1,2,4-triazol-3-one;

5-isobutyl-2-pentyl-2,4-dihydro-4-[[2-(1H-tetrazol-5-yl) [1,1′-biphenyl]-4′-yl]methyl]-3H-1,2,4-triazol-3-one;

5-isobutyl-2-isopentyl-2,4-dihydro-4-[[2-(1H-tetrazol-5-yl) [1,1′-biphenyl]-4′-yl]methyl]-3H-1,2,4-triazol-3-one;

5-isobutyl-2-hexyl-2,4-dihydro-4-[[2-(1H-tetrazol-5-yl) [1,1′-biphenyl]-4′-yl]methyl]-3H-1,2,4-triazol-3-one;

5-isobutyl-2-heptyl-2,4-dihydro-4-[[2-(1H-tetrazol-5-yl) [1,1′-biphenyl]-4′-yl]methyl]-3H-1,2,4-triazol-3-one;

5-isobutyl-2-octyl-2,4-dihydro-4-[[2-(1H-tetrazol-5-yl) [1,1′-biphenyl]-4′-yl]methyl]-3H-1,2,4-triazol-3-one;

5-isobutyl-2-cyclohexyl-2,4-dihydro-4-[[2-(1H-tetrazol-5-yl) [1,1′-biphenyl]-4′-yl]methyl]-3H-1,2,4-triazol-3-one;

5-isobutyl-2-cyclohexylmethyl-2,4-dihydro-4-[[2-(1H-tetrazol-5-yl) [1,1′-biphenyl]-4′-yl]methyl]-3H-1,2,4-triazol-3-one;

5-isobutyl-2-(2-cyclohexylethyl)-2,4-dihydro-4-[[2-(1H-tetrazol-5-yl) [1,1′-biphenyl]-4′-yl]methyl]-3H-1,2,4-triazol-3-one;

5-isobutyl-2-cyclohexanoyl-2,4-dihydro-4-[[2- (1H-tetrazol-5-yl) [1,1′-biphenyl]-4′-yl]methyl]-3H-1,2,4-triazol-3-one;

5-isobutyl-2-(1-oxo-2-cyclohexylethyl)-2,4-dihydro-4-[2-(1H-tetrazol-5-yl) [1,1′-biphenyl]-4′-yl]methyl]-3H-1,2,4-triazol-3-one;

5-isobutyl-2-phenyl-2,4-dihydro-4-[[2-(1H-tetrazol-5-yl) [1,1′-biphenyl]-4′-yl]methyl]-3H-1,2,4-triazol-3-one;

5-isobutyl-2-phenylmethyl-2,4-dihydro-4-[[2-(1H-tetrazol-5-yl) [1,1′-biphenyl]-4′-yl]methyl]-3H-1,2,4-triazol-3-one;

5-isobutyl′2-(2-phenylethyl)-2,4-dihydro-4-[[2-(1H-tetrazol-5-yl) [1,1′-biphenyl]-4′-yl]methyl]-3H-1,2,4-triazol-3-one;

5-isobutyl-2-benzoyl-2,4-dihydro-4-[[2-(1H-tetrazol-5-yl) [1,1′-biphenyl]-4′-yl]methyl]-3H-1,2,4-triazol-3-one;

5-isobutyl-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;

5-isobutyl-2-(1-oxopropyl)-2,4-dihydro-4-[[2-(1H-tetrazol-5-yl) [1,1′-biphenyl]-4′-yl]methyl]-3H-1,2,4-triazol-3-one;

5-isobutyl-2-(1-oxobutyl)-2,4-dihydro-4-[[2-(1H-tetrazol-5-yl) [1,1′-biphenyl]-4′-yl]methyl]-3H-1,2,4-triazol-3-one;

5-isobutyl-2-(1-oxopentyl)-2,4-dihydro-4-[[2-(1H-tetrazol-5-yl) [1,1-biphenyl]-4′-yl]methyl]-3H-1,2,4-triazol-3-one;

5-isobutyl-2-(2-butenyl)-2,4-dihydro-4-[[2-(1H-tetrazol-5-yl) [1,1′-biphenyl]-4′-yl]methyl]-3H-1,2,4-triazol-3-one;

5-isobutyl-2-(3-butenyl)-2,4-dihydro-4-[[2-(1H-tetrazol-5-yl) [1,1′-biphenyl]-4′-yl]methyl]-3H-1,2,4-triazol-3-one;

5-isobutyl-2-(2-butynyl)-2,4-dihydro-4-[[2-(1H-tetrazol-5-yl) [1,1′-biphenyl]-4′-yl]methyl]-3H-1,2,4-triazol-3-one;

5-isobutyl-2-(3-butynyl)-2,4-dihydro-4-[[2-(1H-tetrazol-5-yl) [1,1′-biphenyl]-4′-yl methyl]-3H-1,2,4-triazol-3-one;

5-tertbutyl2-ethyl-2,4-dihydro-4-[[2-(1H-tetrazol-5-yl) [1,1′-biphenyl]-4′-yl]methyl]-3H-1,2,4-triazol-3-one;

5-tertbutyl2-propyl-2,4-dihydro-4-[[2-(1H-tetrazol-5-yl) [1,1′-biphenyl]-4′-yl]methyl]-3H-1,2,4-triazol-3-one;

5-tertbutyl2-isopropyl-2,4-dihydro-4-[[2-(1H-tetrazol-5-yl) [1,1′-biphenyl]-4′-yl]methyl]-3H-1,2,4-triazol-3-one;

5-tertbutyl2-butyl-2,4-dihydro-4-[[2-(1H-tetrazol-5-yl) [1,1′-biphenyl]-4′-yl]methyl]-3H-1,2,4-triazol-3-one;

5-tertbutyl2-secbutyl-2,4-dihydro-4-([2-(1H-tetrazol-5-yl) [1,1′-biphenyl]-4′-yl]methyl]-3H-1,2,4-triazol-3-one;

5-tertbutyl2-isobutyl-2,4-dihydro-4-[[2-(1H-tetrazol-5-yl) [1,1′-biphenyl]-4′-yl]methyl]-3H-1,2,4-triazol-3-one;

2,5-ditertbutyl-2,4-dihydro-4-[[2-(1H-tetrazol-5-yl) [1,1′-biphenyl]-4′-yl]methyl]-3H-1,2,4-triazol-3-one;

5-tertbutyl2-pentyl-2,4-dihydro-4-[[2-(1H-tetrazol-5-yl) [1,1′-biphenyl]-4′-yl]methyl]-3H-1,2,4-triazol-3-one;

5-tertbutyl2-isopentyl-2,4-dihydro-4-[[2-(1H-tetrazol-5-yl) [1,1′-biphenyl]-4′-yl]methyl]-3H-1,2,4-triazol-3-one;

5-tertbutyl2-hexyl-2,4-dihydro-4-[[2-(1H-tetrazol-5-yl) [1,1′-biphenyl]-4′-yl]methyl]-3H-1,2,4-triazol-3-one;

5-tertbutyl2-heptyl-2,4-dihydro-4-[[2-(1H-tetrazol-5-yl) [1,1′-biphenyl]-4′-yl]methyl]-3H-1,2,4-triazol-3-one;

5-tertbutyl2-octyl-2,4-dihydro-4-[[2-(1H-tetrazol-5-yl) [1,1′-biphenyl]-4′-yl]methyl]-3H-1,2,4-triazol-3-one;

5-tertbutyl2-cyclohexyl-2,4-dihydro-4-[[2-(1H-tetrazol-5-yl) [1,1′-biphenyl]-4′-yl]methyl]-3H-1,2,4-triazol-3-one;

5-tertbutyl2-cyclohexylmethyl-2,4-dihydro-4-[[2-(1H-tetrazol-5-yl) [1,1′-biphenyl]-4′-yl]methyl]-3H-1,2,4-triazol-3-one;

5-tertbutyl2-(2-cyclohexylethyl)-2,4-dihydro-4-[[2-(1H-tetrazol-5-yl) [1,1′-biphenyl]-4′-yl]methyl]-3H-1,2,4-triazol-3-one;

5-tertbutyl2-cyclohexanoyl-2,4-dihydro-4-[[2-(1H-tetrazol-5-yl) [1,1′-biphenyl]-4′-yl]methyl]-3H-1,2,4-triazol-3-one;

5-tertbutyl2-(1-oxo-2-cyclohexylethyl)-2,4-dihydro-4-[[2-[1H-tetrazol-5-yl) [1,1′-biphenyl]-4′-yl]methyl]-3H-1,2,4-triazol-3-one;

5-tertbutyl2-phenyl-2,4-dihydro-4-[[2-(1H-tetrazol-5-yl) [1,1′-biphenyl]-4′-yl]methyl]-3H-1,2,4-triazol-3-one;

5-tertbutyl2-phenylmethyl-2,4-dihydro-4-[[2-(1H-tetrazol-5-yl) [1,1′-biphenyl]-4′-yl]methyl]-3H-1,2,4-triazol-3-one;

5-tertbuty2-(2-phenylethyl)-2,4-dihydro-4-[[2-(1H-tetrazol-5-yl) [1,1′-biphenyl]-4′-yl]methyl]-3H-1,2,4-triazol-3-one;

5-tertbutyl2-benzoyl-2,4-dihydro-4-[[2-(1H-tetrazol-5-yl) [1,1′-biphenyl]-4′-yl]methyl]-3H-1,2,4-triazol-3-one;

5-tertbutyl2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;

5-tertbutyl2-(1-oxopropyl)-2,4-dihydro-4-[[2-(1H-tetrazol-5-yl) [1,1′-biphenyl]-4′-yl]methyl]-3H-1,2,4-triazol-3-one;

5-tertbutyl2-(1-oxobutyl)-2,4-dihydro-4-[[2-(1H-tetrazol-5-yl) [1,1′-biphenyl]-4′-yl]methyl3-3H-1,2,4-triazol-3-one;

5-tertbutyl2-(1-oxopentyl)-2,4-dihydro-4-[[2-(1H-tetrazol-5-yl) [1,1′-biphenyl]-4′-yl]methyl]-3H-1,2,4-triazol-3-one;

5-tertbutyl2-(2-butenyl)-2,4-dihydro-4-[[2-(1H-tetrazol-5-yl) [1,1′-biphenyl]-4′-yl]methyl]-3H-1,2,4-triazol-3-one;

5-tertbutyl2-(3-butenyl)-2,4-dihydro-4-[(2-(1H-tetrazol-5-yl) [1,1′-biphenyl]-41-yl]methyl]-3H-1,2,4-triazol-3-one;

5-tertbutyl2-(2-butynyl)-2,4-dihydro-4-([2-(1H-tetrazol-5-yl) [1,1′-biphenyl]-4′-yl]methyl]-3H-1,2,4-triazol-3-one;

5-tertbutyl2-(3-butynyl)-2,4-dihydro-4-[[2-(1H-tetrazol-5-yl) [1,1′-biphenyl]-4′-yl]methyl]-3H-1,2,4-triazol-3-one;

5-pentyl-2-ethyl-2,4-dihydro-4-[[2-(1H-tetrazol-5-yl) [1,1′-biphenyl]-4′-yl]methyl]-3H-1,2,4-triazol-3-one;

5-pentyl-2-propyl-2,4-dihydro-4-[[2-(1H-tetrazol-5-yl) [1,1′-biphenyl]-4′-yl]methyl]-3H-1,2,4-triazol-3-one;

5-pentyl-2-isopropyl-2,4-dihydro-4-[[2-(1H-tetrazol-5-yl) [1,1′-biphenyl]-4′-yl]methyl]-3H-1,2,4-triazol-3-one;

5-pentyl-2-butyl-2,4-dihydro-4-[[2-(1H-tetrazol-5-yl) [1,1′-biphenyl]-4′-yl]methyl]-3H-1,2,4-triazol-3-one;

5-pentyl-2-secbutyl-2,4-dihydro-4-[[2-(1H-tetrazol-5-yl) [1,1′-biphenyl]-4′-yl]methyl]-3H-1,2,4-triazol-3-one;

5-pentyl-2-isobutyl-2,4-dihydro-4-[[2-(1H-tetrazol-5-yl) [1,1′-biphenyl]-4′-yl]methyl]-3H-1,2,4-triazol-3-one;

5-pentyl-2-tertbutyl-2,4-dihydro-4-[[2-(1H-tetrazol-5-yl) [1,1′-biphenyl]-4′-yl]methyl]-3H-1,2,4-triazol-3-one; 2,5-dipentyl-2,4-dihydro-4-[[2-(1H-tetrazol-5-yl) [1,1′-biphenyl]-4′-yl]methyl]-3H-1,2,4-triazol-3-one;

5-pentyl-2-isopentyl-2,4-dihydro-4-[[2-(1H-tetrazol-5-yl) [1,1′-biphenyl]-4′-yl]methyl]-3H-1,2,4-triazol-3-one;

5-pentyl-2-hexyl-2,4-dihydro-4-[[2-(1H-tetrazol-5-yl) [1,1′-biphenyl]-4′-yl]methyl]-3H-1,2,4-triazol-3-one;

5-pentyl-2-heptyl-2,4-dihydro-4-[[2-(1H-tetrazol-5-yl) [1,1′-biphenyl]-4′-yl]methyl]-3H-1,2,4-triazol-3-one;

5-pentyl-2-octyl-2,4-dihydro-4-[[2-(1H-tetrazol-5-yl) [1,1′-biphenyl]-4′-yl]methyl]-3H-1,2,4-triazol-3-one;

5-pentyl-2-cyclohexyl-2,4-dihydro-4-[[2-(1H-tetrazol-5-yl) [1,1′-biphenyl]-4′-yl]methyl]-3H-1,2,4-triazol-3-one;

5-pentyl-2-cyclohexylmethyl-2,4-dihydro-4-[[2-(1H-tetrazol-5-yl) [1,1′-biphenyl]-4′-yl]methyl]-3H-1,2,4-triazol-3-one;

5-pentyl-2-(2-cyclohexylethyl)-2,4-dihydro-4-[[2-(1H-tetrazol-5-yl) [1,1′-biphenyl]-4′-yl]methyl]-3H-1,2,4-triazol-3-one;

5-pentyl-2-cyclohexanoyl-2,4-dihydro-4-[[2-(1H-tetrazol-5-yl) [1,1′-biphenyl]-4′-yl]methyl]-3H-1,2,4-triazol-3-one;

5-pentyl-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;

5-pentyl-2-phenyl-2,4-dihydro-4-[[2-(1H-tetrazol-5-yl) [1,1′-biphenyl]-4′-yl]methyl]-3H-1,2,4-triazol-3-one;

5-pentyl-2-phenylmethyl-2,4-dihydro-4-[[2-(1H-tetrazol-5-yl) [1,1′-biphenyl]-4′-yl]methyl]-3H-1,2,4-triazol-3-one;

5-pentyl-2-(2-phenylethyl)-2,4-dihydro-4-[[2-(1H-tetrazol-5-yl) [1,1′-biphenyl]-4′-yl]methyl]-3H-1,2,4-triazol-3-one;

5-pentyl-2-benzoyl-2,4-dihydro-4-[[2-(1H-tetrazol-5-yl) [1,1′-biphenyl]-4′-yl]methyl]-3H-1,2,4-triazol-3-one;

5-pentyl-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;

5-pentyl-2-(1-oxopropyl)-2,4-dihydro-4-[[2-(1H-tetrazol-5-yl) [1,1′-biphenyl]-4′-yl]methyl]-3H-1,2,4-triazol-3-one;

5-pentyl-2-(1-oxobutyl)-2,4-dihydro-4-[[2-(1H-tetrazol-5-yl) [1,1′-biphenyl]-4′-yl]methyl]-3H-1,2,4-triazol-3-one;

5-pentyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-pentyl-2-(2-butenyl)-2,4-dihydro-4-[[2-(1H-tetrazol-5-yl) [1,1′-biphenyl]-4′-yl]methyl]-3H-1,2,4-triazol-3-one;

5-pentyl-2-(3-butenyl)-2,4-dihydro-4-[[2-(1H-tetrazol-5-yl) [1,1′-biphenyl]-4′-yl]methyl]-3H-1,2,4-triazol-3-one;

5-pentyl-2-(2-butynyl)-2,4-dihydro-4-[[2-(1H-tetrazol-5-yl) [1,140 -biphenyl]-4′-yl]methyl]-3H-1,2,4-triazol-3-one;

5-pentyl-2-(3-butynyl)-2,4-dihydro-4-[[2-(1H-tetrazol-5-yl) [1,1′-biphenyl]-4′-yl]methyl]-3H-1,2,4-triazol-3-one;

5-isopentyl-2-ethyl-2,4-dihydro-4-[[2-(1H-tetrazol-5-yl) [1,1′-biphenyl]-4′-yl]methyl]-3H-1,2,4-triazol-3-one;

5-isopentyl-2-propyl-2,4-dihydro-4-[[2-(1H-tetrazol-5-yl) [1,1′-biphenyl]-4′-yl]methyl]-3H-1,2,4-triazol-3-one;

5-isopentyl-2-isopropyl-2,4-dihydro-4-[[2-(1H-tetrazol-5-yl) [1,1′-biphenyl]-4′-yl]methyl]-3H-1,2,4-triazol-3-one;

5-isopentyl-2-butyl-2,4-dihydro-4-[[2-(1H-tetrazol-5-yl) [1,1′-biphenyl]-4′-yl]methyl]-3H-1,2,4-triazol-3-one;

5-isopentyl-2-secbutyl-2,4-dihydro-4-[[2-(1H-tetrazol-5-yl) [1,1′-biphenyl]-4′-yl]methyl]-3H-1,2,4-triazol-3-one;

5-isopentyl-2-isobutyl-2,4-dihydro-4-[[2-(1H-tetrazol-5-l) [1,1′-biphenyl]-4′-yl]methyl]-3H-1,2,4-triazol-3-one;

5-isopentyl-2-tertbutyl-2,4-dihydro-4-[[2-(1H-tetrazol-5-yl) [1,1′-biphenyl]-4′-yl]methyl]-3H-1,2,4-triazol-3-one;

5-isopentyl-2-pentyl-2,4-dihydro-4-[[2-(1H-tetrazol-5-yl) [1,1′-biphenyl]-4′-yl]methyl]-3H-1,2,4-triazol-3-one;

5-isopentyl-2-pentyl-2,4-dihydro-4-[[2-(1H-tetrazol-5-yl) [1,1′-biphenyl]-4′-yl]methyl]-3H-1,2,4-triazol-3-one; 2,5-diisopentyl-2,4-dihydro-4-[[2-(1H-tetrazol-5-yl) [1,1′-biphenyl]-4′-yl]methyl]-3H-1,2,4-triazol-3-one;

5-isopentyl-2-heptyl-2,4-dihydro-4-[[2-(1H-tetrazol-5-yl) [1,1′-biphenyl]-4′-yl]methyl]-3H-1,2,4-triazol-3-one;

5-isopentyl-2-octyl-2,4-dihydro-4-[[2-(1H-tetrazol-5-yl) [1,1′-biphenyl]-4′-yl]methyl]-3H-1,2,4-triazol-3-one;

5-isopentyl-2-cyclohexyl-2,4-dihydro-4-[[2-(1H-tetrazol-5-yl) [1,1′-biphenyl]-4′-yl]methyl]-3H-1,2,4-triazol-3-one;

5-isopentyl-2-cyclohexylmethyl-2,4-dihydro-4-[[2-(1H-tetrazol-5-yl) [1,1′-biphenyl]-4′-yl]methyl]-3H-1,2,4-triazol-3-one;

5-isopentyl-2-(2-cyclohexylethyl)-2,4-dihydro-4-[[2-(1H-tetrazol-5-yl) [1,1′-biphenyl]-4′-yl]methyl]-3H-1,2,4-triazol-3-one;

5-isopentyl-2-cyclohexanoyl-2,4-dihydro-4-[[2-(1H-tetrazol-5-yl) [1,1′-biphenyl]-4′-yl]methyl7-3H-1,2,4-triazol-3-one;

5-isopentyl-2-(1-oxo-2-cyclohexylethyl)-2,4-dihydro-4-[[2-(1H-tetrazol-5-yl) [1,1′-biphenyl)-4′-yl]methyl]-3H-1,2,4-triazol-3-one;

5-isopentyl-2-phenyl-2,4-dihydro-4-[[2-(1H-tetrazol-5-yl) [1,1′-biphenyl]-4′-yl]methyl]-3H-1,2,4-triazol-3-one;

5-isopentyl-2-phenylmethyl-2,4-dihydro-4-[[2-(1H-tetrazol-5-yl) [1,1′-biphenyl]-4′-yl]methyl]-3H-1,2,4-triazol-3-one;

5-isopentyl-2-(2-phenylethyl)-2,4-dihydro-4-[[2-(1H-tetrazol-5-yl) [1,1′-biphenyl]-4′-yl]methyl)-3H-1,2,4-triazol-3-one;

5-isopentyl-2-benzoyl-2,4-dihydro-4-[[2-(1H-tetrazol-5-yl) [1,1′-biphenyl)-4′-yl]methyl]-3H-1,2,4-triazol-3-one;

5-isopentyl-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;

5-isopentyl-2-(l-oxopropyl)-2,4-dihydro-4-[[2-(1H-tetrazol-5-yl) [1,1′-biphenyl]-4′-yl]methyl]-3H-1,2,4-triazol-3-one;

5-isopentyl-2-(1-oxobutyl)-2,4-dihydro-4-[(2-(1H-tetrazol-5-yl) [1,1′-biphenyl]-4′-yl]methyl]-3H-1,2,4-triazol-3-one;

5-isopentyl-2-(1-oxopentyl)-2,4-dihydro-4-([2-(1H-tetrazol-5-yl) [1,1′-biphenyl]-4′-yl]methyl]-3H-1,2,4-triazol-3-one;

5-isopentyl-2-(2-butenyl)-2,4-dihydro-4-[[2-(1H-tetrazol-5-yl) [1,1′-biphenyl]-4′-yl]methyl]-3H-1,2,4-triazol-3-one;

5-isopentyl-2-(3-butenyl)-2,4-dihydro-4-[[2-(1H-tetrazol-5-yl) [1,1′-biphenyl]-4′-yl]methyl]-3H-1,2,4-triazol-3-one;

5-isopentyl-2-(2-butynyl)-2,4-dihydro-4- [[2-(1H-tetrazol-5-yl) [1,1′-biphenyl]-4′-yl]methyl]-3H-1,2,4-triazol-3-one;

5-isopentyl-2-(3-butynyl)-2,4-dihydro-4-[[2-(1H-tetrazol-5-yl) [1,1′-biphenyl]-4′-yl]methyl]-3H-1,2,4-triazol-3-one;

5-hexyl-2-ethyl-2,4-dihydro-4-[[2-(1H-tetrazol-5-yl) [1,1′-biphenyl]-4′-yl]methyl]-3H-1,2,4-triazol-3-one;

5-hexyl-2-propyl-2,4-dihydro-4-[[2-(1H-tetrazol-5-yl) [1,1′-biphenyl]-4′-yl]methyl]-3H-1,2,4-triazol-3-one;

5-hexyl-2-isopropyl-2,4-dihydro-4-[[2-(1H-tetrazol-5-yl) [1,1′-biphenyl]-4′-yl]methyl]-3H-1,2,4-triazol-3-one;

5-hexyl-2-butyl-2,4-dihydro-4-[[2-(1H-tetrazo1-5-yl) [1,1′-biphenyl]-4′-yl]methyl]-3H-1,2,4-triazol-3-one;

5-hexyl-2-secbutyl-2,4-dihydro-4-[[2-(1H-tetrazol-5-yl) [1,1′-biphenyl]-4′-yl]methyl]-3H-1,2,4-triazol-3-one;

5-hexyl-2-isobutyl-2,4-dihydro-4-[[2-(1H-tetrazol-5-yl) [1,1′-biphenyl]-4′-yl]methyl]-3H-1,2,4-triazoi-3-one;

5-hexyl-2-tertbutyl-2,4-dihydro-4-[[2-(1H-tetrazol-5-yl) [1,1′-biphenyl]-4¹-yl]methyl]-3H-1,2,4-triazol-3-one;

5-hexyl-2-pentyl-2,4-dihydro-4-[[2-(1H-tetrazol-5-yl) [1,1′-biphenyl]-4′-yl]methyl]-3H-1,2,4-triazol-3-one;

5-hexyl-2-isopentyl-2,4-dihydro-4-[[2-(1H-tetrazol-5-yl) [1,1′-biphenyl]-4′-yl]methyl]-3H-1,2,4-triazol-3-one; 2,5-dihexyl-2,4-dihydro-4-[[2-(1H-tetrazol-5-yl) [1,1′-biphenyl]-4′-yl]methyl]-3H-1,2,4-triazol-3-one;

5-hexyl-2-heptyl-2,4-dihydro-4-([2-(1H-tetrazol-5-yl) [1,1′-biphenyl)-4′-yl]methyl]-3H-1,2,4-triazol-3-one;

5-hexyl-2-octyl-2,4-dihydro-4-[[2-(1H-tetrazol-5-yl) [1,1′-biphenyl]-4′-yl]methyl]-3H-1,2,4-triazol-3-one;

5-hexyl-2-cyclohexyl-2,4-dihydro-4-[[2-(1H-tetrazol-5-yl) [1,1′-biphenyl]-4′-yl]methyl]-3H-1,2,4-triazol-3-one;

5-hexyl-2-cyclohexylmethyl-2,4-dihydro-4-[[2-(1H-tetrazol-5-yl) [1,1′-biphenyl]-4′-yl]methyl]-3H-1,2,4-triazol-3-one;

5-hexyl-2-(2-cyclohexylethyl)-2,4-dihydro-4-[[2-(1H-tetrazol-5-yl) [1,1′-biphenyl]-4′-yl]methyl]-3H-1,2,4-triazol-3-one;

5-hexyl-2-cyclohexanoyl-2,4-dihydro-4-[[2-(1H-tetrazol-5-yl) [1,1′-biphenyl]-4′-yl]methyl]-3H-1,2,4-triazol-3-one;

5-hexyl-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;

5-hexyl-2-phenyl-2,4-dihydro-4-[[2-(1H-tetrazol-5-yl) [1,1′-biphenyl]-4′-yl]methyl]-3H-1,2,4-triazol-3-one;

5-hexyl-2-phenylmethyl-2,4-dihydro-4-[[2-(1H-tetrazol-5-yl) [1,1′-biphenyl]-4′-yl]methyl-3H-1,2,4-triazol-3-one;

5-hexyl-2-(2-phenylethyl)-2,4-dihydro-4-[[2-(1H-tetrazol-⁵-yl) [1,1′-biphenyl]-4¹-yl]methyl]-3H-1,2,4-triazol-3-one;

5-hexyl-2-benzoyl-2,4-dihydro-4-[[2-(1H-tetrazol-5-yl) [1,1′-biphenyl]-4′-yl]methyl]-3H-1,2,4-triazol-3-one;

5-hexyl-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;

5-hexyl-2-(1-oxopropyl)-2,4-dihydro-4-[[2-(1H-tetrazol-5-yl) [1,1′-biphenyl]-4′-yl]methyl]-3H-1,2,4-triazol-3-one;

5-hexyl-2-(1-oxobutyl)-2,4-dihydro-4-[[2-(1H-tetrazol-5-yl) [1,1′-biphenyl]-4′-yl]methyl]-3H-1,2,4-triazol-3-one;

5-hexyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-hexyl-2-(2-butenyl)-2,4-dihydro-4-[[2-(1H-tetrazol-5-yl) [1,1′-biphenyl]-4′-yl]methyl]-3H-1,2,4-triazol-3-one;

5-hexyl-2-(3-butenyl) -2, 4-dihydro-4-[[2- (1H-tetrazol-5-yl) [1,1′-biphenyl]-4′-yl]methyl]-3H-1,2,4-triazol-3-one;

5-hexyl-2-(2-butynyl)-2,4-dihydro-4-[[2-(1H-tetrazol-5-yl) [1,1′-biphenyl]-4′-yl]methyl]-3H-1,2,4-triazol-3-one;

5-hexyl-2-(3-butynyl)-2,4-dihydro-4-[[2-(1H-tetrazol-5-yl) [1,1-biphenyl]-4′-yl]methyl]-3H-1,2,4-triazol-3-one;

5-heptyl-2-ethyl-2,4-dihydro-4-[[2-(1H-tetrazol-5-yl) [1,1′-biphenyl] -4′-yl]methyl]-3H-1,2,4-triazol-3-one;

5-heptyl-2-propyl-2,4-dihydro-4-[[2-(1H-tetrazol-5-yl) [1,1′- biphenyl]-4′-yl]methyl]-3H-1,2,4-triazol-3-one;

5-heptyl-2-isopropyl-2,4-dihydro-4-[[2-(1H-tetrazol-5-yl) [1,1′-biphenyl]-4′-yl]methyl]-3H-1,2,4-triazol-3-one;

5-heptyl-2-butyl-2,4-dihydro-4-[[2-(1H-tetrazol-5-yl) [1,1′-biphenyl]-4′-yl]methyl]-3H-1, 2,4-triazo1-3-one;

5-heptyl-2-secbutyl-2,4-dihydro-4-[[2-(1H-tetrazol-5-yl) [1,1′-biphenyl]-4′-yl]methhyl]-3H-1,2,4-triazol-3-one;

5-heptyl-2-isobutyl-2,4-dihydro-4-[[2-(1H-tetrazol-5-yl) [1,1′-biphenyl]-4′-yl]methyl]-3H-1,2,4-triazol-3-one;

5-heptyl-2-tertbutyl-2,4-dihydro-4-[[2-(1H-tetrazol-5-yl) [1,1′-biphenyl]-4′-yl]methyl]-3H-1,2,4-triazol-3-one;

5-heptyl-2-pentyl-2,4-dihydro-4-[[2-(1H-tetrazol-5-yl) [1,1′-biphenyl]-4′-yl]methyl]-3H-1,2,4-triazol-3-one;

5-heptyl-2-isopentyl-2,4-dihydro-4-[[2-(1H-tetrazol-5-yl) [1,1′-biphenyl]-4′-yl]methyl]-3H-1,2,4-triazol-3-one;

5-heptyl-2-hexyl-2,4-dihydro-4-[[2-(1H-tetrazol-5-yl) [1,1′-biphenyl]-4′-yl]methyl]-3H-1,2,4-triazol-3-one;

2,5-diheptyl-2,4-dihydro-4-([2-(1H-tetrazol-5-yl) [1,1′-biphenyl]-4′-yl]methyl]-3H-1,2,4-triazol-3-one;

5-heptyl-2-octyl-2,4-dihydro-4-[[2-(1H-tetrazol-5-yl) [1,1′-biphenyl]-4′-yl]methyl]-3H-1,2,4-triazol-3-one;

5-heptyl-2-cyclohexyl-2,4-dihydro-4-[[2-(1H-tetrazol-5-yl) [1,1′-biphenyl]-4′-yl]methyl]-3H-1,2,4-triazol-3-one;

5-heptyl-2-cyclohexylmethyl-2,4-dihydro-4-[[2-(1H-tetrazol-5-yl) [1,1′-biphenyl]-4′-yl]methyl]-3H-1,2,4-triazol-3-one;

5-heptyl-2-(2-cyclohexylethyl)-2,4-dihydro-4-[[2-(1H-tetrazol-5-yl) [1,1′-biphenyl]-4′-yl]methyl]-3H-1,2,4-triazol-3-one;

5-heptyl-2-cyclohexanoyl-2,4-dihydro-4-[[2-(1H-tetrazol-5-yl) [1,1′-biphenyl]-4′-yl]methyl]-3H-1,2,4-triazol-3-one;

5-heptyl-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;

5-heptyl-2-phenyl-2,4-dihydro-4- [[2- (1H-tetrazol-5-yl) [1,1′-biphenyl]-4′-yl]methyl]-3H-1,2,4-triazol-3-one;

5-heptyl-2-phenylmethyl-2,4-dihydro-4-[[2-(1H-tetrazol-5-yl) [1,1′-biphenyl]-4′-yl]methyl]-3H-1,2,4-triazol-3-one;

5-heptyl-2-(2-phenylethyl)-2,4-dihydro-4-[[2-(1H-tetrazol-5-yl) [1,1′-biphenyl]-4′-yl]methyl]-3H-1,2,4-triazol-3-one;

5-heptyl-2-benzoyl-2,4-dihydro-4-[(2-(1H-tetrazol-5-yl) [1,1′-biphenyl]-4′-yl]methyl]-3H-1,2,4-triazol-3-one;

5-heptyl-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;

5-heptyl-2-(1-oxopropyl)-2,4-dihydro-4-[[2-(1H-tetrazol-5-yl) [1,1′-biphenyl]-4′-yl]methyl]-3H-1,2,4-triazol-3-one;

5-heptyl-2-(1-oxobutyl)-2,4-dihydro-4-[(2-(1H-tetrazol-5-yl) [1,1′-biphenyl]-4′-yl]methyl]-3H-1,2,4-triazol-3-one;

5-heptyl-2-(1-oxopentyl)-2,4-dihydro-4-[[2-(1H-tetrazol-5-yl) [1,1′-biphenyl]-4t-yl]methyl]-3H-1,2,4-triazol-3-one;

5-heptyl-2-(2-butenyl)-2,4-dihydro-4-[[2-(1H-tetrazol-5-yl) [1,1′-biphenyl]-4′-yl]methyl]-3H-1,2,4-triazol-3-one;

5-heptyl-2-(3-butenyl)-2,4-dihydro-4-[[2-(1H-tetrazol-5-yl) [1,1′-biphenyl]-4′-yl]methyl]-3H-1,2,4-triazol-3-one;

5-heptyl-2-(2-butynyl)-2,4-dihydro-4-[[2-(1H-tetrazol-5-yl) [1,1′-biphenyl]-4′-yl]methyl]-3H-1,2,4-triazol-3-one;

5-heptyl-2-(3-butynyl)-2,4-dihydro-4-[[2-(1H-tetrazol-5-yl) [1,1′-biphenyl]-4′-yl]methyl]-3H-1,2,4-triazol-3-one;

5-octyl-2-ethyl-2,4-dihydro-4-[[2-(1H-tetrazol-5-yl) [1,1′-biphenyl]-4′-yl]methyl]-3H-1,2,4-triazol-3-one;

5-octyl-2-propyl-2,4-dihydro-4-[[2-(1H-tetrazol-5-yl) [1,1′-biphenyl]-4′-yl]methyl]-3H-1,2,4-triazol-3-one;

5-octyl-2-isopropyl-2,4-dihydro-4-[[2-(1H-tetrazol-5-yl) [1,1′-biphenyl]-4′-yl]methyl]-3H-1,2,4-triazol-3-one;

5-octyl-2-butyl-2,4-dihydro-4-[[2-(1H-tetrazol-5-yl) [1,1′-biphenyl]-4′-yl]methyl]-3H-1,2,4-triazol-3-one;

5-octyl-2-secbutyl-2,4-dihydro-4-[[2-(1H-tetrazol-5-yl) [1,1′-biphenyl]-4′-yl]methyl]-3H-l,2,4-triazol-3-one;

5-octyl-2-isobutyl-2,4-dihydro-4-[[2-(1H-tetrazol-5-yl) [1,1′-biphenyl]-4′-yl]methyl]-3H-1,2,4-triazol-3-one;

5-octyl-2-tertbutyl-2,4-dihydro-4-[[2-(1H-tetrazol-5-yl) [1,1′-biphenyl]-4′-yl]methyl]-3H-1,2,4-triazol-3-one;

5-octyl-2-pentyl-2,4-dihydro-4-[[2-(1H-tetrazol-5-yl) [1,1′-biphenyl]-4′-yl]methyl]-3H-1,2,4-triazol-3-one;

5-octyl-2-isopentyl-2,4-dihydro-4-[[2-(1H-tetrazol-5-yl) [1,1′-biphenyl]-4′-yl]methyl]-3H-1,2,4-triazol-3-one;

5-octyl-2-hexyl-2,4-dihydro-4-[[2-(1H-tetrazol-5-yl) [1,1′-biphenyl]-4′-yl]methyl]-3H-1,2,4-triazol-3-one;

5-octyl-2-heptyl-2,4-dihydro-4-[[2-(1H-tetrazol-5-yl) [1,1′-biphenyl]-4′-yl]methyl]-3H-1,2,4-triazol-3-one;

2,5-dioctyl-2,4-dihydro-4-[[2-(1H-tetrazol-5-yl) [1,1′-biphenyl]-4′-yl]methyl]-3H-1,2,4-triazol-3-one;

5-octyl-2-cyclohexyl-2,4-dihydro-4-[[2-(1H-tetrazol-5-yl) [1,1′-biphenyl]-4′-yl1methyl]-3H-1,2,4-triazol-3-one;

5-octyl-2-cyclohexylmethyl-2,4-dihydro-4-[[2-(1H-tetrazol-5-yl) [1,1′-biphenyl]-4′-yl]methyl]-3H-1,2,4-triazol-3-one;

5-octyl-2-(2-cyclohexylethyl)-2,4-dihydro-4-[[2-(1H-tetrazol-5-yl) [1,1′-biphenyl]-4′-yl]methyl]-3H-1,2,4-triazol-3-one;

5-octyl-2-cyclohexanoyl-2,4-dihydro-4-[[2-(1H-tetrazol-5-yl) [1,1′-biphenyl]-4′-yl]methyl]-3H-1,2,4-triazol-3-one;

5-octyl-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;

5-octyl-2-phenyl-2,4-dihydro-4-[[2-(1H-tetrazol-5-yl) [1,1′-biphenyl]-4′-yl]methyl]-3H-1,2,4-triazol-3-one;

5-octyl-2-phenylmethyl-2,4-dihydro-4-[[2-(1H-tetrazol-5-yl) [1,1′-biphenyl]-4′-yl]methyl]-3H-1,2,4-triazol-3-one;

5-octyl-2-(2-phenylethyl)-2,4-dihydro-4-[[2-(1H-tetrazol-5-yl) [1,1′-biphenyl]-4′-yl]methyl]-3H-1,2,4-triazol-3-one;

5-octyl-2-benzoyl-2,4-dihydro-4-[[2-(1H-tetrazol-5-yl) [1,1′-blphenyl]-4′-yl]methyl]-3H-1,2,4-triazol-3-one;

5-octyl-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;

5-octyl-2-(1-oxopropyl)-2,4-dihydro-4-[[2-(1H-tetrazol-5-yl) [1,1′-biphenyl]-4′-yl]methyl]-3H-1,2,4-triazol-3-one;

5-octyl-2-(1-oxobutyl)-2,4-dihydro-4-[[2-(1H-tetrazol-5-yl) [1,1′-biphenyl]-4′-yl]methyl]-3H-1,2,4-triazol-3-one;

5-octyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-octyl-2-(2-butenyl)-2,4-dihydro-4-[[2-(1H-tetrazol-5-yl) [1,1′-biphenyl]-4′-yl]methyl]-3H-1,2,4-triazol-3-one;

5-octyl-2-(3-butenyl)-2,4-dihydro-4-[[2-(1H-tetrazol-5-yl) [1,1′-biphenyl]-4′-yl]methyl]-3H-1,2,4-triazol-3-one;

5-octyl-2-(2-butynyl)-2,4-dihydro-4-[[2-(1H-tetrazol-5-yl) [1,1′-biphenyl]-4′-yl]methyl]-3H-1,2,4-triazol-3-one;

5-octyl-2-(3-butynyl)-2,4-dihydro-4-[[2-(1H-tetrazol-5-yl) [1,1′-biphenyl]-4′-yl]methyl]-3H-1,2,4-triazol-3-one;

5-(1-butenyl)-2-ethyl-2,4-dihydro-4-[[2-(1H-tetrazol-5-yl) [1,1′-biphenyl]-4′-yl]methyl]-3H-1,2,4-triazol-3-one;

5-(1-butenyl)-2-propyl-2,4-dihydro-4-[[2-(1H-tetrazol-5-yl) [1,1′-biphenyl]-4′-yl]methyl]-3H-1,2,4-triazol-3-one;

5-(1-butenyl)-2-isopropyl-2,4-dihydro-4-[[2-(1H-tetrazol-5-yl) [1,1′-biphenyl-4′-yl]methyl]-3H-1,2,4-triazol-3-one;

5-(1-butenyl)-2-butyl-2,4-dihydro-4-[[2-(1H-tetrazol-5-yl) [1,1′-biphenyl]-4′-yl]methyl]-3H-1,2,4-triazol-3-one;

5-(1-butenyl)-2-secbutyl-2,4-dihydro-4-[[2-(1H-tetrazol-5-yl) [1,1′-biphenyl]-4′-yl]methyl]-3H-1,2,4-triazol-3-one;

5-(1-butenyl)-2-isobutyl-2,4-dihydro-4-[[2-(1H-tetrazol-5-yl) [1,1′-biphenyl]-4′-yl]methyl]-3H-1,2,4-triazol-3-one;

5-(1-butenyl)-2-tertbutyl-2,4-dihydro-4-[[2-(1H-tetrazol-5-yl) [1,1′-biphenyl]-4′-yl]methyl]-3H-1,2,4-triazol-3-one;

5-(1-butenyl)-2-pentyl-2,4-dihydro-4-[[2-(1H-tetrazol-5-yl) [1,1′-biphenyl]-4′-yl]methyl]-3H-1,2,4-triazol-3-one;

5-(1-butenyl)-2-isopentyl-2,4-dihydro-4-[[2-(1H-tetrazol-5-yl) [1,1′-biphenyl]-4′-yl]methyl]-3H-1,2,4-triazol-3-one;

5-(1-butenyl)-2-hexyl-2,4-dihydro-4-[[2-(1H-tetrazol-5-yl) [1,1′-biphenyl]-4′-yl]methyl]-3H-1,2,4-triazol-3-one;

5-(1-butenyl)-2-heptyl2,4-dihydro-4-[[2-(1H-tetrazol-5-yl) [1,1′-biphenyl]-4′-yl]methyl]-3H-1,2,4-triazol-3-one;

5-(1-butenyl)-2-octyl-2,4-dihydro-4-[(2-(1H-tetrazol-5-yl) [1,1′-biphenyl]-4′-yl]methyl]-3H-1,2,4-triazol-3-one;

5-(1-butenyl)-2-cyclohexyl-2,4-dihydro-4-[(2-(1H-tetrazol-5-yl) [1,1′-biphenyl]-4′-yl]methyl]-3H-1,2,4-triazol-3-one;

5-(1-butenyl)-2-cyclohexylmethyl-2,4-dihydro-4-[[2-(1H-tetrazol-5-yl) [1,1′-biphenyl]-4′-yl]methyl]-3H-1,2,4-triazol-3-one;

5-(1-butenyl)-2-(2-cyclohexylethyl)-2,4-dihydro-4-[[2-(1H-tetrazol-5-yl) [1,1′-biphenyl]-4′-yl]methyl]-3H-1,2,4-triazol-3-one;

5-(1-butenyl)-2-cyclohexanoyl-2,4-dihydro-4-[[2-(1H-tetrazol-5-yl) [1,1′-biphenyl]-4′-yl]methyl]-3H-1,2,4-triazol-3-one;

5-(1-butenyl)-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;

5-(1-butenyl)-2-phenyl-2,4-dihydro-4-[[2-(1H-tetrazol-5-yl) [1,1′-biphenyl]-4′-yl]methyl]-3H-1,2,4-triazol-3-one;

5-(1-butenyl)-2-phenylmethyl-2,4-dihydro-4-[[2-(1H-tetrazol-5-yl) [1,1′-biphenyl]-4′-yl]methyl]-3H-1,2,4-triazol-3-one;

5-(1-butenyl)-2-benzoyl-2,4-dihydro-4-[[2-(1H-tetrazol-5-yl) [1,1′-biphenyl]-4′-yl]methyl]-3H-1,2,4-triazol-3-one;

5-(1-butenyl)-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;

5-(1-butenyl)-2-(1-oxopropyl)-2,4-dihydro-4-[[2-(1H-tetrazol-5-yl) [1,1′-biphenyl]-4′-yl]methyl]-3H-1,2,4-triazol-3-one;

5-(1-butenyl)-2-(1-oxobutyl)-2,4-dihydro-4-[[2-(1H-tetrazol-5-yl) [1,1′-biphenyl]-4′-yl]methyl]-3H-1,2,4-triazol-3-one;

5-(1-butenyl)-2-(1-oxopentyl)-2,4-dihydro-4-[[2-(1H-tetrazol-5-yl) [1,1′-biphenyl]-4′-yl]methyl]-3H-1,2,4-triazol-3-one;

5-(1-butenyl)-2-(2-butenyl)-2,4-dihydro-4-[[2-(1H-tetrazol-5-yl) [1,1′-biphenyl]-4′-yl]methyl]-3H-1,2,4-triazol-3-one;

5-(1-butenyl)-2-(3-butenyl)-2,4-dihydro-4-[[2-(1H-tetrazol-5-yl) [1,1′-biphenyl]-4′-yl]methyl]-3H-1,2,4-triazol-3-one;

5-(1-butenyl)-2-(2-butynyl)-2,4-dihydro-4-[[2-(1H-tetrazol-5-yl) [1,1′-biphenyl]-4′-yl]methyl]-3H-1,2,4-triazol-3-one;

5-(1butenyl)-2-(3-butynyl)-2,4-dihydro-4-[[2-(1H -tetrazol-5-yl) [1,1′-biphenyl]-4′-yl]methyl]-3H-1,2,4-triazol-3-one;

5-(2-butenyl)-2-ethyl-2,4-dihydro-4-[[2-(1H-tetrazol-5-yl) [1,1′-biphenyl]-4′-yl]methyl]-3H-1,2,4-triazol-3-one;

5-(2-butenyl)-2-propyl-2,4-dihydro-4-[[2-(1H-tetrazol-5-yl) [1,1′-biphenyl]-4′-yl]methyl]-3H-1,2,4-triazol-3-one;

5-(2-butenyl)-2-isopropyl-2,4-dihydro-4-[[2-(1H-tetrazol-5-yl) [1,1′-biphenyl]-4′-yl]methyl]-3H-1,2,4-triazol-3-one;

5-tertbutyl(2-butenyl)-2-butyl-2,4-dihydro-4-[[2-(1H-tetrazol-5-yl) [1,1′-biphenyl]-4′-yl]methyl]-3H-1,2,4-triazol-3-one;

5-(2-butenyl)-2-secbutyl-2,4-dihydro-4-[[2-(1H-tetrazol-5-yl) [1,1′-biphenyl]-4′-yl]methyl]-3H-1,2,4-triazol-3-one;

5-(2-butenyl)-2-isobutyl-2,4-dihydro-4-[[2-(1H-tetrazol-5-yl) [1,1′-biphenyl]-4′-yl]methyl]-3H-1,2,4-triazol-3-one;

5-(2-butenyl)-2-tertbutyl-2,4-dihydro-4-[[2-(1H-tetrazol-5-yl) [1,1′-biphenyl]-4′-yl]methyl]-3H-1,2,4-triazol-3-one;

5-(2-butenyl)-2-pentyl-2,4-dihydro-4-[[2-(1H-tetrazol-5-yl) [1,1′-biphenyl]-4′-yl]methyl]-3H-1,2,4-triazol-3-one;

5-(2-butenyl)-2-isopentyl-2,4-dihydro-4-[[2-(1H-tetrazol-5-yl) [1,1′-biphenyl]-4′-yl]methyl]-3H-1,2,4-triazol-3-one;

5-(2-butenyl)-2-hexyl-2,4-dihydro-4-[[2-(1H-tetrazol-5-yl) [1,1′-biphenyl]-4′-yl]methyl]-3H-1,2,4-triazol-3-one;

5-(2-butenyl)-2-heptyl2,4-dihydro-4-[[2-(1H-tetrazol-5-yl) [1,1′-biphenyl]-4′-yl]methyl]-3H-1,2,4-triazol-3-one;

5-(2-butenyl)-2-octyl-2,4-dihydro-4-[[2-(1H-tetrazol-5-yl) [1,1′-biphenyl]-4′-yl]methyl]-3H-1,2,4-triazol-3-one;

5-(2-butenyl)-2-cyclohexyl-2,4-dihydro-4-[[2-(1H-tetrazol-5-yl) [1,1′-biphenyl]-4′-yl]methyl]-3H-1,2,4-triazol-3-one;

5-(2-butenyl)-2-cyclohexylmethyl-2,4-dihydro-4-[[2-(1H-tetrazol-5-yl) [1,1′-biphenyl]-4′-yl]methyl]-3H-1,2,4-triazol-3-one;

5-(2-butenyl)-2-(2-cyclohexylethyl)-2,4-dihydro-4-[[2-(1H-tetrazol-5yl) [1,1′-biphenyl]-4′-yl]methyl]-3H-1,2,4-triazol-3-one;

5-(2-butenyl)-2-cyclohexanoyl-2,4-dihydro-4-[[2-(1H-tetrazol-5-yl) [1,1′-biphenyl]-4′-yl]methyl]-3H-1,2,4-triazol-3-one;

5-(2-butenyl)-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;

5-(2-butenyl)-2-phenyl-2,4-dihydro-4-[[2-(1H-tetrazol-5-yl) [1,1′-biphenyl]-4′-yl]methyl]-3H-1,2,4-triazol-3-one;

5-(2-butenyl)-2-phenylmethyl-2,4-dihydro-4-[[2-(1H-tetrazol-5-yl) [1,1′-biphenyl]-4′-yl]methyl]-3H-1,2,4-triazol-3-one;

5-(2-butenyl)-2-(2-phenylethyl)-2,4-dihydro-4-[[2-(1H-tetrazol-5-yl) [1,1′-biphenyl]-4′-yl]methyl]-3H-1,2,4-triazol-3-one;

5-(2-butenyl)-2-(1-oxopropyl)-2,4-dihydro-4-[[2-(1H-tetrazol-5-yl) [1,1′-biphenyl]-4′-yl]methyl]-3H-1,2,4-triazol-3-one;

5-(2-butenyl)-2-(1-oxobutyl)-2,4-dihydro-4-[[2-(1H-tetrazol-5-yl) [1,1′-biphenyl]-4′-yl]methyl]-3H-1,2,4-triazol-3-one;

5-(2-butenyl)-2-(1-oxopentyl)-2,4-dihydro-4-[[2-(1H-tetrazol-5-yl) 1,1′-biphenyl]-4′-yl]methyl]-3H-1,2,4-triazol-3-one;

2,5-di(2-butenyl)-2,4-dihydro-4-[[2-(1H-tetrazol-5-yl) [1,1′-biphenyl]-4′-yl]methyl]-3H-1,2,4-triazol-3-one;

5-(2-butenyl)-2-(3-butenyl)-2,4-dihydro-4-[[2-(1H-tetrazol-5-yl) [1,1′-biphenyl]-4′-yl]methyl]-3H-1,2,4-triazol-3-one;

5-(2-butenyl)-2-(2-butynyl)-2,4-dihydro-4-[[2-(1H-tetrazol-5-yl) [1,1′-biphenyl]-4′-yl]methyl]-3H-1,2,4-triazol-3-one;

5-(2-butenyl)-2-(3-butynyl)-2,4-dihydro-4-[[2-(1H-tetrazol-5-yl) [1,1′-biphenyl]-4′-yl]methyl]-3H-1,2,4-triazol-3-one;

5-(3-butenyl)-2-ethyl-2,4-dihydro-4-[[2-(1H-tetrazol-5-yl) [1,1′-biphenyl]-4′-yl]methyl]-3H-1,2,4-triazol-3-one;

5-(3-butenyl)-2-propyl-2,4-dihydro-4-[[2-(1H-tetrazol-5-yl) [1,1′-biphenyl]-4′-yl]methyl]-3H-1,2,4-triazol-3-one;

5-(3-butenyl)-2-isopropyl-2,4-dihydro-4-[[2-(1H-tetrazol-5-yl) [1,1′-biphenyl]-4′-yl]methyl]-3H-1,2,4-triazol-3-one;

5-(3-butenyl)-2-butyl-2,4-dihydro-4-[[2-(1H-tetrazol-5-yl) [1,1′-biphenyl]-4′-yl]methyl]-3H-1,2,4-triazol-3-one;

5-(3-butenyl)-2-secbutyl-2,4-dihydro-4-[[2-(1H-tetrazol-5-yl) [1,1′-biphenyl]-4′-yl]methyl]-3H-1,2,4-triazol-3-one;

5-(3-butenyl)-2-isobutyl-2,4-dihydro-4-[[2-(1H-tetrazol-5-yl) [1,1′-biphenyl]-4′-yl]methyl]-3H-1,2,4-triazol-3-one;

5-(3-butenyl)-2-tertbutyl-2,4-dihydro-4-[[2-(1H-tetrazol-5-yl) [1,1′-biphenyl]-4′-yl]methyl]-3H-1,2,4-triazol-3-one;

5-(3-butenyl)-2-pentyl-2,4-dihydro-4-[[2-(1H-tetrazol-5-yl) [1,1′-biphenyl]-4′-yl]methyl]-3H-1,2,4-triazol-3-one;

5-(3-butenyl)-2-isopentyl-2,4-dihydro-4-[[2-(1H-tetrazol-5-yl) [1,1′-biphenyl]-4′-yl]methyl]-3H-1,2,4-triazol-3-one;

5-(3-butenyl)-2-hexyl-2,4-dihydro-4-[[2-(1H-tetrazol-5-yl) [1,1′-biphenyl]-4′-yl]methyl]-3H-1,2,4-triazol-3-one;

5-(3-butenyl)-2-heptyl2,4-dihydro-4-[[2-(1H-tetrazol-5-yl) [1,1′-biphenyl]-4′-yl]methyl]-3H-1,2,4-triazol-3-one;

5-(3-butenyl)-2-octyl-2,4-dihydro-4-[[2-(1H-tetrazol-5-yl) [1,1′-biphenyl]-4′-yl]methyl]-3H-1,2,4-triazol-3-one;

5-(3-butenyl)-2-cyclohexyl-2,4-dihydro-4-[[2-(1H-tetrazol-5-yl) [1,1′-biphenyl]-4′-yl]methyl]-3H-1,2,4-triazol-3-one;

5-(3-butenyl)-2-cyclohexylmethyl-2,4-dihydro-4-[[2-(1H-tetrazol-5-yl) [1,1′-biphenyl]-4′-yl]methyl]-3H-1,2,4-triazol-3-one;

5-(3-butenyl)-2-(2-cyclohexylethyl)-2,4-dihydro-4-[[2-(1H-tetrazol-5-yl) [1,1′-biphenyl]-4′-yl]methyl]-3H-1,2,4-triazol-3-one;

5-(3-butenyl)-2-cyclohexanoyl-2,4-dihydro-4-[[2-(1H-tetrazol-5-yl) [1,1′-biphenyl]-4′-yl]methyl]-3H-1,2,4-triazol-3-one;

5-(3-butenyl)-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;

5-(3-butenyl)-2-phenyl-2,4-dihydro-4-[[2-(1H-tetrazol-5-yl) [1,1′-biphenyl]-4′-yl]methyl]-3H-1,2,4-triazol-3-one;

5-(3-butenyl)-2-phenylmethyl-2,4-dihydro-4-[[2-(1H-tetrazol-5yl) [1,1′-biphenyl]-4′-yl]methyl]-3H-1,2,4-triazol-3-one;

5-(3-butenyl)-2-(2-phenylethyl)-2,4-dihydro-4-[[2-(1H-tetrazol-5-yl) [1,1′-biphenyl]-4′-yl]methyl]-3H-1,2,4-triazol-3-one;

5-(3-butenyl)-2-benzoyl-2,4-dihydro-4-[[2-(1H-tetrazol-5-yl) [1,1′-biphenyl]-4′-yl]methyl]-3H-1,2,4-triazol-3-one;

5-(3-butenyl)-2-(1-oxo-2-phenylethyl)-2,4-dihydro-4-[[2-(1H-′tetrazol-5-yl) [1,1′-biphenyl]-4′-yl]methyl]-3H-1,2,4-triazol-3-one;

5-(3-butenyl)-2-(1-oxopropyl)-2,4-dihydro-4-[[2-(1H-tetrazol-5-yl) [1,1′-biphenyl]-4′-yl]methyl]-3H-1,2,4-triazol-3-one;

5-(3-butenyl)-2-(1-oxobutyl)-2,4-dihydro-4-[[2-(1H-tetrazol-5-yl) [1,1′-biphenyl]-4′-yl]methyl]-3H-1,2,4-triazol-3-one;

5-(3-butenyl)-2-(1-oxopentyl)-2,4-dihydro-4-[[2-(1H-tetrazol-5-yl) [1,1′-biphenyl]-4′-yl]methyl]-3H-1,2,4-triazol-3-one;

5-(3-butenyl)-2-(2-butenyl)-2,4-dihydro-4-[[2-(1H-tetrazol-5-yl) [1,1′-biphenyl]-4′-yl]methyl]-3H-1,2,4-triazol-3-one; 2,5-di(3-butenyl)-2,4-dihydro-4-[[2-(1H-tetrazol-5-yl) [1,1′-biphenyl]-4′-yl]methyl]-3H-1,2,4-triazol-3-one;

5-(3-butenyl)-2-(2-butynyl)-2,4-dihydro-4-[[2-(1H-tetrazol-5-yl) [1,1′-biphenyl]-4′-yl]methyl]-3H-1,2,4-triazol-3-one;

5-(3-butenyl)-2-(3-butynyl)-2,4-dihydro-4-[[2-(1H-tetrazol-5-yl) [1,1′-biphenyl]-4′-yl]methyl]-3H-1,2,4-triazol-3-one;

5-(1-butynyl)-2-ethyl-2,4-dihydro-4-[[2-(1H-tetrazol-5-yl) [1,1′-biphenyl]-4′-yl]methyl]-3H-1,2,4-triazol-3-one;

5-(1-butynyl)-2-propyl-2,4-dihydro-4-[[2-(1H-tetrazol-5-yl) [1,1′-biphenyl]-4′-yl]methyl]-3H-1,2,4-triazol-3-one;

5-(1-butynyl)-2-butyl-2,4-dihydro-4-[[2-(1H-tetrazol-5-yl) [1,1′-biphenyl]-4′-yl]methyl]-3H-1,2,4-triazol-3-one;

5-(1-butynyl)-2-secbutyl-2,4-dihydro-4-[[2-(1H-tetrazol-5-yl) [1,1′-biphenyl]-4′-yl]methyl]-3H-1,2,4-triazol-3-one;

5-(1-butynyl)-2-isobutyl-2,4-dihydro-4-[[2-(1H-tetrazol-5-yl) [1,1′-biphenyl]-4′-yl]methyl]-3H-1,2,4-triazol-3-one;

5-(1-butynyl)-2-tertbutyl-2,4-dihydro-4-[[2-(1H-tetrazol-5-yl) [1,1′-biphenyl]-4′-yl]methyl]-3H-1,2,4-triazol-3-one;

5-(1-butynyl)-2-pentyl-2,4-dihydro-4-[[2-(1H-tetrazol-5-yl) [1,1′-biphenyl]-4′-yl]methyl]-3H-1,2,4-triazol-3-one;

5-(1-butynyl)-2-isopentyl-2,4-dihydro-4-[[2-(1H-tetrazol-5-yl) [1,1′-biphenyl]-4′-yl]methyl]-3H-1,2,4-triazol-3-one;

5-(1-butynyl)-2-hexyl-2,4-dihydro-4-[[2-(1H-tetrazol-5-yl) [1,1′-biphenyl]-4′-yl]methyl]-3H-1,2,4-triazol-3-one;

5-(1-butynyl)-2-heptyl-2,4-dihydro-4-[[2-(1H-tetrazol-5-yl) [1,1′-biphenyl]-4′-yl]methyl]-3H-1,2,4-triazol-3-one;

5-(1-butynyl)-2-octyl-2,4-dihydro-4-[[2-(1H-tetrazol-5-yl) [1,1′-biphenyl]-4′-yl]methyl]-3H-1,2,4-triazol-3-one;

5-(1-butynyl)-2-cyclohexyl-2,4-dihydro-4-[[2-(1H-tetrazol-5-yl) [1,1′-biphenyl]-4′-yl]methyl]-3H-1,2,4-triazol-3-one;

5-(1-butynyl)-2-cyclohexylmethyl-2,4-dihydro -4 -[[2 -(1H-tetrazol-5-yl) [1,1′-biphenyl]-4′-yl]methyl]-3H-1,2,4-triazol-3-one;

5-(1-butynyl)-2-(2-cyclohexylethyl)-2,4-dihydro-4-[[2-(1H-tetrazol-5-yl) [1,1′-biphenyl]-4′-yl]methyl]-3H-1,2,4-triazol-3-one;

5-(1-butynyl)-2-cyclohexanoyl-2,4-dihydro-4-[[2-(1H-tetrazol-5-yl) [1,1′-biphenyl]-4′-yl]methyl]-3H-1,2,4-triazol-3-one;

5-(1-butynyl)-2-(1-oxo-2-cyclohex ylethyl)-2,4-dihydro-4-[[2-(1H-tetrazol-5-yl) [1,1′-biphenyl]-4-yl]methyl]-3H-1,2,4-triazol-3-one;

5-(1-butynyl)-2-phenyl-2,4-dihydro-4-[[2-(1H-tetrazol-5-yl) [1,1′-biphenyl]-4′-yl]methyl]-3H-1,2,4-triazol-3-one;

5-(1butynyl)-2-phenylmethyl-2,4-dihydro-4-[[2-(1H-tetrazol-5-yl) [1,1′-biphenyl]-4′-yl]methyl3-3H-1,2,4-5 triazol-3-one;

5-(1-butynyl)-2-(2-phenylethyl)-2,4-dihydro-4-[[2-(1H-tetrazol-5-yl) [1,1′-biphenyl1-4′-yl]methyl]-3H-1,2,4-triazol-3-one;

5-(1-butynyl)-2-benzoyl-2,4-dihydro-4-[[2-(1H-tetrazol-5-yl) [1,1′-biphenyl]-4′-yl]methyl]-3H-1,2,4-triazol-3-one;

5-(1-butynyl)-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;

5-(1-butynyl)-2-(1-oxopropyl)-2,4-dihydro-4-[[2-(1H-tetrazol-5-yl) [1,1′-biphenyl]-4′-yl]methyl]-3H-1,2,4-triazol-3-one;

5-(1-butynyl)-2-(1-oxobutyl)-2,4-dihydro-4-[[2-(1H-tetrazol-5-yl) [1,1′-biphenyl]-4′-yl]methyl]-3H-1,2,4-triazol-3-one;

5-(1-butynyl)-2-(1-oxopentyl)-2,4-dihydro-4-[[2-(1H-tetrazol-5-yl) [1,1′-biphenyl]-4′-yl]methyl]-3H-1,2,4-triazol-3-one;

5-(1butynyl)-2-(2-butenyl)-2,4-dihydro-4-[[2-(1H-tetrazol-5-yl) [1,1′-biphenyl]-4′-yl]methyl]-3H-1,2,4-triazol-3-one;

5-(1-butynyl)-2-(3-butenyl)-2,4-dihydro-4-[[2-(1H-tetrazol-5-yl) [1,1′-biphenyl]-4′-yl]methyl]-3H-1,2,4-triazol-3-one;

5-(1-butynyl)-2-(2-butynyl)-2,4-dihydro-4-[[2-(1H-tetrazol-5-yl) [1,1′-biphenyl]-4′-yl]methyl]-3H-1,2,4-triazol-3-one;

5-(1-butynyl)-2-(3-butynyl)-2,4-dihydro-4-[[2-(1H-tetrazol-5-yl) [1,1′-biphenyl]-4′-yl]methyl]-3H-1,2,4-triazol-3-one;

5-(2-butynyl)-2-ethyl-2,4-dihydro-4-[[2-(1H-tetrazol-5-yl) [1,1′-biphenyl]-4′-yl]methyl]-3H-1,2,4-triazol-3-one;

5-(2-butynyl)-2-propyl-2,4-dihydro-4-[[2-(1H-tetrazol-5-yl) [1,1′-biphenyl]-4′-yl]methyl]-3H-1,2,4-triazol-3-one;

5-(2-butynyl)-2-butyl-2,4-dihydro-4-[[2-(1H-tetrazol-5-yl) [1,1′-biphenyl]-4′-yl]methyl]-3H-1,2,4-triazol-3-one;

5-(2-butynyl)-2-secbutyl-2,4-dihydro-4-[[2-(1H-tetrazol-5-yl) [1,1′-biphenyl]-4′-yl]methyl]-3H-1,2,4-triazol-3-one;

5-(2-butynyl)-2-isobutyl-2,4-dihydro-4-([2-(1H-tetrazol-5-yl) [1,1′-biphenyl]-4′-yl]methyl]-3H-1,2,4-triazol-3-one;

5-(2-butynyl)-2-tertbutyl-2,4-dihydro-4-[[2-(1H-tetrazol-5-yl) [1,1′-biphenyl]-4′-yl]methyl]-3H-1,2,4-triazol-3-one;

5-(2-butynyl)-2-pentyl-2,4-dihydro-4-[[2-(1H-tetrazol-5-yl) [1,1′-biphenyl]-4′-yl]methyl]-3H-1,2,4-triazol-3-one;

5-(2-butynyl)-2-isopentyl-2,4-dihydro-4-[[2-(1H-tetrazol-5-yl) [1,1′-biphenyl]-4′-yl]methyl]-3H-1,2,4-triazol-3-one;

5-(2-butynyl)-2-hexyl-2,4-dihydro-4-[[2-(1H-tetrazol-5-yl) [1,1′-biphenyl]-4′-yl]methyl]-3H-1,2,4-triazol-3-one;

5-(2-butynyl)-2-heptyl2,4-dihydro-4-[[2-(1H-tetrazol-5-yl) [1,1′-biphenyl]-4′-yl]methyl]-3H-1,2,4-triazol-3-one;

5-(2-butynyl)-2-octyl-2,4-dihydro-4-[[2-(1H-tetrazol-5-yl) [1,1′-biphenyl]-4′-yl]methyl]-3H-1,2,4-triazol-3-one;

5-(2-butynyl)-2-cyclohexyl-2,4-dihydro-4-[[2-(1H-tetrazol-5-yl) [1,1′-biphenyl]-4′-yl]methyl]-3H-1,2,4-triazol-3-one;

5-(2-butynyl)-2-cyclohexylmethyl-2,4-dihydro-4-[(2-(1H-tetrazol-5-yl) [1,1′-biphenyl]-4′-yl]methyl]-3H-1,2,4-triazol-3-one;

5-(2-butynyl)-2-(2-cyclohexylethyl)-2,4-dihydro-4-[[2-(1H-triazol-3-one;

5-(2-butynyl)-2-cycohexanoyl-2,4-dihydro-4-[[2-(1H-tetrazol-5-yl) [1,1′-biphenyl]-4′-yl]methyl]-3H-1,2,4-triazol-3-one;

5-(2-butynyl)-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one; b 5-(2-butynyl)-2-phenyl-2,4-dihydro-4-[2-(1H-tetrazol-5-yl) [1,1′-biphenyl]-4′-yl]methyl]-3H-1,2,4-triazol-3-one;

5-(2-butynyl)-2-phenylmethyl-2,4-dihydro-4 -[[2-(1H-tetrazol-5-yl) [1,1′-biphenyl)-4 1-yl]methyl]-3H-1,2,4-triazol-3-one;

5-(2-butynyl)-2-(2-phenylethyl)-2,4-dihydro-4-[[2-(1H-tetrazol-5-yl) [1,1′-biphenyl]-4′-yl]methyl]-3H-1,2,4-triazol-3-one;

5-(2-butynyl)-2-benzoyl-2,4-dihydro-4-[[2-(1H-tetrazol-5-yl) [1,1′-biphenyl]-4′-yl]methyl]-3H-1,2,4-triazol-3-one;

5-(2-butynyl)-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;

5-(2-butynyl)-2-(1-oxopropyl)-2,4-dihydro-4-[[2-(1H-tetrazol-5-yl) [1,1′-biphenyl]-4′-yl]methyl]-3H-1,2,4-triazol-3-one;

5-(2-butynyl)-2-(1-oxobutyl)-2,4-dihydro-4-[[2-(1H-tetrazol-5-yl) [1,1′-biphenyl]-4′-yl]methyl]-3H-1,2,4-triazol-3-one;

5-(2-butynyl)-2-(1-oxopentyl)-2,4-dihydro-4-[[2-(1H-tetrazol-5-yl) [1,1′-biphenyl]-4′-yl]methyl)-3H-1,2,4-triazol-3-one;

5-(2-butynyl)-2-(2-butenyl)-2,4-dihydro-4-[[2-(1H-tetrazol-5-yl) [1,1′-biphenyl]-4′-yl]methyl]-3H-1,2,4-triazol-3-one;

5-(2-butynyl)-2-(3-butenyl)-2,4-dihydro-4-[[2-(1H-tetrazol-5-yl) [1,1′-biphenyl]-4′-yl]methyl]-3H-1,2,4-triazol-3-one; 2,5-di(2-butynyl)-2,4-dihydro-4-[[2-(1H-tetrazol-5-yl) [1,1′-biphenyl]-4′-yl]methyl]-3H-1,2,4-triazol-3-one;

5-(2-butynyl)-2-(3-butynyl)-2,4-dihydro-4-[[2-(1H-tetrazol-5-yl) [1,1′-biphenyl]-4′-yl]methyl]-3H-1,2,4-triazol-3-one;

5-(3-butynyl)-2-ethyl-2,4-dihydro-4-[[2-(1H-tetrazol-5-yl) [1,1′-biphenyl]-4′-yl]methyl]-3H-1,2,4-triazol-3-one;

5-(3-butynyl)-2-propyl-2,4-dihydro-4-[[2-(1H-tetrazol-5-yl) [1,1′-biphenyl]-4′-yl]methyl]-3H-1,2,4-triazol-3-one;

5-(3-butynyl)-2-isopropyl-2,4-dihydro-4-[[2-(1H-tetrazol-5-yl) [1,1′-biphenyl]-4′-yl]methyl]-3H-1,2,4-triazol-3-one;

5-(3-butynyl)-2-butyl-2,4-dihydro-4-[[2-(1H-tetrazol-5-yl) [1,1′-biphenyl]-4′-yl]methyl]-3H-1,2,4-triazol-3-one;

5-(3-butynyl)-2-secbutyl-2,4-dihydro-4-[[2-(1H-tetrazol-5-yl) [1,1′-biphenyl]-4′-yl]methyl]-3H-1,2,4-triazol-3-one;

5-(3-butynyl)-2-isobutyl-2,4-dihydro-4-[[2-(1H-tetrazol-5-yl) [1,1′-biphenyl]-4′-yl]methyl]-3H-1,2,4-triazol-3-one;

5-(3-butynyl)-2-tertbutyl-2,4-dihydro-4-[[2-(1H-tetrazol-5-yl) [1,1′-biphenyl]-4′-yl]methyl]-3H-1,2,4-triazol-3-one;

5-(3-butynyl)-2-isopentyl-2,4-dihydro-4-[[2-(1H-tetrazol-5-yl) [1,1′-biphenyl]-4′-yl]methyl]-3H-1,2,4-triazol-3-one;

5-(3-butynyl)-2-pentyl-2,4-dihydro-4-[[2-(1H-tetrazol-5-yl) [1,1′-biphenyl]-4′-yl]methyl]-3H-1,2,4-triazol-3-one;

5-(3-butynyl)-2-hexyl-2,4-dihydro-4-([2-(1H-tetrazol-5-yl) [1,1′-biphenyl]-4′-yl]methyl]-3H-1,2,4-triazol-3-one;

5-(3-butynyl)-2-heptyl2,4-dihydro-4-[[2-(1H-tetrazol-5-yl) [1,1′-biphenyl]-4′-yl]methyl]-3H-1,2,4-triazol-3-one;

5-(3-butynyl)-2-octyl-2,4-dihydro-4-[[2-(1H-tetrazol-5-yl) [1,1′-biphenyl]-4′-yl]methyl]-3H-1,2,4-triazol-3-one;

5-(3-butynyl)-2-cyclohexyl-2,4-dihydro-4-[[2-(1H-tetrazol-5-yl) [1,1′-biphenyl]-4′-yl]methyl]-3H-1,2,4-triazol-3-one;

5-(3-butynyl)-2-cyclohexylmethyl-2,4-dihydro-4-[[2-(1H-tetrazol-5-yl) [1,1′-biphenyl]-4′-yl]methyl]-3H-1,2,4-triazol-3-one;

5-(3-butynyl)-2-(2-cyclohexylethyl)-2,4-dihydro-4-[[2-(1H-tetrazol-5yl) [1,1′-biphenyl]-4′-yl]methyl]-3H-1,2,4-triazol-3-one;

5-(3-butynyl)-2-cyclohexanoyl-2,4-dihydro-4-[[2-(1H-tetrazol-5-yl) [1,1′-biphenyl]-4′-yl]methyl]-3H-1,2,4-triazol-3-one;

5-(3-butynyl)-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;

5-(3-butynyl)-2-phenyl-2,4-dihydro-4-[[2-(1H-tetrazol-5-yl) [1,1′-biphenyl]-4′-yl]methyl]-3H-1,2,4-triazol-3-one;

5-(3-butynyl)-2-phenylmethyl-2,4-dihydro-4-[[2-(1H-tetrazol-5-yl) [1,1′-biphenyl]-4′-yl]methyl]-3H-1,2,4-triazol-3-one;

5-(3-butynyl)-2-(2-phenylethyl)-2,4-dihydro-4-[[2-(1H-tetrazol-5-yl) [1,1′-biphenyl]-4′-yl]methyl]-3H-1,2,4-triazol-3-one;

5-(3-butynyl)-2-benzoyl-2,4-dihydro-4-[[2-(1H-tetrazol-5-yl) [1,1′-biphenyl]-4′-yl]methyl]-3H-1,2,4-triazol-3-one;

5-(3-butynyl)-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;

5-(3-butynyl)-2-(1-oxopropyl)-2,4-dihydro-4-[[2-(1H-tetrazol-5-yl) [1,1′-biphenyl]-4′-yl]methyl]-3H-1,2,4-triazol-3-one;

5-(3-butynyl)-2-(1-oxobutyl)-2,4-dihydro-4-[[2-(1H-tetrazol-5-yl) [1,1′-biphenyl]-4′-yl]methyl]-3H-1,2,4-triazol-3-one;

5-(3-butynyl)-2-(1-oxopentyl)-2,4-dihydro-4-[[2-(1H-tetrazol-5-yl) [1,1′-biphenyl]-4′-yl]methyl]-3H-1,2,4-triazol-3-one;

5-(3-butynyl)-2-(2-butenyl)-2,4-dihydro-4-[[2-(1H-tetrazol-5-yl) [1,1′-biphenyl]-4′-yl]methyl]-3H-1,2,4-triazol-3-one;

5-(3-butynyl)-2-(3-butenyl)-2,4-dihydro-4-[[2-(1H-tetrazol-5-yl) [1,1′-biphenyl3-4′-yl]methyl]-3H-1,2,4-triazol-3-one;

5-(3-butynyl)-2-(2-butynyl)-2,4-dihydro-4-[[2-(1H-tetrazol-5-yl) [1,1′-biphenyl]-4′-yl]methyl]-3H-1,2,4-triazol-3-one; and 2,5-di(3-butynyl)-2,4-dihydro-4-[[2-(1H-tetrazol-5-yl) [1,1′-biphenyl]-4′-yl]methyl]-3H-1,2,4-triazol-3-one.

A family of specific compounds of more particular interest within Formula I consists of compounds having an acidic group attached at the R⁵ or R⁹ “ortho” position of the biphenyl moiety, as follows:

2,5-dipropyl-2,4-dihydro-4-[(2-(1H-tetrazol-5-yl) [1,1′-biphenyl]-4′-yl]methyl]-3H-l,2,4-triazol-3-one;

5-propyl-2-butyl-2,4-dihydro-4-[[2-(1H-tetrazol-5-yl) [1,1′-biphenyl]-4′-yl]methyl]-3H-l,2,4-triazol-3-one;

5-propyl-2-secbutyl-2,4-dihydro-4-[[2-(1H-tetrazol-5-yl) [1,1′-biphenyl]-4′-yl]methyl]-3H-l,2,4-triazol-3-one;

5-propyl-2-isobutyl-2,4-dihydro-4-[[2-(1H-tetrazol-5-yl) [1,1′-biphenyl]-4′-yl]methyl]-3H-l,2,4-triazol-3-one;

5-propyl-2-tertbutyl-2,4-dihydro-4-[[2-(1H-tetrazol-5-yl) [1,1′-biphenyl]-4′-yl]methyl]-3H-l,2,4-triazol-3-one;

5-propyl-2-pentyl-2,4-dihydro-4-[[2-(1H-tetrazol-5-yl) [1,1′-biphenyl]-4′-yl]methyl]-3H-1,2,4-triazol-3-one;

5-propyl-2-isopentyl-2,4-dihydro-4-[[2-(1H-tetrazol-5-yl) [1,1′-biphenyl]-4′-yl]methyl]-3H-l,2,4-triazol-3-one;

5-propyl-2-(1-oxopropyl)-2,4-dihydro-4-[[2-(1H-tetrazol-5-yl) [1,1′-biphenyl]-4′-yl]methyl]-3H-1,2,4-triazol-3-one;

5-propyl-2-(1-oxobutyl)-2,4-dihydro-4-[[2-(1H-tetrazol-5-yl) [1,1′-biphenyl]-4′-yl]methyl]-3H-l,2,4-triazol-3-one;

5-propyl-2-(1-oxopentyl)-2,4-dihydro-4-[[2-(1H-tetrazol-5-yl) [1,1′-biphenyl]-4′-yl]methyl]-3H-l,2,4-triazol-3-one;

5-isopropyl-2-propyl-2,4-dihydro-4-[[2-(1H-tetrazol-5-yl) [1,1′-biphenyl]-4′-yl]methyl]-3H1,2,4-triazol-3-one; 2,5-diisopropyl-2,4-dihydro-4-[[2-(1H-tetrazol-5-yl) [1,1′-biphenyl]-4′-yl]methyl]-3H-l,2,4-triazol-3-one;

5-isopropyl-2-butyl-2,4-dihydro-4-[[2-(1H-tetrazol-5-yl) [1,1′-biphenyl]-4′-yl]methyl]-3H-l,2,4-triazol-3-one;

5-isopropyl-2-secbutyl-2,4-dihydro-4-[[2-(1H-tetrazol-5-yl) [1,1′-biphenyl]-4′-yl]methyl]-3H-l,2,4-triazol-3-one;

5-isopropyl-2-isobutyl-2,4-dihydro-4-[[2-(1H-tetrazol-5-yl) [1,1′-biphenyl]-4′-yl]methyl]-3H-1,2,4-triazol-3-one;

5-isopropyl-2-tertbutyl-2,4-dihydro-4-[[2-(1H-tetrazol-5-yl) [1,1′-biphenyl]-4′-yl]methyl]-3H-l,2,4-triazol-3-one;

5-isopropyl-2-pentyl-2,4-dihydro-4-[[2-(1H-tetrazol-5-yl) [1,1′-biphenyl]-4′-yl]methyl]-3H-1,2,4-triazol-3-one;

5-isopropyl-2-isopentyl-2,4-dihydro-4-[[2-(1H-tetrazol-5-yl) [1,1′-biphenyl]-4′-yl]methyl3-3H-1,2,4-triazol-3-one;

5-isopropyl-2-(1-oxopropyl)-2,4-dihydro-4-[[2-(1H-tetrazol-5-yl) [1,1′-biphenyl]-4′-yl]methyl]-3H-1,2,4-triazol-3-one;

5-isopropyl-2-(1-oxobutyl)-2,4-dihydro-4- ([2-(1H-tetrazol-5-yl) [1,1′-biphenyl1-4′-yl]methyl]-3H-1,2,4-triazol-3-one;

5-isopropyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-butyl-2-ethyl-2,4-dihydro-4-([2-(1H-tetrazol-5-yl) [1,1′-biphenyl]-4′-yl]methyl]-3H-1,2,4-triazol-3-one;

5-butyl-2-propyl-2,4-dihydro-4-[[2-(1H-tetrazol-5-yl) [1,1′-biphenyl]-4′-yl]methyl]-3H-1,2,4-triazol-3-one;

5-butyl-2-isopropyl-2,4-dihydro-4-[[2-(1H-tetrazol-5-yl) [1,1′-biphenyl]-4′-yl]methyl]-3H-1,2,4-triazol-3-one; 2,5-dibutyl-2,4-dihydro-4-[[2-(1H-tetrazol-5-yl) [1,1′-biphenyl]-4′-yl]methyl]-3H-1,2,4-triazol-3-one;

5-butyl-2-secbutyl-2,4-dihydro-4-[[2-(1H-tetrazol-5-yl) [1,1′-biphenyl]-4′-yl]methyl]-3H-1,2,4-triazol-3-one;

5-butyl-2-isobutyl-2,4-dihydro-4-[[2-(1H-tetrazol-5-yl) [1,1′-biphenyl]-4′-yl]methyl]-3H-1,2,4-triazol-3-one;

5-butyl-2-tertbutyl-2,4-dihydro-4-[[2-(1H-tetrazol-5-yl) [1,1′-biphenyl]-4′-yl]methyl]-3H-1,2,4-triazol-3-one;

5-butyl-2-pentyl-2,4-dihydro-4-[[2-(1H-tetrazol-5-yl) [1,1′-biphenyl]-4′-yl]methyl]-3H-1,2,4-triazol-3-one;

5-butyl-2-isopentyl-2,4-dihydro-4-[[2-(1H-tetrazol-5-yl) [1,1′-biphenyl]-4′-yl]methyl]-3H-1,2,4-triazol-3-one;

5-butyl-2-hexyl-2,4-dihydro-4-[[2-(1H-tetrazol-5-yl) [1,1′-biphenyl]-4′-yl]methyl]-3H-1,2,4-triazol-3-one;

5-butyl-2-heptyl-2,4-dihydro-4-[[2-(1H-tetrazol-5-yl) [1,1′-biphenyl]-4′-yl]methyl]-3H-1,2,4-triazol-3-one;

5-butyl-2-octyl-2,4-dihydro-4-[[2-(1H-tetrazol-5-yl) [1,1′-biphenyl]-4′-yl]methyl]-3H-1,2,4-triazol-3-one;

5-butyl-2-cyclohexyl-2,4-dihydro-4-[[2-(1H-tetrazol-5-yl) [1,1′-biphenyl]-4′-yl]methyl]-3H-,2,4-triazol-3-one;

5-butyl-2-cyclohexylmethyl-2,4-dihydro-4-[[2-(1H-tetrazol-5-yl) [1,1′-biphenyl]-4′-yl]methyl]-3H-1,2,4-triazol-3-one;

5-butyl-2-(2-cyclohexylethyl)-2,4-dihydro-4-[[2-(1H-tetrazol-5-yl) [1,1′-biphenyl]-4′-yl]methyl]-3H-1,2,4-triazol-3-one;

5-butyl-2-cyclohexanoyl-2,4-dihydro-4-[[2-(1H-tetrazol-5-yl) [1,1′-biphenyl]-4′-yl]methyl]-3H-1,2,4-triazol-3-one;

5-butyl-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;

5-butyl-2-phenyl-2,4-dihydro-4-[[2-(1H-tetrazol-5-yl) [1,1′-biphenyl]-4′-yl]methyl]-3H-1,2,4-triazol-3-one;

5-butyl-2-phenylmethyl-2,4-dihydro-4-[[2-(1H-tetrazol-5-yl) [1,1′-biphenyl]-4′-yl]methyl]-3H-1,2,4-triazol-3-one;

5-butyl-2- 2-phenylethyl)-2,4-dihydro-4-[(2-(1H-tetrazol-5-yl) [1,1′-biphenyl]-4′-yl]methyl]-3H-1,2,4-triazol-3-one;

5-butyl-2-benzoyl-2,4-dihydro-4-[[2-(1H-tetrazol-5-yl) [1,1′-biphenyl]-4′-yl]methyl]-3H-1,2,4-triazol-3-one;

5-butyl-2-(1-oxo-2-phenylethyl)-2,4-dihydro-4-([2-(1H-tetrazol-5-yl) [1,1′-biphenyl]-4′-yl]methyl]-3H-1,2,4-triazol-3-one;

5-butyl-2-(1-oxopropyl)-2,4-dihydro-4-[(2-(1H-tetrazol-5-yl) [1,1′-biphenyl]-4′-yl]methyl]-3H-1,2,4-triazol-3-one;

5-butyl-2-(1-oxobutyl)-2,4-dihydro-4-[[2-(1H-tetrazol-5-yl) [1,1′-biphenyl]-4′-yl]methyl]-3H-1,2,4-triazol-3-one;

5-butyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-butyl-2-(2-butenyl)-2,4-dihydro-4-([2-(1H-tetrazol-5-yl) [1,1′-biphenyl]-4′-yl]methyl]-3H-1,2,4-triazol-3-one;

5-butyl-2-(3-butenyl)-2,4-dihydro-4-[[2-(1H-tetrazol-5-yl) [1,1′-biphenyl]-4′-yl]methyl]-3H-1,2,4-triazol-3-one;

5-butyl-2-(2-butynyl)-2,4-dihydro-4-[[2-(1H-tetrazol-5-yl) [1,1′-biphenyl]-4′-yl]methyl]-3H-1,2,4-triazol-3-one;

5-butyl-2-(3-butynyl)-2,4-dihydro-4-[[2-(1H-tetrazol-5-yl) [1,1′-biphenyl]-4′-yl]methyl]-3H-1,2,4-triazol-3-one;

5-secbutyl-2-propyl-2,4-dihydro-4-[[2-(1H-tetrazol-5-yl) [1,1′-biphenyl]-4′-yl]methyl]-3H-1,2,4-triazol-3-one;

5-secbutyl-2-isopropyl-2,4-dihydro-4-[[2-(1H-tetrazol-5-yl) [1,1′-biphenyl]-4′-yl]methyl]-3H-1,2,4-triazol-3-one;

5-secbutyl-2-butyl-2,4-dihydro-4-[[2-(1H-tetrazol-5-yl) [1,1′-biphenyl]-4′-yl]methyl]-3H-1,2,4-triazol-3-one; 2,5-disecbutyl-2,4-dihydro-4-[[2-(1H-tetrazol-5-yl) [1,1′-biphenyl]-4′-yl]methyl]-3H-1,2,4-triazol-3-one;

5-secbutyl-2-isobutyl-2,4-dihydro-4-[[2-(1H-tetrazol-5-yl) [1,1′-biphenyl]-4′-yl]methyl]-3H-1,2,4-triazol-3-one;

5-secbutyl-2-tertbutyl-2,4-dihydro-4-[[2-(1H-tetrazol-5-yl) [1,1′-biphenyl]-4′-yl]methyl]-3H-1,2,4-triazol-3-one;

5-secbutyl-2-pentyl-2,4-dihydro-4-[[2-(1H-tetrazol-5-yl) [1,1′-biphenyl]-4′-yl]methyl]-3H-1,2,4-triazol-3-one;

5-secbutyl-2-isopentyl-2,4-dihydro-4-[[2-(1H-tetrazol-5-yl) [1,1′-biphenyl]-4′-yl]methyl]-3H-1,2,4-triazol-3-one;

5-secbutyl-2-(1-oxopropyl)-2,4-dihydro-4-[[2-(1H-tetrazol-5-yl) [1,1′-biphenyl]-4′-yl]methyl]-3H-1,2,4-triazol-3-one;

5-secbutyl-2-(1-oxobutyl)-2,4-dihydro-4-[[2-(1H-tetrazol-5-yl) [1,1′-biphenyl]-4′-yl]methyl]-3H-1,2,4-triazol-3-one;

5-secbutyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-isobutyl-2-propyl-2,4-dihydro-4-[[2-(1H-tetrazol-5-yl) [1,1′-biphenyl]-4′-yl]methyl]-3H-1,2,4-triazol-3-one;

5-isobutyl-2-isopropyl-2,4-dihydro-4-[[2-(1H-tetrazol-5-yl) [1,1′-biphenyl]-4′-yl]methyl]-3H-1,2,4-triazol-3-one;

5-isobutyl-2-butyl-2,4-dihydro-4-[[2-(1H-tetrazol-5-yl) [1,1′-biphenyl]-4′-yl]methyl]-3H-1,2,4-triazol-3-one;

5-isobutyl-2-secbutyl-2,4-dihydro-4-[[2-(1H-tetrazol-5-yl) [1,1′-biphenyl]-4′-yl]methyl]-3H-1,2,4-triazol-3-one; 2,5-diisobutyl-2,4-dihydro-4-[[2-(1H-tetrazol-5-yl) [1,1′-biphenyl]-4′-yl]methyl]-3H-1,2,4-triazol-3-one;

5-isobutyl-2-tertbutyl-2,4-dihydro-4-[[2-(1H-tetrazol-5-yl) [1,1′-biphenyl]-4′-yl]methyl]-3H-1,2,4-triazol-3-one;

5-isobutyl-2-pentyl-2,4-dihydro-4-[[2-(1H-tetrazol-5-yl) [1,1′-biphenyl]-4′-yl]methyl]-3H-1,2,4-triazol-3-one;

5-isobutyl-2-isopentyl-2,4-dihydro-4-[[2-(1H-tetrazol-5-yl) [1,1′-biphenyl]-4′-yl]methyl]-3H-1,2,4-triazol-3-one;

5-isobutyl-2-(1-oxopropyl)-2,4-dihydro-4-[[2-(1H-tetrazol-5-yl) [1,1′-biphenyl]-4′-yl]methyl]-3H-1,2,4-triazol-3-one;

5-isobutyl-2-(1-oxobutyl)-2,4-dihydro-4-[[2-(1H-tetrazol-5-yl) [1, 1-biphenyl]-4′-yl]methyl]-3H-1,2,4-triazol-3-one;

5-isobutyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-2-propyl-2,4-dihydro-4-([2-(1H-tetrazol-5-yl) [1,1′-biphenyl3-4′-yl]methyl]-3H-1,2,4-triazol-3-one;

5-2-isopropyl-2,4-dihydro-4-[[2-(1H-tetrazol-5-yl) [1,1′-biphenyl]-4′-yl]methyl]-3H-1,2,4-triazol-3-one;

5-2-butyl-2,4-dihydro-4-[[2-(1H-tetrazol-5-yl) [1,1′-biphenyl]-4′-yl]methyl]-3H-1,2,4-triazol-3-one;

5-2-secbutyl -2,4-dihydro-4-[[2-(1H-tetrazol-5-yl) [1,1′-biphenyl]-4′-yl]methyl]-3H-1,2,4-triazol-3-one;

5-2-isobutyl-2,4-dihydro-4-[[2-(1H-tetrazol-5-yl) [1,1′-biphenyl]-4′-yl]methyl)-3H-1,2,4-triazol-3-one; 2,5-ditertbutyl-2,4-dihydro-4-[[2-(1H-tetrazol-5-yl) [1,1′-biphenyl)-4′-yl]methyl]-3H-1,2,4-triazol-3-one;

5-2-pentyl-2,4-dihydro-4-[[2-(1H-tetrazol-5-yl) [1,1′-biphenyl]-4′-yl]methyl]-3H-1,2,4-triazol-3-one;

5-2-isopentyl-2,4-dihydro-4-[[2-(1H-tetrazol-5-yl) [1,1′-biphenyl]-4′-yl]methyl]-3H-1,2,4-triazol-3-one;

5-2-(1-oxopropyl)-2,4-dihydro-4-[[2-(1H-tetrazol-5-yl) [1,1′-biphenyl]-4′-yl]methyl]-3H-1,2,4-triazol-3-one;

5-2-(1-oxobutyl)-2,4-dihydro-4-[[2-(1H-tetrazol-5-yl) [1,1′-biphenyl]-4′-yl]methyl]-3H-1,2,4-triazol-3-one;

5-2-(1-oxopentyl)-2,4-dihydro-4-[[2-(1H-tetrazol-5-yl) [1,1′-biphenyl]-4′-yl]methyl]-3H-1,2,4-triazol-3-one;

5-pentyl-2-propyl-2,4-dihydro-4-[[2-(1H-tetrazol-5-yl) [1,1′-biphenyl]-4′-yl]methyl]-3H-1,2,4-triazol-3-one;

5-pentyl-2-isopropyl-2,4-dihydro-4-[[2-(1H-tetrazol-5-yl) [1,1′-biphenyl]-4′-yl]methyl]-3H-1,2,4-triazol-3-one;

5-pentyl-2-butyl-2,4-dihydro-4-[[2-(1H-tetrazol-5-yl) [1,1′-biphenyl]-4′-yl]methyl3-3H-1,2,4-triazol-3-one;

5-pentyl-2-secbutyl-2,4-dihydro-4-[[2-(1H-tetrazol-5-yl) [1,1′-biphenyl]-4′-yl]methyl]-3H-l,2,4-triazol-3-one;

5-pentyl-2-isobutyl-2,4-dihydro-4-[[2-(1H-tetrazol-5-yl) [1,1′-biphenyl]-4′-yl]methyl]-3H-1,2,4-triazol-3-one;

5-pentyl-2-tertbutyl-2,4-dihydro-4-[[2-(1H-tetrazol-5-yl) [1,1′-biphenyl]-4′-yl]methyl]-3H-1,2,4-triazol-3-one; 2,5-dipentyl-2,4-dihydro-4-[[2-(1H-tetrazol-5-yl) [1,1′-biphenyl]-4′-yl]methyl]-3H-1,2,4-triazol-3-one;

5-pentyl-2-isopentyl-2,4-dihydro-4-([2-(1H-tetrazol-5-yl) [1,1′-biphenyl]-4′-yl]methyl]-3H-1,2,4-triazol-3-one;

5-pentyl-2-(1-oxopropyl)-2,4-dihydro-4- [2-(1H-tetrazol-5-yl) [1,1′-biphenyl]-4′-yl]methyl]-3H-1,2,4-triazol-3-one;

5-pentyl-2-(1-oxobutyl)-2,4-dihydro-4-[[2-(1H-tetrazol-5-yl) [1,1′-biphenyl]-4′-yl]methyl]-3H-1,2,4-triazol-3-one;

5-pentyl-2-(1-oxopentyl)-2,4-dihydro-4-[[2-(1H-tetrazol-5-yl) [1,1′-biphenyl]-4′-yl]methyl]-3H-1,2,4-triazol-3-one; 5-isopentyl-2-propyl-2,4-dihydro-4-[[2-(1H-tetrazol-5-yl) [1,1′-biphenyl]-4′-yl]methyl]-3H-1,2,4-triazol-3-one; 5-isopentyl-2-isopropyl-2,4-dihydro-4-[[2-(1H-tetrazol-5-yl) [1,1′-biphenyl]-4′-yl]methyl]-3H-1,2,4-triazol-3-one; 5-isopentyl-2-butyl-2,4-dihydro-4-[[2-(1H-tetrazol-5-yl) [1,1′-biphenyl]-4′-yl]methyl]-3H-1,2,4-triazol-3-one; 5-isopentyl-2-secbutyl-2,4-dihydro-4-([2-(1H-tetrazol-5-yl) [1,1′-biphenyl]-4′-yl]methyl]-3H-1,2,4-triazol-3-one; 5-isopentyl-2-isobutyl-2,4-dihydro-4-[[2-(1H-tetrazol-5-yl) [1,1′-biphenyl]-4′-yl]methyl]-3H-1,2,4-triazol-3-one; 5-isopentyl-2-tertbutyl-2,4-dihydro-4-[[2-(1H-tetrazol-5-yl) [1,1′-biphenyl]-4′-yl]methyl]-3H-1,2,4-triazol-3-one; 5-isopentyl-2-pentyl-2,4-dihydro-4-[[2-(1H-tetrazol-5-yl) [1,1′-biphenyl]-4′-yl]methyl]-3H-1,2,4-triazol-3-one; 2,5-diisopentyl-2,4-dihydro-4-[[2-(1H-tetrazol-5-yl) [1,1′-biphenyl3-4′-yl]methyl]-3H-1,2,4-triazol-3-one; 5-isopentyl-2-(1-oxopropyl)-2,4-dihydro-4-[[2-(1H-tetrazol-5-yl) [1,1′-biphenyl]-4′-yl]methyl]-3H-1,2,4-triazol-3-one; 5-isopentyl-2-(1-oxobutyl)-2,4-dihydro-4-[[2-(1H-tetrazol-5-yl) [1,1′-biphenyl]-4′-yl]methyl]-3H-1,2,4-triazol-3-one; 5-isopentyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-hexyl-2-propyl-2,4-dihydro-4-[[2-(1H-tetrazol-5-yl) [1,1′-biphenyl]-4′-yl]methyl]-3H-1,2,4-triazol-3-one;

5-hexyl-2-isopropyl-2,4-dihydro-4-[[2-(1H-tetrazol-5-yl) [1, 1-biphenyl]-4′-yl]methyl]-3H-1,2,4-triazol-3-one;

5-hexyl-2-butyl-2,4-dihydro-4-[[2-(1H-tetrazol-5-yl) [1,1′-biphenyl]-4′-yl]methyl]-3H-1,2,4-triazol-3-one;

5-hexyl-2-secbutyl-2,4-dihydro-4-[[2-(1H-tetrazol-5-yl) [1,1′-biphenyl]-4′-yl]methyl]-3H-1,2,4-triazol-3-one;

5-hexyl-2-isobutyl-2,4-dihydro-4-[[2-(1H-tetrazol-5-yl) [1,1′-biphenyl]-4′-yl]methyl]-3H-1,2,4-triazol-3-one;

5-hexyl-2-tertbutyl-2,4-dihydro-4-[[2-(1H-tetrazol-5-yl) [1,1′-biphenyl]-4′-yl]methyl]-3H-1,2,4-triazol-3-one;

5-hexyl-2-pentyl-2,4-dihydro-4-[[2-(1H-tetrazol-5-yl) [1,1′-biphenyl]-4′-yl]methyl]-3H-1,2,4-triazol-3-one;

5-hexyl-2-isopentyl-2,4-dihydro-4-[[2-(1H-tetrazol-5-yl) [1,1′-biphenyl]-4′-yl]methyl]-3H-1,2,4-triazol-3-one; 2,5-dihexyl-2,4-dihydro-4-[[2-(1H-tetrazol-5-yl) [1,1′-biphenyl]-4′-yl]methyl]-3H-1,2,4-triazol-3-one;

5-hexyl-2-(1-oxopropyl)-2,4-dihydro-4-[[2-(1H-tetrazol-5-yl) [1,1′-biphenyl]-4′-yl]methyl]-3H-1,2,4-triazol-3-one;

5-hexyl-2-(1-oxobutyl)-2,4-dihydro-4-[[2-(1H-tetrazol-5-yl) [1,1′-biphenyl]-4′-yl]methyl]-3H-1,2,4-triazol-3-one;

5-hexyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-heptyl-2-butyl-2,4-dihydro-4-[[2-(1H-tetrazol-5-yl) [1,1′-biphenyl]-4′-yl]methyl]-3H-1,2,4-triazol-3-one; 2,5-diheptyl-2,4-dihydro-4-[[2-(1H-tetrazol-5-yl) [1,1′-biphenyl]-4′-yl]methyl]-3H-1,2,4-triazol-3-one;

5-heptyl-2- (1-oxopropyl) -2,4-dihydro-4-[[2-(1H-tetrazol-5-yl) [1,1′-biphenyl]-4′-yl]methyl]-3H-1,2,4-triazol-3-one;

5-heptyl-2-(1-oxobutyl)-2,4-dihydro-4-[[2-(1H-tetrazol-5-yl) [1,1′-biphenyl]-4′-yl]methyl]-3H-1,2,4-triazol-3-one;

5-heptyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-octyl-2-butyl-2,4-dihydro-4-[[2-(1H-tetrazol-5-yl) [1,1′-biphenyl]-4′-yl]methyl]-3H-1,2,4-triazol-3-one; 2,5-dioctyl-2,4-dihydro-4-[[2-(1H-tetrazol-5-yl) [1,1′-biphenyl]-4′-yl]methyl]-3H-1,2,4-triazol-3-one;

5-octyl-2-(1-oxopropyl)-2,4-dihydro-4-[[2-(1H-tetrazol-5-yl) [1,1′-biphenyl]-4′-yl]methyl]-3H-1,2,4-triazol-3-one;

5-octyl-2-(1-oxobutyl)-2,4-dihydro-4-[[2-(1H-tetrazol-5-yl) [1,1′-biphenyl]-4′-yl]methyl]-3H-1,2,4-triazol-3-one;

5-octyl-2-(1-oxopentyl)-2,4-dihydro-4-[[2-(1H-tetrazol-5-yl) [1,1′-biphenyl]-4′-yl]methyl]-3H-1,2,4-triazol-3-one;

5-(1-butenyl)-2-propyl-2,4-dihydro-4-[[2-(1H-tetrazol-5-yl) [1,1′-biphenyl]-4′-yl]methyl]-3H-1,2,4-triazol-3-one;

5-(1-butenyl)-2-isopropyl-2,4-dihydro-4-[(2-(1H-tetrazol-5-yl) [1,1′-biphenyl]-4′-yl]methyl]-3H-1,2,4-triazol-3-one;

5-(1-butenyl)-2-butyl-2,4-dihydro-4-[[2-(1H-tetrazol-5-yl) [1,1′-biphenyl]-4′-yl]methyl]-3H-1,2,4-triazol-3-one;

5-(1-butenyl)-2-secbutyl-2,4-dihydro-4-[[2-(1H-tetrazol-5-yl) [1,1′-biphenyl]-4′-yl3methyl]-3H-1,2,4-triazol-3-one;

5-(1-butenyl)-2-isobutyl-2,4-dihydro-4-[[2-(1H-tetrazol-5-yl) [1,1′-biphenyl]-4t-yl]methyl]-3H-1,2,4-triazol-3-one;

5-(1-butenyl)-2-tertbutyl-2,4-dihydro-4-[[2-(1H-tetrazol-5-yl) [1,1′-biphenyl]-4′-yl]methyl]-3H-1,2,4-triazol-3-one;

5-(1-butenyl)-2-pentyl-2,4-dihydro-4-[[2-(1H-tetrazol-5-yl) [1,1′-biphenyl]-4′-yl]methyl]-3H-1,2,4-triazol-3-one;

5-(1-butenyl)-2-isopentyl-2,4-dihydro-4-[[2-(1H-tetrazol-5-yl) [1,1-biphenyl]-4′-yl]methyl]-3H-1,2,4-triazol-3-one;

5-(1-butenyl)-2-hexyl-2,4-dihydro-4-[[2-(1H-tetrazol-5-yl) [1,1′-biphenyl]-4′-yl]methyl]-3H-1,2,4-triazol-3-one;

5-(1-butenyl)-2-heptyl2,4-dihydro-4-[[2-(1H-tetrazol-5-yl) [1,1′-biphenyl]-4′-yl]methyl]-3H-1,2,4-triazol-3-one;

5-(1-butenyl)-2-octyl-2,4-dihydro-4-[[2-(1H-tetrazol-5-yl) [1,1′-biphenyl]-4′-yl]methyl]-3H-1,2,4-triazol-3-one;

5-(1-butenyl)-2-cyclohexyl-2,4-dihydro-4-[[2-(1H-tetrazol-5-yl) [1,1′-biphenyl]-4′-yl]methyl]-3H-1,2,4-triazol-3-one;

5-(1-butenyl)-2-cyclohexylmethyl-2,4-dihydro-4-[[2-(1H-tetrazol-5-yl) [1,1′-biphenyl]-4′-yl]methyl]-3H-1,2,4-triazol-3-one;

5-(1butenyl)-2-(2-cyclohexylethyl)-2,4-dihydro-4-[[2-(1H-tetrazol-5-yl) [1,1′-biphenyl]-4′-yl]methyl]-3H-1,2,4-triazol-3-one;

5-(1butenyl)-2-cyclohexanoyl-2,4-dihydro-4-[[2-(1H-tetrazol-5-yl) [1,1′-biphenyl]-4′-yl]methyl]-3H-1,2,4-triazol-3-one;

5-(1-butenyl)-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;

5-(1-butenyl)-2-phenyl-2,4-dihydro-4-[[2-(1H-tetrazol-5-yl) [1,1′-biphenyl3-4′-yl]methyl]-3H-1,2,4-triazol-3-one;

5-(1-butenyl)-2-phenylmethyl-2,4-dihydro-4-[[2-(1H-tetrazol-5-yl) [1,1′-biphenyl]-4′-yl]methyl]-3H-1,2,4-triazol-3-one;

5-(1-butenyl)-2-benzoyl-2,4-dihydro-4-[[2-(1H-tetrazol-5-yl) [1,1′-biphenyl]-4′-yl]methyl]-3H-1,2,4-triazol-3-one;

5-(1-butenyl)-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;

5-(1-butenyl)-2-(1-oxopropyl)-2,4-dihydro-4-[[2-(1H-tetrazol-5-yl) [1,1′-biphenyl]-4′-yl]methyl]-3H-1,2,4-triazol-3-one;

5-(1butenyl)-2-(1-oxobutyl)-2,4-dihydro-4-[[2-(1H-tetrazol-5′yl) [1,1′-biphenyl]-4′-yl]methyl]-3H-1,2,4-triazol-3-one;

5-(1-butenyl)-2-(1-oxopentyl)-2,4-dihydro-4-[[2-(1H-tetrazol-5-yl) [1,1′-biphenyl]-4′-yl]methyl]-3H-1,2,4-triazol-3-one;

5-(1-butenyl)-2-(2-butenyl)-2,4-dihydro-4-[[2-(1H-tetrazol-5-yl) [1,1′-biphenyl]-4′-yl]methyl]-3H-1,2,4-triazol-3-one;

5-(1-butenyl)-2-(3-butenyl)-2,4-dihydro-4-[[2-(1H-tetrazol-5-yl) [1,1′-biphenyl]-4′-yl]methyl]-3H-1,2,4-triazol-3-one;

5-(1-butenyl)-2-(2-butynyl)-2,4-dihydro-4-[[2-(1H-tetrazol-5-yl) [1,1′-biphenyl]-4′-yl]methyl]-3H-1,2,4-triazol-3-one;

5-(1-butenyl)-2-(3-butynyl)-2,4-dihydro-4-[[2-(1H-tetrazol-5-yl) [1,1′-biphenyl]-4′-yl]methyl]-3H-1,2,4-triazol-3-one;

5-(2-butenyl)-2-propyl-2,4-dihydro-4-[[2-(1H-tetrazol-5-yl) [1,1′-biphenyl]-4′-yl]methyl]-3H-1,2,4-triazol-3-one;

5-(2-butenyl)-2-isopropyl-2,4-dihydro-4-[[2-(1H-tetrazol-5-yl) [1,1′-biphenyl]-4′-yl]methyl]-3H-1,2,4-triazol-3-one;

5-(2-butenyl)-2-butyl-2,4-dihydro-4-[[2-(1H-tetrazol-5-yl) [1,1′-biphenyl]-4′-yl]methyl]-3H-1,2,4-triazol-3-one;

5-(2-butenyl)-2-secbutyl-2,4-dihydro-4-[[2-(1H-tetrazol-5-yl) [1,1′-biphenyl]-4′-yl]methyl]-3H-1,2,4-triazol-3-one;

5-(2-butenyl)-2-isobutyl-2,4-dihydro-4-[[2-(1H-tetrazol-5-yl) [1,1′-biphenyl]-4′-yl]methyl]-3H-1,2,4-triazol-3-one;

5-(2-butenyl)-2-tertbutyl-2,4-dihydro-4-[[2-(1H-tetrazol-5-yl) [1,1′-biphenyl]-4′-yl]methyl]-3H-1,2,4-triazol-3-one;

5-(2-butenyl)-2-pentyl-2,4-dihydro-4-[[2-(1H-tetrazol-5-yl) [1,1′-biphenyl]-4′-yl]methyl]-3H-1,2,4-triazol-3-one;

5-(2-butenyl)-2-isopentyl-2,4-dihydro-4-[[2-(1H-tetrazol-5-yl) [1,1′-biphenyl]-4′-yl]methyl]-3H-1,2,4-triazol-3-one;

5-(2-butenyl)-2-(1-oxopropyl)-2,4-dihydro-4-[[2-(1H-tetrazol-5-yl) [1,1′-biphenyl]-4′-yl]methyl]-3H-1,2,4-triazol-3-one;

5-(2-butenyl)-2-(1-oxobutyl)-2,4-dihydro-4-[[2-(1H-tetrazol-5-yl) [1,1′-biphenyl]-4′-yl]methyl]-3H-1,2,4-triazol-3-one;

5-(2-butenyl)-2-(1-oxopentyl)-2,4-dihydro-4-[[2-(1H-tetrazol-5-yl) [1,1′-biphenyl]-4′-yl]methyl]-3H-1,2,4-triazol-3-one;

5-(3-butenyl)-2-propyl-2,4-dihydro-4-[[2-(1H-tetrazol-5-yl) [1,1′-biphenyl]-4′-yl]methyl]-3H-1,2,4-triazol-3one;

5-(3-butenyl)-2-butyl-2,4-dihydro-4-[[2-(1H-tetrazol-5-yl) [1,1′-biphenyl]-4′-yl]methyl]-3H-1,2,4-triazol-3-one;

5-(3-butenyl)-2-pentyl-2,4-dihydro-4-[[2-(1H-tetrazol-5-yl) [1,1′-biphenyl]-4′-yl]methyl]-3H-1,2,4-triazol-3-one;

5-(3-butenyl)-2-isopentyl-2,4-dihydro-4-[[2-(1H-tetrazol-5-yl) [1,1′-biphenyl]-4′-yl]methyl]-3H-1,2,4-triazol-3-one;

5-(3-butenyl)-2-(1-oxopropyl)-2,4-dihydro-4-[[2-(1H-tetrazol-5-yl) [1,1′-biphenyl]-4′-yl]methyl]-3H-1,2,4-triazol-3-one;

5-(3-butenyl)-2-(1-oxobutyl)-2,4-dihydro-4-[[2-(1H-tetrazol-5-yl) [1,1′-biphenyl]-4′-yl]methyl]-3H-1,2,4-triazol-3-one;

5-(3-butenyl)-2-(1-oxopentyl)-2,4-dihydro-4- [2-(1H-tetrazol-5-yl) [1,1′-biphenyl]-4′-yl]methyl]-3H-1,2,4-30 triazol-3-one;

5-(1butynyl)-2-propyl-2,4-dihydro-4-[[2- (H-tetrazol -5-yl) [1,1′- biphenyl]-4′-yl methyl]-3H-1,2,4-triazol-3-one;

5-(1-butynyl)-2-butyl-2,4-dihydro-4-[[2-(1H-tetrazol-5-yl) [1,1′-biphenyl]-4′-yl]methyl]-3H-1,2,4-triazol-3-one;

5-(1butynyl)-2-secbutyl-2,4-dihydro-4-[[2-(1H-tetrazol-5-yl) [1,1′-biphenyl]-4′-yl]methyl]-3H-1,2,4-triazol-3-one;

5-(1-butynyl)-2-isobutyl-2,4-dihydro-4-[[2-(1H-tetrazol-5-yl) [1,1′-biphenyl]-4′-yl]methyl]-3H-1,2,4-triazol-3-one;

5-(1-butynyl)-2-tertbutyl-2,4-dihydro-4-[(2-(1H-tetrazol-5-yl) [1,1′-biphenyl)-4′-yl]methyl]-3H-1,2,4-triazol-3-one;

5-(1-butynyl)-2-pentyl-2,4-dihydro-4- (2-(1H-tetrazol-5-yl) [1,1′-biphenyl]-4′-yl]methyl]-3H-1,2,4-triazol-3-one;

5-(1-butynyl)-2-isopentyl-2,4-dihydro-4-[[2-(1H-tetrazol-5-yl) [1,1′-biphenyl]-4′-yl]methyl]-3H-1,2,4-triazol-3-one;

5-(1-butynyl)-2-hexyl-2,4-dihydro-4-[[2-(1H-tetrazol-5-yl) [1,1′-biphenyl]-4′-yl]methyl]-3H-1,2,4-triazol-3-one;

5-(1-butynyl)-2-(1-oxopropyl)-2,4-dihydro-4-[[2-(1H-tetrazol-5-yl) [1,1′-biphenyl]-4′-yl]methyl]-3H-1,2,4-triazol-3-one;

5-(1butynyl)-2-(1-oxobutyl)-2,4-dihydro-4-[[2-(1H-tetrazol-5-yl) [1,1′-biphenyl]-4′-yl]methyl]-3H-1,2,4-triazol-3-one;

5-(1-butynyl)-2-(1-oxopentyl)-2,4-dihydro-4-[[2-(1H-tetrazol-5-yl) [1,1′-biphenyl]-4′-yl]methyl]-3H-1,2,4-triazol-3-one;

5-(2-butynyl)-2-propyl-2,4-dihydro-4-[[2-(1H-tetrazol-5-yl) [1,1′-biphenyl]-4′-yl]methyl]-3H-1,2,4-triazol-3-one;

5-(2-butynyl)-2-butyl-2,4-dihydro-4-[[2-(1H-tetrazol-5-yl) [1,1′-biphenyl]-4′-yl]methyl]-3H-1,2,4-triazol-3-one;

5-(2-butynyl)-2-pentyl-2,4-dihydro-4-[[2-(1H-tetrazol-5-yl) [1,1′-biphenyl]-4′-yl]methyl]-3H-1,2,4-triazol-3-one;

5-(2-butynyl)-2-isopentyl-2,4-dihydro-4-[[2-(1H-tetrazol-5-yl) [1,1′-biphenyl]-4′-yl]methyl]-3H-1,2,4-triazol-3-one;

5-(2-butynyl)-2-(1-oxopropyl)-2,4-dihydro-4-[[2-(1H-tetrazol-5-yl) [1,1′-biphenyl]-4′-yl]methyl]-3H-1,2,4-triazol-3-one;

5-(2-butynyl)-2-(1-oxobutyl)-2,4-dihydro-4-[[2-(1H-tetrazol-5-yl) [1,1′-biphenyl]-4′-yl]methyl]-3H-1,2,4-triazol-3-one;

5-(2-butynyl)-2-(1-oxopentyl)-2,4-dihydro-4-[[2-(1H-tetrazol-5-yl) [1,1′-biphenyl]-4′-yl]methyl]-3H-1,2,4-triazol-3-one;

5-(3-butynyl)-2-propyl-2,4-dihydro-4-[[2-(1H-tetrazol-5-yl) [1,1′-biphenyl]-4′-yl]methyl]-3H-1,2,4-triazol-3-one;

5-(3-butynyl)-2-butyl-2,4-dihydro-4-[[2-(1H-tetrazol-5-yl) [1,1′-biphenyl]-4′-yl]methyl]-3H-1,2,4-triazol-3-one;

5-(3-butynyl)-2-isopentyl-2,4-dihydro-4-[[2-(1H-tetrazol-5 yl) [1,1′-biphenyl]-4′-yl]methyl]-3H-1,2,4-triazol-3-one;

5-(3-butynyl)-2-pentyl-2,4-dihydro-4-[[2-(1H-tetrazol-5-yl) [1,1′-biphenyl]-4′-yl]methyl]-3H-1,2,4-triazol-3-one;

5-(3-butynyl)-2-(1-oxopropyl)-2,4-dihydro-4-[[2-(1H-tetrazol-5-yl) [1,1′-biphenyl]-4′-yl]methyl]-3H-1,2,4-triazol-3-one;

5-(3-butynyl)-2-(1-oxobutyl)-2,4-dihydro-4-[[2-(1H-tetrazol-5-yl) [1,1′-biphenyl]-4′-yl]methyl]-3H-1,2,4-triazol-3-one;

5-(3-butynyl)-2-(1-oxopentyl)-2,4-dihydro-4-[[2-(1H-tetrazol-5-yl) [1,1′-biphenyl]-4′-yl]methyl]-3H-1,2,4-triazol-3-one; and 4′-[(1,3-dibutyl-4,5-dihydro-5-oxo-1H-1,2,4-triazol-4-yl) methyl] [1,1′-biphenyl]-2-carboxylic acid.

Another family of specific compounds of more particular interest within Formula I consists of compounds having an acidic group attached at the R⁶ or R⁸ “meta” position of the biphenyl moiety as follows: 2,5-dipropyl-2,4-dihydro-4-[[3-(1H-tetrazol-5-yl) [1,1′-biphenyl]-4′-yl]methyl]-3H-1,2,4-triazol-3-one;

5-propyl-2-butyl-2,4-dihydro-4-[[3-(1H-tetrazol-5-yl) [1,1′-biphenyl]-4′-yl]methyl]-3H-1,2,4-triazol-3-one;

5-propyl-2-secbutyl-2,4-dihydro-4-[[3-(1H-tetrazol-5-yl) [1,1′-biphenyl]-4′-yl]methyl]-3H-l,2,4-triazol-3-one;

5-propyl-2-isobutyl-2,4-dihydro-4-[[3-(1H-tetrazol-5-yl) [1,1′-biphenyl]-4′-yl]methyl]-3H-1,2,4-triazol-3-one;

5-propyl-2-tertbutyl-2,4-dihydro-4-[[3-(1H-tetrazol-5-yl) [1,1′-biphenyl]-4′-yl]methyl]-3H-1,2,4-triazol-3-one;

5-propyl-2-pentyl-2,4-dihydro-4-[[3-(1H-tetrazol-5-yl) [1,l′-biphenyl]-4′-yl]methyl]-3H-1,2,4-triazol-3-one;

5-propyl-2-isopentyl-2,4-dihydro-4-[[3-(1H-tetrazol-5-yl) [1,1′-biphenyl]-4′-yl]methyl]-3H-l,2,4-triazol-3-one;

5-propyl-2-(1-oxopropyl)-2,4-dihydro-4-[[3-(1H-tetrazol-5-yl) [1,1′-biphenyl]-4′-yl]methyl]-3H-1,2,4-triazol-3-one;

5-propyl-2-(1-oxobutyl)-2,4-dihydro-4-[[3-(1H-tetrazol-5-yl) [1,1′-biphenyl]-4′-yl]methyl]-3H-1,2,4-triazol-3-one;

5-propyl-2-(1-oxopentyl)-2,4-dihydro-4-[[3-(1H-tetrazol-5-yl) [1,1′-biphenyl]-4′-yl]methyl]-3H-1,2,4-triazol-3-one;

5-isopropyl-2-propyl-2,4-dihydro-4-[[3-(1H-tetrazol-5-yl) [1,1′-biphenyl]-4′-yl]methyl]-3H-1,2,4-triazol-3-one; 2,5-diisopropyl-2,4-dihydro-4-[[3-(1H-tetrazol-5-yl) [1,1′-biphenyl]-4′-yl]methyl]-3H-1,2,4-triazol-3-one;

5-isopropyl-2-butyl-2,4-dihydro-4-[[3-(1H-tetrazol-5-yl) [1,1′-biphenyl]-4′-yl]methyl]-3H-1,2,4-triazol-3-one;

5-isopropyl-2-secbutyl-2,4-dihydro-4-[[3-(1H-tetrazol-5-yl) [1,1′-biphenyl]-4′-yl]methyl]-3H-1,2,4-triazol-3-one;

5-isopropyl-2-isobutyl-2,4-dihydro-4-[[3-(1H-tetrazol-5-yl) [1,1′-biphenyl]-4′-yl]methyl]-3H-1,2,4-triazol-3-one;

5-isopropyl-2-tertbutyl-2,4-dihydro-4-[[3-(1H-tetrazol-5-yl) [1,1′-biphenyl]-4′-yl]methyl]-3H-1,2,4-triazol-3-one;

5-isopropyl-2-pentyl-2,4-dihydro-4-[[3-(1H-tetrazol-5-yl) [1,1′-biphenyl]-4′-yl]methyl]-3H-1,2,4-triazol-3-one;

5-isopropyl-2-isopentyl-2,4-dihydro-4-[[3-(1H-tetrazol-5-yl) [1,1′-biphenyl]-4′-yl]methyl]-3H-1,2,4-triazol-3-one;

5-isopropyl-2-(1-oxopropyl)-2,4-dihydro-4-[[3-(1H-tetrazol-5-yl) [1,1′-biphenyl]-4′-yl]methyl]-3H-1,2,4-triazol-3-one;

5-isopropyl-2-(1-oxobutyl)-2,4-dihydro-4-[[3-(1H-tetrazol-5-yl) [1,1′-biphenyl]-4′-yl]methyl]-3H-1,2,4-triazol-3-one;

5-isopropyl-2-(1-oxopentyl)-2,4-dihydro-4-[[3-(1H-tetrazol-5-yl) [1,1′-biphenyl]-4′-yl]methyl]-3H-1,2,4-triazol-3-one;

5-butyl-2-ethyl-2,4-dihydro-4-[[3-(1H-tetrazol-5-yl) [1,1′-biphenyl]-4′-yl]methyl]-3H-1,2,4-triazol-3-one;

5-butyl-2-propyl-2,4-dihydro-4-[[3-(1H-tetrazol-5-yl) [1,1′-biphenyl]-4′-yl]methyl]-3H-1,2,4-triazol-3-one;

5-butyl-2-isopropyl-2,4-dihydro-4-[[3-(1H-tetrazol-5-yl) [1,1′-biphenyl]-4′-yl]methyl]-3H-1,2,4-triazol-3-one; 2,5-dibutyl-2,4-dihydro-4-[[3-(1H-tetrazol-5-yl) [1,1′-biphenyl]-4′-yl]methyl]-3H-1,2,4-triazol-3-one;

5-butyl-2-secbutyl-2,4-dihydro-4-[[3-(1H-tetrazol-5-yl) [1,1′-biphenyl]-4′-yl]methyl]-3H-1,2,4-triazol-3-one;

5-butyl-2-isobutyl-2,4-dihydro-4-[[3-(1H-tetrazol-5-yl) [1,1′-biphenyl]-4′-yl]methyl]-3H-1,2,4-triazol-3-one;

5-butyl-2-tertbutyl-2,4-dihydro-4-[[3-(1H-tetrazol-5-yl) [1,1′-biphenyl]-4′-yl]methyl]-3H-1,2,4-triazol-3-one;

5-butyl-2-pentyl-2,4-dihydro-4-[[3-(1H-tetrazol-5-yl) [1,1′-biphenyl]-4′-yl]methyl]-3H-1,2,4-triazol-3-one;

5-butyl-2-isopentyl-2,4-dihydro-4-[[3-(1H-tetrazol-5-yl) [1,1′-biphenyl]-4′-yl]methyl]-3H-1,2,4-triazol-3-one;

5-butyl-2-hexyl-2,4-dihydro-4-[[3-(1H-tetrazol-5-yl) [1,1′-biphenyl]-4′-yl]methyl]-3H-1,2,4-triazol-3-one;

5-butyl-2-heptyl-2,4-dihydro-4-[[3-(1H-tetrazol-5-yl) [1,1′-biphenyl]-4′-yl]methyl]-3H-1,2,4-triazol-3-one;

5-butyl-2-octyl-2,4-dihydro-4-[[3-(1H-tetrazol-5-yl) [1,1′-biphenyl]-4′-yl]methyl]-3H-1,2,4-triazol-3-one;

5-butyl-2-cyclohexyl-2,4-dihydro-4-[[3-(1H-tetrazol-5-yl) [1,1′-biphenyl]-4′-yl]methyl]-3H-1,2,4-triazol-3-one;

5-butyl-2-cyclohexylmethyl-2,4-dihydro-4-[[3-(1H-tetrazol-5-yl) [1,1′-biphenyl]-4′-yl]methyl]-3H-1,2,4-triazol-3-one;

5-butyl-2-(2-cyclohexylethyl)-2,4-dihydro-4-[[3-(1H-tetrazol-5-yl) [1,1′-biphenyl]-4′-yl]methyl]-3H-1,2,4-triazol-3-one;

5-butyl-2-cyclohexanoyl-2,4-dihydro-4-[[3-(1H-tetrazol-5-yl) [1,1′-biphenyl]-4′-yl]methyl]-3H-1,2,4-triazol-3-one;

5-butyl-2-(1-oxo-2-cyclohexylethyl)-2,4-dihydro-4-[[3-(1H-tetrazol-5-yl) [1,1′-biphenyl]-4′-yl]methyl]-3H-1,2,4-triazol-3-one;

5-butyl-2-phenyl-2,4-dihydro-4-[[3-(1H-tetrazol-5-yl) [1,1′-biphenyl]-4′-yl]methyl]-3H-1,2,4-triazol-3-one;

5-butyl-2-phenylmethyl-2,4-dihydro-4-[[3-(1H-tetrazol-5-yl) [1,1′-biphenyl]-4′-yl]methyl]-3H-1,2,4-triazol-3-one;

5-butyl-2-(2-phenylethyl)-2,4-dihydro-4-[[3-(1H-tetrazol-5-yl) [1,1′-biphenyl]-4′-yl]methyl]-3H-1,2,4-triazol-3-one;

5-butyl-2-benzoyl-2,4-dihydro-4-[[3-(1H-tetrazol-5-yl) [1,1′-biphenyl]-4′-yl]methyl]-3H-1,2,4-triazol-3-one;

5-butyl-2-(1-oxo-2-phenylethyl)-2,4-dihydro-4-[[3-(1H-tetrazol-5-yl) [1,1′-biphenyl]-4′-yl]methyl]-3H-1,2,4-triazol-3-one;

5-butyl-2-(1-oxopropyl)-2,4-dihydro-4-[[3-(1H-tetrazol-5-yl) [1,1′-biphenyl]-4′-yl methyl]-3H-1,2,4-triazol-3-one;

5-butyl-2-(1-oxobutyl)-2,4-dihydro-4-[[3-(1H-tetrazol-5-yl) [1,1′-biphenyl]-4′-yl]methyl]-3H-1,2,4-triazol-3-one;

5-butyl-2-(1-oxopentyl)-2,4-dihydro-4-[[3-(1H-tetrazol-5-yl) [1,1′-biphenyl]-4′-yl]methyl]-3H-1,2,4-triazol-3-one;

5-butyl-2-(2-butenyl)-2,4-dihydro-4-[[3-(1H-tetrazol-5-yl) [1,1′-biphenyl]-4′-yl]methyl]-3H-1,2,4-triazol-3-one;

5-butyl-2-(3-butenyl)-2,4-dihydro-4-[[3-(1H-tetrazol-5-yl) [1,1′-biphenyl]-4′-yl]methyl]-3H-1,2,4-triazol-3-one;

5-butyl-2-(2-butynyl)-2,4-dihydro-4-[[3-(1H-tetrazol-5-yl) [1,1′-biphenyl]-4′-yl]methyl]-3H-1,2,4-triazol-3-one;

5-butyl-2-(3-butynyl)-2,4-dihydro-4-[[3-(1H-tetrazol-5-yl) [1,1′-biphenyl]-4′-yl]methyl]-3H-1,2,4-triazol-3-one;

5-secbutyl-2-propyl-2,4-dihydro-4-[[3-(1H-tetrazol-5-yl) [1,1′-biphenyl]-4′-yl]methyl]-3H-1,2,4-triazol-3-one;

5-secbutyl-′2-isopropyl-2,4-dihydro-4-[[3-(1H-tetrazol-5-yl) [1,1′-biphenyl]-4′-yl]methyl]-3H-1,2,4-triazol-3-one;

5-secbutyl-2-butyl-2,4-dihydro-4-[[3-(1H-tetrazol-5-yl) [1,1′-biphenyl]-4′-yl]methyl]-3H-1,2,4-triazol-3-one; 2,5-disecbutyl-2,4-dihydro-4-[[3-(1H-tetrazol-5-yl) [1,1′-biphenyl3-4′-yl]methyl]-3H-1,2,4-triazol-3-one;

5-secbutyl-2-isobutyl-2,4-dihydro-4-[[3-(1H-tetrazol-5-yl) [1,1′-biphenyl]-4′-yl]methyl]-3H-1,2,4-triazol-3-one;

5-secbutyl-2-tertbutyl-2,4-dihydro-4-[[3-(1H-tetrazol-5-yl) [1,1′-biphenyl]-4′-yl]methyl]-3H-1,2,4-triazol-3-one;

5-secbutyl-2-pentyl-2,4-dihydro-4-[[3-(1H-tetrazol-5-yl) [1,1′-biphenyl]-4′-yl]methyl]-3H-1,2,4-triazol-3-one;

5-secbutyl-2-isopentyl-2,4-dihydro-4-[[3-(1H-tetrazol-5-yl) [1,1′-biphenyl]-4′-yl]methyl]-3H-1,2,4-triazol-3-one;

5-secbutyl-2-(1-oxopropyl)-2,4-dihydro-4-[[3-(1H-tetrazol-5-yl) [1,1′-biphenyl]-4′-yl]methyl]-3H-1,2,4-triazol-3-one;

5-secbutyl-2-(1-oxobutyl)-2,4-dihydro-4-[[3-(1H-tetrazol-5-yl) [1,1′-biphenyl]-4′-yl]methyl]-3H-1,2,4-triazol-3-one;

5-secbutyl-2-(1-oxopentyl)-2,4-d3hydro-4-[[3-(1H-tetrazol-5-yl) [1,1′-biphenyl]-4′-yl]methyl]-3H-1,2,4-triazol-3-one;

5-isobutyl-2-propyl-2,4-dihydro-4-[[3-(1H-tetrazol-5-yl) [1,1′-biphenyl]-4′-yl]methyl]-3H-1,2,4-triazol-3-one;

5-isobutyl-2-isopropyl-2,4-dihydro-4-[[3-(1H-tetrazol-5-yl) [1,1′-biphenyl]-4′-yl]methyl]-3H-1,2,4-triazol-3-one;

5-isobutyl-2-butyl-2,4-dihydro-4-[[3-(1H-tetrazol-5-yl) [1,1′-biphenyl]-4′-yl]methyl]-3H-1,2,4-triazol-3-one;

5-isobutyl-2-secbutyl-2,4-dihydro-4-[[3-(1H-tetrazol-5-yl) [1,1′-biphenyl]-4′-yl]methyl]-3H-1,2,4-triazol-3-one; 2,5-diisobutyl-2,4-dihydro-4-[[3-(1H-tetrazol-5-yl) [1,1′-biphenyl]-4′-yl]methyl]-3H-1,2,4-triazol-3-one; 5 5-isobutyl-2-tertbutyl-2,4-dihydro-4-[[3-(1H-tetrazol-5-yl) [1,1′-biphenyl]-4′-yl]methyl]-3H-1,2,4-triazol-3-one;

5-isobutyl-2-pentyl-2,4-dihydro-4-[[3-(1H-tetrazol-5-yl) [1,1′-biphenyl]-4′-yl]methyl]-3H-1,2,4-triazol-3-one;

5-isobutyl-2-isopentyl-2,4-dihydro-4-[[3-(1H-tetrazol-5-yl) [1,1′-biphenyl]-4′-yl]methyl]-3H-1,2,4-triazol-3-one;

5-isobutyl-2-(1-oxopropyl)-2,4-dihydro-4-[[3-(1H-tetrazol-5-yl) [1,1′-biphenyl]-4′-yl]methyl]-3H-1,2,4-triazol-3-one;

5-isobutyl-2-(1-oxobutyl)-2,4-dihydro-4-[[3-(1H-tetrazol-5-yl) [1,1′-biphenyl]-4′-yl]methyl]-3H-1,2,4-triazol-3-one;

5-isobutyl-2-(1-oxopentyl)-2,4-dihydro-4-[[3-(1H-tetrazol-5-yl) [1,1′-biphenyl]-4′-yl]methyl]-3H-1,2,4-triazol-3-one;

5-tertbutyl-2-propyl-2,4-dihydro-4-[[3-(1H-tetrazol-5-yl) [1,1′-biphenyl]-4′-yl]methyl]-3H-1,2,4-triazol-3-one;

5-tertbutyl-2-isopropyl-2,4-dihydro-4-[[3-(1H-tetrazol-5-yl) [1,1′-biphenyl]-4′-yl]methyl]-3H-1,2,4-triazol-3-one;

5-tertbutyl-2-butyl-2,4-dihydro-4-[[3-(1H-tetrazol-5-yl) [1,1′-biphenyl]-4′-yl]methyl]-3H-1,2,4-triazol-3-one;

5-tertbutyl-2-secbutyl-2,4-dihydro-4-[[3-(1H-tetrazol-5-yl) [1,1′-biphenyl]-4′-yl]methyl]-3H-1,2,4-triazol-3-one;

5-tertbutyl-2-isobutyl-2,4-dihydro-4-[[3-(1H-tetrazol-5-yl) [1,1′-biphenyl]-4′-yl]methyl--3H-1,2,4-triazol-3-one;

2,5-ditertbutyl-2,4-dihydro-4-[[3-(1H-tetrazol-5-yl) [1,1′-biphenyl]-4′-yl]methyl]-3H-1,2,4-triazol-3-one;

5-tertbutyl-2-pentyl-2,4-dihydro-4-[[3-(1H-tetrazol-5-yl) [1,1′-biphenyl]-4′-yl]methyl]-3H-1,2,4-triazol-3-one;

5-tertbutyl-2-isopentyl-2,4-dihydro-4-[[3-(1H-tetrazol-5-yl) [1,1′-biphenyl]-4′-yl]methyl]-3H-1,2,4-triazol-3-one;

5-tertbutyl-2-(1-oxopropyl)-2,4-dihydro-4-[[3-(1H-tetrazol-5-yl) [1,1′-biphenyl]-4′-yl]methyl]-3H-1,2,4-triazol-3-one;

5-tertbutyl-2-(1-oxobutyl)-2,4-dihydro-4-[[3-(1H-tetrazol-5-yl) [1,1′-biphenyl]-4′-yl]methyl]-3H-1,2,4-triazol-3-one;

5-tertbutyl-2- (1-oxopentyl) -2, 4-dihydro-4- [[3- (1H-tetrazol-5-yl) [1,1′-biphenyl]-4′-yl]methyl]-3H-1,2,4-triazol-3-one;

5-pentyl-2-propyl-2,4-dihydro-4-([3-(1H-tetrazol-5-yl) [1,1′-biphenyl]-4′-yl]methyl]-3H-1,2,4-triazol-3-one;

5-pentyl-2-isopropyl-2,4-dihydro-4-[[3-(1H-tetrazol-5-yl) [1,1′-biphenyl]-4′-yl]methyl]-3H-1,2,4-triazol-3-one;

5-pentyl-2-butyl-2,4-dihydro-4-[[3-(1H-tetrazol-5-yl) [1,1′-biphenyl]-4′-yl]methyl]-3H-1,2,4-triazol-3-one;

5-pentyl-2-secbutyl-2,4-dihydro-4-[[3-(1H-tetrazol-5-yl) [1,1′-biphenyl]-4′-yl]methyl]-3H-1,2,4-triazol-3-one;

5-pentyl-2-isobutyl-2,4-dihydro-4-[[3-(1H-tetrazol-5-yl) [1,1′-biphenyl]-4′-yl]methyl]-3H-1,2,4-triazol-3-one;

5-pentyl-2-tertbutyl-2,4-dihydro-4-[[3-(1H-tetrazol-5-yl) [1,1′-biphenyl]-4′-yl]methyl]-3H-1,2,4-triazol-3-one; 2,5-dipenty1-2,4-dihydro-4-[[3-(1H-tetrazol-5-yl) [1,1′-biphenyl]-4′-yl]methyl]-3H-1,2,4-triazol-3-one;

5-pentyl-2-isopentyl-2,4-dihydro-4-[[3-(1H-tetrazol-5-yl) [1,1′-biphenyl]-4′-yl]methyl]-3H-1,2,4-triazol-3-one;

5-pentyl-2-(1-oxopropyl)-2,4-dihydro-4-[[3-(1H-tetrazol-5-yl) [1,1′-biphenyl]-4′-yl]methyl]-3H-1,2,4-triazol-3-one;

5-pentyl-2-(1-oxobutyl)-2,4-dihydro-4-[[3-(1H-tetrazol-5-yl) [1,1′-biphenyl]-4′-yl]methyl]-3H-1,2,4-triazol-3-one;

5-pentyl-2-(1-oxopentyl)-2,4-dihydro-4-[[3-(1H-tetrazol-5-yl) [1,1′-biphenyl]-4′-yl]methyl]-3H-1,2,4-triazol-3-one; 5-isopentyl-2-propyl-2,4-dihydro-4-[[3-(1H-tetrazol-5-yl) [1,1′-biphenyl]-4′-yl]methyl]-3H-1,2,4-triazol-3-one; 5-isopentyl-2-isopropyl-2,4-dihydro-4-[[3-(1H-tetrazol-5-yl) [1,1′-biphenyl]-4′-yl]methyl]-3H-1,2,4-triazol-3-one; 5-isopentyl-2-butyl-2,4-dihydro-4-[[3-(1H-tetrazol-5-yl) [1,1′-biphenyl]-4′-yl]methyl]-3H-1,2,4-triazol-3-one; 5-isopentyl-2-secbutyl-2,4-dihydro-4-[[3-(1H-tetrazol-5-yl) [1,1′-biphenyl]-4′-yl]methyl]-3H-1,2,4-triazol-3-one; 5-isopentyl-2-isobutyl-2,4-dihydro-4-[[3-(1H-tetrazol-5-yl) [1,1′-biphenyl]-4′-yl]methyl]-3H-1,2,4-triazol-3-one; 5-isopentyl-2-tertbutyl-2,4-dihydro-4-[[3-(1H-tetrazol-5-yl) [1,1′-biphenyl]-4′-yl]methyl]-3H-1,2,4-triazol-3-one; 5-isopentyl-2-pentyl-2,4-dihydro-4-[[3-(1H-tetrazol-5-yl) [1,1′-biphenyl]-4′-yl]methyl]-3H-1,2,4-triazol-3-one; 2,5-diisopentyl-2,4-dihydro-4-[[3-(1H-tetrazol-5-yl) [1,1′-biphenyl]-4′-yl]methyl]-3H-1,2,4-triazol-3-one; 5-isopentyl-2-(1-oxopropyl)-2,4-dihydro-4-[[3-(1H-tetrazol-5-yl) [1,1′-biphenyl]-4′-yl]methyl]-3H-1,2,4-triazol-3-one; 5-isopentyl-2-(1-oxobutyl)-2,4-dihydro-4-([3-(1H-tetrazol-5-yl) [1,1′-biphenyl]-4′-yl]methyl]-3H-1,2,4-triazol-3-one; 5-isopentyl-2-(1-oxopentyl)-2,4-dihydro-4-[[3-(1H-tetrazol-5-yl) [1,1′-biphenyl]-4′-yl]methyl]-3H-1,2,4-triazol-3-one;

5-hexyl-2-propyl-2,4-dihydro-4-[[3-(1H-tetrazol-5-yl) [1,1′-biphenyl]-4′-yl]methyl]-3H-1,2,4-triazol-3-one;

5-hexyl-2-isopropyl-2,4-dihydro-4-[[3-(1H-tetrazol-5-yl) [1,1′-biphenyl]-4′-yl]methyl]-3H-1,2,4-triazol-3-one;

5-hexyl-2-butyl-2,4-dihydro-4-[[3-(1H-tetrazol-5-yl) [1,1′-biphenyl]-4′-yl]methyl]-3H-1,2,4-triazol-3-one;

5-hexyl-2-secbutyl-2,4-dihydro-4-[[3-(1H-tetrazol-5-yl) [1,1′-biphenyl]-4′-yl]methyl]-3H-1,2,4-triazol-3-one;

5-hexyl-2-isobutyl-2,4-dihydro-4-[[3-(1H-tetrazol-5-yl) [1,1′-biphenyl]-4′-yl]methyl]-3H-1,2,4-triazol-3-one;

5-hexyl-2-tertbutyl-2,4-dihydro-4-[[3-(1H-tetrazol-5-yl) [1,1′-biphenyl]-4′-yl]methyl]-3H-1,2,4-triazol-3-one;

5-hexyl-2-pentyl-2,4-dihydro-4-[[3-(1H-tetrazol-5-yl) [1,1′-biphenyl]-4′-yl]methyl]-3H-1,2,4-triazol-3-one;

5-hexyl-2-isopentyl-2,4-dihydro-4-[[3-(1H-tetrazol-5-yl) [1,1′-biphenyl]-4′-yl]methyl]-3H-1,2,4-triazol-3-one; 2,5-dihexyl-2,4-dihydro-4-[[3-(1H-tetrazol-5-yl) [1,1′-biphenyl]-4′-yl]methyl]-3H-1,2,4-triazol-3-one;

5-hexyl-2-(1-oxopropyl)-2,4-dihydro-4-[[3-(1H-tetrazol-5-yl) [1,1′-biphenyl]-4′-yl]methyl]-3H-1,2,4-triazol-3-one;

5-hexyl-2-(1-oxobutyl)-2,4-dihydro-4-[[3-(1H-tetrazol-5-yl) [1,1′-biphenyl]-4′-yl]methyl]-3H-1,2,4-triazol-3-one;

5-hexyl-2-(1-oxopentyl)-2,4-dihydro-4-[[3-(1H-tetrazol-5-yl) [1,1′-biphenyl]-4′-yl]methyl]-3H-1,2,4-triazol-3-one;

5-heptyl-2-butyl-2,4-dihydro-4-[[3-(1H-tetrazol-5-yl) [1,1′-biphenyl]-4′-yl]methyl]-3H-1,2,4-triazol-3-one; 2,5-diheptyl-2,4-dihydro-4-[[3-(1H-tetrazol-5-yl) [1,1′-biphenyl]-4′-yl]methyl]-3H-1,2,4-triazol-3-one;

5-heptyl-2-(1-oxopropyl)-2,4-dihydro-4-[[3-(1H-tetrazol-5-yl) [1,1′-biphenyl]-4′-yl]methyl]-3H-1,2,4-triazol-3-one;

5-heptyl-2-(1-oxobutyl)-2,4-dihydro-4-[[3-(1H-tetrazol-5-yl) [1,1′-biphenyl]-4′-yl]methyl]-3H-1,2,4-triazol-3-one;

5-heptyl-2-(1-oxopentyl)-2,4-dihydro-4-[[3-(1H-tetrazol-5-yl) [1,1′-biphenyl]-4′-yl]methyl]-3H-1,2,4-triazol-3-one;

5-octyl-2-butyl-2,4-dihydro-4-[[3-(1H-tetrazol-5-yl) [1,1′-biphenyl]-4′-yl]methyl]-3H-1,2,4-triazol-3-one; 5 2,5-dioctyl-2,4-dihydro-4-[[3-(1H-tetrazol-5-yl) [1,1′-biphenyl]-4′-yl]methyl]-3H-1,2,4-triazol-3-one;

5-octyl-2-(1-oxopropyl)-2,4-dihydro-4-[[3-(1H-tetrazol-5-yl) [1,1′-biphenyl]-4′-yl]methyl]-3H-1,2,4-triazol-3-one;

5-octyl-2-(1-oxobutyl)-2,4-dihydro-4-[[3-(1H-tetrazol-5-yl) [1,1′-biphenyl]-4′-yl]methyl]-3H-1,2,4-triazol-3-one;

5-octyl-2-(1-oxopentyl)-2,4-dihydro-4-[[3-(1H-tetrazol-5-yl) [1,1′-biphenyl]-4′-yl]methyl]-3H-1,2,4-triazol-3-one;

5-(1butenyl)-2-propyl-2,4-dihydro-4-[[3-(1H-tetrazol-5-yl) [1,1′-biphenyl]-4′-yl]methyl]-3H-1,2,4-triazol-3-one;

5-(1-butenyl)-2-isopropyl-2,4-dihydro-4-[[3-(1H-tetrazol-5-yl) [1,1′-biphenyl]-4′-yl]methyl]-3H-1,2,4-triazol-3-one;

5-(1butenyl)-2-butyl-2,4-dihydro-4-[[3-(1H-tetrazol-5-yl) [1,l1-biphenyl]-4′-yl]methyl]-3H-l,2,4-triazol-3-one;

5-(1butenyl)-2-secbutyl-2,4-dihydro-4-[[3-(1H-tetrazol-5-yl) [1,1′-biphenyl]-4′-yl]methyl]-3H-1,2,4-triazol-3-one;

5-(1-butenyl)-2-isobutyl-2,4-dihydro-4-[[3-(1H-tetrazol-5-yl) [1,1′-biphenyl]-4′-yl]methyl]-3H-1,2,4-triazol-3-one;

5-(1-butenyl)-2-tertbutyl-2,4-dihydro-4-[[3-(1H-tetrazol-5-yl) [1,1′-biphenyl]-4′-yl]methyl]-3H-1,2,4-triazol-3-one;

5-(1butenyl)-2-pentyl-2,4-dihydro-4-[[3-(1H-tetrazol-5-yl) [1,1′-biphenyl]-4′-yl]methyl]-3H-1,2,4-triazol-3-one;

5-(1-butenyl)-2-isopentyl-2,4-dihydro-4-[[3-(1H-tetrazol-5-yl) [1,1′-biphenyl]-4′-yl]methyl]-3H-1,2,4-triazol-3-one;

5-(1-butenyl)-2-hexyl-2,4-dihydro-4-[[3-(1H-tetrazol-5-yl) [1,1′-biphenyl]-4′-yl]methyl]-3H-1,2,4-triazol-3-one;

5-(1-butenyl)-2-heptyl2,4-dihydro-4-[[3-(1H-tetrazol-5-yl) [1,1′-biphenyl]-4′-yl]methyl]-3H-1,2,4-triazol-3-one;

5-(1-butenyl)-2-octyl-2,4-dihydro-4-[[3-(1H-tetrazol-5-yl) [1,l′-biphenyl]-4′-yl]methyl]-3H-1,2,4-triazol-3-one;

5-(1-butenyl)-2-cyclohexyl-2,4-dihydro-4-[[3-(1H-tetrazol-5-yl) [1,1′-biphenyl]-4′-yl]methyl]-3H-1,2,4-triazol-3-one;

5-(1-butenyl)-2-cyclohexylmethyl-2,4-dihydro-4-[[3-(1H-tetrazol-5-yl) [1,1′-biphenyl]-4′-yl]methyl]-3H-1,2,4-triazol-3-one;

5-(1-butenyl)-2-(2-cyclohexylethyl)-2,4-dihydro-4-[[3-(1H-tetrazol-5-yl) [1,1′-biphenyl]-4′-yl]methyl]-3H-1,2,4-triazol-3-one;

5-(1-butenyl)-2-cyclohexanoyl-2,4-dihydro-4-[[3-(1H-tetrazol-5-yl) [1,1′-biphenyl]-4′-yl]methyl]-3H-1,2,4-triazol-3-one;

5-(1-butenyl)-2-(1-oxo-2-cyclohexylethyl)-2,4-dihydro-4-[[3-(1H-tetrazol-5-yl) [1,1′-biphenyl3-4′-yl]methyl]-3H-1,2,4-triazol-3-one;

5-(1-butenyl)-2-phenyl-2,4-dihydro-4-[[3-(1H-tetrazol-5-yl) [1,1′-biphenyl]-4′-yl]methyl]-3H-1,2,4-triazol-3-one;

5-(1-butenyl)-2-phenylmethyl-2,4-dihydro-4-[[3-(1H-tetrazol-5-yl) [1,1′-biphenyl]-4′-yl]methyl]-3H-1,2,4-triazol-3-one;

5-(1butenyl)-2-benzoyl-2,4-dihydro-4-[[3-(1H-tetrazol-5-yl) [1,1′-biphenyl]-4′-yl]methyl]-3H-1,2,4-triazol-3-one;

5-(1-butenyl)-2-(1-oxo-2-phenylethyl)-2,4-dihydro-4-[[3-(1H-tetrazol-5-yl) [1,1′-biphenyl]-4′-yl]methyl]-3H-1,2,4-triazol-3-one;

5-(1-butenyl)-2-(1-oxopropyl)-2,4-dihydro-4-[[3-(1H-tetrazol-5-yl) [1,1′-biphenyl]-4′-yl]methyl]-3H-1,2,4-triazol-3-one;

5-(1-butenyl)-2-(1-oxobutyl)-2,4-dihydro-4-[[3-(1H-tetrazol-5-yl) [1,1′-biphenyl]-4′-yl]methyl]-3H-1,2,4-triazol-3-one;

5-(1-butenyl)-2-(1-oxopentyl)-2,4-dihydro-4-[[3-(1H-tetrazol-5-yl) [1,1′-biphenyl]-4′-yl]methyl]-3H-1,2,4-triazol-3-one;

5-(1butenyl)-2-(2-butenyl)-2,4-dihydro-4-[[3-(1H-tetrazol-5-yl) [1,1′-biphenyl]-4′-yl]methyl]-3H-1,2,4-triazol-3-one;

5-(1-butenyl)-2-(3-butenyl)-2,4-dihydro-4-[[3-(1H-tetrazol-5-yl) [1,1′-biphenyl]-4′-yl]methyl]-3H-1,2,4-triazol-3-one;

5-(1-butenyl)-2-(2-butynyl)-2,4-dihydro-4-[[3-(1H-tetrazol-5-yl) [1,1′-biphenyl]-4′-yl]methyl]-3H-1,2,4-triazol-3-one;

5-(1-butenyl)-2-(3-butynyl)-2,4-dihydro-4-[[3-(1H-tetrazol-5-yl) [1,1′-biphenyl]-4′-yl]methyl]-3H-1,2,4-triazol-3-one;

5-(2-butenyl)-2-propyl-2,4-dihydro-4-[[3-(1H-tetrazol-5-yl) [1,1′-biphenyl]-4′-yl]methyl]-3H-1,2,4-triazol-3-one;

5-(2-butenyl)-2-isopropyl-2,4-dihydro-4-[[3-(1H-tetrazol-5-yl) [1,1′-biphenyl]-4′-yl]methyl]-3H-1,2,4-triazol-3-one;

5-(2-butenyl)-2-butyl-2,4-dihydro-4-[[3-(1H-tetrazol-5-yl) [1,1′-biphenyl]-4′-yl]methyl]-3H-1,2,4-triazol-3-one;

5-(2-butenyl)-2-secbutyl-2,4-dihydro-4-[[3-(1H-tetrazol-5-yl) [1,1′-biphenyl]-4′-yl]methyl]-3H-1,2,4-triazol-3-one;

5-(2-butenyl)-2-isobutyl-2,4-dihydro-4-[[3-(1H-tetrazol-5-yl) [1,1′-biphenyl]-4′-yl]methyl]-3H-1,2,4-triazol-3-one;

5-(2-butenyl)-2-tertbutyl-2,4-dihydro-4-[[3-(1H-tetrazol-5-yl) [1,1′-biphenyl]-4′-yl]methyl]-3H-1,2,4-triazol-3-one;

5-(2-butenyl)-2-pentyl-2,4-dihydro-4-[[3-(1H-tetrazol-5-yl) [1,1′-biphenyl]-4′-yl]methyl]-3H-1,2,4-triazol-3-one;

5-(2-butenyl)-2-isopentyl-2,4-dihydro-4-[[3-(1H-tetrazol-5-yl) [1,1′-biphenyl]-4′-yl]methyl]-3H-1,2,4-triazol-3-one;

5-(2-butenyl)-2-(1-oxopropyl)-2,4-dihydro-4-[[3-(1H-tetrazol-5-yl) [1,1′-biphenyl]-4′-yl]methyl]-3H-1,2,4-triazol-3-one;

5-(2-butenyl)-2-(1-oxobutyl)-2,4-dihydro-4-[[3-(1H-tetrazol-5-yl) [1,1′-biphenyl]-4′-yl]methyl]-3H-1,2,4-triazol-3-one;

5-(2-butenyl)-2-(1-oxopentyl)-2,4-dihydro-4-[[3-(1H-tetrazol-5-yl) [1,1′-biphenyl]-4′-yl]methyl]-3H-1,2,4-triazol-3-one;

5-(3-butenyl)-2-propyl-2,4-dihydro-4-[[3-(1H-tetrazol-5-yl) [1,1′-biphenyl]-4′-yl]methyl]-3H-1,2,4-triazol-3-one;

5-(3-butenyl)-2-butyl-2,4-dihydro-4-[[3-(1H-tetrazol-5-yl) [1,1′-biphenyl]-4′-yl]methyl]-3H-1,2,4-triazol-3-one;

5-(3-butenyl)-2-pentyl-2,4-dihydro-4-[[3-(1H-tetrazol-5-yl) [1,1′-biphenyl]-4′-yl]methyl]-3H-1,2,4-triazol-3-one;

5-(3-butenyl)-2-isopentyl-2,4-dihydro-4-[[3-(1H-tetrazol-5-yl) [1,1′-biphenyl]-4′-yl]methyl]-3H-1,2,4-triazol-3-one;

5-(3-butenyl)-2-(1-oxopropyl)-2,4-dihydro-4-[[3-(1H-tetrazol-5-yl) [1,1′-biphenyl]-4′-yl]methyl]-3H-1,2,4-triazol-3-one;

5-(3-butenyl)-2-(1-oxobutyl)-2,4-dihydro-4-[[3- (1H-tetrazol-5-yl) [1,1′-biphenyl]-4′-yl]methyl]-3H-1,2,4-triazol-3-one;

5-(3-butenyl)-2-(1-oxopentyl)-2,4-dihydro-4-[[3-(1H-tetrazol-5-yl) [1,1′-biphenyl]-4′-yl]methyl]-3H-1,2,4-triazol-3-one;

5-(1-butynyl)-2-propyl-2,4-dihydro-4-[[3-(1H-tetrazol-5-yl) [1,1′-biphenyl]-4′-yl]methyl]-3H-1,2,4-triazol-3-one;

5-(1-butynyl)-2-butyl-2,4-dihydro-4-[[3-(1H-tetrazol-5-yl) [1,1′-biphenyl]-4′-yl]methyl]-3H-1,2,4-triazol-3-one;

5-(1-butynyl)-2-secbutyl-2,4-dihydro-4-[[3-(1H-tetrazol-5-yl) [1,1′-biphenyl]-4′-yl]methyl]-3H-1,2,4-triazol-3-one;

5-(1-butynyl)-2-isobutyl-2,4-dihydro-4-[[3-(1H-tetrazol-5-yl) [1,1′-biphenyl]-4′-yl]methyl]-3H-1,2,4-triazol-3-one;

5-(1-butynyl)-2-tertbutyl-2,4-dihydro-4-[[3-(1H-tetrazol-5-yl) [1,1′-biphenyl]-4′-yl]methyl]-3H-1,2,4-triazol-3-one;

5-(1-butynyl)-2-pentyl-2,4-dihydro-4-[[3-(1H-tetrazol-5-yl) [1,1′-biphenyl]-4′-yl]methyl]-3H-1,2,4-triazol-3-one;

5-(1-butynyl)-2-isopentyl-2,4-dihydro-4-[[3-(1H-tetrazol-5-yl) [1,1′-biphenyl]-4′-yl]methyl]-3H-1,2,4-triazol-3-one;

5-(1-butynyl)-2-hexyl-2,4-dihydro-4-[[3-l(H-tetrazol-5-yl) [1,1′-biphenyl]-4′-yl]methyl]-3H-1,2,4-triazol-3-one;

5-(1-butynyl)-2-(1-oxopropyl)-2,4-dihydro-4-[[3-(1H-tetrazol-5-yl) [1,1′-biphenyl]-4′-yl]methyl]-3H-1,2,4-triazol-3-one;

5-(1-butynyl)-2-(1-oxobutyl)-2,4-dihydro-4-[[3-(1H-tetrazol-5-yl) [1,1′-biphenyl]-4′-yl]methyl]-3H-1,2,4-triazol-3-one;

5-(1-butynyl)-2-(1-oxopentyl)-2,4-dihydro-4-[[3-(1H-tetrazol-5-yl) [1,1′-biphenyl]-4′-yl]methyl]-3H-1,2,4-triazol-3-one;

5-(2-butynyl)-2-propyl-2,4-dihydro-4-[[3-(1H-tetrazol-5-yl) [1,1′-biphenyl]-4′-yl]methyl]-3H-1,2,4-triazol-3-one;

5-(2-butynyl)-2-butyl-2,4-dihydro-4-[[3-(1H-tetrazol-5-yl) [1,1′-biphenyl]-4′-yl]methyl]-3H-1,2,4-triazol-3-one;

5-(2-butynyl)-2-pentyl-2,4-dihydro-4-[[3-(1H-tetrazol-5-yl) [1,1′-biphenyl]-4′-yl]methyl]-3H-1,2,4-triazol-3-one;

5-(2-butynyl)-2-isopentyl-2,4-dihydro-4-[[3-(1H-tetrazol-5-yl) [1,1′-biphenyl]-4′-yl]methyl]-3H-1,2,4-triazol-3-one;

5-(2-butynyl)-2-(1-oxopropyl)-2,4-dihydro-4-([3-(1H-tetrazol-5-yl) [1,1′-biphenyl]-4′-yl]methyl]-3H-1,2,4-triazol-3-one;

5-(2-butynyl)-2-(1-oxobutyl)-2,4-dihydro-4-[[3-(1H-tetrazol-5-yl) [1,1′-biphenyl]-4′-yl]methyl]-3H-1,2,4-triazol-3-one;

5-(2-butynyl)-2-(1-oxopentyl)-2,4-dihydro-4-[[3-(1H-tetrazol-5-yl) [1,1′-biphenyl]-4′-yl]methyl]-3H-1,2,4-triazol-3-one;

5-(3-butynyl)-2-propyl-2,4-dihydro-4-[[3-(1H-tetrazol-5-yl) [1,1′-biphenyl]-4′-yl]methyl]-3H-1,2,4-triazol-3-one;

5-(3-butynyl)-2-butyl-2,4-dihydro-4-[[3-(1H-tetrazol-5-yl) [1,1′-biphenyl]-4′-yl]methyl]-3H-1,2,4-triazol-3-one;

5-(3-butynyl) -2-isopentyl-2, 4-dihydro-4- [[3- (1H-tetrazol-5-yl) [1,1′-biphenyl]-4′-yl]methyl]-3H-1,2,4-triazol-3-one;

5-(3-butynyl)-2-pentyl-2,4-dihydro-4-[[3-(1H-tetrazol-5-yl) [1,1′-biphenyl]-4′-yl]methyl]-3H-1,2,4-triazol-3-one;

5-(3-butynyl)-2-(1-oxopropyl)-2,4-dihydro-4 -[[3-(1H -tetrazol-5-yl) [1,1′-biphenyl]-4 ′-yl]methyl]-3H-1,2,4-triazol-3-one;

5-(3-butynyl)-2-(1-oxobutyl)-2,4-dihydro-4-[[3-(1H-tetrazol-5-yl) [1,1′-biphenyl]-4′-yl]methyl]-3H-l,2,4-triazol-3-one;

5-(3-butynyl)-2-(1-oxopentyl)-2,4-dihydro-4-[[3-(1H-tetrazol-5-yl) [1,1′-biphenyl]-4′-yl]methyl]-3H-1,2,4-triazol-3-one; and 4′-[(1,3-dibutyl-4,5-dihydro-5-oxo-1H-1,2,4-triazol-4-yl) methyl] [1,1′-biphenyl]-3-carboxylic acid.

The term “hydrido” denotes a single hydrogen atom (H) which may be attached, for example, to a carbon atom to form a hydrocarbyl group or attached to an oxygen atom to form an hydroxyl group. Where the term “alkyl” is used, either alone or within other terms such as “haloalkyl” and “hydroxyalkyl”, the term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about five carbon atomms. The term “cycloalkyl” embraces cyclic radicals having three to about ten ring carbon atoms, preferably three to about six carbon atoms, such as cyclopropyl and cyclobutyl. The term “haloalkyl” embraces radicals wherein any one or more of the alkyl carbon atoms is substituted with one or more halo groups, preferably selected from bromo, chloro and fluoro. Specifically embraced by the term “haloalkyl” are monohaloalkyl, dihaloalkyl and polyhaloalkyl groups. A monohaloalkyl group, for example, may have either a bromo, a chloro, or a fluoro atom within the group. Dihaloalkyl and polyhaloalkyl groups may be substituted with two or more of the same halo groups, or may have a combination of different halo groups. A dihaloalkyl group, for example, may have two fluoro atoms, such as difluoromethyl and difluorobutyl groups, or two chloro atoms, such as a dichloromethyl group, or one fluoro atom and one chloro atom, such as a fluorochloromethyl group. Examples of a polyhaloalkyl are trifluoromethyl, 1,1-difluoroethyl, 2,2,2-trifluoroethyl, perfluoroethyl and 2,2,3,3-tetrafluoropropyl groups. The term “difluoroalkyl” embraces alkyl groups having two fluoro atoms substituted on any one or two of the alkyl group carbon atoms. Preferably, when the difluoroalkyl group is attached at the triazole ring R¹ and R² positions of Formula I, the two fluoro atoms are substituted on the carbon atom which is attached directly to the triazole ring. Such preferred difluoroalkyl group may be characterized as an “alpha-carbon difluoro-substituted difluoroalkyl group” The terms “alkylol” and “hydroxyalkyl” embrace linear or branched alkyl groups having one to about ten carbon atoms any one of which may be substituted with one or more hydroxyl groups. The term “alkenyl” embraces linear or branched radicals having two to about twenty carbon atoms, preferably three to about ten carbon atoms, and containing at least one carbon-carbon double bond, which carbon-carbon double bond may have either cis or trans geometry within the alkenyl moiety. The term “alkynyl” embraces linear or branched radicals having two to about twenty carbon atoms, preferably two to about ten carbon atoms, and containing at least one carbon-carbon triple bond. The terms “cycloalkenyl” and “cycloalkynyl” embrace cyclic radicals having three to about ten ring carbon atoms including, respectively, one or more double or triple bonds involving adjacent ring carbons. The terms “alkoxy” and “alkoxyalkyl” embrace linear or branched oxy-containing radicals each having alkyl portions of one to about ten carbon atoms, such as methoxy group. The term “alkoxyalkyl” also embraces alkyl radicals having two or more alkoxy groups attached to the alkyl radical, that is, to form monoalkoxyalkyl and dialkoxyalkyl groups. The “alkoxy” or “alkoxyalkyl” radicals may be further substituted with one or more halo atoms, such as fluoro, chloro or bromo, to provide haloalkoxy or haloalkoxyalkyl groups. The term “alkylthio” embraces radicals containing a linear or branched alkyl group, of one to about ten carbon atoms attached to a divalent sulfur atom, such as a methythio group. The term “aryl” embraces aromatic radicals such as phenyl, naphthyl and biphenyl. The terms “aralkyl” and “arylalkyl” embrace aryl-substituted alkyl radicals such as benzyl, diphenylmethyl, triphenylmethyl, phenylethyl, phenylbutyl and diphenylethyl. The terms “benzyl” and “phenylmethyl” are interchangeable. The terms “aryloxy” and “arylthio” denote radical respectively, aryl groups having an oxygen or sulfur atom through which the radical is attached to a nucleus, examples of which are phenoxy and phenylthio. The terms “sulfinyl” and “sulfonyl”, whether used alone or linked to other terms, denotes respectively divalent radicals SO and S₂. The term “aralkoxy”, alone or within another term, embraces an aryl group attached to an alkoxy group to form, for example, benzyloxy. The term “acyl” whether used alone, or within a term such as acyloxy, denotes a radical provided by the residue after removal of hydroxyl from an organic acid, examples of such radical being acetyl and benzoyl. The term “heteroaryl” embraces aromatic ring systems containing one or two hetero atoms selected from oxygen, nitrogen and sulfur in a ring system having five or six ring members, examples of which are thienyl, furanyl, pyridinyl, thiazolyl, pyrimidyl and isoxazolyl. Such heteroaryl may be attached as a substituent through a carbon atom of the heteroaryl ring system, or may be attached through a carbon atom of a moiety substituted on a heteroaryl ring-member carbon atom, for example, through the methylene substituent of imidazolemethyl moiety. Also, such heteroaryl may be attached through a ring nitrogen atom as long as aromaticity of the heteroaryl moiety is preserved after attachment.

Specific examples of alkyl groups are methyl, ethyl, n-propyl, isopropyl, n-butyl, sec-butyl, isobutyl, tert-butyl, n-pentyl, isopentyl and neopentyl. Typical alkenyl and alkynyl groups may have one unsaturated bond, such as an allyl group, or may have a plurality or unsaturated bonds, with such plurality of bonds either adjacent, such as allene-type structures, or in conjugation, or separated by several saturated carbons.

Compounds of Formula I have been found to inhibit the action of angiotensin II in mammals. Angiotensin II is a potent vasoconstrictor and participates in the formation of aldosterone which regulates sodium and water balance in mammals. Thus, compounds of Formula I are therapeutically useful in methods for treating hypertension by administering to a hypertensive patient a therapeutically-effective amount of a compound of Formula I. The phrase “hypertensive patient” means, in this context, a mammalian subject suffering from the effects of hypertension or susceptible to a hypertensive condition if not treated to prevent or control such hypertension.

Included within the family of compounds of Formula I are the tautomeric forms of the described compounds and all diastereoisomers thereof and the pharmaceutically-acceptable salts thereof.

The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of compounds of Formula I may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, p-hydroxybenzoic, salicyclic, phenylacetic, mandelic, embonic (pamoic), methansulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactaric and galacturonic acid. Suitable pharmaceutically-acceptable base addition salts of compounds of Formula I include metallic salts made from aluminium, calcium, lithium, magnesium, potassium, sodium and zinc or organic salts made from N,N′-dibenzylethylene-diamine, chloroprocaine, choline, diethanolamine, ethylenediamine, meglumine (N-methylglucamine) and procaine. All of these salts may be prepared by conventional means from the corresponding compound of Formula I by reacting, for example, the appropriate acid or base with the compound of Formula I.

### GENERAL SYNTHETIC PROCEDURES

The compounds of the invention can be synthesized according to the following procedures of Schemes I-IV, wherein each of the substituents R¹, R², R⁵ and B are as defined for Formula I above, except where further noted.

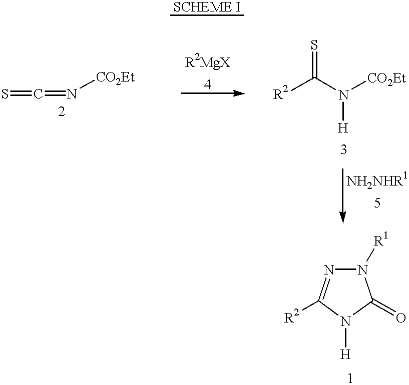

Synthetic Scheme I shows the preparation of 1,2,4-triazolone 1 from isothiocyanate 2 via N-ethoxycarbonyl thioamide 3 according to the general procedure outlined by Papadopoulos, E. P. and George, B., J. Orc. Chem., 41, 3233 (1976). In the first step, N-ethoxycarbonyl thioamide 3 can be prepared from ethoxycarbonyl isothiocyanate 2 and the corresponding organometallic 4 . The N-ethoxycarbonyl thioamide 3 can be converted to triazolone 1 by the action of the corresponding hydrazines a as described in the reference given above.

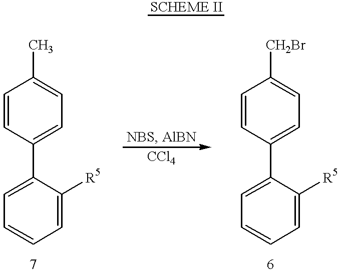

Synthetic Scheme II shows the preparation of the alkylating agent 6 from the corresponding precursor 7. When R⁵ equals CO₂CH₃, 7 was purchased from Chemo Dynamics Inc.

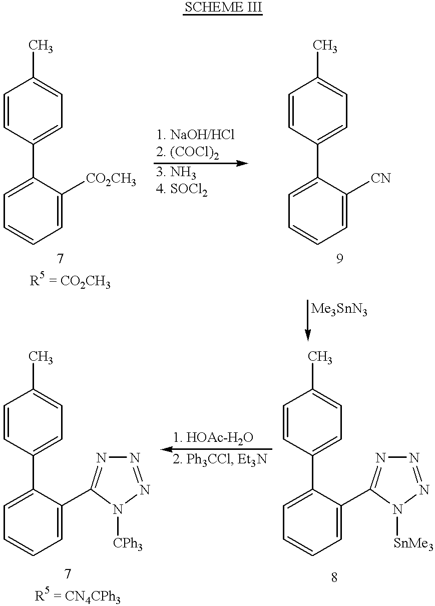

Synthetic Scheme III shows the preparation of the alkylating agent precursor 7 where R⁵ equal CN₄C(C₆H₅)3 from the corresponding methyl ester 7 (R⁵=CO₂CH₃). In step 1, the methyl ester is converted to the corresponding acid (R⁵=CO₂H) by the action of sodium hydroxide/hydrochloric acid. In step 2, the acid is converted to the corresponding acid chloride (R⁵=COCl) by the action of oxalyl chloride. In step 3, the acid chloride is converted to the corresponding primary amide (R⁵=CONH₂) by the action of ammonium. In step 4, the amide is converted to the corresponding nitrile 9 by the action of thionyl chloride at reflux. The nitrile 9 is reacted with trimethyltin azide in toluene at reflux to give the corresponding trimethyltin protected tetrazole 8; deprotection with acetic acid/water and reprotection with triphenylmethyl chloride/triethylamine gives the N-trityl tetrazole 7 (R⁵=CN₄C(C₆H₅)3).

[chemical structure]

Synthetic Scheme IV shows the coupling reaction of the 1,2,4-triazolone 1 with the appropriate alkylating reagent 6 and subsequent conversion to the compounds of the invention. In the first step, 1 is treated with a base, such as potassium t-butoxide, to generate the corresponding anion 10. Anion 10 is reacted with an alkylating agent 6 to give coupled product 11. The trisubstituted 1,2,4-triazolone 11 can be subsequently converted to the corresponding acid 12 or tetrazole 13 by treatment with one of the appropriate reagents as shown in Scheme IV.

Alternatively, the compounds of the invention can be synthesized according to the following procedures of Scheme V and VI.

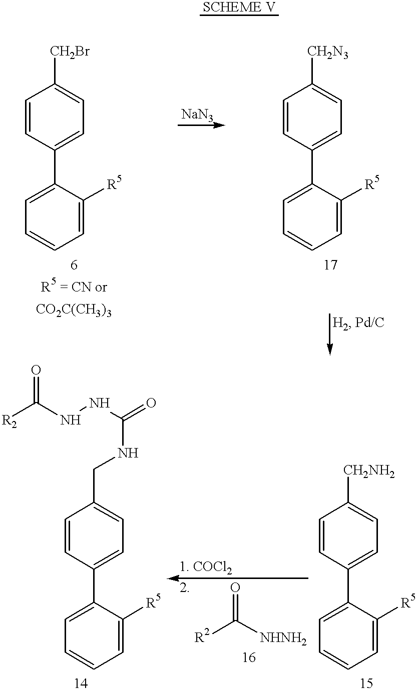

Synthetic Scheme V shows the preparation of semicarbazide 14 from bromide 6. Semicarbazide 14 can be prepared from aminomethylbiphenyl 15 and the corresponding hydrazide 16 which can be obtained by treating its ester precursor with hydrazine. Amine 15 can be prepared by hydrogenation of the corresponding azide 17 which can be obtained from its bromide precursor 6 by treatment with sodium azide.

[chemical structure]

Synthetic Scheme VI shows the cyclization of 14 to 18 and the subsequent alkylation to 11. In the first step, triazolone 18 can be prepared by dehydration of semicarbazide 14 in alcohol with a base catalyst, such as sodium methoxide. Then triazolone 18 is treated with a base, such as potassium t-butoxide, to generate the corresponding anion 19. Anion 19 is reacted with an alkylating agent 20 to give alkylated product 11 which may be converted to the corresponding acid 12 or tetrazole 13 by treatment with one of the appropriate reagents as shown in Scheme IV.

### EXAMPLE 1

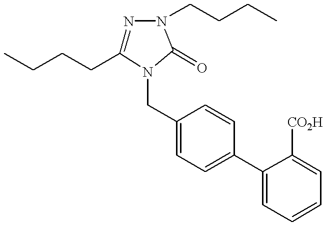

4′-[(1,3-dibutyl-4,5-dihydro-5-oxo-1H-1,2,4-triazol-4-yl) methyl] [1,1′-biphenyl]-2-carboxylic acid

Step 1: Preparation of 2-(p-tolyl benzoic acid

A mixture of 52 g (0.23 mol) of methyl 2-(p-tolyl) benzoate (Chemo Dynamics Inc.) and 100 mL (0.25 mol) of 2.5 N aqueous sodium hydroxide solution in 100 mL of methanol was stirred at room temperature for about 6 h, then at reflux for about 6 h. The resulting solution was concentrated in vacuo to half of its original volume. The aqueous solution was acidified with 3 N hydrochloric acid to about pH 3 and extracted with methylene chloride. The extracts were dried (MgSO₄) and concentrated in vacuo to give 48.7 g (99) of 2-(p-tolyl)benzoic acid as a white solid: ¹H NMR (CDCl₃) δ 2.40 (s, 3H), 7.00-8.05 (m, 8H), 10.4-11.4 (broad s, 1H).

Step 2: Preparation of tert-butyl 2-(p-tolyl)benzoate

To 48 g (0.226 mol) of 2-(p-tolyl)benzoic acid from step 1 and 5 mL of concentrated sulfuric acid in 100 mL of anhydrous ether cooled in a dry ice-acetone bath was transferred 100 mL of 2-methylpropene. The resulting mixture was agitated on a Parr apparatus at room temperature for about 15 h, and the excess of 2-methylpropene was allowed to evaporate at room temperature. The ethereal solution was neutralized with 2.5 N aqueous sodium hydroxide solution, and extracted with three 200 mL-portions of ether. The combined extracts were dried (MgSO₄) and concentrated in vacuo to give 57.2 g (94%) of tert-butyl 2-(p-tolyl)benzoate as a solid: ¹H NMR (CDCl₃) δ 1.29 (s, 9H), 2.40 (s, 3H), 7.21 (s, 4H), 7.25-7.55 (m, 3H), 7.7-7.8 (m, 1H).

Step 3: Preparation of 4-bromomethyl-2′-tert-butoxycarbonylbiphenyl

To a mixture of 57 g (0.213 mol) of tert-butyl 2-(p-tolyl)benzoate from step 2 in 1.7 L of carbon tetrachloride at about 65° C. was added 36.4 g (0.204 mol) of N-bromosuccinimide (NBS) and 0.82 g (5 mmol) of azobisisobutyronitrile (AIBN) in one portion. The resulting solution was stirred at reflux for about 6 h followed by addition of another 1.7 g (9.6 mmol) of N-bromosuccinimide and 0.1 g (0.6 mmol) of AIBN. The mixture was stirred at reflux for additional 13 h, and concentrated in vacuo. The residue was dissolved in ethyl acetate-hexane (1:3) and filtered through a pad of silica gel, eluted with ethyl acetate-hexane (1:3) to give 79 g (quantitative) of a sample which contained mostly desired product, 4-bromomethyl-2′-tert-butoxycarbonylbiphenyl, with a small amount of dibrominated product and starting material. No further purification, however, was attempted and the mixture was used directly in subsequent reactions: ¹H NMR (CDC₁₃) δ 1.26 (s, 9H), 4.56 (s, 2H), 7.2-7.9 (m, 8H)

Step 4: Preparation of 4-azidomethyl-2′-tert-butoxycarbonylbiphenyl

The mixture of 11.0 g (0.032 mol) of 4-bromomethyl-2′-tert-butoxycarbonyl-biphenyl from step 3 and 4.6 g (0.071 mol) of sodium azide in 42 mL of dimethylformamide and 4.2 mL of water was stirred at room temperature for about 24 h and concentrated in vacuo. The residue was extracted with ethyl acetate, and the combined extracts were washed with water, dried (MgSO₄) and concentrated in vacuo to give 10 g (quantitative) of 4-azidomethyl-2′-tert-butoxycarbonyl biphenyl as a yellow oil: ¹H NMR (CDCl₃) δ 1.26 (s, 9H), 4.38 (s, 2H), 7.2-7.9 (m, 8H).

Step 5: Preparation of 4-aminomethyl-21-tert-butoxycarbonylbiohenyl

A suspension of 10 g (32 mmnol) of the azidomethylbiphenyl from step 4 and 0.6 g of 10% palladium on carbon in 30 miL of absolute ethanol was agitated on a Parr apparatus under a hydrogen atmosphere at 40 psi for about 20 h. The mixture was filtered through a pad of celite and concentrated in vacuo to give 9.2 g (quantitative) of 4-aminomethyl-2′-tert-butoxycarbonylbiphenyl as a yellow oil: ¹H NMR (CDCl₃) δ 1.28 (s, 9H) , 1.71 (broad s, 2H) , 3.92 (s, 2H), 7.2-7.9 (m, 8H).

Step 6: Preparation of tert-butyl 4′-[[[[2-(1-oxopentyl) hydrazino]carbonyl]amino]methyl] [1,1′-biphenyl]-2-carboxylate

To 6.4 miL of 1M (6.4 mmol) phosgene in toluene and 5 mL of toluene at 0° C. was added dropwise a solution of 1.0 g (3.53 mmol) of 4-aminomethyl-2′-tert-butoxycarbonylbiphenyl in 5 mL of methylene chloride along with dropwise addition of 1.5 mL (18.5 mmol) of pyridine. The resulting mixture was stirred at 0° C. for about 30 min, at room temperature for about 1.5 h, and the excess of phosgene was removed under a stream of nitrogen. To the mixture was added 800 mg (6.9 mmol) of valeric acid hydrazide (Lancaster Synthesis), 1.0 mL (12.3 mmol) of pyridine and 2 mL of ether, and the resulting mixture was stirred at room temperature for about 2.5 h. The reaction mixture was concentrated in vacuo, and the residue was chromatographed over silica gel (eluted with isopropanol-hexane, 1:5) to give 780 mg (52%) of semicarbazide as a white solid: ¹H NMR (CDCl₃) δ 0.86 (t, J=7.2 Hz, 3H), 1.28 (s, 9H), 1.20-1.40 (m, 2H), 1.56 (quintet, J 7.2 Hz, 2H), 2.20 (t, J=7.5 Hz, 2H), 4.38 (d, J=6 Hz, 2H), 6.27 (broad t, 1H), 7.2-7.5 (m, 7H), 7.76 (dd, J=7.5, 1.0 Hz, 1H), 7.95-8.05 (m, 1H), 8.85 (broad s, 1H); ¹³C NMR (CDCl₃) δ 13.7, 22.3, 27.4, 27.7, 33.6, 43.7, 127.0, 128.8, 129.7, 130.7, 132.7, 127.7, 140.7, 141.7, 158.3, 167.9, 173.0.

Step 7: Preparation of tert-butyl 4′-[(3-butyl-4,5-dihydro-5-oxo-1,2,4-triazol-4-yl) methyl] [1,1′-biphenyl]-2-carboxylate

To 500 mg (1.18 mmol) of the semicarbazide from step 6 in 10 mL of methanol and 5 mL of toluene was added 126 mg (2.33 mmol) of sodium methoxide and the resulting mixture was stirred under vigorous reflux for about 48 h with a moisture trap (molecular sieve, 3A) attached between a condenser and the reaction vessel. The mixture was acidified with acetic acid and concentrated in vacuo. The residue was dissolved in chloroform and washed with water, saturated sodium bicarbonate and brine. The extract was dried (MgSO₄) and concentrated in vacuo. The residue was chromatographed over silica gel (eluted with ethyl acetate-hexane, 2:1) to give 480 mg (90%) of 3-butyl-1,2,4-triazolone as an oil: ¹H NMR (CDCl₃) δ 0.90 (t, J=7.2 Hz, 3H), 1.24 (s, 9H), 1.30-1.45 (m, 2H), 1.62 (quintet, J=7.8 Hz, 2H), 2.43 (t, JL= 7.5 Hz, 2H), 4.87 (s, 2H), 7.2-7.55 (m, 7H), 7.78 (dd, J=7.5, 1.2 Hz, 1H).

Step 8: Preparation of tert-butyl 4′-[(1,3-dibutyl-4,5-dihydro-5-oxo-1H-1,2.4-triazol-4-yl) methyl](1,1′-biphenyl]-2-carboxylate

To 226 mg (0.555 mmol) of the 3-butyl-1,2,4-triazolone from step 7 in 5 mL of N,N-dimethylformamide at about 5° C. was added 0.66 mL (0.66 mmol) of 1 M potassium tert-butoxide in tetrahydrofuran. The resulting solution was stirred at about 5° C. for 20 min, followed by addition of 200 μL (1.76 mmol) of 1-iodobutane. The mixture was stirred at room temperature for 30 min, quenched with acetic acid and concentrated in vacuo. The residue was chromatographed (eluted with ethyl acetate-hexane, 1:2) to give 210 mg (82%) of dibutyl-1,2,4-triazolone as an oil: ¹H NMR (CDCl₃) δ 0.8-1.0 (m, 6H), 1.24 (s, 9H), 1.24-1.45 (m, 4H), 1.50-1.68 (m, 2H), 1.68-1.80 (m, 2H), 2.41 (t, J=7.5 Hz, 2H), 3.80 (t, J=7.2 Hz, 2H), 4.86 (s, 2H), 7.20-7.34 [m (with s at 7.28), 5H], 7.35-7.55 (m, 2H), 7.78 (dd, J=7.5, 1.2 Hz, 1H); ¹³C NMR (CDCl₃) δ 4.2, 20.3, 22.8, 26.3, 28.1, 28.5, 31.4, 45.0, 45.6, 127.4, 127.8, 129.7, 130.2, 131.0, 131.2, 133.4, 135.5, 141.8, 142.2, 146.7, 154.6, 168.3; mass spectrum (FAB) m/e (relative intensity) 470(40), 414(100).

Step 9: Preparation of 4′-[(1 3-dibutyl-4,5-dihydro-5-oxo-1H-1,2,4-triazol-4-yl) methyl] [1,1′-biphenyl]-2-carboxylic acid

To 165 mg (0.356 mmol) of the tert-butyl ester from step 8 in 2 mL of chloroform was added 1 mL (13 mmol) of trifluoroacetic acid (TFA). The resulting solution was stirred at room temperature for about 15 h, and concentrated in vacuo. The residue was chromatographed over silica gel (ethyl acetate-methanol-acetic acid, 85:10:5) to give 152 mg (quantitative) of product compound of example 1 as a solid: ¹H NMR (CDCl₃) δ 0.75-1.0 (m, 6H), 1.2-1.4 (m, 4H), 1.52 (quintet, J=7.8 Hz, 2H), 1.69 (quintet, J =7.5 Hz, 2H), 2.37 (t, J=7.5 Hz, 2H), 3.78 (t, J= 6.9 Hz, 2H), 4.85 (s, 2H), 7.12 (d, J=7.8 Hz, 2H), 7.2-7.32 (m, 4H), 7.37 (t, J=7.2 Hz, 1H) , 7.50 (t, J =7.5 Hz, 1H), 7.88 (d, J=7.5 Hz, 1H); ¹³C NMR (CDCl₃) δ 14.11, 14.13, 20.2, 22.6, 26.1, 28.4, 31.2, 45.0, 45.7, 127.1, 127.8, 129.6, 130.7, 131.0, 131.4, 132.2, 135.0, 141.5, 142.9, 147.4, 154.5, 172.5; mass spectrum (FAB) m/e (relative intensity) 408(33), 390(20), 211(100).

### EXAMPLE 2

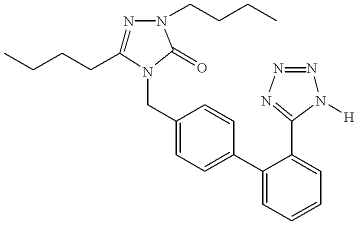

2,5-dibutyl-2,4-dihydro-4-[[2-(1H-tetrazol-5-yl)[1,1′-biphenyl]-4′-yl] methyl]-3H-1,2.4-triazol-3-one

Step 1: Preparation of N-ethoxycarbonyl thiovaleramide

To 95 mL of-2 M (0.190 mol) butylmagnesium chloride in tetrahydrofuran and 55 mL of anhydrous ether at about −60° C. (chloroform-dry ice) was added dropwise 24.4 g (0.162 mol) of ethoxycarbonyl isothiocyanate in 205 mL of anhydrous ether over a period of 2 h. The resulting mixture was stirred cold for about 3 h and allowed to slowly warm to room temperature. The solvent was decanted and the solid was washed with four 50 mL-portions of ether. The solid was partitioned between 200 mL of ether and 200 mL of saturated ammonium chloride. The aqueous layer was extracted with two 50 mL-portions of ether and the combined extracts were dried (MgSO₄) and concentrated in vacuo. The red oily residue was distilled under reduced pressure to give 18 g (58%) of N-ethoxycarbonyl thiovaleramide as a yellow oil: bp 86° C./ 1.5 torr; ¹H NMR (CDCl₃) δ 0.90 (t, J=7.5 Hz, 3H), 1.20-1.5 (m, 5H), 1.65 -1.8 (m, 2H), 3.20 (t, J= 7.5 Hz, 2H), 4.2 (q, J=7.2 Hz, 2H), 9.2 (broad s, 1H).

Step 2: Preparation of

5-butyl-2,4-dihydro-1,2,4-triazol-3-one

To 567 mg (3 mmol) of N-ethoxycarbonyl thiovaleramide in 6 mL of absolute ethanol at room temperature was added 190 mL (6 mmol) of 98% hydrazine in 1.5 mL of ethanol. The resulting solution was stirred at about 84° C. for 30 min, and concentrated in vacuo. The resulting solid was rinsed with ether-hexane, and collected by filtration to give 320 mg (76%) of 5-butyl-triazol-3-one as a white solid: ¹H NMR (CDCl₃) δ 0.85 (t, J=7.8 Hz, 3H), 1.2-1.4 (m, 2H), 1.55 (quintet, J=7.8 Hz, 2H), 2.0-3.0 [m (with t at 2.42, J=7.8 Hz), 3H], 9.6-11 (broad s, 1H).

Step 3: Preparation of N-triphenylmethyl-5-[2-(4′-bromomethylbiphen-2-yl]tetrazole.

A 542.5 g (2.4 mol) sample of methyl 2-(p-tolyl) benzoate (Chemo Dynamics Inc.) was dissolved in 5.5 L of ethanol and treated with 3 L (7.5 mol) of 2.5 N sodium hydroxide. The reaction was stirred overnight at ambient temperature and treated with an additional 480 mL (6.0 mol) of sodium hydroxide; stirring was continued for an additional 24 h and the ethanol removed in vacuo. The remaining solution was cooled in ice and acidified to pH 1 with hydrochloric acid which caused the product to precipitate; filtration and drying in vacuo gave 510 g (100%) of crude 2-(p-tolyl)benzoic acid: mp 145.0-147.5° C.; NMR (CDCl₃) δ 2.40 (s, 3H), 7.17-7.28 (m, 4H), 7.35-7.45 (m, 2H), 7.51-7.59 (m, 1H), 7.90-7.97 (m, 1H). The crude acid was suspended in 1 L of toluene and slowly treated with 400 g (3.15 mol) of oxalyl chloride under nitrogen. The resulting solution was allowed to stir at ambient temperature for 4.5 h and concentrated in vacuo to remove excess oxalyl chloride. The residue was redissolved in 2 L of toluene and treated with 92.8 g (5.46 mol) of anhydrous ammonia. The reaction was filtered and the filtrate concentrated in vacuo producing 424 g (84%) of crude 2-(p-tolyl)benzamide: mp 128-130° C.; NMR (CDCl ₃) 5 2.40 (s, 3H), 5.28 (br s, 1H), 5.77 (br s, 1H), 7.21-7.53 (m, 7H), 7.76-7.83 (m, 1H). The crude amide was treated with 1420 mL (19.5 mol) of thionyl chloride at reflux for 3.5 h. The reaction was filtered and thionyl chloride removed in vacuo. The residue was dissolved in 800 mL of toluene and reconcentrated in vacuo. On standing overnight, the residue crystallized. The crystals were collected and washed with hexane to give 296 g (64%) of 2-(p-tolyl) benzonitrile: mp 50.5-52.0 ° C.; NMR (CDCl₃) 5 2.42 (s, 3H), 7.22-7.34 (m, 2H), 7.37-7.52 (m, 3H), 7.58-7.66 (m, 1H), 7.72-7.78 (m, 1H). A 286 g (1.48 mol) sample of the crude nitrile was dissolved in 1630 mL of toluene and treated with 377 g (1.8 mol) of trimethyltin azide at reflux for 24 h. The reaction was cooled; filtration gave 600 g of crude N-trimethylstannyl-5-[2-(4′-methylbiphen-2-yl]tetrazole: mp 271-272° C. (dec.); NMR (DMSO-d₆) δ 0.36 (br t, J =34 Hz, 9H), 2.24 (s, 3H), 6.89-7.06 (m, 4H), 7.35-7.55 (m, 4H). The crude N-trimethylstannyl tetrazole was suspended in 4270 mL of toluene and 287 mL of anhydrous tetrahydrofuran (THF) and treated with 63.4 g (173 mol) of anhydrous hydrogen chloride at ambient temperature under nitrogen with stirring. The reaction was allowed to stand overnight and filtered; recrystallization from toluene gave 217 g (62%) of 5-[2-(4′ methylbiphen-2-yl)]tetrazole as a solid: mp 149-152° C.; NMR (DMSO-d₆) δ 2.28 (s, 3H), 6.94-7.02 (m, 2H), 7.08-7.15 (m, 2H), 7.50-7.59 (m, 2H), 7.62-7.72 (m, 2H). A 200 g (0.85 mol) sample of the tetrazole was suspended in 3.3 L of dichloromethane and treated with 262 g (0.91 mol) of triphenylmethyl chloride and 141 mL (1.0 mol) of anhydrous triethylamine. The reaction was stirred at reflux for 3 h under nitrogen, washed with water, dried (MgSO4), and concentrated in vacuo. Recrystallization gave 338 g (83%) of N-triphenylmethyl-5-[2-(4′-methylbiphen-2-yl)]tetrazole as a colorless solid: mp 170-173° C.; NMR (CDC13) δ 2.27 (s, 3H), 6.86-6.96 (m, 8H), 6.98-7.04 (m, 2H), 7.09-7.52 (m, 12H), 7.86-7.94 (m, 1H). The N-triphenylmethyl tetrazole was dissolved in 4260 mL of carbon tetrachloride and treated with 126.4 g (0.71 mol) of N-bromosuccinimide (NBS) and 11.9 g (49 mmol) of benzoyl peroxide at reflux for 3.5 h. The reaction was filtered and the solvent removed in vacuo. Recrystallization from toluene gave 277 g (59%) of N-triphenylmethyl-5-[2-(4′-bromomethylbiphen-2-yl)] tetrazole as a colorless solid: mp 140-142° C.; NMR (CDCl₃) δ 4.39 (s, 2H), 6.85-6.95 (m, 7H), 7.06-7.15 (m, 4H), 7.22-7.43 (m, 9H), 7.45-7.55 (m, 2H), 7.94-8.01 (m, 1H). NMR indicated that this material was only 85% pure; it contained 7% of corresponding dibromo- compound (δ 6.50) and 8% of starting material (δ 2.27); however, no further attempts at purification were made and this mixture was used in all subsequent alkylation reactions.

Step 4: Preparation of 5-butyl-2,4-dihydro-4-[[2-(1-triphenylmethyl-1H-tetrazol-5-yl) [1,1′-biphenyl]-4′-yl]methyl]-3H-1,2,4-triazol-3-one

To 247 mg (1.75 mmol) of the 5-butyl-triazol-3-one from step 2 in 17 mL of N,N-dimethylformamide was added dropwise 1.8 mL (1.8 mmol) of 1 M potassium tert-butoxide in tetrahydrofuran. The resulting light brown solution was stirred at room temperature for 10 min, followed by addition of 952 mg (1.7 mmol) of N-triphenylmethyl-5-[2-(4′-bromomethylbiphen-2-yl)] tetrazole from step 3 in two portions, 30 min apart. The resulting solution was stirred at room temperature for about 1 h, quenched with 160 mL of acetic acid and concentrated in vacuo. The residue was chromatographed (eluted with isopropanol-hexane, 1:7) to give 290 mg (27%) of N-substituted triazolone as a solid: ¹H NMR (CDCl₃) δ 0.85 (t, J=7.3 Hz, 3H), 1.28 (septet, J=7.5 Hz, 2H), 1.53 (quintet, J=7.7 Hz, 2H), 2.29 (t, J=7.3 Hz, 2H), 4.71 (s, 2H), 6.90 (d, J=7.2 Hz, 6H), 7.01 (d, J=8.3 Hz, 2H), 7.12 (d, J=8.2 Hz, 2H), 7.2-7.4 (m, 10H), 7.4-7.55 (m, 2H), 7.9-8.0 (m, 1H), 9.0 (s, 1H); mass spectrum (FAB) m/e (relative intensity) 624(22), 568(4), 388(30), 382(18), 339(100).

Step 5: Preparation of 2.5-dibutyl-2,4-dihydro-4-[[2-(1-triphenylmethyl-1H-tetrazol-5-yl) [1,1′-biphenyl]-4′-yl]methyl]-3H-1,2 4-triazol-3-one

To 240 mg (0.389 mmol) of the N-substituted triazolone from step 4 in 4.5 mL of N,N-dimethylformamide was added 460 μL (0.46 mmol) of 1 M potassium tert-butoxide in tetrahydrofuran. The resulting solution was stirred at room temperature for 10 min, followed by addition of 150 μL (1.32 mmol) of 1-iodobutane. The mixture was stirred at room temperature for about 1 h, quenched with acetic acid and concentrated in vacuo. The residue was filtered through a pad of silica gel (eluted with ethyl acetate-hexane, 1:3) to give 255 mg (97%) of N,N′-disubstituted triazolone as an oil: ¹H NMR (CDC13) δ 0.84 (t, J=7.3 Hz, 3H), 0.96 (t, J=7.4 Hz, 3H), 1.27 (septet, J=7.3 Hz, 2H), 1.38 (septet, J=7.7 Hz, 2H), 1.50 (quintet, J=7.9 Hz, 2H), 1.75 (quintet, J=7.5 Hz, 2H), 2.28 (t, J=8.0 Hz, 2H), 3.80 (t, J=7.3 Hz, 2H), 4.70 (s, 2H), 6.87-6.96 (m, 6H), 7.01 (d, J=8.3 Hz, 2H), 7.11 (d, J=8.3 Hz, 2H), 7.20-7.40 (m, 10H), 7.40-7.53 (m, 2H), 7.88-7.95 (m, 1H); mass spectrum (FAB) m/e (relative intensity) 680(20), 624(4), 444(25), 410(20), 395(100), 381(15).

Step 6; Preparation of 2.5-dibutyl-2.4-dihydro-4-[[2-(1H-tetrazol-5-yl) [1,1′-biphenyl]-4′-yl]methyl]-3H-1,2,4-triazol-3-one

A solution of 245 mg (0.364 mmol) of the N,N′-disubstituted triazolone from step 5 in 4 mL of acetic acid and 0.4 mL of water was stirred at room temperature for about 17 h and concentrated in vacuo. The oily residue was dissolved in saturated sodium bicarbonate and washed with three 5 mL-portions of ether-hexane. The aqueous layer was acidified to about pH 3 whith 3 N hydrochloric acid and extracted with chloroform and ethyl acetate. The combined extracts were dried (MgSO₄) and concentrated in vacuo to give 150 mg (quantitative) of the product compound of example 2 as a solid: ¹H NMR (CDCl₃) δ 0.85 (t, J= 7.1 Hz, 6H), 1.15-1.42 (m, 4H), 1.42-1.70 [m (with two quintet at 1.50 and 1.61, J=7.4 and 7.3 Hz), 4H], 2.34 (t, J=7.5 Hz, 2H), 3.64 (t, J=7.2 Hz, 2H) , 4.69 (s, 2H), 7.06 (q, J=8.1 Hz, 4H), 7.27 (broad s, 1H), 7.41 (d, J=7.5 Hz, 1H), 7.45-7.65 (m, 2H), 7.82 (d, J=7.2 Hz, 1H); mass spectrum (FAB) m/e (relative intensity) 432(40), 207(100), 178(25); HRMS. calcd for M+H: 432.2512. Found: 432.2510.

### BIOLOGICAL EVALUATION

Assay A: Anaiotensin II Binding Activity

Compounds of the invention were tested for ability to bind to the smooth muscle angiotensin II receptor using a rat uterine membrane preparation. Angiotensin II (AII) was purchased from Peninsula Labs. ¹²⁵I-angiotensin II (specific activity of 2200 Ci/mmol) was purchased from Du Pont-New England Nuclear. Other chemicals were obtained from Sigma Chemical Co. This assay was carried out according to the method of Douglas et al [Endocrinlogoy, 106, 120-124 (1980)]. Rat uterine membranes were prepared from fresh tissue. All procedures were carried out at 4° C. Uteri were stripped of fat and homogenized in phosphate-buffered saline at pH 7.4 containing 5 mM EDTA. The homogenate was centrifuged at 1500×g for 20 min., and the supernatant was recentrifuged at 100,000×g for 60 min. The pellet was resuspended in buffer consisting of 2 mM EDTA and 50 mM Tris-HCl (pH 7.5) to a final protein concentration of 4 mg/ml. Assay tubes were charged with 0.25 ml of a solution containing 5 mM MgCl₂, 2 mM EDTA, 0.5% bovine serum albumin, 50 mM Tris-HCl, pH 7.5 and ¹²⁵I-AII (approximately 10⁵ cpm) in the absence or in the presence of unlabelled ligand. The reaction was initiated by the addition of membrane protein and the mixture was incubated at 25° C. for 60 min. The incubation was terminated with ice-cold 50 mM Tris-HCl (pH 7.5) and the mixture was filtered to separate membrane-bound labelled peptide from the free ligand. The incubation tube and filter were washed with ice-cold buffer. Filters were assayed for radioactivity in a Micromedic gamma counter. Nonspecific binding was defined as binding in the presence of 10 μM of unlabelled AII. Specific binding was calculated as total binding minus nonspecific binding. The receptor binding affinity of an AII antagonist compound was indicated by the concentration (IC₅₀) of the tested AII antagonist which gives 50% displacement of the total specifically bound ¹²⁵I-AII from the high affinity AII receptor. Binding data were analyzed by a nonlinear least-squares curve fitting program. Results are reported in Table I.

Assay B: In Vitro Vascular Smooth Muscle-Resionse for AII

The compounds of the invention were tested for antagonist activity in rabbit aortic rings. Male New Zealand white rabbits (2-2.5 kg) were sacrificed using an overdose of pentobarbital and exsanguinated via the carotid arteries. The thoracic aorta was removed, cleaned of adherent fat and connective tissue and then cut into 3-mm ring segments. The endothelium was removed from the rings by gently sliding a rolled-up piece of filter paper into the vessel lumen. The rings were then mounted in a water-jacketed tissue bath, maintained at 37° C., between moveable and fixed ends of a stainless steel wire with the moveable end attached to an FT03 Grass transducer coupled to a Model 7D Grass Polygraph for recording isometric force responses. The bath was filled with 20 ml of oxygenated (95% oxygen/5% carbon dioxide) Krebs solution of the following composition (nM) : 130 NaCl, 15 NaHCO₃, 15 KCl, 1.2 NaH₂PO₄, 1.2 MgSO₄, 2.5 CaCl₂, and 11.4 glucose. The preparations were equilibrated for one hour before approximately one gram of passive tension was placed on the rings. Angiotensin II concentration-response curves were then recorded (3×10⁻¹⁰ to 1×10⁻⁵ M). Each concentration of AII was allowed to elicit its maximal contraction, and then AII was washed out repeatedly for 30 minutes before rechallenging with a higher concentration of AII. Aorta rings were exposed to the test antagonist at 10⁻⁵ M for 5 minutes before challenging with AII. Adjacent segments of the same aorta ring were used for all concentration-response curves in the presence or absence of the test antagonist. The effectiveness of the test compound was expressed in terms of pA₂ values and were calculated according to H. O. Schild [Br. J. Pharmacol. Chemother., 2,189-206 (1947)]. The pA₂ value is the concentration of the antagonist which increases the EC₅₀ value for AII by a factor of two. Each test antagonist was evaluated in aorta rings from two rabbits. Results are reported in Table I.

Assay C: In Vivo Intraduodenal Pressor Assay Response for AII Antagonists

Male Sprague-Dawley rats weighing 225-300 grams were anesthetized with Inactin (100 mg/kg, i.p.) and catheters were implanted into the trachea, femoral artery, femoral vein and duodenum. Arterial pressure was recorded from the femoral artery catheter on a Gould chart recorder (Gould, Cleveland, OH). The femoral vein catheter was used for injections of angiotensin II, mecamylamine and atropine. The tracheal catheter allows for airway patency, and the duodenal catheter was used for intraduodenal (i.d.) administration of test compounds. After surgery, the rats were allowed to equilibrate for 30 minutes. Mecamylamine (3 mg/kg, 0.3 ml/kg) and atropine (400 ug/kg, 0.3 ml/kg) were then given i.v. to produce ganglion blockade. These compounds were administered every 90 minutes throughout the test procedure. Angiotensin II was given in bolus does i.v. (30 ng/kg in saline with 0.5% bovine serum albumin, 0.1 ml/kg) every 10 minutes three times or until the increase in arterial pressure produced was within 3 mmHg for two consecutive AII injections. The last two AII injections were averaged and were taken as the control AII pressor response. Ten minutes after the final control AII injection, the test compound (dissolved in sodium bicarbonate) was administered i.d. at a dose of 30 mg/kg in a volume of 0.2 ml. Angiotensin II injections were then given 5, 10, 20, 30, 45, 60, 75, 90, and 120 minutes after administration of the test compound and response of arterial pressure was monitored. The response to AII was calculated as percent of the control response and then the percent inhibition is calculated as 100 minus the percent control response. Duration of action of a test compound was defined as the time from peak percent inhibition to 50% of peak. One compound at one dose was tested in each rat. Each test compound was tested in two rats and the values for the two rats were averaged. Results are reported in Table I.

TABLE I
In Vivo and In Vitro Angiotensin II
Activity of Compounds of the Invention

| | ¹Assay A | | ³Assay C | |
|---|---|---|---|---|
| Test Compound | IC₅₀ | ²Assay B | Inhibition | Duration |
| Example # | (nM) | PA₂ | (%) | (min.) |
| 1 | 101 | 7.3/6.7 | 93 | 60-100 |
| 2 | 4.9 | 9.0/9.4 | 100 | >200 |

NT = NOT TESTED
¹Assay A: Angiotensin II Binding Activity
²Assay B: In Vitro Vascular Smooth Muscle Response
³Assay C: In Vivo Intraduodenal Pressor Response

Also embraced within this invention is a class of pharmaceutical compositions comprising one or more compounds of Formula I in association with one or more non-toxic, pharmaceutically acceptable carriers and/or diluents and/or adjuvants (collectively referred to herein as “carrier” materials) and, if desired, other active ingredients. The compounds of the present invention may be administered by any suitable route, preferably in the form of a pharmaceutical composition adapted to such a route, and in a dose effective for the treatment intended. Therapeutically effective doses of the compounds of the present invention required to prevent or arrest the progress of the medical condition are readily ascertained by one of ordinary skill in the art. The compounds and composition may, for example, be administered intra-vascularly, intraperitoneally, subcutaneously, intra-muscularly or topically.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. These may with advantage contain an amount of active ingredient from about 1 to 250 mg, preferably from about 25 to 150 mg. A suitable daily dose for a mammal may vary widely depending on the condition of the patient and other factors. However, a dose of from about 0.1 to 3000 mg/kg body weight, particularly from about 1 to 100 mg/kg body weight, may be appropriate.

The active ingredient may also be administered by injection as a composition wherein, for example, saline, dextrose or water may be used as a suitable carrier. A suitable daily dose is from about 0.1 to 100 mg/kg body weight injected per day in multiple doses depending on the disease being treated. A preferred daily dose would be from about 1 to 30 mg/kg body weight. Compounds indicated for prophylactic therapy will preferably be administered in a daily dose generally in a range from about 0.1 mg to about 100 mg per kilogram of body weight per day. A more preferred dosage will be a range from about 1 mg to about 100 mg per kilogram of body weight. Most preferred is a dosage in a range from about 1 to about 50 mg per kilogram of body weight per day. A suitable dose can be administered, in multiple sub-doses per day. These sub-doses may be administered in unit dosage forms. Typically, a dose or sub-dose may contain from about 1 mg to about 100 mg of active compound per unit dosage form. A more preferred dosage will contain from about 2 mg to about 50 mg of active compound per unit dosage form. Most preferred is a dosage form containing from about 3 mg to about 25 mg of active compound per unit dose.

The dosage regimen for treating a disease condition with the compounds and/or compositions of this invention is selected in accordance with a variety of factors, including the type, age, weight, sex and medical condition of the patient, the severity of the disease, the route of administration, and the particular compound employed, and thus may vary widely.

For therapeutic purposes, the compounds of this invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Although this invention has been described with respect to specific embodiments, the details of these embodiments are not to be construed as limitations.

What is claimed is:

1. A compound of Formula I: 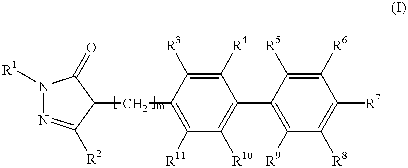
wherein m is a number selected from one to four, inclusive;
wherein R¹ is selected from alkyl, hydroxyalkyl, haloalkyl, cycloalkyl, cycloalkylalkyl, formyl, aralkyl, aryl, aroyl, alkoxyalkyl, alkylcarbonyl, alkylcarbonylalkyl, alkoxycarbonyl, alkenyl, cycloalkenyl, aralkoxycarbonyl, alkynyl, cyano, carboxyl, mercaptocarbonyl, mercaptothiocarbonyl, alkylthiocarbonyl, alkylthiothiocarbonyl, arylthiocarbonyl, arylthiothiocarbonyl, aralkylthiocarbonyl, alkylthiocarbonyl, alkylsulfinyl, alkylsulfonyl, aralkylsulfinyl, aralkylsulfonyl, arylsulfinyl, arylsulfonyl, heteroaryl having one or more ring atoms selected from oxygen, sulfur and nitrogen atoms, and amido radicals of the formula 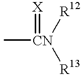
wherein X is oxygen atom or sulfur atom; wherein each of R¹² and R¹³ is independently selected from hydrido, alkyl, cycloalkyl, cyano, hydroxyalkyl, cycloalkylalkyl, alkoxyalkyl, aralkyl and aryl, and wherein R¹² and R¹³ taken together may form a heterocyclic group having five to seven ring members including the nitrogen atom of said amino or amido radical and which heterocyclic group may further contain one or more hetero atoms as ring members selected from oxygen, nitrogen and sulfur atoms and which heterocyclic group may be saturated or partially unsaturated; wherein each of R¹² and R¹³ taken together may form an aromatic heterocyclic group having five ring members including the nitrogen atom of said amino or amido radical and which aromatic heterocyclic group may further contain one or more hetero atoms as ring atoms selected from oxygen, nitrogen and sulfur atoms;
wherein each of R² through R¹¹ is independently selected from hydrido, alkyl, hydroxyalkyl, halo, haloalkyl, cycloalkyl, cycloalkylalkyl, formyl, alkoxy, aralkyl, aryl, aroyl, aryloxy, aralkoxy, aralkoxycarbonyl, alkoxyalkyl, alkylcarbonyl, alkylcarbonylalkyl, alkoxycarbonyl, alkenyl, cycloalkenyl, alkynyl, cyano, nitro, carboxyl, alkylcarbonyloxy, mercaptocarbonyl, mercaptothiocarbonyl, alkoxycarbonyloxy, alkylthio, alkylthiocarbonyl, alkylcarbonylthio, alkylthiocarbonyloxy, alkylthiocarbonylthio, alkylthiothiocarbonyl, alkylthiothiocarbonylthio, arylthio, arylthiocarbonyl, arylcarbonylthio, arylthiocarbonyloxy, arylthiocarbonylthio, arylthiothiocarbonyl, arylthiothiocarbonylthio, aralkylthio, aralkylthiocarbonyl, aralkylcarbonylthio, aralkylthiocarbonyloxy, aralkylthiocarbonylthio, alkylthiocarbonyl, aralkylthiocarbonylthio, mercapto, alkylsulfinyl, alkylsulfonyl, aralkylsulfinyl, aralkylsulfonyl, arylsulfinyl, arylsulfonyl, heteroaryl having one or more ring atoms selected from oxygen, sulfur and nitrogen atoms, and amino and amido radicals of the formula 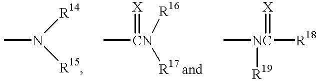
wherein X is oxygen atom or sulfur atom; wherein each of R¹⁴, R¹⁵, R¹⁶, R¹⁷, R¹⁸ and R¹⁹ is independently selected from hydrido, alkyl, cycloalkyl, cyano, hydroxyalkyl, cycloalkylalkyl, alkoxyalkyl, aralkyl and aryl, and wherein R¹⁴ and R¹⁵ taken together, R¹⁶ and R¹⁷ taken together and R¹⁸ and R¹⁹ taken together may form a heterocyclic group having five to seven ring members including the nitrogen atom of said amino or amido radical and which heterocyclic group may further contain one or more hetero atoms as ring members selected from oxygen, nitrogen and sulfur atoms and which heterocyclic group may be saturated or partially unsaturated; wherein each of R¹⁴ and R¹⁵ taken together and each of R¹⁶ and R¹⁷ taken together may form an aromatic heterocyclic group having five ring members including the nitrogen atom of said amino or amido radical and which aromatic heterocyclic group may further contain one or more hetero atoms as ring atoms selected from oxygen, nitrogen and sulfur atoms;
and wherein each of R³ through R¹¹ may be further independently selected from hydroxy and acidic moieties of the formula
−YₙA
wherein n is a number selected from zero through three, inclusive, and wherein A is an acidic group selected to contain at least one acidic hydrogen atom, and the amide, ester and salt derivatives of said acidic moieties; wherein Y is a spacer group independently selected from one or more of alkyl, cycloalkyl, cycloalkylalkyl, alkenyl, alkynyl, aryl, aralkyl and heteroaryl having one or more ring atoms selected from oxygen, sulfur and nitrogen atoms; and wherein any of the foregoing R¹ through R¹⁹, Y and A groups having a substitutable position may be substituted by one or more groups selected from hydroxy, alkyl, alkenyl, alkynyl, aralkyl, hydroxyalkyl, haloalkyl, halo, oxo, alkoxy, aryloxy, aralkoxy, aralkylthio, alkoxyalkyl, cycloalkyl, cycloalkylalkyl, aryl, aroyl, cycloalkenyl, cyano, cyanoamino, nitro, alkylcarbonyloxy, alkoxycarbonyloxy, alkylcarbonyl, alkoxycarbonyl, aralkoxycarbonyl, carboxyl, mercapto, mercaptocarbonyl, alkylthio, arylthio, alkylthiocarbonyl, alkylsulfinyl, alkylsulfonyl, haloalkylsulfonyl, aralkylsulfinyl, aralkylsulfonyl, arylsulfinyl, arylsulfonyl, heteroaryl having one or more ring atoms selected from oxygen, sulfur and nitrogen atoms, and amino and amido radicals of the formula 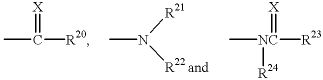
wherein X is oxygen atom or sulfur atom; wherein R²⁰ is selected from hydrido, alkyl, cycloalkyl, cycloalkylalkyl, aralkyl, aryl, DR²⁵ and 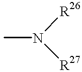
wherein D is selected from oxygen atom and sulfur atom and R²⁵ is selected from hydrido, alkyl, cycloalkyl, cycloalkylalkyl, aralkyl and aryl; wherein each of R²¹, R²², R²³, R²⁴, R²⁶ and R²⁷ is independently selected from hydrido, alkyl, cycloalkyl, cyano, hydroxyalkyl, haloalkyl, cycloalkylalkyl, alkoxyalkyl, alkylcarbonyl, alkoxycarbonyl, carboxyl, alkylsulfinyl, alkylsulfonyl, arylsulfinyl, arylsulfonyl, haloalkylsulfinyl, haloalkylsulfonyl, aralkyl and aryl, and wherein each of R²¹, R²², R²³, R²⁴, R²⁶ and R²⁷ is further independently selected from amino and amido radicals of the formula 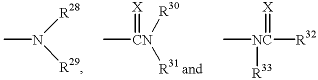
wherein each of R²⁸, R²⁹, R³⁰, R³¹ R³² and R³³ is independently selected from hydrido, alkyl, cycloalkyl, cyano, hydroxyalkyl, cycloalkylalkyl, alkoxyalkyl, haloalkylsulfinyl, haloalkylsulfonyl, aralkyl and aryl, and wherein each of R²¹ and R²² taken together and each of R²³ and R²⁴ taken together may form a heterocyclic group having five to seven ring members including the nitrogen atom of said amino or amido radical, which heterocyclic group may further contain one or more hetero atoms as ring members selected from oxygen, nitrogen and sulfur atoms and which heterocyclic group may be saturated or partially unsaturated; wherein each of R²¹ and R²² taken together and each of R²⁶ and R²⁷ taken together may form an aromatic heterocyclic group having five ring members including the nitrogen atom of said amino or amido radical and which aromatic heterocyclic group may further contain one or more hetero atoms as ring atoms selected from oxygen, nitrogen and sulfur atoms; or a tautomer thereof or a pharmaceutically-acceptable salt thereof.

2. Compound of claim 1 wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkoxycarbonyl, alkenyl, cycloalkenyl, alkynyl, cycloalkynyl, carboxyl, mercaptocarbonyl, mercaptothiocarbonyl, alkylthiocarbonyl, arylthiocarbonyl, arylthiothiocarbonyl, aralkylthiocarbonyl, alkylsulfonyl, aralkylsulfonyl, arylsulfonyl, heteroaryl having one or more ring atoms selected from oxygen, sulfur and nitrogen atoms, and amido radicals of the formula 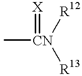
wherein X is oxygen atom or sulfur atom; wherein each of R¹² and R¹³ is independently selected from hydrido, alkyl, cycloalkyl, cyano, hydroxyalkyl, cycloalkylalkyl, alkoxyalkyl, aralkyl and aryl;
wherein R² is selected from hydrido, alkyl, hydroxyalkyl, halo, haloalkyl, cycloalkyl, cycloalkylalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cycloalkynyl, cyano, carboxyl, alkylcarbonyloxy, alkylthio, alkylthiocarbonyl, alkylcarbonylthio, alkylthiocarbonyloxy, alkylthiocarbonylthio, alkylthiothiocarbonyl, alkylthiothiocarbonylthio, arylthio, arylthiocarbonyl, arylcarbonylthio, arylthiocarbonyloxy, arylthiocarbonylthio, arylthiothiocarbonyl, arylthiothiocarbonylthio, aralkylthio, aralkylthiocarbonyl, arylkylcarbonylthio, aralkylthiocarbonyloxy, aralkylthiocarbonylthio, aralkylthiocarbonyl, aralkylthiocarbonylthio, mercapto, alkylsulfonyl, aralkylsulfonyl, arylsulfonyl and heteroaryl having one or more ring atoms selected from oxygen, sulfur and nitrogen atoms;
wherein each of R³ through R¹¹ is independently selected from hydrido, hydroxy, alkyl, hydroxyalkyl, halo, haloalkyl, cycloalkyl, cycloalkylalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cycloalkynyl, cyano, nitro, carboxyl, alkylcarbonyloxy, mercaptocarbonyl, mercaptothiocarbonyl, alkoxycarbonyloxy, alkylthio, alkylthiocarbonyl, alkylcarbonylthio, alkylthiocarbonyloxy, alkylthiocarbonylthio, alkylthiothiocarbonyl, arylthio, arylthiocarbonyl, arylcarbonylthio, arylthiocarbonyloxy, arylthiothiocarbonyl, aralkylthio, aralkylthiocarbonyl, aralkylcarbonylthio, aralkylthiocarbonyloxy, aralkylthiocarbonylthio, aralkylthiocarbonyl, aralkylthiocarbonylthio, mercapto, alkylsulfonyl, aralkylsulfonyl and arylsulfonyl, and amino and amido radicals of the formula 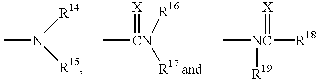
wherein X is oxygen atom or sulfur atom; wherein each of R¹⁴, R¹⁵, R¹⁶, R¹⁷, R¹⁸ and R¹⁹ is independently selected from hydrido, alkyl, cycloalkyl, cyano, hydroxyalkyl, cycloalkylalkyl, alkoxyalkyl, aralkyl and aryl;
and wherein each of R³ through R¹¹ may be further independently selected from acidic moieties of the formula
−YₙA
wherein n is a number selected from zero through three, inclusive; wherein A is an acidic group selected from acids containing one or more atoms selected from oxygen, sulfur, phosphorus and nitrogen atoms, and wherein said acidic group is selected to contain at least one acidic hydrogen atom, and the amide, ester and salt derivatives of said acidic moieties; wherein Y is a spacer group independently selected from one or more of alkyl, cycloalkyl, cycloalkylalkyl, alkenyl, alkynyl, aryl, aralkyl and heteroaryl having one or more ring atoms selected from oxygen, sulfur and nitrogen atoms;
and wherein any of the foregoing R¹ through R¹⁹, Y and A groups having a substitutable position may be substituted by one or more groups selected from hydroxy, alkyl, alkenyl, aralkyl, hydroxyalkyl, halo, haloalkyl, oxo, alkoxy, aryloxy, aralkoxy, alkoxyalkyl, alkylcarbonyl, alkoxycarbonyl, carboxyl, cyano, nitro, alkylsulfonyl, haloalkylsulfonyl, aryl, aralkyl, mercaptocarbonyl, alkylthio and alkylthiocarbonyl, and amino and amido radicals of the formula 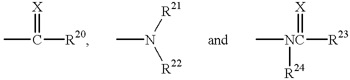
wherein X is oxygen atom or sulfur atom; wherein R¹⁹ is selected from hydrido, alkyl, cycloalkyl, cycloalkylalkyl, aralkyl, aryl, and DR²³ and 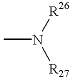
wherein D is selected from oxygen atom and sulfur atom, and R²⁵ is selected from hydrido, alkyl, cycloalkyl, cycloalkylalkyl, aralkyl and aryl; wherein each of R²¹, R²², R²³, R²⁴, R²⁶ and R²⁷ is independently selected from hydrido, alkyl, cycloalkyl, cyano, hydroxyalkyl, haloalkyl, cycloalkylalkyl, alkoxyalkyl, alkanoyl, alkoxycarbonyl, carboxyl, haloalkylsulfinyl, haloalkylsulfonyl, aralkyl and aryl;
or a tautomer thereof or a pharmaceutically-acceptable salt thereof.

3. Compound of claim 2 wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkoxycarbonyl, alkenyl, cycloalkenyl, alkynyl, cycloalkynyl, carboxyl, alkylsulfonyl, aralkylsulfonyl and arylsulfonyl, and amido radicals of the formula 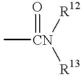
wherein each of R¹² and R¹³ is independently selected from hydrido, alkyl, cycloalkyl, cyano, hydroxyalkyl, cycloalkylalkyl, alkoxyalkyl, aralkyl and aryl;
wherein R² is selected from hydrido, alkyl, hydroxyalkyl, halo, haloalkyl, cycloalkyl, cycloalkylalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cycloalkynyl, carboxyl, alkylcarbonyloxy, alkylthio, arylthio, aralkylthio, aralkylthiocarbonylthio, alkylsulfonyl, aralkylsulfonyl and arylsulfonyl;
wherein each of R³ through R¹¹ is independently selected from hydrido, hydroxy, alkyl, hydroxyalkyl, halo, haloalkyl, cycloalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cyano, nitro, carboxyl, alkylcarbonyloxy, mercaptocarbonyl, alkoxycarbonyloxy, alkylthio, arylthio, aralkylthio, mercapto, alkylsulfonyl, aralkylsulfonyl and arylsulfonyl, and amino and amido radicals of the formula 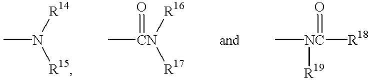
wherein each of R¹⁴, R¹⁵, R¹⁶, R¹⁷, R¹⁸ and R¹⁹ is independently selected from hydrido, alkyl, cycloalkyl, cyano, amino, monoalkylamino, dialkylamino, hydroxyalkyl, cycloalkylalkyl, alkoxyalkyl, aralkyl and aryl;
and wherein each of R³ through R¹¹ may be an acidic moiety further independently selected from acidic moieties of the formula
−YₙA
wherein n is a number selected from zero through three, inclusive;
wherein A is selected from carboxylic acid and bioisosteres of carboxylic acid selected from 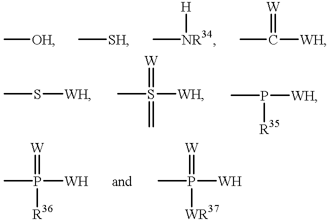
wherein each W is independently selected from oxygen atom, sulfur atom and NR³⁸; wherein each of R³⁴, R³⁵, R³⁶, R³⁷ and R³⁸ is independently selected from hydrido, alkyl, haloalkyl, haloalkylsulfonyl, haloalkylcarbonyl, cycloalkyl, cycloalkylalkyl, aryl and aralkyl; wherein each of R³⁴, R³⁵, R³⁶ and R³⁷ may be further independently selected from amino radical of the formula 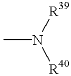
wherein each of R³⁹ and R⁴⁰ is independently selected from hydrido, alkyl, cycloalkyl, hydroxyalkyl, haloalkyl, cycloalkylalkyl, alkoxyalkyl, aralkyl and aryl, and wherein R³⁹ and R⁴⁰ taken together may form a heterocyclic group having five to seven ring members including the nitrogen atom of said amino radical, which heterocyclic group may further contain one or more hetero atoms as ring members selected from oxygen, nitrogen and sulfur atoms and which heterocyclic group may be saturated or partially unsaturated; wherein R³⁹ and R⁴⁰ taken together may form an aromatic heterocyclic group having five ring members including the nitrogen atom of said amino radical and which aromatic heterocyclic group may further contain one or more hetero atoms as ring atoms selected from oxygen, nitrogen and sulfur atoms; wherein each of R³⁵ and R³⁶ may be further independently selected from hydroxy, alkoxy, alkylthio, aryloxy, arylthio, aralkylthio and aralkoxy; and the amide, ester and salt derivatives of said acidic groups;
wherein said bioisostere of carboxylic acid may be further selected from heterocyclic acidic groups consisting of heterocyclic rings of four to about nine ring members, which heterocyclic ring contains at least one hetero atom selected from oxygen, sulfur and nitrogen atoms, which heterocyclic ring may be saturated, fully unsaturated or partially unsaturated, and which heterocyclic ring may be attached at a single position selected from R³ through R¹¹ or may be attached at any two adjacent positions selected from R³ through R¹¹ so as to form a fused-ring system with one of the phenyl rings of Formula I; and the amide, ester and salt derivatives of said heterocyclic acidic groups;
wherein Y is a spacer group independently selected from one or more of alkyl, cycloalkyl, cycloalkylalkyl, alkenyl, aryl and aralkyl;
and wherein any of the foregoing R¹ through R¹⁹ and R³⁴ through R⁴⁰, Y and A groups having a substitutable position may be substituted by one or more groups selected from hydroxy, alkyl, alkenyl, aralkyl, hydroxyalkyl, halo, oxo, haloalkyl, alkoxy, aryloxy, aralkoxy, alkoxyalkyl, alkylcarbonyl, alkoxycarbonyl, carboxyl, cyano, nitro, alkylsulfonyl, haloalkylsulfonyl, aryl, aralkyl, mercaptocarbonyl, alkylthio and alkylthiocarbonyl, and amino and amido radicals of the formula 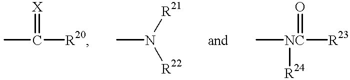
wherein X is selected from oxygen atom and sulfur atom; wherein R²⁰ is selected from hydrido, alkyl, cycloalkyl, cycloalkylalkyl, aralkyl, aryl and DR²⁵ and 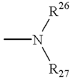
wherein D is selected from oxygen atom and sulfur atom, wherein R²⁵ is selected from hydrido, alkyl, cycloalkyl, cycloalkylalkyl, aralkyl and aryl; wherein each of R²¹, R²², R²³, R²⁴, R²⁶ and R²⁷ is independently selected from hydrido, alkyl, cycloalkyl, cyano, hydroxyalkyl, haloalkyl, cycloalkylalkyl, alkoxyalkyl, alkanoyl, alkoxycarbonyl, carboxyl, haloalkylsulfinyl, haloalkylsulfonyl, aralkyl and aryl; or a tautomer thereof or a pharmaceutically-acceptable salt thereof.

4. Compound of claim 3 wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkenyl, cycloalkenyl, alkynyl, mercaptocarbonyl, alkylsulfonyl,. aralkylsulfonyl, arylsulfonyl and amido radicals of the formula 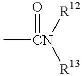
wherein each of R¹² and R¹³ is independently selected from hydrido, alkyl, cycloalkyl, cyano, amino, monoalkylamino, dialkylamino, hydroxyalkyl, cycloalkylalkyl, alkoxyalkyl, aralkyl and aryl;
wherein each of R² is selected from hydrido, alkyl, hydroxyalkyl, halo, halooalkyl, cycloalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, carboxyl, alkylcarbonyloxy, alkylthio, arylthio, aralkylthio, alkylsulfonyl, aralkylsulfonyl and arylsulfonyl;
wherein each of R³ through R¹¹ is independently selected from hydrido, hydroxy, alkyl, hydroxyalkyl, halo, haloalkyl, cycloalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cyano, nitro, carboxyl, alkylthio, aralkylthio and mercapto;
and wherein each of R³ through R¹¹ may be an acidic moiety further independently selected from acidic moieties of the formula
−YₙA
wherein n is a number selected from zero through three, inclusive; wherein A is selected from carboxylic acid and bioisosteres of carboxylic acid selected from 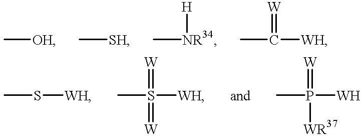
wherein each W is independently selected from oxygen atom, sulfur atom and NR³⁸; wherein each of R³⁴, R³⁷ and R³⁸ is independently selected from hydrido, alkyl, haloalkyl, haloalkylsulfonyl, haloalkylcarbonyl, cycloalkyl, cycloalkylalkyl, aryl and aralkyl; wherein each of R³⁴ and R³⁷ may be further independently selected from amino radical of the formula 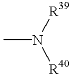
wherein each of R³⁹ and R⁴⁰ is independently selected from hydrido, alkyl, cycloalkyl, hydroxyalkyl, haloalkyl, cycloalkylalkyl, alkoxyalkyl, aralkyl and aryl, and wherein R³⁹ and R⁴⁰ taken together may form a heterocyclic group having five to seven ring members including the nitrogen atom of said amino radical, which heterocyclic group may further contain one or more hetero atoms as ring members selected from oxygen, nitrogen and sulfur atoms, and which heterocyclic group may be saturated or partially unsaturated; wherein R³⁹ and R⁴⁰ taken together may form an aromatic heterocyclic group having five ring members including the nitrogen atom of said amino radical and which aromatic heterocyclic group may further contain one or more hetero atoms as ring atoms selected from oxygen, nitrogen and sulfur atoms; and the amide, ester and salt derivatives of said acidic groups; wherein said bioisostere of carboxylic acid may be further selected from heterocyclic acidic groups consisting of heterocyclic rings of four to about nine ring members, which ring contains at least one hetero atom, selected from oxygen, sulfur and nitrogen atoms, which heterocyclic ring may be saturated, fully unsaturated or partially unsaturated, and which heterocyclic ring may be attached at a single position selected from R³ through R¹¹ or may be attached at any two adjacent positions selected from R³ through R¹¹ so as to form a fused-ring system with one of the phenyl rings of Formula I; and the amide, ester and salt derivatives of said heterocyclic acidic groups;
wherein Y is a spacer group independently selected from one or more of alkyl, cycloalkyl, cycloalkylalkyl, alkenyl, aryl and aralkyl;
wherein each of R¹ through R¹⁹, R³⁴ and R³⁷ through R⁴⁰, Y and A independently may be substituted at any substitutable position with one or more groups selected from alkyl, hydroxy, halo, oxo, haloalkyl, alkoxycarbonyl, cyano, nitro, alkylsulfonyl, haloalkylsulfonyl, aryl, aralkyl, alkoxy, aryloxy and aralkoxy;
or a tautomer thereof or a pharmaceutically-acceptable salt thereof.

5. Compound of claim 4 wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkenyl, alkynyl, alkylsulfonyl, aralkylsulfonyl, arylsulfonyl and amido radicals of the formula 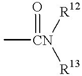
wherein each of R¹² and R¹³ is independently selected from hydrido, alkyl, cycloalkyl, cyano, amino, hydroxyalkyl, alkoxyalkyl, phenalkyl and phenyl;
wherein R² is selected from hydrido, alkyl, hydroxyalkyl, halo, haloalkyl, cycloalkyl, alkoxy, aralkyl, aryl, aryloxy, aralkoxy, alkoxyalkyl, alkylcarbonyl, alkoxycarbonyl, alkenyl, cycloalkenyl, alkynyl, alkylthio, arylthio, aralkylthio and arylsulfonyl,
wherein each of R³ through R¹¹ is independently selected from hydrido, hydroxy, alkyl, hydroxyalkyl, halo, haloalkyl, cycloalkyl, alkoxy, phenalkyl, phenyl, benzoyl, phenoxy, phenalkyloxy, alkoxyalkyl, alkylcarbonyl, alkoxycarbonyl, alkenyl, cyano, nitro, carboxyl, alkylthio and mercapto;
and wherein each of R³ through R¹¹ may be an acidic moiety further independently selected from acidic moieties of the formula
−YₙA
wherein n is a number selected from zero through two, inclusive; wherein A is selected from carboxylic acid and bioisosteres of carboxylic acid selected from 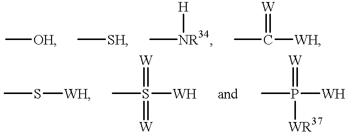
wherein each W is independently selected from oxygen atom, sulfur atom and NR^{38;} wherein each of R³⁴, R³ and R³⁸ is independently selected from hydrido, alkyl, haloalkyl, haloalkylsulfonyl, haloalkylcarbonyl, cycloalkyl, phenyl and benzyl; wherein each of R³⁴ and R³⁷ may be further independently selected from amino radical of the formula 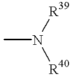
wherein each of R³⁹ and R⁴⁰ is independently selected from hydrido, alkyl, cycloalkyl, hydroxyalkyl, haloalkyl, alkoxyalkyl, benzyl and phenyl; and the amide, ester and salt derivatives of said acidic groups;
wherein said bioisostere of carboxylic acid may be further selected from heterocyclic acidic groups consisting of heterocyclic rings of four to about nine ring members, which ring contains at least one hetero atom, selected from oxygen, sulfur and nitrogen atoms, which heterocyclic ring may be saturated, fully unsaturated or partially unsaturated, and which heterocyclic ring may be attached at a single position selected from R³ through R¹¹ or may be attached at any two adjacent positions selected from R³ through R¹¹ so as to form a fused-ring system with one of the phenyl rings of Formula I; and the amide, ester and salt derivatives of said heterocyclic acidic groups;
wherein Y is a spacer group independently selected from one or more of alkyl, cycloalkyl, cycloalkylalkyl, alkenyl, phenyl, phenalkyl and aralkyl;
wherein each of R¹ through R¹⁹, R³⁴ and R³⁷ through R⁴⁰, Y and A and independently may be substituted at any substitutable position with one or more groups selected from alkyl, cycloalkyl, cycloalkylalkyl, hydroxy, halo, oxo, haloalkyl, alkoxycarbonyl, cyano, nitro, alkylsulfonyl, haloalkylsulfonyl, aryl, aralkyl, alkoxy, aryloxy and aralkoxy;
or a tautomer thereof or a pharmaceutically-acceptable salt thereof.

6. Compound of claim 5 wherein m is one; wherein R¹ is selected from alkyl, hydroxyalkyl, haloalkyl, cycloalkyl, cycloalkylalkyl, aralkyl, aryl, benzoyl, alkoxyalkyl, alkylcarbonyl, alkoxycarbonyl, alkenyl and alkynyl;
where R² is selected from alkyl, hydroxyalkyl, cycloalkyl, haloalkyl, alkoxy, phenalkyl, phenyl, phenoxy, phenalkoxy, alkoxyalkyl, alkylcarbonyl, alkoxycarbonyl, alkenyl, cycloalkenyl, alkynyl, alkylthio, phenthio and phenalkylthio;
wherein each of R³ through R¹¹ is independently selected from hydrido, hydroxy, alkyl, hydroxyalkyl, halo, haloalkyl, alkoxy, phenyl, benzoyl, phenoxy, alkoxyalkyl, acetyl, alkoxycarbonyl, alkenyl, cyano, nitro, carboxyl, alkylthio and mercapto;
and wherein each of R³ through R¹¹ may be an acidic moiety further independently selected from acidic moieties consisting of CO₂H, CO₂CH_{3,} SH, CH₂SH, C₂H₄SH, PO₃H₂, NHSO₂CF₃, NHSO₂C₆F₅, SO₃H, CONHNH₂, CONHNHSO₂CF₃, CONHOCH₃, CONHOC₂H₅, CONHCF₃, OH, CH₂OH, C₂H₄OH, OPO₃H₂, OSO₃H 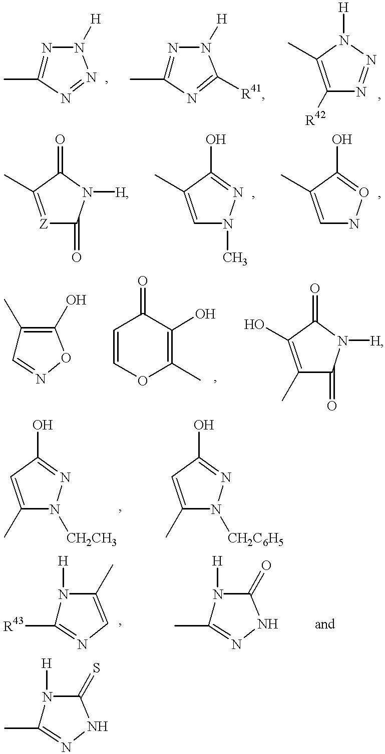
wherein each of R⁴¹, R⁴² and R⁴³ is independently selected from H, Cl, CN, NO₂, CF₃, C₂F₅, C₃F₇, CHF₂, CH₂F, CO₂CH₃, CO₂C₂H₅, SO₂CH₃, SO₂CF₃ and SO₂C₆F₅; wherein Z is selected from O, S, NR⁴⁴ and CH₂; wherein R⁴⁴ is selected from hydrido, CH₃ and CH₂C₆H₅; and wherein said acidic moiety may be a heterocyclic acidic group attached at any two adjacent positions of R³ through R¹¹ so as to form a fused ring system with one of the phenyl rings of the biphenyl moiety of Formula I, said biphenyl fused ring system selected from 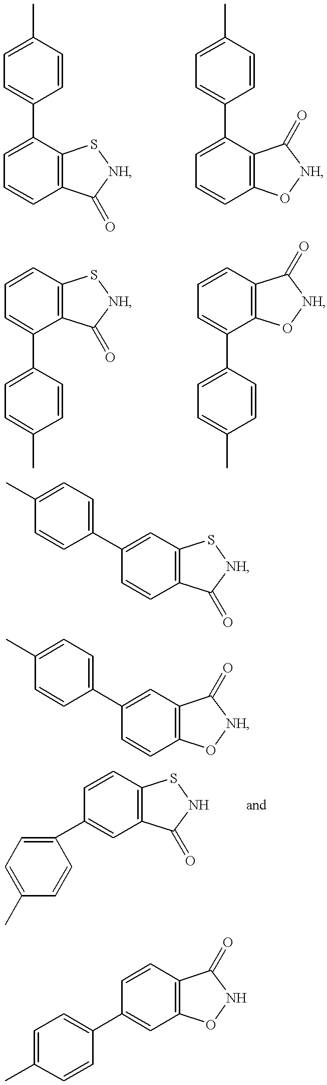
and the esters, amides and salts of said acidic moieties; or a tautomer thereof or a pharmaceutically-acceptable salt thereof.

7. Compound of claim 6 wherein m is one; wherein R¹ is selected from methyl, ethyl, n-propyl, isopropyl, n-butyl, sec-butyl, isobutyl, tert-butyl, n-pentyl, isopentyl, neopentyl, phenyl, benzyl, phenethyl, cyclohexyl, cyclohexylmethyl, 1-oxoethyl, 1-oxopropyl, 1-oxobutyl, 1-oxopentyl and hydroxyalkyl; wherein R² is selected from hydroxy, methyl, ethyl, n-propyl, isopropyl, n-butyl, sec-butyl, isobutyl, tert-butyl, n-pentyl, isopentyl, neopentyl, phenyl, benzyl, phenethyl, cyclohexyl, cyclohexylmethyl, propylthio, butylthio, 1-oxoethyl, 1-oxopropyl, 1-oxobutyl, 1-oxopentyl, 1,1-dimethoxypropyl, 1, 1-dimethoxybutyl, 1, 1-dimethoxypentyl, hydroxyalkyl, difluoromethyl, 1,1-difluoroethyl, 1,1-difluoropropyl, 1,1-difluorobutyl and 1,1-difluoropentyl; wherein at least one of R⁵, R⁶, R⁸ and R⁹ is an acidic group selected from CO₂H, SH, PO₃H₂, SO₃H, CONHNH₂, CONHNHSO₂CF₃, OH, 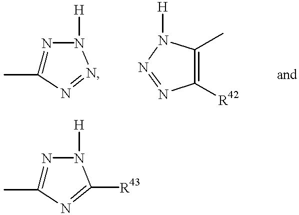
wherein each of R⁴² and R⁴³ is independently selected from Cl, CN, NO₂, CF₃, CO₂CH₃ and SO₂CF₃;
or a tautomer thereof or a pharmaceutically-acceptable salt thereof.

8. Compound of claim 7 wherein m is one;
wherein R¹ is selected from methyl, ethyl, n-propyl, isopropyl, n-butyl, sec-butyl, isobutyl, tert-butyl, 4-methylbutyl, n-pentyl, neopentyl, phenyl, benzyl, phenethyl, cyclohexyl, cyclohexylmethyl, 1-oxoethyl, 1-oxopropyl, 1-oxobutyl, 1-oxopentyl and hydroxyalkyl; wherein R² is selected from ethyl, n-propyl, isopropyl, n-butyl, sec-butyl, isobutyl, 4-methylbutyl, tert-butyl, n-pentyl, neopentyl, propylthio and butylthio; wherein at least one of R⁵, R⁶, R⁸ and R⁹ is an acidic group selected from CO₂H, SH, PO₃H₂, SO₃H, CONHNH₂, CONHNHSO₂CF₃, OH, 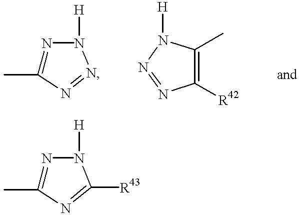
wherein each of R ⁴² and R⁴³ is independently selected from Cl, CN, NO₂, CF₃, CO₂CH₃ and SO₂CF₃;
or a tautomer thereof or a pharmaceutically-acceptable salt thereof.

9. Compound of claim 8 wherein m is one; wherein R¹ is selected from methyl, ethyl, n-propyl, isopropyl, n-butyl, sec-butyl, isobutyl, tert-butyl, 4-methylbutyl, n-pentyl, neopentyl, phenyl, benzyl, phenethyl, cyclohexyl, cyclohexylmethyl and hydroxyalkyl; wherein R² is selected from ethyl, n-propyl, isopropyl, n-butyl, sec-butyl, isobutyl, 4-methylbutyl, n-pentyl, propylthio and butylthio; wherein each of R³, R⁴, R⁶, R⁷, R⁸, R¹⁰ and R¹¹ is hydrido; wherein one of R⁵ and R⁹ is hydrido and the other of R⁵ and R⁹ is an acidic group selected from CO₂H and 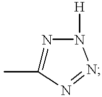
or a tautomer thereof or a pharmaceutically-acceptable salt thereof.

10. Compound of claim 8 selected from the group consisting of
2,5-dipropyl-2,4-dihydro-4-[[2-(1H-tetrazol-5-yl) [1,1′biphenyl]-4 ′-yl]methyl]-3H-1,2,4-triazol-3-one;
5-propyl-2-butyl-2,4-dihydro-4-[[2-(1H-tetrazol-5-yl) [1,1′-biphenyl]-4′-yl]methyl]-3H-1,2,4-triazol-3-one;
5-propyl-2-secbutyl-2,4-dihydro-4-[[2-(1H-tetrazol-5-yl) [1,1′-biphenyl]-4′-yl]methyl]-3H-1,2,4-triazol-3-one;
5-propyl-2-isobutyl-2,4-dihydro-4-[[2-(1H-tetrazol-5-yl) [1,1′-biphenyl]-4′-yl]methyl]-3H-1,2,4-triazol-3-one;
5-propyl-2-tertbutyl-2,4-dihydro-4-[[2-(1H-tetrazol-5-yl) [1,1′-biphenyl]-4′-yl]methyl]-3H-1,2,4-triazol-3-one;
5-propyl-2-pentyl-2,4-dihydro-4-[[2-(1H-tetrazol-5-yl) [1,1′-biphenyl]-4′-yl]methyl]-3H-1,2,4-triazol-3-one;
5-propyl-2-isopentyl-2,4-dihydro-4-[[2-(1H-tetrazol-5-yl) [1,1′-biphenyl]-4′-yl]methyl]-3H-1,2,4-triazol-3-one;
5-propyl-2-(1-oxopropyl)-2, 4-dihydro-4-[[2- (1H-tetrazol-5-yl) [1,1′-biphenyl]-4′-yl]methyl]-3H-1,2,4-triazol-3-one;
5-propyl-2-(1-oxobutyl)-2,4-dihydro-4-[[2-(1H-tetrazol-5-yl) [1,1′-biphenyl]-4′-yl]methyl]-3H-1,2,4-triazol-3-one;
5-propyl-2-(1-oxopentyl)-2,4-dihydro-4-[[2-(1H-tetrazol-5-yl) [1,1′-biphenyl]-4′-yl]methyl]-3H-1,2,4-triazol-3-one;
5-isopropyl-2-propyl-2,4-dihydro-4-[[2-(1H-tetrazol-5-yl) [1,1′-biphenyl]-4′-yl]methyl]-3H-1,2,4-triazol-3-one; 2,5-diisopropyl-2,4-dihydro-4-[[2-(1H-tetrazol-5-yl) [1,1′-biphenyl]-4′-yl]methyl]-3H-1,2,4-triazol-3-one;
5-isopropyl-2-butyl-2,4-dihydro-4-[[2-(1H-tetrazol-5-yl) [1,1′-biphenyl]-4′-yl]methyl]-3H-1,2,4-triazol-3-one;
5-isopropyl-2-secbutyl-2,4-dihydro-4-[[2-(1H-tetrazol-5-yl) [1,1′-biphenyl]-4′-yl]methyl]-3H-1,2,4-triazol-3-one;
5-isopropyl-2-isobutyl-2,4-dihydro-4-[[2-(1H-tetrazol-5-yl) [1,1′-biphenyl]-4′-yl]methyl]-3H-1,2,4-triazol-3-one;
5-isopropyl-2-tertbutyl-2,4-dihydro-4-[[2-(1H-tetrazol-5-yl) [1,1′-biphenyl]-4′-yl]methyl]-3H-1,2,4-triazol-3-one;
5-isopropyl-2-pentyl-2,4-dihydro-4-[[2-(1H-tetrazol-5-yl) [1,1′-biphenyl]-4′-yl]methyl]-3H-1,2,4-triazol-3-one;
5-isopropyl-2-isopentyl-2,4-dihydro-4-[[2-(1H-tetrazol-5-yl) [1,1′-biphenyl]-4′-yl]methyl]-3H-1,2,4-triazol-3-one;
5-isopropyl-2-(1-oxopropyl)-2,4-dihydro-4-[[2-(1H-tetrazol-5-yl) [1,1′-biphenyl]-4′-yl]methyl]-3H-1,2,4-triazol-3-one;
5-isopropyl-2-(1-oxobutyl)-2,4-dihydro-4-[[2-(1H-tetrazol-5-yl) [1,1′-biphenyl]-4′-yl]methyl]-3H-1,2,4-triazol-3-one;
5-isopropyl-2-(1-oxopentyl)-2,4-dihydro-4-[[2-(1H-tetrazol-5-yl) [1,1′-biphenyl]-4′-yl]methyl]-3H-1,2,4-triazol-3-one;
5-butyl-2-ethyl-2,4-dihydro-4-[[2-(1H-tetrazol-5-yl) [1,1′-biphenyl]-4′-yl]methyl]-3H-1,2,4-triazol-3-one;
5-butyl-2-propyl-2,4-dihydro-4-[[2-(1H-tetrazol-5-yl) [1,1′-biphenyl]-4′-yl]methyl]-3H-1,2,4-triazol-3-one;
5-butyl-2-isopropyl-2,4-dihydro-4-[[2-(1H-tetrazol-5-yl) [1,1′-biphenyl]-4′-yl]methyl]-3H-1,2,4-triazol-3-one; 2,5-dibutyl-2,4-dihydro-4-[[2-(1H-tetrazol-5-yl) [1,1′-biphenyl]-4′-yl]methyl]-3H-1,2,4-triazol-3-one;
5-butyl-2-secbutyl-2,4-dihydro-4-[[2-(1H-tetrazol-5-yl) [1,1′-biphenyl]-4′-yl]methyl]-3H-1,2,4-triazol-3-one;
5-butyl-2-isobutyl-2,4-dihydro-4-[[2-(1H-tetrazol-5-yl) [1,1′-biphenyl]-4′-yl]methyl]-3H-1,2,4-triazol-3-one;
5-butyl-2-tertbutyl-2,4-dihydro-4-[[2-(1H-tetrazol-5-yl) [1,1′-biphenyl]-4′-yl]methyl]-3H-1,2,4-triazol-3-one;
5-butyl-2-pentyl-2,4-dihydro-4-[[2-(1H-tetrazol-5-yl) [1,1′-biphenyl]-4′-yl]methyl]-3H-1,2,4-triazol-3-one;
5-butyl-2-isopentyl-2,4-dihydro-4-[[2-(1H-tetrazol-5-yl) [1,1′-biphenyl]-4′-yl]methyl]-3H-1,2,4-triazol-3-one;
5-butyl-2-hexyl-2,4-dihydro-4-[[2-(1H-tetrazo1-5-yl) [1,1′-biphenyl]-4′-yl]methyl]-3H-1,2,4-triazol-3-one;
5-butyl-2-heptyl-2,4-dihydro-4-[[2-(1H-tetrazol-5-yl) [1,1′-biphenyl]-4′-yl]methyl]-3H-1,2,4-triazol-3-one;
5-butyl-2-octyl-2,4-dihydro-4-[[2-(1H-tetrazol-5-yl) [1,1′-biphenyl]-4′-yl]methyl]-3H-1,2,4-triazol-3-one;
5-butyl-2-cyclohexyl-2,4-dihydro-4-[[2-(1H-tetrazol-5-yl) [1,1′-biphenyl]-4′-yl]methyl]-3H-1,2,4-triazol-3-one;
5-butyl-2-cyclohexylmethyl-2,4-dihydro-4-[[2-(1H-tetrazol-5-yl) [1,1′-biphenyl]-4′-yl]methyl]-3H-1,2,4-triazol-3-one;
5-butyl-2-(2-cyclohexylethyl)-2,4-dihydro-4-[[2-(1H-tetrazol-5-yl) [1,1′-biphenyl]-4′-yl]methyl]-3H-1,2,4-triazol-3-one;
5-butyl-2-cyclohexanoyl-2,4-dihydro-4-[[2-(1H-tetrazol-5-yl) [1,1′-biphenyl]-4′-yl]methyl]-3H-1,2,4-triazol-3-one;
5-butyl-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;
5-butyl-2-phenyl-2,4-dihydro-4-[[2-(1H-tetrazol-5-yl) [1,1′-biphenyl]-4′-yl]methyl]-3H-1,2,4-triazol-3-one;
5-butyl-2-phenylmethyl-2,4-dihydro-4-[[2-(1H-tetrazol-5-yl) [1,1′-biphenyl]-4′-yl]methyl]-3H-1,2,4-triazol-3-one;
5-butyl-2-(2-phenylethyl)-2,4-dihydro-4-[[2-(1H-tetrazol-5-yl) [1,1′-biphenyl]-4′-yl]methyl]-3H-1,2,4-triazol-3-one;
5-butyl-2-benzoyl-2,4-dihydro-4-[[2-(1H-tetrazol-5-yl) [1,1′-biphenyl]-4′-yl]methyl]-3H-1,2,4-triazol-3-one;
5-butyl-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;
5-butyl-2-(1-oxopropyl)-2,4-dihydro-4-[[2-(1H-tetrazol-5-yl) [1,1′-biphenyl]-4′-yl]methyl]-3H-1,2,4-triazol-3-one;
5-butyl-2-(1-oxobutyl)-2,4-dihydro-4-[[2-(1H-tetrazol-5-yl) [1,1′-biphenyl]-4′-yl]methyl]-3H-1,2,4-triazol-3-one;
5-butyl-2-(1-oxopentyl)-2,4-dihydro-4-[[2-(1H-tetrazol-5-yl) [1,1′-biphenyl]-4′-yl]methyl]-3H-1,2,4-triazol-3-one;
5-butyl-2-(2-butenyl)-2,4-dihydro-4-[[2-(1H-tetrazol-5-yl) [1,1′-biphenyl]-4′-yl]methyl]-3H-1,2,4-triazol-3-one;
5-butyl-2-(3-butenyl)-2,4-dihydro-4-[[2-(1H-tetrazol-5-yl) [1,1′-biphenyl]-4′-yl]methyl]-3H-1,2,4-triazol-3-one;
5-butyl-2-(2-butynyl)-2,4-dihydro-4-[[2-(1H-tetrazol-5-yl) [1,1′-biphenyl]-4′-yl]methyl]-3H-1,2,4-triazol-3-one;
5-butyl-2-(3-butynyl)-2,4-dihydro-4-[[2-(1H-tetrazol-5-yl) [1,1′-biphenyl]-4′-yl]methyl]-3H-1,2,4-triazol-3-one;
5-secbutyl-2-propyl-2,4-dihydro-4-[[2-(1H-tetrazol-5-yl) [1,1′-biphenyl]-4′-yl]methyl]-3H-1,2,4-triazol-3-one;
5-secbutyl-2-isopropyl-2,4-dihydro-4-[[2-(1H-tetrazol-5-yl) [1,1′-biphenyl]-4′-yl]methyl]-3H-1,2,4-triazol-3-one;
5-secbutyl-2-butyl-2,4-dihydro-4-[[2-(1H-tetrazol-5-yl) [1,1′-biphenyl]-4′-yl]methyl]-3H-1,2,4-triazol-3-one; 2,5-disecbutyl-2,4-dihydro-4-[[2-(1H-tetrazol-5-yl) [1,1′-biphenyl]-4′-yl]methyl]-3H-1,2,4-triazol-3-one;
5-secbutyl-2-isobutyl-2,4-dihydro-4-[[2-(1H-tetrazol-5-yl) [1,1′-biphenyl]-4′-yl]methyl]-3H-1,2,4-triazol-3-one;
5-secbutyl-2-tertbutyl-2,4-dihydro-4-[[2-(1H-tetrazol-5-yl) [1,1′-biphenyl]-4′-yl]methyl]-3H-1,2,4-triazol-3-one;
5-secbutyl-2-pentyl-2,4-dihydro-4-[[2-(1H-tetrazol-5-yl) [1,1′-biphenyl]-4′-yl]methyl]-3H-1,2,4-triazol-3-one;
5-secbutyl-2-isopentyl-2,4-dihydro-4-[[2-(1H-tetrazol-5-yl)[1,1′-biphenyl]-4′-yl]methyl]-3H-1,2,4-triazol-3-one;
5-secbutyl-2-(1-oxopropyl)-2,4-dihydro-4-[[2-(1H-tetrazol-5-yl) [1,1′-biphenyl]-4′-yl]methyl]-3H-1,2,4-triazol-3-one;
5-secbutyl-2-(1-oxobutyl)-2,4-dihydro-4-[[2-(1H-tetrazol-5-yl) [1,1′-biphenyl]-4′-yl]methyl]-3H-1,2,4-triazol-3-one;
5-secbutyl-2-(1-oxopentyl)-2,4-dihydro-4-[[2-(1H-tetrazol-5-yl) [1,1′-biphenyl]-4′-yl]methyl]-3H-1,2,4-triazol-3-one;
5-isobutyl-2-propyl-2,4-dihydro-4-[[2-(1H-tetrazol-5-yl) [1,1′-biphenyl]-4′-yl]methyl]-3H-1,2,4-triazol-3-one;
5-isobutyl-2-isopropyl-2,4-dihydro-4-[[2-(1H-tetrazol-5-yl) [1,1′-biphenyl]-4′-yl]methyl]-3H-1,2,4-triazol-3-one;
5-isobutyl-2-butyl-2,4-dihydro-4-[[2-(1H-tetrazol-5-yl) [1,1′-biphenyl]-4′-yl]methyl]-3H-1,2,4-triazol-3-one;
5-isobutyl-2-secbutyl-2, 4-dihydro-4-[[2-(1H-tetrazol-5-yl) [1,1′-biphenyl]-4′-yl]methyl]-3H-1,2,4-triazol-3-one; 2,5-diisobutyl-2,4-dihydro-4-[[2-(1H-tetrazol-5-yl) [1,1′-biphenyl]-4′-yl]methyl]-3H-1,2,4-triazol-3-one;
5-isobutyl-2-tertbutyl-2,4-dihydro-4-[[2-(1H-tetrazol-5-yl) [1,1′-biphenyl]-4′-yl]methyl]-3H-1,2,4-triazol-3-one;
5-isobutyl-2-pentyl-2,4-dihydro-4-[[2-(1H-tetrazol-5-yl) [1,1′-biphenyl]-4′-yl]methyl]-3H-1,2,4-triazol-3-one;
5-isobutyl-2-isopentyl-2,4-dihydro-4-[[2-(1H-tetrazol-5-yl) [1,1′-biphenyl]-4′-yl]methyl]-3H-1,2,4-triazol-3-one;
5-isobutyl-2-(1-oxopropyl)-2,4-dihydro-4-[[2-(1H-tetrazol-5-yl) [1,1′-biphenyl]-4′-yl]methyl]-3H-1,2,4-triazol-3-one;
5-isobutyl-2-(1-oxobutyl)-2,4-dihydro-4-[[2-(1H-tetrazol-5-yl) [1,1′-biphenyl]-4′-yl]methyl]-3H-1,2,4-triazol-3-one;
5-isobutyl-2-(1-oxopentyl)-2,4-dihydro-4-[[2-(1H-tetrazol-5-yl) [1,1′-biphenyl]-4′-yl]methyl]-3H-1,2,4-triazol-3-one;
5-2-propyl-2,4-dihydro-4-[[2-(1H-tetrazol-5-yl) [1,1′-biphenyl]-4′-yl]methyl]-3H-1,2,4-triazol-3-one;
5-2-isopropyl-2,4-dihydro-4-[[2-(1H-tetrazol-5-yl) [1,1′-biphenyl]-4′-yl]methyl]-3H-1,2,4-triazol-3-one;
5-2-butyl-2,4-dihydro-4-[[2-(1H-tetrazol-5-yl) [1,1′-biphenyl]-4′-yl]methyl]-3H-1,2,4-triazol-3-one;
5-2-secbutyl-2,4-dihydro-4-[[2-(1H-tetrazol-5-yl) [1,1′-biphenyl]-4′-yl]methyl]-3H-1,2,4-triazol-3-one;
5-2-isobutyl-2,4-dihydro-4-[[2-(1H-tetrazol-5-yl) [1,1′-biphenyl]-4′-yl]methyl]-3H-1,2,4-triazol-3-one; 2,5-ditertbutyl-2,4-dihydro-4-[[2-(1H-tetrazol-5-yl) [1,1′-biphenyl]-4′-yl]methyl]-3H-1,2,4-triazol-3-one;
5-2-pentyl-2,4-dihydro-4-[[2-(1H-tetrazol-5-yl) [1,1′-biphenyl]-4′-yl]methyl]-3H-1,2,4-triazol-3-one;
5-2-isopentyl-2,4-dihydro-4-[[2-(1H-tetrazol-5-yl) [1,1′-biphenyl]-4′-yl]methyl]-3H-1,2,4-triazol-3-one;
5-2-(1-oxopropyl)-2,4-dihydro-4-[[2-(1H-tetrazol-5-yl) [1,1′-biphenyl]-4′-yl]methyl]-3H-1,2,4-triazol-3-one;
5-2-(1-oxobutyl)-2,4-dihydro-4-[[2-(1H-tetrazol-5-yl) [1,1′-biphenyl]-4′-yl]methyl]-3H-1,2,4-triazol-3-one;
5-2-(1-oxopentyl)-2,4-dihydro-4-[[2-(1H-tetrazol-5-yl) [1,1′-bipheny1]-4′-yl)methyl]-3H-1,2,4-triazol-3-one;
5-pentyl-2-propyl-2,4-dihydro-4-[[2-(1H-tetrazol-5-yl) [1,1′-biphenyl]-4′-yl]methyl]-3H-1,2,4-triazol-3-one;
5-pentyl-2-isopropyl-2,4-dihydro-4-[[2-(1H-tetrazol-5-yl) [1,1′-biphenyl]-4′-yl]methyl]-3H-1,2,4-triazol-3-one;
5-pentyl-2-butyl-2,4-dihydro-4-[[2-(1H-tetrazol-5-yl) [1,1′-biphenyl]-4′-yl]methyl]-3H-1,2,4-triazol-3-one;
5-pentyl-2-secbutyl-2,4-dihydro-4-[[2-(1H-tetrazol-5-yl) [1,1′-biphenyl]-4′-yl]methyl]-3H-1,2,4-triazol-3-one;
5-pentyl-2-isobutyl-2,4-dihydro-4-[[2-(1H-tetrazol-5-yl) [1,1′-biphenyl]-4′-yl]methyl]-3H-1,2,4-triazol-3-one;
5-pentyl-2-tertbutyl-2,4-dihydro-4-[[2-(1H-tetrazol-5-yl) [1,1′-biphenyl]-4′-yl]methyl]-3H-1,2,4-triazol-3-one; 2,5-dipentyl-2,4-dihydro-4-[[2-(1H-tetrazol-5-yl) [1,1′-biphenyl]-4′-yl]methyl]-3H-1,2,4-triazol-3-one;
5-pentyl-2-isopentyl-2,4-dihydro-4-[[2-(1H-tetrazol-5-yl) [1,1′-biphenyl]-4′-yl]methyl]-3H-1,2,4-triazol-3-one;
5-pentyl-2-(1-oxopropyl)-2,4-dihydro-4-[[2-(1H-tetrazol-5-yl) [1,1′-biphenyl]-4′-yl]methyl]-3H-1,2,4-triazol-3-one;
5-pentyl-2-(1-oxobutyl)-2,4-dihydro-4-[[2-(1H-tetrazol-5-yl) [1,1′-biphenyl]-4′-yl]methyl]-3H-1,2,4-triazol-3-one;
5-pentyl-2-(1-oxopentyl)-2,4-dihydro-4-[[2-(1H-tetrazol-5-yl) [1,1′-biphenyl]-4′-yl]methyl]-3H-1,2,4-triazol-3-one; 5-isopentyl-2-propyl-2,4-dihydro-4-[[2-(1H-tetrazol-5-yl) [1,1′-biphenyl]-4′-yl]methyl]-3H-1,2,4-triazol-3-onei 5-isopentyl-2-isopropyl-2,4-dihydro-4-[[2-(1H-tetrazol-5-yl) [1,1′-biphenyl]-4′-yl]methyl]-3H-1,2,4-triazol-3-one; 5-isopentyl-2-butyl-2,4-dihydro-4-[[2-(1H-tetrazol-5-yl) [1,1′-biphenyl]-4′-yl]methyl]-3H-1,2,4-triazol-3-one; 5-isopentyl-2-secbutyl-2,4-dihydro-4-[[2-(1H-tetrazol-5-yl) [1,1′-biphenyl]-4′-yl]methyl]-3H-1,2,4-triazol-3-one; 5-isopentyl-2-isobutyl-2,4-dihydro-4-[[2-(1H-tetrazol-5-yl) [1,1′-biphenyl]-4′-yl]methyl]-3H-1,2,4-triazol-3-one; 5-isopentyl-2-tertbutyl-2,4-dihydro-4-[[2-(1H-tetrazol-5-yl) [1,1′-biphenyl]-4′-yl]methyl]-3H-1,2,4-triazol-3-one; 5-isopentyl-2-pentyl-2,4-dihydro-4-[[2-(1H-tetrazol-5-yl) [1,1′-biphenyl]-4′-yl]methyl]-3H-1,2,4-triazol-3-one; 2,5-diisopentyl-2,4-dihydro-4-[[2-(1H-tetrazol-5-yl) [1,1′-biphenyl]-4′-yl]methyl]-3H-1,2,4-triazol-3-one; 5-isopentyl-2-(1-oxopropyl)-2,4-dihydro-4-[[2-(1H-tetrazol-5-yl) [1,1′-biphenyl]-4′-yl]methyl]-3H-1,2,4-triazol-3-one; 5-isopentyl-2-(1-oxobutyl)-2,4-dihydro-4-[[2-(1H-tetrazol-5-yl) [1,1′-biphenyl]-4′-yl]methyl]-3H-1,2,4-triazol-3-one; 5-isopentyl-2-(1-oxopentyl)-2,4-dihydro-4-[[2-(1H-tetrazol-5-yl) [1,1′-biphenyl]-4′-yl]methyl]-3H-1,2,4-triazol-3-one;
5-hexyl-2-propyl-2,4-dihydro-4-[[2-(1H-tetrazol-5-yl) [1,1′-biphenyl]-4′-yl]methyl]-3H-1,2,4-triazol-3-one;
5-hexyl-2-isopropyl-2,4-dihydro-4-[[2-(1H-tetrazol-5-yl) [1,1′-biphenyl]-4′-yl]methyl]-3H-1,2,4-triazol-3-one;
5-hexyl-2-butyl-2,4-dihydro-4-[[2-(1H-tetrazol-5-yl) [1,1′-biphenyl]-4′-yl]methyl]-3H-1,2,4-triazol-3-one;
5-hexyl-2-secbutyl-2,4-dihydro-4-[[2-(1H-tetrazol-5-yl) [1,1′-biphenyl]-4′-yl]methyl]-3H-1,2,4-triazol-3-one;
5-hexyl-2-isobutyl-2,4-dihydro-4-[[2-(1H-tetrazol-5-yl) [1,1′-biphenyl]-4′-yl]methyl]-3H-1,2,4-triazol-3-one;
5-hexyl-2-tertbutyl-2,4-dihydro-4-[[2-(1H-tetrazol-5-yl) [1,1′-biphenyl]-4′-yl]methyl]-3H-1,2,4-triazol-3-one;
5-hexyl-2-pentyl-2,4-dihydro-4-[[2-(1H-tetrazol-5-yl) [1,1′-biphenyl]-4′-yl]methyl]-3H-1,2,4-triazol-3-one;
5-hexyl-2-isopentyl-2,4-dihydro-4-[[2-(1H-tetrazol-5-yl) [1,l′-biphenyl]-4′-yl]methyl]-3H-1,2,4-triazol-3-one; 2,5-dihexyl-2,4-dihydro-4-[[2-(1H-tetrazol-5-yl) [1,1′-biphenyl]-4′-yl]methyl]-3H-1,2,4-triazol-3-one;
5-hexyl-2-(1-oxopropyl)-2,4-dihydro-4-[[2-(1H-tetrazol-5-yl) [1,1′-biphenyl]-4′-yl]methyl]-3H-1,2,4-triazol-3-one;
5-hexyl-2-(1-oxobutyl)-2,4-dihydro-4-[12-(1H-tetrazol-5-yl) [1,1′-biphenyl]-4′-yl]methyl3-3H-1,2,4-triazol-3-one;
5-hexyl-2-(1-oxopentyl)-2,4-dihydro-4-[[2-(1H-tetrazol-5-yl) [1,1′-biphenyl]-4′-yl]methyl]-3H-1,2,4-triazol-3-one;
5-heptyl-2-butyl-2,4-dihydro-4-[[2-(1H-tetrazol-5-yl) [1,1′-biphenyl]-4′-yl]methyl]-3H-1,2,4-triazol-3-one; 2,5-diheptyl-2,4-dihydro-4-[[2-(1H-tetrazol-5-yl) [1,1′-biphenyl]-4′-yl]methyl]-3H-1,2,4-triazol-3-one;
5-heptyl-2-(1-oxopropyl)-2,4-dihydro-4-[[2-(1H-tetrazol-5-yl) [1,1′-biphenyl]-4′-yl]methyl]-3H-1,2,4-triazol-3-one;
5-heptyl-2-(1-oxobutyl)-2,4-dihydro-4-[[2-(1H-tetrazol-5-yl) [1,1′-biphenyl]-4′-yl]methyl]-3H-1,2,4-triazol-3-one;
5-heptyl-2-(1-oxopentyl)-2,4-dihydro-4-[[2-(1H-tetrazol-5-yl) [1,1′-biphenyl]-4′-yl]methyl]-3H-1,2,4-triazol-3-one;
5-octyl-2-butyl-2,4-dihydro-4-[[2-(1H-tetrazol-5-yl) [1,1′-biphenyl]-4′-yl]methyl]-3H-1,2,4-triazol-3-one; 2,5-dioctyl-2,4-dihydro-4-[[2-(1H-tetrazol-5-yl) [1,1′-biphenyl]-4′-yl]methyl]-3H-1,2,4-triazol-3-one;
5-octyl-2-(1-oxopropyl)-2,4-dihydro-4-[[2-(1H-tetrazol-5-yl) [1,1′-biphenyl]-4′-yl]methyl]-3H-1,2,4-triazol-3-one;
5-octyl-2-(1-oxobutyl)-2,4-dihydro-4-[[2-(1H-tetrazol-5-yl) [1,1′-biphenyl]-4 -yl]methyl]-3H-1,2,4-triazol-3-one;
5-octyl-2-(1-oxopentyl)-2,4-dihydro-4-[[2-(1H-tetrazol-5-yl) [1,1′-biphenyl]-4′-yl]methyl]-3H-1,2,4-triazol-3-one;
5-(1-butenyl)-2-propyl-2,4-dihydro-4-[[2-(1H-tetrazol-5-yl) [1,1′-biphenyl]-4′-yl]methyl]-3H-1,2,4-triazol-3-one;
5-(1-butenyl)-2-isopropyl-2,4-dihydro-4-([2-(1H-tetrazol-5-yl) [1,1′-biphenyl]-4′-yl]methyl]-3H-1,2,4-triazol-3-one;
5-(1-butenyl)-2-butyl-2,4-dihydro-4-[[2-(1H-tetrazol-5-yl) [1,1′-biphenyl]-4′-yl]methyl]-3H-1,2,4-triazol-3-one;
5-(1-butenyl)-2-secbutyl-2,4-dihydro-4-[[2-(1H-tetrazol-5-yl) [1,1′-biphenyl]-4′-yl]methyl]-3H-1,2,4-triazol-3-one;
5-(1-butenyl)-2-isobutyl-2,4-dihydro-4-[[2-(1H-tetrazol-5-yl) [1,1′-biphenyl]-4′-yl]methyl]-3H-1,2,4-triazol-3-one;
5-(1-butenyl)-2-tertbutyl-2,4-dihydro-4-[[2-(1H-tetrazol-5-yl) [1,1′-biphenyl]-4′-yl]methyl]-3H-1,2,4-triazol-3-one;
5-(1-butenyl)-2-pentyl-2,4-dihydro-4-[[2-(1H-tetrazol-5-yl) [1,1′-biphenyl]-4′-yl]methyl]-3H-1,2,4-triazol-3-one;
5-(1-butenyl)-2-isopentyl-2,4-dihydro-4-[[2-(1H-tetrazol-5-yl) [1,1′-biphenyl]-4′-yl]methyl]-3H-1,2,4-triazol-3-one;
5-(1-butenyl)-2-hexyl-2,4-dihydro-4-[[2-(1H-tetrazol-5-yl) [1,1′-biphenyl]-4′-yl]methyl]-3H-1,2,4-triazol-3-one;
5-(1-butenyl)-2-heptyl2,4-dihydro-4-[[2-(1H-tetrazol-5-yl) [1,1′-biphenyl]-4′-yl]methyl]-3H-1,2,4-triazol-3-one;
5-(1-butenyl)-2-octyl-2,4-dihydro-4-[[2-(1H-tetrazol-5-yl) [1,1′-biphenyl]-4′-yl]methyl]-3H-1,2,4-triazol-3-one;
5-(1butenyl)-2-cyclohexyl-2,4-dihydro-4-[[2-(1H-tetrazol-5-yl) [1,1′-biphenyl]-4′-yl]methyl]-3H-1,2,4-triazol-3-one;
5-(1-butenyl)-2-cyclohexylmethyl-2,4-dihydro-4-[[2-(1H-tetrazol-5-yl) [1,1′-biphenyl]-4′-yl]methyl]-3H-1,2,4-triazol-3-one;
5-(1-butenyl)-2-(2-cyclohexylethyl)-2,4-dihydro-4-[[2-(1H-tetrazol-5-yl) [1,1′-biphenyl]-4′-yl]methyl]-3H-1,2,4-triazol-3-one;
5-(1-butenyl)-2-cyclohexanoyl-2,4-dihydro-4-[[2-(1H-tetrazol-5-yl) [1,1′-biphenyl]-4′-yl]methyl]-3H-1,2,4-triazol-3-one;
5-(1-butenyl)-2-(1-oxo-2-cyclohexylethyl)-2,4-dihydro-4-[[2-(1H-tetrazol-5-yl) [1,1′-biphenyl]-4′-yl]methyl]-3H-1,2,4-triazol-3-one;
5-(1-butenyl)-2-phenyl-2,4-dihydro-4-[[2-(1H-tetrazol-5-yl) [1,1′-biphenyl]-4′-yl]methyl]-3H-1,2,4-triazol-3-one;
5-(1-butenyl)-2-phenylmethyl-2,4-dihydro-4-[[2-(1H-tetrazol-5-yl) [1,1′-biphenyl]-4′-yl]methyl]-3H-1,2,4-triazol-3-one;
5-(1-butenyl)-2-benzoyl-2,4-dihydro-4-[[2-(1H-tetrazol-5-yl) [1,1′-biphenyl]-4′-yl]methyl]-3H-1,2,4-triazol-3-one;
5-(1-butenyl)-2-(1-oxo-2-phenylethyl)-2,4-dihydro-4-[[2-(1H-tetrazol-5-yl) [1,1′-biphenyl]-4′-yl]methyl]-3H-1,2,4-triazol-3-one;
5-(1-butenyl)-2-(1-oxopropyl)-2,4-dihydro-4-[[2-(1H-tetrazol-5-yl) [1,1′-biphenyl]-4′-yl]methyl]-3H-1,2,4-triazol-3-one;
5-(1-butenyl)-2-(1-oxobutyl)-2,4-dihydro-4-[[2-(1H-tetrazol-5-yl) [1,1′-biphenyl]-4′-yl]methyl]-3H-1,2,4-triazol-3-one;
5-(1-butenyl)-2-(1-oxopentyl)-2,4-dihydro-4-[[2-(1H-tetrazol-5-yl) [1,1′-biphenyl]-4′-yl]methyl]-3H-1,2,4-triazol-3-one;
5-(1-butenyl)-2-(2-butenyl)-2,4-dihydro-4-[[2-(1H-tetrazol-5-yl) [1,1′-biphenyl]-4′-yl]methyl]-3H-1,2,4-triazol-3-one;
5-(1-butenyl)-2-(3-butenyl)-2,4-dihydro-4-[[2-(1H-tetrazol-5-yl) [1,1′-biphenyl]-4′-yl]methyl]-3H-1,2,4-triazol-3-one;
5-(1-butenyl)-2-(2-butynyl)-2,4-dihydro-4-[[2-(1H-tetrazol-5-yl) [1,1′-biphenyl]-4′-yl]methyl]-3H-1,2,4-triazol-3-one;
5-(1-butenyl)-2-(3-butynyl)-2,4-dihydro-4-[[2-(1H-tetrazol-5-yl) [1,1′-biphenyl]-4′-yl]methyl]-3H-1,2,4-triazol-3-one;
5-(2-butenyl)-2-propyl-2,4-dihydro-4-[[2-(1H-tetrazol-5-yl) [1,1′-biphenyl]-4′-yl]methyl]-3H-1,2,4-triazol-3-one;
5-(2-butenyl)-2-isopropyl-2,4-dihydro-4-[[2-(1H-tetrazol-5-yl) [1,1′-biphenyl]-4′-yl]methyl]-3H-1,2,4-triazol-3-one;
5-(2-butenyl)-2-butyl-2,4-dihydro-4-[[2-(1H-tetrazol-5-yl) [1,1′-biphenyl]-4′-yl]methyl]-3H-1,2,4-triazol-3-one;
5-(2-butenyl)-2-secbutyl-2,4-dihydro-4-[[2-(1H-tetrazol-5-yl) [1,1′-biphenyl]-4′-yl]methyl]-3H-1,2,4-triazol-3-one;
5-(2-butenyl)-2-isobutyl-2,4-dihydro-4-[[2-(1H-tetrazol-5-yl) [1,1′-biphenyl]-4′-yl]methyl]-3H-1,2,4-triazol-3-one;
5-(2-butenyl)-2-tertbutyl-2,4-dihydro-4-[[2-(1H-tetrazol-5-yl) [1,1′-biphenyl]-4′-yl]methyl]-3H-1,2,4-triazol-3-one;
5-(2-butenyl)-2-pentyl-2,4-dihydro-4-[[2-(1H-tetrazol-5-yl) [1,1′-biphenyl]-4′-yl]methyl]-3H-1,2,4-triazol-3-one;
5-(2-butenyl)-2-isopentyl-2,4-dihydro-4-[[2-(1H-tetrazol-5-yl) [1,1′-biphenyl]-4′-yl]methyl]-3H-1,2,4-triazol-3-one;
5-(2-butenyl)-2-(1-oxopropyl)-2,4-dihydro-4-[[2-(1H-tetrazol-5-yl) [1,1′-biphenyl]-4′-yl]methyl]-3H-1,2,4-triazol-3-one;
5-(2-butenyl)-2-(1-oxobutyl)-2,4-dihydro-4-[[2-(1H-3tetrazol-5-yl) [1,1′-biphenyl]-4′-yl]methyl]-3H-1,2,4-triazol-3-one;
5-(2-butenyl)-2-(1-oxopentyl)-2,4-dihydro-4-[[2-(1H-tetrazol-5-yl) [1,1′-biphenyl]-4′-yl]methyl]-3H-1,2,4-triazol-3-one;
5-(3-butenyl)-2-propyl-2,4-dihydro-4-[[2-(1H-tetrazol-5-yl) [1,1′-biphenyl]-4′-yl]methyl]-3H-1,2,4-triazol-3-one;
5-(3-butenyl)-2-butyl-2,4-dihydro-4-[[2-(1H-tetrazol-5-yl) [1,1′-biphenyl]-4′-yl]methyl]-3H-1,2,4-triazol-3-one;
5-(3-butenyl)-2-pentyl-2,4-dihydro-4-[[2-(1H-tetrazol-5-yl) [1,1′-biphenyl]-4′-yl]methyl]-3H-1,2,4-triazol-3-one;
5-(3-butenyl)-2-isopentyl-2,4-dihydro-4-[[2-(1H-tetrazol-5-yl) [1,1′-biphenyl]-4′-yl]methyl]-3H-1,2,4-triazol-3-one;
5-(3-butenyl)-2-(1-oxopropyl)-2,4-dihydro-4-[[2-(1H-tetrazol-5-yl) [1,1′-biphenyl]-4′-yl]methyl]-3H-1,2,4-triazol-3-one;
5-(3-butenyl)-2-(1-oxobutyl)-2,4-dihydro-4-[[2-(1H-tetrazol-5-yl) [1,1′-biphenyl]-4′-yl]methyl]-3H-1,2,4-triazol-3-one;
5-(3-butenyl)-2-(1-oxopentyl)-2,4-dihydro-4-[[2-(1H-tetrazol-5-yl) [1,1′-biphenyl]-4′-yl]methyl]-3H-1,2,4-triazol-3-one;
5-(1-butynyl)-2-propyl-2,4-dihydro-4-[[2-(1H-tetrazol-5-yl) [1,1′-biphenyl]-4′-yl]methyl]-3H-1,2,4-triazol-3-one;
5-(1butynyl)-2-butyl-2,4-dihydro-4-[[2-(1H-tetrazol-5-yl) [1,1′-biphenyl]-4′-yl]methyl]-3H-1,2,4-triazol-3-one;
5-(1-butynyl)-2-secbutyl-2,4-dihydro-4-[[2-(1H-tetrazol-5-yl) [1,1′-biphenyl]-4′-yl]methyl]-3H-1,2,4-triazol-3-one;
5-(1-butynyl)-2-isobutyl-2,4-dihydro-4-[[2-(1H-tetrazol-5-yl) [1,1′-biphenyl]-4′]mehtyl]-3H-1,2,4-trizaol-3one;
5-(1-butynyl)-2-tertbutyl-2,4-dihydro-4-[[2-(1H-tetrazol-5-yl) [1,1′-biphenyl]-4′-yl]methyl]-3H-1,2,4-triazol-3-one;
5-(1-butynyl)-2-pentyl-2,4-dihydro-4-[[2-(1H-tetrazol-5-yl) [1,1′-biphenyl]-4′-yl]methyl]-3H-1,2,4-triazol-3-one;
5-(1butynyl)-2-isopentyl-2,4-dihydro-4-[[2-(1H-tetrazol-5-yl) [1,1′-biphenyl]-4′-yl]methyl]-3H-1,2,4-triazol-3-one;
5-(1-butynyl)-2-hexyl-2,4-dihydro-4-[[2-(1H-tetrazol-5-yl) [1,1′-biphenyl]-4′-yl]methyl]-3H-1,2,4-triazol-3-one;
5-(1-butynyl)-2-(1-oxopropyl) -2,4-dihydro-4-[[2-(1H-tetrazol-5-yl) [1,1′-biphenyl]-4′-yl]methyl]-3H-1,2,4-triazol-3-one;
5-(1-butynyl)-2-(1-oxobutyl)-2,4-dihydro-4-[[2-(1H-tetrazol-5-yl) [1,1′-biphenyl]-4′-yl]methyl]-3H-1,2,4-triazol-3-one;
5-(1-butynyl)-2-(1-oxopentyl)-2,4-dihydro-4-[[2-(1H-tetrazol-5-yl) [1,1′-biphenyl]-4′-yl]methyl]-3H-1,2,4-triazol-3-one;
5-(2-butynyl)-2-propyl- 2,4-dihydro-4-[[2-(1H-tetrazol-5-yl) [1,1 ′-biphenyl]-4 ′-yl ]methyl-3H-1,2,4-triazol-3-one;
5-(2-butynyl)-2-butyl-2,4-dihydro-4-[[2-(1H-tetrazol-5-yl) [1,1′-biphenyl]-4′-yl]methyl]-3H-1,2,4-triazol-3-one;
5-(2-butynyl)-2-pentyl-2,4-dihydro-4-[[2-(1H-tetrazol-5-yl) [1,1′-biphenyl]-4′-yl]methyl]-3H-1,2,4-triazol-3-one;
5-(2-butynyl)-2-isopentyl-2,4-dihydro-4-[[2-(1H-tetrazol-5-yl) [1,1′-biphenyl]-4′-yl]methyl]-3H-1,2,4-triazol-3-one;
5-(2-butynyl)-2-(1-oxopropyl)-2,4-dihydro-4-[[2-(1H-tetrazol-5-yl) [1,1′-biphenyl]-4′-yl]methyl]-3H-1,2,4-triazol-3-one;
5-(2-butynyl)-2-(1-oxobutyl)-2,4-dihydro-4-[[2-(1H-tetrazol-5-yl) [1,1′-biphenyl]-4′-yl]methyl]-3H-1,2,4-triazol-3-one;
5-(2-butynyl)-2-(1-oxopentyl)-2,4-dihydro-4-[[2-(1H-tetrazol-5-yl) [1,1′-biphenyl]-4′-yl]methyl]-3H-1,2,4-triazol-3-one;
5-(3-butynyl)-2-propyl-2,4-dihydro-4-[[2-(1H-tetrazol-5-yl) [1,1′-biphenyl]-4′-yl]methyl]-3H-1,2,4-triazol-3-one;
5-(3-butynyl)-2-butyl-2,4-dihydro-4-[[2-(1H-tetrazol-5-yl) [1,1′-biphenyl]-4′-yl]methyl]-3H-1,2,4-triazol-3-one;
5-(3-butynyl)-2-isopentyl-2,4-dihydro-4-[[2-(1H-tetrazol-5-yl) [1,1′-biphenyl]-4′-yl]methyl]-3H-1,2,4-triazol-3-one;
5-(3-butynyl)-2-pentyl-2,4-dihydro-4-[[2-(1H-tetrazol-5-yl) [1,1′-biphenyl]-4′-yl]methyl]-3H-1,2,4-triazol-3-one;
5-(3-butynyl)-2-(1-oxopropyl)-2,4-dihydro-4-[[2-(1H-tetrazol-5-yl) [1,1′-biphenyl]-4′-yl]methyl]-3H-1,2,4-triazol-3-one;
5-(3-butynyl)-2-(1-oxobutyl)-2,4-dihydro-4-[[2-(1H-tetrazol-5-yl) [1,1′-biphenyl]-4′-yl]methyl]-3H-1,2,4-triazol-3-one;
5-(3-butynyl)-2-(1-oxopentyl)-2,4-dihydro-4-[[2-(1H-tetrazol-5-yl) [1,1′-biphenyl]-4′-yl]methyl]-3H-1,2,4-triazol-3-one; and 4′-[(1,3-dibutyl-4,5-dihydro-5-oxo-1H-1,2,4-triazol-4-yl) methyl] [1,1′-biphenyl]-2-carboxylic acid.

11. Compound of claim 10 which is 4′-[(1,3-dibutyl-4,5-dihydro-5-oxo-1H-1,2,4-triazol-4-yl) methyl] [1,1′-biphenyl]-2-carboxylic acid.

12. Compound of claim 10 which is
2,5-dibutyl-2,4-dihydro-4-[2-(1H-tetrazol-5-yl)[1,1′-biphenyl]-4′-yl] methyl]-3H-1,2,4-triazol-3-one.

13. A pharmaceutical composition comprising a therapeutically-effective amount of an angiotensin II antagonist compound and a pharmaceutically-acceptable carrier or diluent, said antagonist compound selected from a family of compounds of Formula I: 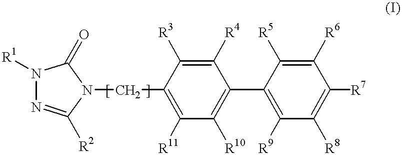
wherein m is a number selected from one to four, inclusive;
wherein R¹ is selected from alkyl, hydroxyalkyl, haloalkyl, cycloalkyl, cycloalkylalkyl, formyl, aralkyl, aryl, aroyl, alkoxyalkyl, alkylcarbonyl, alkylcarbonylalkyl, alkoxycarbonyl, alkenyl, cycloalkenyl, aralkoxycarbonyl, alkynyl, cyano, carboxyl, mercaptocarbonyl, mercaptothiocarbonyl, alkylthiocarbonyl, alkylthiothiocarbonyl, arylthiocarbonyl, arylthiothiocarbonyl, aralkylthiocarbonyl, alkylthiocarbonyl, alkylsulfinyl, alkylsulfonyl, aralkylsulfinyl, aralkylsulfonyl, arylsulfinyl, arylsulfonyl, heteroaryl having one or more ring atoms selected from oxygen, sulfur and nitrogen atoms, and amido radicals of the formula 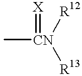
wherein X is oxygen atom or sulfur atom;
wherein each of R¹² and R¹³ is independently selected from hydrido, alkyl, cycloalkyl, cyano, hydroxyalkyl, cycloalkylalkyl, alkoxyalkyl, aralkyl and aryl, and wherein R¹² and R¹³ taken together may form a heterocyclic group having five to seven ring members including the nitrogen atom of said amino or amido radical and which heterocyclic group may further contain one or more hetero atoms as ring members selected from oxygen, nitrogen and sulfur atoms and which heterocyclic group may be saturated or partially unsaturated; wherein each of R¹² and R¹³ taken together may form an aromatic heterocyclic group having five ring members including the nitrogen atom of said amino or amido radical and which aromatic heterocyclic group may further contain one or more hetero atoms as ring atoms selected from oxygen, nitrogen and sulfur atoms;
wherein each of R² through R¹¹ is independently selected from hydrido, alkyl, hydroxyalkyl, halo, haloalkyl, cycloalkyl, cycloalkylalkyl, formyl, alkoxy, aralkyl, aryl, aroyl, aryloxy, aralkoxy, aralkoxycarbonyl, alkoxyalkyl, alkylcarbonyl, alkylcarbonylalkyl, alkoxycarbonyl, alkenyl, cycloalkenyl, alkynyl, cyano, nitro, carboxyl, alkylcarbonyloxy, mercaptocarbonyl, mercaptothiocarbonyl, alkoxycarbonyloxy, alkylthio, alkylthiocarbonyl, alkylcarbonylthio, alkylthiocarbonyloxy, alkylthiocarbonylthio, alkylthiothiocarbonyl, alkylthiothiocarbonylthio, arylthio, arylthiocarbonyl, arylcarbonylthio, arylthiocarbonyloxy, arylthiocarbonylthio, arylthiothiocarbonyl, arylthiothiocarbonylthio, aralkylthio, aralkylthiocarbonyl, aralkylcarbonylthio, aralkylthiocarbonyloxy, aralkylthiocarbonylthio, alkylthiocarbonyl, aralkylthiocarbonylthio, mercapto, alkylsulfinyl, alkylsulfonyl, aralkylsulfinyl, aralkylsulfonyl, arylsulfinyl, arylsulfonyl, heteroaryl having one or more ring atoms selected from oxygen, sulfur and nitrogen atoms, and amino and amido radicals of the formula 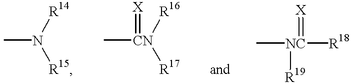
wherein X is oxygen atom or sulfur atom; wherein each of R¹⁴, R¹⁵, R¹⁶, R¹⁷, R¹⁸ and R¹⁹ is independently selected from hydrido, alkyl, cycloalkyl, cyano, hydroxyalkyl, cycloalkylalkyl, alkoxyalkyl, aralkyl and aryl, and wherein R¹⁴ and R¹⁵ taken together, R¹⁶ and R¹⁷ taken together and R¹⁸ and R¹⁹ taken together may form a heterocyclic group having five to seven ring members including the nitrogen atom of said amino or amido radical and which heterocyclic group may further contain one or more hetero atoms as ring members selected from oxygen, nitrogen and sulfur atoms and which heterocyclic group may be saturated or partially unsaturated; wherein each of R¹⁴ and R¹⁵ taken together and each of R¹⁶ and R¹⁷ taken together may form an aromatic heterocyclic group having five ring members including the nitrogen atom of said amino or amido radical and which aromatic heterocyclic group may further contain one or more hetero atoms as ring atoms selected from oxygen, nitrogen and sulfur atoms; and wherein each of R³ through R¹¹ may be further independently selected from hydroxy and acidic moieties of the formula
−YₙA
wherein n is a number selected from zero through three, inclusive, and wherein A is an acidic group selected to contain at least one acidic hydrogen atom, and the amide, ester and salt derivatives of said acidic moieties; wherein Y is a spacer group independently selected from one or more of alkyl, cycloalkyl, cycloalkylalkyl, alkenyl, alkynyl, aryl, aralkyl and heteroaryl having one or more ring atoms selected from oxygen, sulfur and nitrogen atoms;
and wherein any of the foregoing R¹ through R¹⁹, Y and A groups having a substitutable position may be substituted by one or more groups selected from hydroxy, alkyl, alkenyl, alkynyl, aralkyl, hydroxyalkyl, haloalkyl, halo, oxo, alkoxy, aryloxy, aralkoxy, aralkylthio, alkoxyalkyl, cycloalkyl, cycloalkylalkyl, aryl, aroyl, cycloalkenyl, cyano, cyanoamino, nitro, alkylcarbonyloxy, alkoxycarbonyloxy, alkylcarbonyl, alkoxycarbonyl, aralkoxycarbonyl, carboxyl, mercapto, mercaptocarbonyl, alkylthio, arylthio, alkylthiocarbonyl, alkylsulfinyl, alkylsulfonyl, haloalkylsulfonyl, aralkylsulfinyl, aralkylsulfonyl, arylsulfinyl, arylsulfonyl, heteroaryl having one or more ring atoms selected from oxygen, sulfur and nitrogen atoms, and amino and amido radicals of the formula 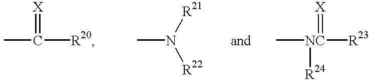
wherein X is oxygen atom or sulfur atom; wherein R²⁰ is selected from hydrido, alkyl, cycloalkyl, cycloalkylalkyl, aralkyl, aryl, DR²⁵ and 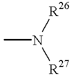
wherein D is selected from oxygen atom and sulfur atom and R²⁵ is selected from hydrido, alkyl, cycloalkyl, cycloalkylalkyl, aralkyl and aryl; wherein each of R²¹, R²², R²³, R²⁴, R²⁶ and R²⁷ is independently selected from hydrido, alkyl, cycloalkyl, cyano, hydroxyalkyl, haloalkyl, cycloalkylalkyl, alkoxyalkyl, alkylcarbonyl, alkoxycarbonyl, carboxyl, alkylsulfinyl, alkylsulfonyl, arylsulfinyl, arylsulfonyl, haloalkylsulfinyl, haloalkylsulfonyl, aralkyl and aryl, and wherein each of R²¹, R²², R²³, R²⁴, R²⁶ and R²⁷ is further independently selected from amino and amido radicals of the formula 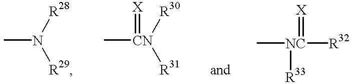
wherein each of R²⁸, R²⁹, R³⁰, R³¹ R³² and R³³ is independently selected from hydrido, alkyl, cycloalkyl, cyano, hydroxyalkyl, cycloalkylalkyl, alkoxyalkyl, haloalkylsulfinyl, haloalkylsulfonyl, aralkyl and aryl, and wherein each of R²¹ and R²² taken together and each of R²³ and R²⁴ taken together may form a heterocyclic group having five to seven ring members including the nitrogen atom of said amino or amido radical, which heterocyclic group may further contain one or more hetero atoms as ring members selected from oxygen, nitrogen and sulfur atoms and which heterocyclic group may be saturated or partially unsaturated; wherein each of R²¹ and R²² taken together and each of R²⁶ and R²⁷ taken together may form an aromatic heterocyclic group having five ring members including the nitrogen atom of said amino or amido radical and which aromatic heterocyclic group may further contain one or more hetero atoms as ring atoms selected from oxygen, nitrogen and sulfur atoms; or a tautomer thereof or a pharmaceutically-acceptable salt thereof.

14. The composition of claim 13 wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkoxycarbonyl, alkenyl, cycloalkenyl, alkynyl, cycloalkynyl, carboxyl, mercaptocarbonyl, mercaptothiocarbonyl, alkylthiocarbonyl, arylthiocarbonyl, arylthiothiocarbonyl, aralkylthiocarbonyl, alkylsulfonyl, aralkylsulfonyl, arylsulfonyl, heteroaryl having one or more ring atoms selected from oxygen, sulfur and nitrogen atoms, and amido radicals of the formula 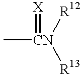
wherein X is oxygen atom or sulfur atom; wherein each of R¹² and R¹³ is independently selected from hydrido, alkyl, cycloalkyl, cyano, hydroxyalkyl, cycloalkylalkyl, alkoxyalkyl, aralkyl and aryl;
wherein R²,is selected from hydrido, alkyl, hydroxyalkyl, halo, haloalkyl, cycloalkyl, cycloalkylalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cycloalkynyl, cyano, carboxyl, alkylcarbonyloxy, alkylthio, alkylthiocarbonyl, alkylcarbonylthio, alkylthiocarbonyloxy, alkylthiocarbonylthio, alkylthiothiocarbonyl, alkylthiothiocarbonylthio, arylthio, arylthiocarbonyl, arylcarbonylthio, arylthiocarbonyloxy, arylthiocarbonylthio, arylthiothiocarbonyl, arylthiothiocarbonylthio, aralkylthio, aralkylthiocarbonyl, aralkylcarbonylthio, aralkylthiocarbonyloxy, aralkylthiocarbonylthio, aralkylthiocarbonyl, aralkylthiocarbonylthio, mercapto, alkylsulfonyl, aralkylsulfonyl, arylsulfonyl and heteroaryl having one or more ring atoms selected from oxygen, sulfur and nitrogen atoms;
wherein each of R³ through R¹¹ is independently selected from hydrido, hydroxy, alkyl, hydroxyalkyl, halo, haloalkyl, cycloalkyl, cycloalkylalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cycloalkynyl, cyano, nitro, carboxyl, alkylcarbonyloxy, mercaptocarbonyl, mercaptothiocarbonyl, alkoxycarbonyloxy, alkylthio, alkylthiocarbonyl, alkylcarbonylthio, alkylthiocarbonyloxy, alkylthiocarbonylthio, alkylthiothiocarbonyl, arylthio, arylthiocarbonyl, arylcarbonylthio, arylthiocarbonyloxy, arylthiothiocarbonyl, aralkylthio, aralkylthiocarbonyl, aralkylcarbonylthio, aralkylthiocarbonyloxy, aralkylthiocarbonylthio, aralkylthiocarbonyl, aralkylthiocarbonylthio, mercapto, alkylsulfonyl, aralkylsulfonyl and arylsulfonyl, and amino and amido radicals of the formula 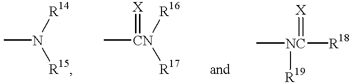
wherein X is oxygen atom or sulfur atom; wherein each of R¹⁴, R¹⁵, R¹⁶, R¹⁷, R¹⁸ and R¹⁹ is independently selected from hydrido, alkyl, cycloalkyl, cyano, hydroxyalkyl, cycloalkylalkyl, alkoxyalkyl, aralkyl and aryl;
and wherein each of R³ through R¹¹ may be further independently selected from acidic moieties of the formula
−YₙA
wherein n is a number selected from zero through three, inclusive; wherein A is an acidic group selected from acids containing one or more atoms selected from oxygen, sulfur, phosphorus and nitrogen atoms, and wherein said acidic group is selected to contain at least one acidic hydrogen atom, and the amide, ester and salt derivatives of said acidic moieties; wherein Y is a spacer group independently selected from one or more of alkyl, cycloalkyl, cycloalkylalkyl, alkenyl, alkynyl, aryl, aralkyl and heteroaryl having one or more ring atoms selected from oxygen, sulfur and nitrogen atoms;
and wherein any of the foregoing R¹ through R¹⁹, Y and A groups having a substitutable position may be substituted by one or more groups selected from hydroxy, alkyl, alkenyl, aralkyl, hydroxyalkyl, halo, haloalkyl, oxo, alkoxy, aryloxy, aralkoxy, alkoxyalkyl, alkylcarbonyl, alkoxycarbonyl, carboxyl, cyano, nitro, alkylsulfonyl, haloalkylsulfonyl, aryl, aralkyl, mercaptocarbonyl, alkylthio and alkylthiocarbonyl, and amino and amido radicals of the formula 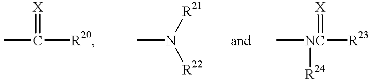
wherein X is oxygen atom or sulfur atom; wherein R¹⁹ is selected from hydrido, alkyl, cycloalkyl, cycloalkylalkyl, aralkyl, aryl, and DR²³ and 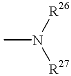
wherein D is selected from oxygen atom and sulfur atom, and R²⁵ is selected from hydrido, alkyl, cycloalkyl, cycloalkylalkyl, aralkyl and aryl; wherein each of R²¹, R²², R²³, R²⁴, R²⁶ and R²⁷ is independently selected from hydrido, alkyl, cycloalkyl, cyano, hydroxyalkyl, haloalkyl, cycloalkylalkyl, alkoxyalkyl, alkanoyl, alkoxycarbonyl, carboxyl, haloalkylsulfinyl, haloalkylsulfonyl, aralkyl and aryl;
or a tautomer thereof or a pharmaceutically-acceptable salt thereof.

15. The composition of claim 14 wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkoxycarbonyl, alkenyl, cycloalkenyl, alkynyl, cycloalkynyl, carboxyl, alkylsulfonyl, aralkylsulfonyl and arylsulfonyl, and amido radicals of the formula 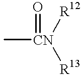
wherein each of R¹² and R¹³ is independently selected from hydrido, alkyl, cycloalkyl, cyano, hydroxyalkyl, cycloalkylalkyl, alkoxyalkyl, aralkyl and aryl;
wherein R² is selected from hydrido, alkyl, hydroxyalkyl, halo, haloalkyl, cycloalkyl, cycloalkylalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cycloalkynyl, carboxyl, alkylcarbonyloxy, alkylthio, arylthio, aralkylthio, aralkylthiocarbonylthio, alkylsulfonyl, aralkylsulfonyl and arylsulfonyl;
wherein each of R³ through R¹¹ is independently selected from hydrido, hydroxy, alkyl, hydroxyalkyl, halo, haloalkyl, cycloalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cyano, nitro, carboxyl, alkylcarbonyloxy, mercaptocarbonyl, alkoxycarbonyloxy, alkylthio, arylthio, aralkylthio, mercapto, alkylsulfonyl, aralkylsulfonyl and arylsulfonyl, and amino and amido radicals of the formula 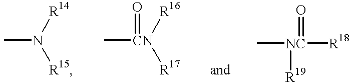
wherein each of R¹⁴, R¹⁵, R¹⁶, R¹⁷, R¹⁸ and R¹⁹ is independently selected from hydrido, alkyl, cycloalkyl, cyano, amino, monoalkylamino, dialkylamino, hydroxyalkyl, cycloalkylalkyl, alkoxyalkyl, aralkyl and aryl;
and wherein each of R³ through R¹¹ may be an acidic moiety further independently selected from acidic moieties of the formula
−YₙA
wherein n is a number selected from zero through three, inclusive;
wherein A is selected from carboxylic acid and bioisosteres of carboxylic acid selected from 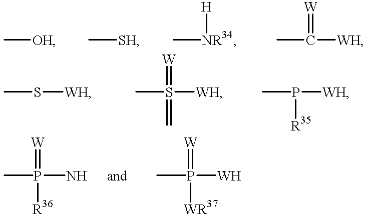
wherein each W is independently selected from oxygen atom, sulfur atom and NR³⁸; wherein each of R³⁴, R³⁵, R³⁶, R³⁷ and R³⁸ is independently selected from hydrido, alkyl, haloalkyl, haloalkylsulfonyl, haloalkylcarbonyl, cycloalkyl, cycloalkylalkyl, aryl and aralkyl; wherein each of R³⁴, R³⁵, R³⁶ and R³⁷ may be further independently selected from amino radical of the formula 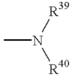
wherein each of R³⁹ and R⁴⁰ is independently selected from hydrido, alkyl, cycloalkyl, hydroxyalkyl, haloalkyl, cycloalkylalkyl, alkoxyalkyl, aralkyl and aryl, and wherein R³⁹ and R⁴⁰ taken together may form a heterocyclic group having five to seven ring members including the nitrogen atom of said amino radical, which heterocyclic group may further contain one or more hetero atoms as ring members selected from oxygen, nitrogen and sulfur atoms and which heterocyclic group may be saturated or partially unsaturated; wherein R³⁹ and R⁴⁰ taken together may form an aromatic heterocyclic group having five ring members including the nitrogen atom of said amino radical and which aromatic heterocyclic group may further contain one or more hetero atoms as ring atoms selected from oxygen, nitrogen and sulfur atoms; wherein each of R³⁵ and R³⁶ may be further independently selected from hydroxy, alkoxy, alkylthio, aryloxy, arylthio, aralkylthio and aralkoxy; and the amide, ester and salt derivatives of said acidic groups;
wherein said bioisostere of carboxylic acid may be further selected from heterocyclic acidic groups consisting of heterocyclic rings of four to about nine ring members, which heterocyclic ring contains at least one hetero atom selected from oxygen, sulfur and nitrogen atoms, which heterocyclic ring may be saturated, fully unsaturated or partially unsaturated, and which heterocyclic ring may be attached at a single position selected from R³ through R¹¹ or may be attached at any two adjacent positions selected from R³ through R¹¹ so as to form a fused-ring system with one of the phenyl rings of Formula I; and the amide, ester and salt derivatives of said heterocyclic acidic groups;
wherein Y is a spacer group independently selected from one or more of alkyl, cycloalkyl, cycloalkylalkyl, alkenyl, aryl and aralkyl;
and wherein any of the foregoing R¹ through R¹⁹ and R³⁴ through R⁴⁰, Y and A groups having a substitutable position may be substituted by one or more groups selected from hydroxy, alkyl, alkenyl, aralkyl, hydroxyalkyl, halo, oxo, haloalkyl, alkoxy, aryloxy, aralkoxy, alkoxyalkyl, alkylcarbonyl, alkoxycarbonyl, carboxyl, cyano, nitro, alkylsulfonyl, haloalkylsulfonyl, aryl, aralkyl, mercaptocarbonyl, alkylthio and alkylthiocarbonyl, and amino and amido radicals of the formula 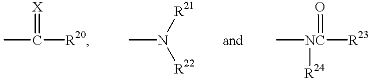
wherein X is selected from oxygen atom and sulfur atom; wherein R²⁰ is selected from hydrido, alkyl, cycloalkyl, cycloalkylalkyl, aralkyl, aryl and DR²⁵ and 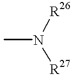
wherein D is selected from oxygen atom and sulfur atom, wherein R²⁵ is selected from hydrido, alkyl, cycloalkyl, cycloalkylalkyl, aralkyl and aryl;
wherein each of R²¹, R²², R²³, R²⁴, R²⁶ and R²⁷ is independently selected from hydrido, alkyl, cycloalkyl, cyano, hydroxyalkyl, haloalkyl, cycloalkylalkyl, alkoxyalkyl, alkanoyl, alkoxycarbonyl, carboxyl, haloalkylsulfinyl, haloalkylsulfonyl, aralkyl and aryl;
or a tautomer thereof or a pharmaceutically-acceptable salt thereof.

16. The composition of claim 15 wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkenyl, cycloalkenyl, alkynyl, mercaptocarbonyl, alkylsulfonyl, aralkylsulfonyl, arylsulfonyl and amido radicals of the formula 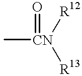
wherein each of R¹² and R¹³ is independently selected from hydrido, alkyl, cycloalkyl, cyano, amino, monoalkylamino, dialkylamino, hydroxyalkyl, cycloalkylalkyl, alkoxyalkyl, aralkyl and aryl;
wherein each of R² is selected from hydrido, alkyl, hydroxyalkyl, halo, halooalkyl, cycloalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, carboxyl, alkylcarbonyloxy, alkylthio, arylthio, aralkylthio, alkylsulfonyl, aralkylsulfonyl and arylsulfonyl;
wherein each of R³ through R¹¹ is independently selected from hydrido, hydroxy, alkyl, hydroxyalkyl, halo, haloalkyl, cycloalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cyano, nitro, carboxyl, alkylthio, aralkylthio and mercapto;
and wherein each of R³ through R¹¹ may be an acidic moiety further independently selected from acidic moieties of the formula
−YₙA
wherein n is a number selected from zero through three, inclusive; wherein A is selected from carboxylic acid and bioisosteres of carboxylic acid selected from 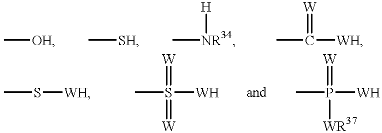
wherein each W is independently selected from oxygen atom, sulfur atom and NR³⁸; wherein each of R³⁴, R³⁷ and R³⁸ is independently selected from hydrido, alkyl, haloalkyl, haloalkylsulfonyl, haloalkylcarbonyl, cycloalkyl, cycloalkylalkyl, aryl and aralkyl; wherein each of R³⁴ and R³⁷ may be further independently selected from amino radical of the formula 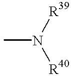
wherein each of R³⁹ and R⁴⁰ is independently selected from hydrido, alkyl, cycloalkyl, hydroxyalkyl, haloalkyl, cycloalkylalkyl, alkoxyalkyl, aralkyl and aryl, and wherein R³⁹ and R⁴⁰ taken together may form a heterocyclic group having five to seven ring members including the nitrogen atom of said amino radical, which heterocyclic group may further contain one or more hetero atoms as ring members selected from oxygen, nitrogen and sulfur atoms, and which heterocyclic group may be saturated or partially unsaturated; wherein R³⁹ and R⁴⁰ taken together may form an aromatic heterocyclic group having five ring members including the nitrogen atom of said amino radical and which aromatic heterocyclic group may further contain one or more hetero atoms as ring atoms selected from oxygen, nitrogen and sulfur atoms; and the amide, ester and salt derivatives of said acidic groups; wherein said bioisostere of carboxylic acid may be further selected from heterocyclic acidic groups consisting of heterocyclic rings of four to about nine ring members, which ring contains at least one hetero atom, selected from oxygen, sulfur and nitrogen atoms, which heterocyclic ring may be saturated, fully unsaturated or partially unsaturated, and which heterocyclic ring may be attached at a single position selected from R³ through R¹¹ or may be attached at any two adjacent positions selected from R³ through R¹ so as to form a fused-ring system with one of the phenyl rings of Formula I; and the amide, ester and salt derivatives of said heterocyclic acidic groups;
wherein Y is a spacer group independently selected from one or more of alkyl, cycloalkyl, cycloalkylalkyl, alkenyl, aryl and aralkyl;
wherein each of R¹ through R¹⁹, R³⁴ and R³⁷ through R⁴⁰, Y and A independently may be substituted at any substitutable position with one or more groups selected from alkyl, hydroxy, halo, oxo, haloalkyl, alkoxycarbonyl, cyano, nitro, alkylsulfonyl, haloalkylsulfonyl, aryl, aralkyl, alkoxy, aryloxy and aralkoxy;
or a tautomer thereof or a pharmaceutically-acceptable salt thereof.

17. The composition of claim 16 wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkenyl, alkynyl, alkylsulfonyl, aralkylsulfonyl, arylsulfonyl and amido radicals of the formula 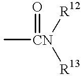
wherein each of R¹² and R¹³ is independently selected from hydrido, alkyl, cycloalkyl, cyano, amino, hydroxyalkyl, alkoxyalkyl, phenalkyl and phenyl;
wherein R² is selected from hydrido, alkyl, hydroxyalkyl, halo, haloalkyl, cycloalkyl, alkoxy, aralkyl, aryl, aryloxy, aralkoxy, alkoxyalkyl, alkylcarbonyl, alkoxycarbonyl, alkenyl, cycloalkenyl, alkynyl, alkylthio, arylthio, aralkylthio and arylsulfonyl,
wherein each of R³ through R¹¹ is independently selected from hydrido, hydroxy, alkyl, hydroxyalkyl, halo, haloalkyl, cycloalkyl, alkoxy, phenalkyl, phenyl, benzoyl, phenoxy, phenalkyloxy, alkoxyalkyl, alkylcarbonyl, alkoxycarbonyl, alkenyl, cyano, nitro, carboxyl, alkylthio and mercapto;
and wherein each of R³ through R¹¹ may be an acidic moiety further independently selected from acidic moieties of the formula
−YₙA
wherein n is a number selected from zero through two, inclusive; wherein A is selected from carboxylic acid and bioisosteres of carboxylic acid selected from 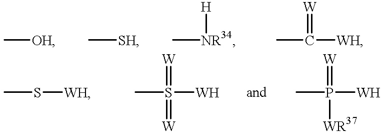
wherein each W is independently selected from oxygen atom, sulfur atom and NR³⁸; wherein each of R³⁴, R³⁷ and R³⁸ is independently selected from hydrido, alkyl, haloalkyl, haloalkylsulfonyl, haloalkylcarbonyl, cycloalkyl, phenyl and benzyl; wherein each of R³⁴ and R³⁷ may be further independently selected from amino radical of the formula 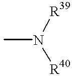
wherein each of R³⁹ and R⁴⁰ is independently selected from hydrido, alkyl, cycloalkyl, hydroxyalkyl, haloalkyl, alkoxyalkyl, benzyl and phenyl; and the amide, ester and salt derivatives of said acidic groups;
wherein said bioisostere of carboxylic acid may be further selected from heterocyclic aci dic groups consisting of heterocyclic rings of four to about nine ring members, which ring contains at least one hetero atom, selected from oxygen, sulfur and nitrogen atoms, which heterocyclic ring may be saturated, fully unsaturated or partially unsaturated, and which heterocyclic ring may be attached at a single position selected from R³ through R¹¹ or may be attached at any two adjacent positions selected from R³ through R¹¹ so as to form a fused-ring system with one of the phenyl rings of Formula I; and the amide, ester and salt derivatives of said heterocyclic acidic groups;
wherein Y is a spacer group independently selected from one or more of alkyl, cycloalkyl, cycloalkylalkyl, alkenyl, phenyl, phenalkyl and aralkyl;
wherein each of R¹ through R¹⁹, R³⁴ and R³⁷ through R⁴⁰, Y and A and independently may be substituted at any substitutable position with one or more groups selected from alkyl, cycloalkyl, cycloalkylalkyl, hydroxy, halo, oxo, haloalkyl, alkoxycarbonyl, cyano, nitro, alkylsulfonyl, haloalkylsulfonyl, aryl, aralkyl, alkoxy, aryloxy and aralkoxy;
or a tautomer thereof or a pharmaceutically-acceptable salt thereof.

18. The composition of claim 17 wherein m is one; wherein R¹ is selected from alkyl, hydroxyalkyl, haloalkyl, cycloalkyl, cycloalkylalkyl, aralkyl, aryl, benzoyl, alkoxyalkyl, alkylcarbonyl, alkoxycarbonyl, alkenyl and alkynyl;
where R² is selected from alkyl, hydroxyalkyl, cycloalkyl, haloalkyl, alkoxy, phenalkyl, phenyl, phenoxy, phenalkoxy, alkoxyalkyl, alkylcarbonyl, alkoxycarbonyl, alkenyl, cycloalkenyl, alkynyl, alkylthio, phenthio and phenalkylthio;
wherein each of R³ through R¹¹ is independently selected from hydrido, hydroxy, alkyl, hydroxyalkyl, halo, haloalkyl, alkoxy, phenyl, benzoyl, phenoxy, alkoxyalkyl, acetyl, alkoxycarbonyl, alkenyl, cyano, nitro, carboxyl, alkylthio and mercapto;
and wherein each of R³ through R¹¹ may be an acidic moiety further independently selected from acidic moieties consisting of CO₂H, CO₂CH₃, SH, CH₂SH, C₂H₄SH, PO₃H₂, NHSO₂CF₃, NHSO₂C₆F₅, SO₃H, CONHNH₂, CONHNHSO₂CF₃, CONHOCH₃, CONHOC₂H₅, CONHCF₃, OH, CH₂OH, C₂H₄OH, OPO₃H₂, OSO₃H 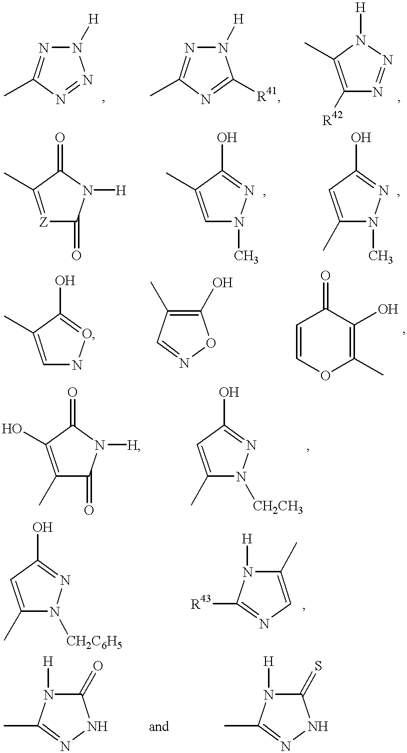
wherein each of R⁴¹, R⁴² and R⁴³ is independently selected from H, Cl, CN, NO₂, CF₃, C₂F₅, C₃F₇, CHF₂, CH₂F, CO₂CH₃, CO₂C₂H₅, SO₂CH₃, SO₂CF₃ and SO₂C₆F₅; wherein Z is selected from O, S, NR⁴⁴ and CH₂; wherein R⁴⁴ is selected from hydrido, CH₃ and CH₂C₆H₅; and wherein said acidic moiety may be a heterocyclic acidic group attached at any two adjacent positions of R³ through R¹¹ so as to form a fused ring system with one of the phenyl rings of the biphenyl moiety of Formula I, said biphenyl fused ring system selected from 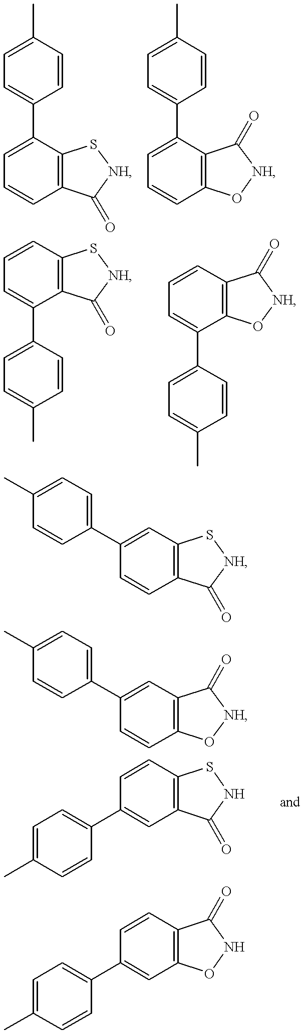
and the esters, amides and salts of said acidic moieties; or a tautomer thereof or a pharmaceutically-acceptable salt thereof.

19. The composition of claim 18 wherein m is one; wherein R¹ is selected from methyl, ethyl, n-propyl, isopropyl, n-butyl, sec-butyl, isobutyl, tert-butyl, n-pentyl, isopentyl, neopentyl, phenyl, benzyl, phenethyl, cyclohexyl, cyclohexylmethyl, 1-oxoethyl, 1-oxopropyl, 1-oxobutyl, 1-oxopentyl and hydroxyalkyl; wherein R² is selected from hydroxy, methyl, ethyl, n-propyl, isopropyl, n-butyl, sec-butyl, isobutyl, tert-butyl, n-pentyl, isopentyl, neopentyl, phenyl, benzyl, phenethyl, cyclohexyl, cyclohexylmethyl, propylthio, butylthio, 1-oxoethyl, 1-oxopropyl, 1-oxobutyl, 1-oxopentyl, 1,1-dimethoxypropyl, 1,1-dimethoxybutyl, 1,1-dimethoxypentyl, hydroxyalkyl, difluoromethyl, 1,1-difluoroethyl, 1,1-difluoropropyl, 1,1-difluorobutyl and 1,1-difluoropentyl; wherein at least one of R⁵, R⁶, R⁸ and R⁹ is an acidic group selected from CO₂H, SH, PO₃H₂, SO₃H, CONHNH₂, CONHNHSO₂CF₃, OH, 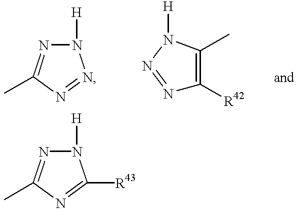
wherein each of R⁴² and R⁴³ is independently selected from Cl, CN, NO₂, CF₃, CO₂CH₃ and SO₂CF₃;
or a tautomer thereof or a pharmaceutically-acceptable salt thereof.

20. The composition of claim 19 wherein m is one; wherein R¹ is selected from methyl, ethyl, n-propyl, isopropyl, n-butyl, sec-butyl, isobutyl, tert-butyl, 4-methylbutyl, n-pentyl, neopentyl, phenyl, benzyl, phenethyl, cyclohexyl, cyclohexylmethyl, 1-oxoethyl, 1-oxopropyl, 1-oxobutyl, 1-oxopentyl and hydroxyalkyl; wherein R² is selected from ethyl, n-propyl, isopropyl, n-butyl, sec-butyl, isobutyl, 4-methylbutyl, tert-butyl, n-pentyl, neopentyl, propylthio and butylthio; wherein at least one of R⁵, R⁶, R⁸ and R⁹ is an acidic group selected from CO₂H, SH, PO₃H₂, SO₃H, CONHNH₂, CONHNHSO₂CF₃, OH, 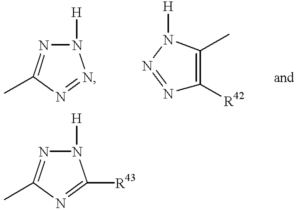
wherein each of R⁴² and R⁴³ is independently selected from Cl, CN, NO₂, CF₃, CO₂CH₃ and SO₂CF₃;
or a tautomer thereof or a pharmaceutically-acceptable salt thereof.

21. The composition of claim 20 wherein m is one; wherein R¹ is selected from methyl, ethyl, n-propyl, isopropyl, n-butyl, sec-butyl, isobutyl, tert-butyl, 4-methylbutyl, n-pentyl, neopentyl, phenyl, benzyl, phenethyl, cyclohexyl, cyclohexylmethyl and hydroxyalkyl; wherein R² is selected from ethyl, n-propyl, isopropyl, n-butyl, sec-butyl, isobutyl, 4-methylbutyl, n-pentyl, propylthio and butylthio; wherein each of R³, R⁴, R⁶, R⁷, R⁸, R¹⁰ and R¹¹ is hydrido; wherein one of R⁵ and R⁹ is hydrido and the other of R⁵ and R⁹ is an acidic group selected from CO₂H and 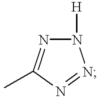
or a tautomer thereof or a pharmaceutically-acceptable salt thereof.

22. The composition of claim 21 wherein said antagonist compound is 4′-[(1,3-dibutyl-4,5-dihydro-5-oxo-1H-1,2,4-triazol-4-yl) methyl] [1,1′-biphenyl]-2-carboxylic acid.

23. The composition of claim 21 wherein said antagonist compound is
2,5-dibutyl-2,4-dihydro-4-[[2-(1H-tetrazol-5-yl) [1,1′-biphenyl]-4′-yl]methyl]-3H-1,2,4-triazol-3-one.

24. A therapeutic method for treating a circulatory disorder, said method comprising administering to a subject having such disorder a therapeutically-effective amount of a compound of Formula I: 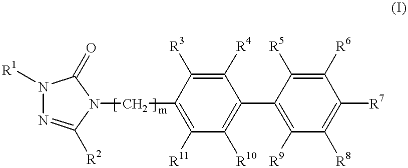
wherein m is a number selected from one to four, inclusive;
wherein R¹ is selected from alkyl, hydroxyalkyl, haloalkyl, cycloalkyl, cycloalkylalkyl, formyl, aralkyl, aryl, aroyl, alkoxyalkyl, alkylcarbonyl, alkylcarbonylalkyl, alkoxycarbonyl, alkenyl, cycloalkenyl, aralkoxycarbonyl, alkynyl, cyano, carboxyl, mercaptocarbonyl, mercaptothiocarbonyl, alkylthiocarbonyl, alkylthiothiocarbonyl, arylthiocarbonyl, arylthiothiocarbonyl, aralkylthiocarbonyl, alkylthiocarbonyl, alkylsulfinyl, alkylsulfonyl, aralkylsulfinyl, aralkylsulfonyl, arylsulfinyl, arylsulfonyl, heteroaryl having one or more ring atoms selected from oxygen, sulfur and nitrogen atoms, and amido radicals of the formula 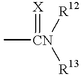
wherein X is oxygen atom or sulfur atom; wherein each of R¹² and R¹³ is independently selected from hydrido, alkyl, cycloalkyl, cyano, hydroxyalkyl, cycloalkylalkyl, alkoxyalkyl, aralkyl and aryl, and wherein R¹² and R¹³ taken together may form a heterocyclic group having five to seven ring members including the nitrogen atom of said amino or amido radical and which heterocyclic group may further contain one or more hetero atoms as ring members selected from oxygen, nitrogen and sulfur atoms and which heterocyclic group may be saturated or partially unsaturated; wherein each of R¹² and R¹³ taken together may form an aromatic heterocyclic group having five ring members including the nitrogen atom of said amino or amido radical and which aromatic heterocyclic group may further contain one or more hetero atoms as ring atoms selected from oxygen, nitrogen and sulfur atoms;
wherein each of R² through R¹¹ is independently selected from hydrido, alkyl, hydroxyalkyl, halo, haloalkyl, cycloalkyl, cycloalkylalkyl, formyl, alkoxy, aralkyl, aryl, aroyl, aryloxy, aralkoxy, aralkoxycarbonyl, alkoxyalkyl, alkylcarbonyl, alkylcarbonylalkyl, alkoxycarbonyl, alkenyl, cycloalkenyl, alkynyl, cyano, nitro, carboxyl, alkylcarbonyloxy, mercaptocarbonyl, mercaptothiocarbonyl, alkoxycarbonyloxy, alkylthio, alkylthiocarbonyl, alkylcarbonylthio, alkylthiocarbonyloxy, alkylthiocarbonylthio, alkylthiothiocarbonyl, alkylthiothiocarbonylthio, arylthio, arylthiocarbonyl, arylcarbonylthio, arylthiocarbonyloxy, arylthiocarbonylthio, arylthiothiocarbonyl, arylthiothiocarbonylthio, aralkylthio, aralkylthiocarbonyl, aralkylcarbonylthio, aralkylthiocarbonyloxy, aralkylthiocarbonylthio, alkylthiocarbonyl, aralkylthiocarbonylthio, mercapto, alkylsulfinyl, alkylsulfonyl, aralkylsulfinyl, aralkylsulfonyl, arylsulfinyl, arylsulfonyl, heteroaryl having one or more ring atoms selected from oxygen, sulfur and nitrogen atoms, and amino and amido radicals of the formula 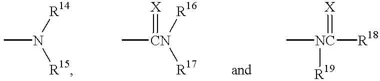
wherein X is oxygen atom or sulfur atom; wherein each of R¹⁴, R¹⁵, R¹⁶, R¹⁷, R¹⁸ and R¹⁹ is independently selected from hydrido, alkyl, cycloalkyl, cyano, hydroxyalkyl, cycloalkylalkyl, alkoxyalkyl, aralkyl and aryl, and wherein R¹⁴ and R¹⁵ taken together, R¹⁶ and R¹⁷ taken together and R¹⁸ and R¹⁹ taken together may form a heterocyclic group having five to seven ring members including the nitrogen atom of said amino or amido radical and which heterocyclic group may further contain one or more hetero atoms as ring members selected from oxygen, nitrogen and sulfur atoms and which heterocyclic group may be saturated or partially unsaturated; wherein each of R¹⁴ and R¹⁵ taken together and each of R¹⁶ and R¹⁷ taken together may form an aromatic heterocyclic group having five ring members including the nitrogen atom of said amino or amido radical and which aromatic heterocyclic group may further contain one or more hetero atoms as ring atoms selected from oxygen, nitrogen and sulfur atoms;
and wherein each of R³ through R¹¹ may be further independently selected from hydroxy and acidic moieties of the formula
−YₙA
wherein n is a number selected from zero through three, inclusive, and wherein A is an acidic group selected to contain at least one acidic hydrogen atom, and the amide, ester and salt derivatives of said acidic moieties; wherein Y is a spacer group independently selected from one or more of alkyl, cycloalkyl, cycloalkylalkyl, alkenyl, alkynyl, aryl, aralkyl and heteroaryl having one or more ring atoms selected from oxygen, sulfur and nitrogen atoms; and wherein any of the foregoing R¹ through R¹⁹, Y and A groups having a substitutable position may be substituted by one or more groups selected from hydroxy, alkyl, alkenyl, alkynyl, aralkyl, hydroxyalkyl, haloalkyl, halo, oxo, alkoxy, aryloxy, aralkoxy, aralkylthio, alkoxyalkyl, cycloalkyl, cycloalkylalkyl, aryl, aroyl, cycloalkenyl, cyano, cyanoamino, nitro, alkylcarbonyloxy, alkoxycarbonyloxy, alkylcarbonyl, alkoxycarbonyl, aralkoxycarbonyl, carboxyl, mercapto, mercaptocarbonyl, alkylthio, arylthio, alkylthiocarbonyl, alkylsulfinyl, alkylsulfonyl, haloalkylsulfonyl, aralkylsulfinyl, aralkylsulfonyl, arylsulfinyl, arylsulfonyl, heteroaryl having one or more ring atoms selected from oxygen, sulfur and nitrogen atoms, and amino and amido radicals of the formula 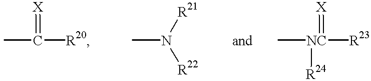
wherein X is oxygen atom or sulfur atom; wherein R²⁰ is selected from hydrido, alkyl, cycloalkyl, cycloalkylalkyl, aralkyl, aryl, DR²⁵ and 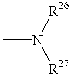
wherein D is selected from oxygen atom and sulfur atom and R²⁵ is selected from hydrido, alkyl, cycloalkyl, cycloalkylalkyl, aralkyl and aryl; wherein each of R²¹, R²², R²³, R²⁴, R²⁶ and R²⁷ is independently selected from hydrido, alkyl, cycloalkyl, cyano, hydroxyalkyl, haloalkyl, cycloalkylalkyl, alkoxyalkyl, alkylcarbonyl, alkoxycarbonyl, carboxyl, alkylsulfinyl, alkylsulfonyl, arylsulfinyl, arylsulfonyl, haloalkylsulfinyl, haloalkylsulfonyl, aralkyl and aryl, and wherein each of R²¹, R²², R²³, R²⁴, R²⁶ and R²⁷ is further independently selected from amino and amido radicals of the formula 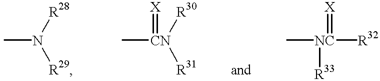
wherein each of R²⁸, R²⁹, R³⁰, R³¹ R³² and R³³ is independently selected from hydrido, alkyl, cycloalkyl, cyano, hydroxyalkyl, cycloalkylalkyl, alkoxyalkyl, haloalkylsulfinyl, haloalkylsulfonyl, aralkyl and aryl, and wherein each of R²¹ and R²² taken together and each of R²³ and R²⁴ taken together may form a heterocyclic group having five to seven ring members including the nitrogen atom of said amino or amido radical, which heterocyclic group may further contain one or more hetero atoms as ring members selected from oxygen, nitrogen and sulfur atoms and which heterocyclic group may be saturated or partially unsaturated; wherein each of R²¹ and R²² taken together and each of R²⁶ and R²⁷ taken together may form an aromatic heterocyclic group having five ring members including the nitrogen atom of said amino or amido radical and which aromatic heterocyclic group may further contain one or more hetero atoms as ring atoms selected from oxygen, nitrogen and sulfur atoms; or a tautomer thereof or a pharmaceutically-acceptable salt thereof.

25. The method of claim 24 wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkoxycarbonyl, alkenyl, cycloalkenyl, alkynyl, cycloalkynyl, carboxyl, mercaptocarbonyl, mercaptothiocarbonyl, alkylthiocarbonyl, arylthiocarbonyl, arylthiothiocarbonyl, aralkylthiocarbonyl, alkylsulfonyl, aralkylsulfonyl, arylsulfonyl, heteroaryl having one or more ring atoms selected from oxygen, sulfur and nitrogen atoms, and amido radicals of the formula 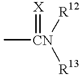
wherein X is oxygen atom or sulfur atom; wherein each of R¹² and R¹³ is independently selected from hydrido, alkyl, cycloalkyl, cyano, hydroxyalkyl, cycloalkylalkyl, alkoxyalkyl, aralkyl and aryl;
wherein R² is selected from hydrido, alkyl, hydroxyalkyl, halo, haloalkyl, cycloalkyl, cycloalkylalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cycloalkynyl, cyano, carboxyl, alkylcarbonyloxy, alkylthio, alkylthiocarbonyl, alkylcarbonylthio, alkylthiocarbonyloxy, alkylthiocarbonylthio, alkylthiothiocarbonyl, alkylthiothiocarbonylthio, arylthio, arylthiocarbonyl, arylcarbonylthio, arylthiocarbonyloxy, arylthiocarbonylthio, arylthiothiocarbonyl, arylthiothiocarbonylthio, aralkylthio, aralkylthiocarbonyl, aralkylcarbonylthio, aralkylthiocarbonyloxy, aralkylthiocarbonylthio, aralkylthiocarbonyl, aralkylthiocarbonylthio, mercapto, alkylsulfonyl, aralkylsulfonyl, arylsulfonyl and heteroaryl having one or more ring atoms selected from oxygen, sulfur and nitrogen atoms;
wherein each of R³ through R¹¹ is independently selected from hydrido, hydroxy, alkyl, hydroxyalkyl, halo, haloalkyl, cycloalkyl, cycloalkylalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cycloalkynyl, cyano, nitro, carboxyl, alkylcarbonyloxy, mercaptocarbonyl, mercaptothiocarbonyl, alkoxycarbonyloxy, alkylthio, alkylthiocarbonyl, alkylcarbonylthio, alkylthiocarbonyloxy, alkylthiocarbonylthio, alkylthiothiocarbonyl, arylthio, arylthiocarbonyl, arylcarbonylthio, arylthiocarbonyloxy, arylthiothiocarbonyl, aralkylthio, aralkylthiocarbonyl, aralkylcarbonylthio, aralkylthiocarbonyloxy, aralkylthiocarbonylthio, aralkylthiocarbonyl, aralkylthiocarbonylthio, mercapto, alkylsulfonyl, aralkylsulfonyl and arylsulfonyl, and amino and amido radicals of the formula 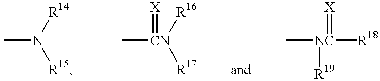
wherein X is oxygen atom or sulfur atom; wherein each of R¹⁴, R¹⁵, R¹⁶, R¹⁷, R¹⁸ and R¹⁹ is independently selected from hydrido, alkyl, cycloalkyl, cyano, hydroxyalkyl, cycloalkylalkyl, alkoxyalkyl, aralkyl and aryl;
and wherein each of R³ through R¹¹ may be further independently selected from acidic moieties of the formula
−YₙA
wherein n is a number selected from zero through three, inclusive; wherein A is an acidic group selected from acids containing one or more atoms selected from oxygen, sulfur, phosphorus and nitrogen atoms, and wherein said acidic group is selected to contain at least one acidic hydrogen atom, and the amide, ester and salt derivatives of said acidic moieties; wherein Y is a spacer group independently selected from one or more of alkyl, cycloalkyl, cycloalkylalkyl, alkenyl, alkynyl, aryl, aralkyl and heteroaryl having one or more ring atoms selected from oxygen, sulfur and nitrogen atoms;
and wherein any of the foregoing R¹ through R¹⁹, Y and A groups having a substitutable position may be substituted by one or more groups selected from hydroxy, alkyl, alkenyl, aralkyl, hydroxyalkyl, halo, haloalkyl, oxo, alkoxy, aryloxy, aralkoxy, alkoxyalkyl, alkylcarbonyl, alkoxycarbonyl, carboxyl, cyano, nitro, alkylsulfonyl, haloalkylsulfonyl, aryl, aralkyl, mercaptocarbonyl, alkylthio and alkylthiocarbonyl, and amino and amido radicals of the formula 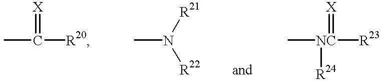
wherein X is oxygen atom or sulfur atom; wherein R¹⁹ is selected from hydrido, alkyl, cycloalkyl, cycloalkylalkyl, aralkyl, aryl, and DR²³ and 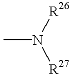
wherein D is selected from oxygen atom and sulfur atom, and R²⁵ is selected from hydrido, alkyl, cycloalkyl, cycloalkylalkyl, aralkyl and aryl; wherein each of R²¹, R²², R²³, R²⁴, R²⁶ and R²⁷ is independently selected from hydrido, alkyl, cycloalkyl, cyano, hydroxyalkyl, haloalkyl, cycloalkylalkyl, alkoxyalkyl, alkanoyl, alkoxycarbonyl, carboxyl, haloalkylsulfinyl, haloalkylsulfonyl, aralkyl and aryl;
or a tautomer thereof or a pharmaceutically-acceptable salt thereof.

26. The method of claim 25 wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkoxycarbonyl, alkenyl, cycloalkenyl, alkynyl, cycloalkynyl, carboxyl, alkylsulfonyl, aralkylsulfonyl and arylsulfonyl, and amido radicals of the formula 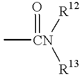
wherein each of R¹² and R¹³ is independently selected from hydrido, alkyl, cycloalkyl, cyano, hydroxyalkyl, cycloalkylalkyl, alkoxyalkyl, aralkyl and aryl;
wherein R² is selected from hydrido, alkyl, hydroxyalkyl, halo, haloalkyl, cycloalkyl, cycloalkylalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cycloalkynyl, carboxyl, alkylcarbonyloxy, alkylthio, arylthio, aralkylthio, aralkylthiocarbonylthio, alkylsulfonyl, aralkylsulfonyl and arylsulfonyl;
wherein each of R³ through R¹¹ is independently selected from hydrido, hydroxy, alkyl, hydroxyalkyl, halo, haloalkyl, cycloalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cyano, nitro, carboxyl, alkylcarbonyloxy, mercaptocarbonyl, alkoxycarbonyloxy, alkylthio, arylthio, aralkylthio, mercapto, alkylsulfonyl, aralkylsulfonyl and arylsulfonyl, and amino and amido radicals of the formula 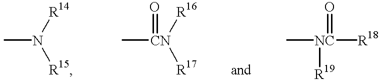
wherein each of R¹⁴, R¹⁵, R¹⁶, R¹⁷, R¹⁸ and R¹⁹ is independently selected from hydrido, alkyl, cycloalkyl, cyano, amino, monoalkylamino, dialkylamino, hydroxyalkyl, cycloalkylalkyl, alkoxyalkyl′, aralkyl and aryl;
and wherein each of R³ through R¹¹ may be an acidic moiety further independently selected from acidic moieties of the formula
−YₙA
wherein n is a number selected from zero through three, inclusive;
wherein A is selected from carboxylic acid and bioisosteres of carboxylic acid selected from 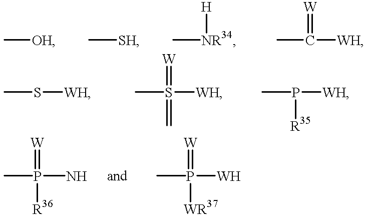
wherein each W is independently selected from oxygen atom, sulfur atom and NR³⁸; wherein each of R³⁴, R³⁵, R³⁶, R³⁷ and R³⁸ is independently selected from hydrido, alkyl, haloalkyl, haloalkylsulfonyl, haloalkylcarbonyl, cycloalkyl, cycloalkylalkyl, aryl and aralkyl; wherein each of R³⁴, R³⁵, R³⁶ and R³⁷ may be further independently selected from amino radical of the formula 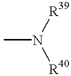
wherein each of R³⁹ and R⁴⁰ is independently selected from hydrido, alkyl, cycloalkyl, hydroxyalkyl, haloalkyl, cycloalkylalkyl, alkoxyalkyl, aralkyl and aryl, and wherein R³⁹ and R⁴⁰ taken together may form a heterocyclic group having five to seven ring members including the nitrogen atom of said amino radical, which heterocyclic group may further contain one or more hetero atoms as ring members selected from oxygen, nitrogen and sulfur atoms and which heterocyclic group may be saturated or partially unsaturated; wherein R³⁹ and R⁴⁰ taken together may form an aromatic heterocyclic group having five ring members including the nitrogen atom of said amino radical and which aromatic heterocyclic group may further contain one or more hetero atoms as ring atoms selected from oxygen, nitrogen and sulfur atoms; wherein each of R³⁵ and R³⁶ may be further independently selected from hydroxy, alkoxy, alkylthio, aryloxy, arylthio, aralkylthio and aralkoxy; and the amide, ester and salt derivatives of said acidic groups;
wherein said bioisostere of carboxylic acid may be further selected from heterocyclic acidic groups consisting of heterocyclic rings of four to about nine ring members, which heterocyclic ring contains at least one hetero atom selected from oxygen, sulfur and nitrogen atoms, which heterocyclic ring may be saturated, fully unsaturated or partially unsaturated, and which heterocyclic ring may be attached at a single position selected from R³ through R¹¹ or may be attached at any two adjacent positions selected from R³ through Rl¹ so as to form a fused-ring system with one of the phenyl rings of Formula I; and the amide, ester and salt derivatives of said heterocyclic acidic groups;
wherein Y is a spacer group independently selected from one or more of alkyl, cycloalkyl, cycloalkylalkyl, alkenyl, aryl and aralkyl;
and wherein any of the foregoing R¹ through R¹⁹ and R³⁴ through R⁴⁰, Y and A groups having a substitutable position may be substituted by one or more groups selected from hydroxy, alkyl, alkenyl, aralkyl, hydroxyalkyl, halo, oxo, haloalkyl, alkoxy, aryloxy, aralkoxy, alkoxyalkyl, alkylcarbonyl, alkoxycarbonyl, carboxyl, cyano, nitro, alkylsulfonyl, haloalkylsulfonyl, aryl, aralkyl, mercaptocarbonyl, alkylthio and alkylthiocarbonyl, and amino and amido radicals of the formula 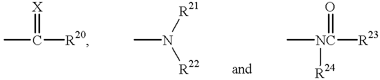
wherein X is selected from oxygen atom and sulfur atom; wherein R²⁰ is selected from hydrido, alkyl, cycloalkyl, cycloalkylalkyl, aralkyl, aryl and DR²⁵ and 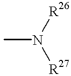
wherein D is selected from oxygen atom and sulfur atom, wherein R²⁵ is selected from hydrido, alkyl, cycloalkyl, cycloalkylalkyl, aralkyl and aryl;
wherein each of R²¹, R²², R²³, R²⁴, R²⁶ and R²⁷ is independently selected from hydrido, alkyl, cycloalkyl, cyano, hydroxyalkyl, haloalkyl, cycloalkylalkyl, alkoxyalkyl, alkanoyl, alkoxycarbonyl, carboxyl, haloalkylsulfinyl, haloalkylsulfonyl, aralkyl and aryl;
or a tautomer thereof or a pharmaceutically-acceptable salt thereof.

27. The method of claim 26 wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkenyl, cycloalkenyl, alkynyl, mercaptocarbonyl, alkylsulfonyl, aralkylsulfonyl, arylsulfonyl and amido radicals of the formula 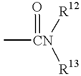
wherein each of R¹² and R¹³ is independently selected from hydrido, alkyl, cycloalkyl, cyano, amino, monoalkylamino, dialkylamino, hydroxyalkyl, cycloalkylalkyl, alkoxyalkyl, aralkyl and aryl;
wherein each of R² is selected from hydrido, alkyl, hydroxyalkyl, halo, halooalkyl, cycloalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, carboxyl, alkylcarbonyloxy, alkylthio, arylthio, aralkylthio, alkylsulfonyl, aralkylsulfonyl and arylsulfonyl;
wherein each of R³ through R¹¹ is independently selected from hydrido, hydroxy, alkyl, hydroxyalkyl, halo, haloalkyl, cycloalkyl, alkoxy, aralkyl, aryl, aroyl, aryloxy, aralkoxy, alkoxyalkyl, alkylcarbonyl, alkoxycarbonyl, alkenyl, cycloalkenyl, alkynyl, cyano, nitro, carboxyl, alkylthio, aralkylthio and mercapto;
and wherein each of R³ through R¹¹ may be an acidic moiety further independently selected from acidic moieties of the formula
−YₙA
wherein n is a number selected from zero through three, inclusive; wherein A is selected from carboxylic acid and bioisosteres of carboxylic acid selected from 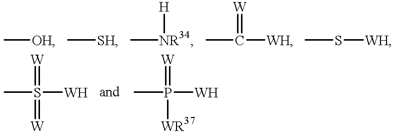
wherein each W is independently selected from oxygen atom, sulfur atom and NR³⁸; wherein each of R³⁴, R³⁷ and R³⁸ is independently selected from hydrido, alkyl, haloalkyl, haloalkylsulfonyl, haloalkylcarbonyl, cycloalkyl, cycloalkylalkyl, aryl and aralkyl; wherein each of R³⁴ and R³⁷ may be further independently selected from amino radical of the formula 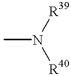
wherein each of R³⁹ and R⁴⁰ is independently selected from hydrido, alkyl, cycloalkyl, hydroxyalkyl, haloalkyl, cycloalkylalkyl, alkoxyalkyl, aralkyl and aryl, and wherein R³⁹ and R⁴⁰ taken together may form a heterocyclic group having five to seven ring members including the nitrogen atom of said amino radical, which heterocyclic group may further contain one or more hetero atoms as ring members selected from oxygen, nitrogen and sulfur atoms, and which heterocyclic group may be saturated or partially unsaturated; wherein R³⁹ and R⁴⁰ taken together may form an aromatic heterocyclic group having five ring members including the nitrogen atom of said amino radical and which aromatic heterocyclic group may further contain one or more hetero atoms as ring atoms selected from oxygen, nitrogen and sulfur atoms; and the amide, ester and salt derivatives of said acidic groups; wherein said bioisostere of carboxylic acid may be further selected from heterocyclic acidic groups consisting of heterocyclic rings of four to about nine ring members, which ring contains at least one hetero atom, selected from oxygen, sulfur and nitrogen atoms, which heterocyclic ring may be saturated, fully unsaturated or partially unsaturated, and which heterocyclic ring may be attached at a single position selected from R³ through R¹ or may be attached at any two adjacent positions selected from R³ through R¹¹ so as to form a fused-ring system with one of the phenyl rings of Formula I; and the amide, ester and salt derivatives of said heterocyclic acidic groups;
wherein Y is a spacer group independently selected from one or more of alkyl, cycloalkyl, cycloalkylalkyl, alkenyl, aryl and aralkyl;
wherein each of R¹ through R¹⁹, R³⁴ and R³⁷ through R⁴⁰, Y and A independently may be substituted at any substitutable position with one or more groups selected from alkyl, hydroxy, halo, oxo, haloalkyl, alkoxycarbonyl, cyano, nitro, alkylsulfonyl, haloalkylsulfonyl, aryl, aralkyl, alkoxy, aryloxy and aralkoxy;
or a tautomer thereof or a pharmaceutically-acceptable salt thereof.

28. The method of claim 27 wherein m is one; wherein R¹ is selected from alkyl, hydroxyalkyl, haloalkyl, cycloalkyl, cycloalkylalkyl, aralkyl, aryl, aroyl, alkoxyalkyl, alkylcarbonyl, alkenyl, alkynyl, alkylsulfonyl, aralkylsulfonyl, arylsulfonyl and amido radicals of the formula 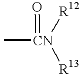
wherein each of R¹² and R¹³ is independently selected from hydrido, alkyl, cycloalkyl, cyano, amino, hydroxyalkyl, alkoxyalkyl, phenalkyl and phenyl;
wherein R² is selected from hydrido, alkyl, hydroxyalkyl, halo, haloalkyl, cycloalkyl, alkoxy, aralkyl, aryl, aryloxy, aralkoxy, alkoxyalkyl, alkylcarbonyl, alkoxycarbonyl, alkenyl, cycloalkenyl, alkynyl, alkylthio, arylthio, aralkylthio and arylsulfonyl,
wherein each of R³ through R¹¹ is independently selected from hydrido, hydroxy, alkyl, hydroxyalkyl, halo, haloalkyl, cycloalkyl, alkoxy, phenalkyl, phenyl, benzoyl, phenoxy, phenalkyloxy, alkoxyalkyl, alkylcarbonyl, alkoxycarbonyl, alkenyl, cyano, nitro, carboxyl, alkylthio and mercapto;
and wherein each of R³ through R¹¹ may be an acidic moiety further independently selected from acidic moieties of the formula
−Yₙ
wherein n is a number selected from zero through two, inclusive; wherein A is selected from carboxylic acid and bioisosteres of carboxylic acid selected from 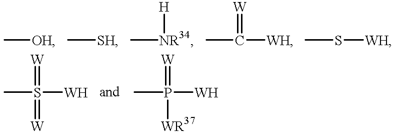
wherein each W is independently selected from oxygen atom, sulfur atom and NR³⁸; wherein each of R³⁴, R³⁷ and R³⁸ is independently selected from hydrido, alkyl, haloalkyl, haloalkylsulfonyl, haloalkylcarbonyl, cycloalkyl, phenyl and benzyl; wherein each of R³⁴ and R³⁷ may be further independently selected from amino radical of the formula 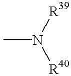
wherein each of R³⁹ and R⁴⁰ is independently selected from hydrido, alkyl, cycloalkyl, hydroxyalkyl, haloalkyl, alkoxyalkyl, benzyl and phenyl; and the amide, ester and salt derivatives of said acidic groups;
wherein said bioisostere of carboxylic acid may be further selected from heterocyclic acidic groups consisting of heterocyclic rings of four to about nine ring members, which ring contains at least one hetero atom, selected from oxygen, sulfur and nitrogen atoms, which heterocyclic ring may be saturated, fully unsaturated or partially unsaturated, and which heterocyclic ring may be attached at a single position selected from R³ through R¹¹ or may be attached at any two adjacent positions selected from R³ through R¹¹ so as to form a fused-ring system with one of the phenyl rings of Formula I; and the amide, ester and salt derivatives of said heterocyclic acidic groups;
wherein Y is a spacer group independently selected from one or more of alkyl, cycloalkyl, cycloalkylalkyl, alkenyl, phenyl, phenalkyl and aralkyl;
wherein each of R¹ through R¹⁹, R³⁴ and R³⁷ through R⁴⁰, Y and A and independently may be substituted at any substitutable position with one or more groups selected from alkyl, cycloalkyl, cycloalkylalkyl, hydroxy, halo, oxo, haloalkyl, alkoxycarbonyl, cyano, nitro, alkylsulfonyl, haloalkylsulfonyl, aryl, aralkyl, alkoxy, aryloxy and aralkoxy;
or a tautomer thereof or a pharmaceutically-acceptable salt thereof.

29. The method of claim 28 wherein m is one; wherein R¹ is selected from alkyl, hydroxyalkyl, haloalkyl, cycloalkyl, cycloalkylalkyl, aralkyl, aryl, benzoyl, alkoxyalkyl, alkylcarbonyl, alkoxycarbonyl, alkenyl and alkynyl;
where R² is selected from alkyl, hydroxyalkyl, cycloalkyl, haloalkyl, alkoxy, phenalkyl, phenyl, phenoxy, phenalkoxy, alkoxyalkyl, alkylcarbonyl, alkoxycarbonyl, alkenyl, cycloalkenyl, alkynyl, alkylthio, phenthio and phenalkylthio;
wherein each of R³ through R¹¹ is independently selected from hydrido, hydroxy, alkyl, hydroxyalkyl, halo, haloalkyl, alkoxy, phenyl, benzoyl, phenoxy, alkoxyalkyl, acetyl, alkoxycarbonyl, alkenyl, cyano, nitro, carboxyl, alkylthio and mercapto;
and wherein each of R³ through R¹¹ may be an acidic moiety further independently selected from acidic moieties consisting of CO₂H, CO₂CH₃, SH, CH₂SH, C₂H₄SH, PO₃H₂, NHSO₂CF3, NHSO₂C₆F₅, SO₃H, CONHNH₂, CONHNHSO₂CF₃, CONHOCH₃, CONHOC₂H₅, CONHCF₃, OH, CH₂OH, C₂H₄OH, OPO₃H₂, OSO₃H 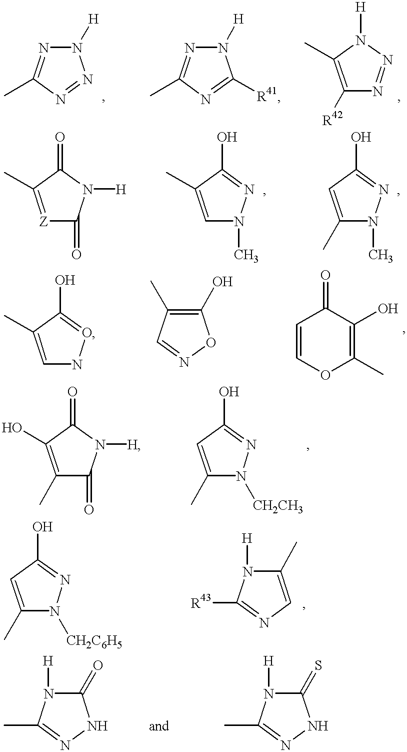
wherein each of R⁴¹, R⁴² and R⁴³ is independently selected from H, Cl, CN, NO₂₁, CF₃₁, C₂F₅, C₃F₇, CHF₂, CH₂F, CO₂CH₃, CO₂C₂H₅ , SO₂CH₃, SO₂CF₃ and SO₂C₆F₅ ; wherein Z is selected from O, S, NR⁴⁴ and CH₂; wherein R⁴⁴ is selected from hydrido, CH₃ and CH₂C₆H₅; and wherein said acidic moiety may be a heterocyclic acidic group attached at any two adjacent positions of R³ through R¹¹ so as to form a fused ring system with one of the phenyl rings of the biphenyl moiety of Formula I, said biphenyl fused ring system selected from 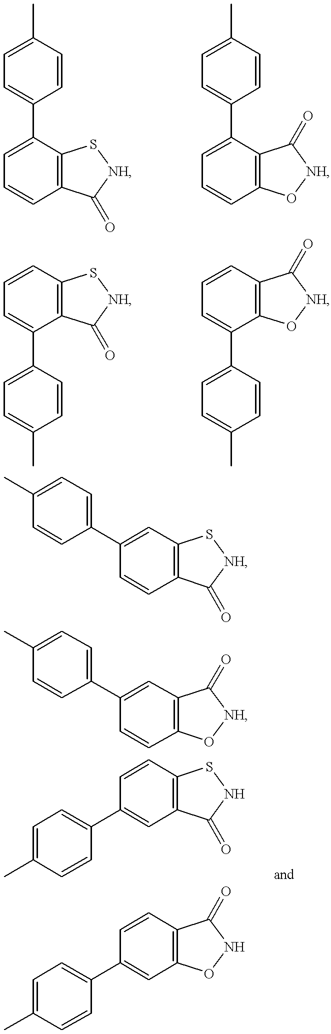
and the esters, amides and salts of said acidic moieties; or a tautomer thereof or a pharmaceutically-acceptable salt thereof.

30. The method of claim 29 wherein m is one; wherein R¹ is selected from methyl, ethyl, n-propyl, isopropyl, n-butyl, sec-butyl, isobutyl, tert-butyl, n-pentyl, isopentyl, neopentyl, phenyl, benzyl, phenethyl, cyclohexyl, cyclohexylmethyl, 1-oxoethyl, 1-oxopropyl, 1-oxobutyl, 1-oxopentyl and hydroxyalkyl; wherein R² is selected from hydroxy, methyl, ethyl, n-propyl, isopropyl, n-butyl, sec-butyl, isobutyl, tert-butyl, n-pentyl, isopentyl, neopentyl, phenyl, benzyl, phenethyl, cyclohexyl, cyclohexylmethyl, propylthio, butylthio, 1-oxoethyl, 1-oxopropyl, 1-oxobutyl, 1-oxopentyl, 1,1-dimethoxypropyl, 1,1-dimethoxybutyl, 1,1-dimethoxypentyl, hydroxyalkyl, difluoromethyl, 1,1-difluoroethyl, 1,1-difluoropropyl, 1,1-difluorobutyl and 1,1-difluoropentyl; wherein at least one of R⁵, R⁶, R⁸ and R⁹ is an acidic group selected from CO₂H, SH, PO₃H₂, SO₃H, CONHNH₂, CONHNHSO₂CF₃, OH, 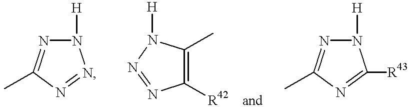
wherein each of R⁴² and R⁴³ is independently selected from Cl, CN, NO₂, CF₃, CO₂CH₃ and SO₂CF₃;
or a tautomer thereof or a pharmaceutically-acceptable salt thereof.

31. The method of claim 30 wherein m is one; wherein R¹ is selected from methyl, ethyl, n-propyl, isopropyl, n-butyl, sec-butyl, isobutyl, tert-butyl, 4-methylbutyl, n-pentyl, neopentyl, phenyl, benzyl, phenethyl, cyclohexyl, cyclohexylmethyl, 1-oxoethyl, 1-oxopropyl, 1-oxobutyl, 1-oxopentyl and hydroxyalkyl; wherein R² is selected from ethyl, n-propyl, isopropyl, n-butyl, sec-butyl, isobutyl, 4-methylbutyl, tert-butyl, n-pentyl, neopentyl, propylthio and butylthio; wherein at least one of R⁵, R⁶, R⁸ and R⁹ is an acidic group selected from CO₂H, SH, PO₃H₂, SO₃H, CONHNH₂, CONHNHSO₂CF₃, OH, 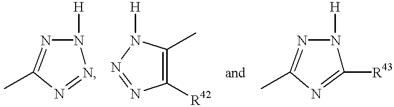
wherein each of R⁴² and R⁴³ is independently selected from Cl, CN, NO₂, CF₃, CO₂CH₃ and SO₂CF₃;
or a tautomer thereof or a pharmaceutically-acceptable salt thereof.

32. The method of claim 31 wherein m is one; wherein R¹ is selected from methyl, ethyl, n-propyl, isopropyl, n-butyl, sec-butyl, isobutyl, tert-butyl, 4-methylbutyl, n-pentyl, neopentyl, phenyl, benzyl, phenethyl, cyclohexyl, cyclohexylmethyl and hydroxyalkyl; wherein R² is selected from ethyl, n-propyl, isopropyl, n-butyl, sec-butyl, isobutyl, 4-methylbutyl, n-pentyl, propylthio and butylthio; wherein each of R³, R⁴, R⁶, R⁷, R⁸, R¹⁰ and R¹¹is hydrido; wherein one of R⁵ and R⁹ is hydrido and the other of R⁵ and R⁹ is an acidic group selected from CO₂H and 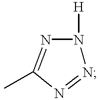
or a tautomer thereof or a pharmaceutically-acceptable salt thereof.

33. The method of claim 32 wherein said compound is 4′-[(1,3-dibutyl-4,5-dihydro-5-oxo-1H-1,2,4-triazol-4-yl) methyl] [1,1′-biphenyl]-2-carboxylic acid.

34. The method of claim 32 wherein said compound is 2,5-dibutyl-2,4-dihydro-4-[[2-(1H-tetrazol-5-yl) [1,1′-biphenyl]-4′-yl]methyl]-3H-1,2,4-triazol-3-one.

35. The method of claim 24 wherein said circulatory disorder is a cardiovascular disorder.

36. The method of claim 35 wherein said cardiovascular disorder is hypertension.

37. The method of claim 35 wherein said cardiovascular disorder is congestive heart failure.
